(19) United States
(12) Patent Application Publication
(10) Pub. No.: US 2012/0291159 A1
(43) Pub. Date: Nov. 15, 2012

(54) Azoline Compounds for Combating Invertebrate Pests

(75) Inventors: Karsten Koerber, Eppelheim (DE); Florian Kaiser, Mannheim (DE); Wolfgang von Deyn, Neustadt (DE); Steffen Gross, Ludwigshafen (DE); Prashant Deshmukh, Mannheim (DE); Joachim Dickhaut, Heidelberg (DE); Nina Gertrud Bandur, Mannheim (DE); Arun Narine, Mannheim (DE); Carsten Beyer, Mainz (DE); Deborah L. Culbertson, Fuquay Varina, NC (US); Douglas D. Anspaugh, Apex, NC (US); Franz Josef Braun, Durham, NC (US); Ronan Le Vezouet, Mannheim (DE)

(73) Assignee: BASF SE, Ludwigshafen (DE)

(21) Appl. No.: 13/515,077

(22) PCT Filed: Dec. 20, 2010
(86) PCT No.: PCT/EP2010/070220

§ 371 Date: Jun. 11, 2012

Related U.S. Application Data
(60) Provisional application No. 61/287,895, filed on Dec. 18, 2009.

(51) Int. Cl. C07D 413/12; C07D 417/12; A01N 43/80; A61K 31/422; A61K 31/427; C07D 413/04; A61K 31/4439; A01N 43/836; A61K 31/506; C07D 261/04; A61K 31/42; C07D 413/14; A01N 43/84; A61K 31/5377; A01C 1/06; A01H 5/00; A01P 7/00; A61P 33/00; A01P 5/00; A01P 7/04; C07D 413/10

(52) U.S. Cl. 800/298; 544/331; 548/240; 548/234; 548/197; 514/275; 514/378; 514/377; 514/370; 546/272.1; 514/340; 548/131; 514/364; 544/122; 514/236.5; 504/100

(57) ABSTRACT

The present invention relates to azoline compounds which are useful for combating or controlling invertebrate pests, in particular arthropod pests and nematodes. The invention also relates to a method for controlling invertebrate pests by using these compounds and to plant propagation material and to an agricultural and a veterinary composition comprising said compounds.

The present invention relates to azoline compounds which are useful for combating or controlling invertebrate pests, in particular arthropod pests and nematodes. The invention also relates to a method for controlling invertebrate pests by using these compounds and to plant propagation material and to an agricultural and a veterinary composition comprising said compounds.

Invertebrate pests and in particular arthropods and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating invertebrate pests, in particular insects, arachnids and nematodes.

Related insecticidal aryl azolines are described in WO 2004/018410, WO 2007/075459, WO 2008/126665, US 2008/262057, WO 2009/077197 and WO 2009/112275. However, these documents do not describe compounds having the characteristic substituents and substituents' arrangement as claimed in the present invention.

It is an object of the present invention to provide compounds that have a good pesticidal activity, in particular insecticidal activity, and show a broad activity spectrum against a large number of different invertebrate pests, especially against difficult to control arthropod pests and/or nematodes.

It has been found that these objectives can be achieved by azoline compounds of the formula I below, by their steroisomers and by their salts, in particular their agriculturally or veterinarily acceptable salts.

Therefore, in a first aspect, the invention relates to iazoline compounds of formula I

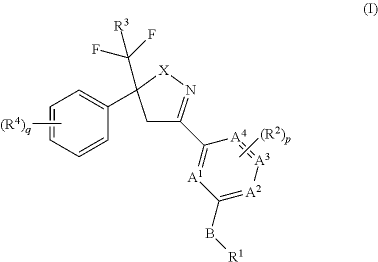

wherein
A¹, A², A³ and A⁴ are independently of each other N or CH;
X is O, S, NR^{5a} or CH₂;
B is O, S, SO, SO₂, NR^{5b}, CR⁶R⁷, C(═W) or NR^{5c}—C(═W);
   with the proviso that B is not NR^{5c}—C(═W) if X is O and simultaneously A¹, A², A³ and A⁴ are CH;
W is O or S;
R¹ is selected from the group consisting of hydrogen, nitro, cyano, C₁-C₆-alkyl which may be partially or fully halogenated and/or may be substituted by one or more, e.g. 1, 2, 3 or 4, preferably 1 or 2, more preferably 1, radicals R⁸, C₃-C₈-cycloalkyl which may be partially or fully halogenated and/or may be substituted by one or more, e.g. 1, 2, 3 or 4, preferably 1 or 2, more preferably 1, radicals R⁹, C₂-C₆-alkenyl which may be partially or fully halogenated and/or may be substituted by one or more, e.g. 1, 2, 3 or 4, preferably 1 or 2, more preferably 1, radicals R⁸, C₂-C₆-alkynyl which may be partially or fully halogenated and/or may be substituted by one or more, e.g. 1, 2, 3 or 4, preferably 1 or 2, more preferably 1, radicals R⁸, —NR^{11a}R^{11b}, —OR¹⁰, —S(O)ₙR¹⁰, —S(O)ₙNR^{11a}R^{11b}, —C(═O)R¹², —C(═O)OR¹³, —C(═O)NR^{11a}R^{11b}, —C(═S)R¹², —C(═S)OR¹³, —C(═S)SR¹³, —C(═S)NR^{11a}R^{11b}, —C(═NR^{11a})R¹², —Si(R¹⁴)₂R¹⁵, phenyl which may be substituted by 1, 2, 3, 4 or 5 radicals, preferably 1, 2 or 3, more preferably 1 or 2 and in particular 1, R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, radicals R¹⁶;
   with the proviso that R¹ is not —OR¹⁰ if B is O and that R¹ is not halogen if B is O, S or NR^{5a};
   with the proviso that R¹ is selected from hydrogen, nitro, cyano, C₁-C₆-alkylthio, C₁-C₆-haloalkylthio, C₃-C₈-cycloalkyl which may be partially or fully halogenated and/or may be substituted by one or more, e.g. 1, 2, 3 or 4, preferably 1 or 2,
   more preferably 1, radicals R⁹, C₂-C₆-alkenyl which may be partially or fully halogenated and/or may be substituted by one or more, e.g. 1, 2, 3 or 4, preferably 1 or 2, more preferably 1, radicals R⁸, C₂-C₆-alkynyl which may be partially or fully halogenated and/or may be substituted by one or more, e.g. 1, 2, 3 or 4, preferably 1 or 2, more preferably 1, radicals R⁸, —NR^{11a}R^{11b}, —S(O)ₙR¹⁰, —S(O)ₙNR^{11a}R^{11b}, —C(═O)R¹², —C(═O)NR^{11a}R^{11b}, —C(═S)R¹², —C(═S)OR¹³ —C(═S)SR¹³, —C(═S)NR^{11a}R^{11b}, —C(═NR^{11a})R¹², and —Si(R¹⁴)₂R¹⁵ if B is NR^{5c}—C(═W), X is O and simultaneously one or two of A¹, A², A³ and A⁴ are N;
   with the proviso that B—R¹ is not CH₂OH, CH₂OC(═O)CH₃, COOCH₃, CONH₂ or CONHCH₃ if p is 1, the radical R² is bound to A² and is selected from CH₂Br, CH₂OH, CH₂OC(═O)CH₃, COOCH₃, CONH₂ and CONHCH₃, A¹, A², A³ and A⁴ are CH, X is O or NR^{5a}, R³ is F, q is 2 and R⁴ is Cl; and
   with the proviso that B—R¹ is not OH, OCH₃, OCH₂CH₃, OCH₂CH₂CH₃, OCH(CH₃)₂, NH₂, NHCH₃, N(CH₃)₂, NHCH₂CH₃, N(CH₂CH₃)₂, NHCH₂CH₂CH₃, N(CH₂CH₂CH₃)₂ or C₁-C₄-alkyl;
each R² is independently selected from the group consisting of halogen, cyano, azido, nitro, —SCN, —SF₅, C₁-C₆-alkyl which may be partially or fully halogenated and/or may be substituted by one or more, e.g. 1, 2, 3 or 4, preferably 1 or 2, more preferably 1, radicals R⁸, C₃-C₈-cycloalkyl which may be partially or fully halogenated and/or may be substituted by one or more, e.g. 1, 2, 3 or 4, preferably 1 or 2, more preferably 1, radicals R⁹, C₂-C₆-alkenyl which may be partially or fully halogenated and/or may be substituted by one or more, e.g. 1, 2, 3 or 4, preferably 1 or 2, more preferably 1, radicals R⁸, C₂-C₆-alkynyl which may be partially or fully halogenated and/or may be substituted by one or more, e.g. 1, 2, 3 or 4, preferably 1 or 2, more preferably 1, radicals R⁸, —Si(R¹⁴)₂R¹⁵, —OR¹⁰, —OS(O)ₙR¹⁰, —S(O)ₙR¹⁰, NR^{11a}R^{11b}, —N(R^{11a})C(═O)R¹², —C(═O)R¹², —C(═O)OR¹³, —C(═NR^{11a})R¹²—C(═S)R¹², phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, radicals R¹⁶,
   with the proviso that R² is not —OR¹⁰, —OS(O)ₙR¹⁰, —S(O)ₙR¹⁰, —NR^{11a}R^{11b}, N(R^{11a})C(═O)R¹² or a 5 or 6-membered aromatic heterocyclic ring comprising 1, 2 or 3 heteroatoms selected from N, O and S as ring members, if R² is bound to A²;
R³ is selected from the group consisting of hydrogen, halogen, C₁-C₄-alkyl and C₁-C₄-haloalkyl,
each R⁴ is independently selected from the group consisting of halogen, cyano, azido, nitro, —SCN, —SF₅, C₁-C₆-alkyl which may be partially or fully halogenated and/or may be substituted by one or more, e.g. 1, 2, 3 or 4, preferably 1 or 2, more preferably 1, radicals R⁸, C₃-C₈-cycloalkyl which may be partially or fully halogenated and/or may be substituted by one or more, e.g. 1, 2, 3 or 4, preferably 1 or 2, more preferably 1, radicals R⁹, C₂-C₆-alkenyl which may be partially or fully halogenated and/or may be substituted by one or more, e.g. 1, 2, 3 or 4, preferably 1 or 2, more preferably 1, radicals R⁸, C₂-C₆-alkynyl which may be partially or fully halogenated and/or may be substituted by one or more, e.g. 1, 2, 3 or 4, preferably 1 or 2, more preferably 1, radicals R⁸, —Si(R¹⁴)₂R¹⁵, —OR¹⁰, —OS(O)ₙR¹⁰, —S(O)ₙR¹⁰, —NR^{11a}R^{11b}, —N(R^{11a})C(═O)R¹², —C(═O)R¹², —C(═O)OR¹³, —C(═NR^{11a})R¹²C(═S)R¹², phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, radicals R¹⁶;
R^{5a} is selected from the group consisting of hydrogen, C₁-C₄-alkyl, C₁-C₄-haloalkyl, C₁-C₄-alkoxy, C₁-C₄-haloalkoxy, C₁-C₄-alkylcarbonyl, C₁-C₄-haloalkylcarbonyl, C₁-C₄-alkoxycarbonyl, C₁-C₄-haloalkoxycarbonyl and benzyl;
R^{5b} and R^{5c}, independently of each other, are selected from the group consisting of hydrogen, cyano, nitro, nitroso, C₁-C₁₀-alkyl which may be partially or fully halogenated and/or may be substituted by one or more, e.g. 1, 2, 3 or 4, preferably 1 or 2, more preferably 1, radicals R⁸, C₃-C₈-cycloalkyl which may be partially or fully halogenated and/or may be substituted by one or more, e.g. 1, 2, 3 or 4, preferably 1 or 2, more preferably 1, radicals R⁹, C₂-C₁₀-alkenyl which may be partially or fully halogenated and/or may be substituted by one or more, e.g. 1, 2, 3 or 4, preferably 1 or 2, more preferably 1, radicals R⁸, C₂-C₁₀-alkynyl which may be partially or fully halogenated and/or may be substituted by one or more, e.g. 1, 2, 3 or 4, preferably 1 or 2, more preferably 1, radicals R⁸, —S(O)ₙR¹⁰, —S(O)ₙNR^{11a}R^{11b}, —C(═O)R¹², —C(═O)OR¹³, —C(═O)NR^{11a}R^{11b}, —C(═S)R¹² —C(═S)SR¹³, —C(═S)NR^{11a}R^{11b}, —C(═NR^{11a})R¹²; phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, radicals R¹⁶;
   or
   R^{5b} and R¹, together with the nitrogen atom to which they are bound, may form a 3-, 4-, 5-, 6-, 7- or 8-membered saturated, partially unsaturated or aromatic heterocyclic ring which may comprise 1 or 2 further heteroatoms or heteroatom groups selected from N, O, S, NO, SO and SO₂, as ring members, where the heterocyclic ring may be substituted by one or more radicals R¹⁶;
   or
   R^{5c} and R¹, together with the atoms to which they are bound, may form a 3-, 4-, 5-, 6-, 7- or 8-membered saturated, partially unsaturated or aromatic heterocyclic ring which may comprise 1 or 2 further heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁶;
R⁶ and R⁷, independently of each other, are selected from the group consisting of hydrogen, halogen, cyano, azido, nitro, —SCN, —SF₅, C₁-C₆-alkyl which may be partially or fully halogenated and/or may be substituted by one or more, e.g. 1, 2, 3 or 4, preferably 1 or 2, more preferably 1, radicals R⁸, C₃-C₈-cycloalkyl which may be partially or fully halogenated and/or may be substituted by one or more, e.g. 1, 2, 3 or 4, preferably 1 or 2, more preferably 1, radicals R⁹, C₂-C₆-alkenyl which may be partially or fully halogenated and/or may be substituted by one or more, e.g. 1, 2, 3 or 4, preferably 1 or 2, more preferably 1, radicals R⁸, C₂-C₆-alkynyl which may be partially or fully halogenated and/or may be substituted by one or more, e.g. 1, 2, 3 or 4, preferably 1 or 2, more preferably 1, radicals R⁸, —Si(R¹⁴)₂R¹⁵, —OR¹⁰, OSO₂R¹⁰, —S(O)ₙR¹⁰, —S(O)ₙNR^{11a}R^{11b}, —NR^{11a}R^{11b}, —C(═O)NR^{11a}R^{11b}, —C(═S)NR^{11a}R^{11b}, —C(═O)OR¹³, phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, als R¹⁶,
   or
   R⁶ and R⁷ together form a group selected from ═CR¹⁷R¹⁸; ═S(O)ₙR¹⁰; ═S(O)ₙNR^{11a}R^{11b}, ═NR^{11a}, ═NOR¹⁰ or ═NNR^{11a}R^{11b}, where R¹⁰, R^{11a}, R^{11b}, R¹⁷, R¹⁸ and n are as defined below; or R¹⁰ and R¹ or R^{11a} and R¹ or R¹⁷ and R¹, together with the atoms to which they are bound, form a 3-, 4-, 5-, 6-, 7- or 8-membered saturated, partially unsaturated or aromatic carbocyclic or heterocyclic ring containing 1, 2 or 3 heteroatoms or heteroatom groups selected from N, O, S, NO, SO and SO₂, as ring members, where the carbocyclic or heterocyclic ring may be substituted by one or more, e.g. 1, 2, 3 or 4, preferably 1 or 2, more preferably 1, radicals R¹⁶;
   or
   R⁶ and R⁷, together with the carbon atom to which they are bound, form a 3-, 4-, 5-, 6-, 7- or 8-membered saturated or partly unsaturated carbocyclic or heterocyclic ring comprising 1, 2 or 3 heteroatoms or heteroatom groups selected from N, O, S, NO, SO and SO₂, as ring members, where the carbocyclic or heterocyclic ring may be substituted by one or more, e.g. 1, 2, 3 or 4, preferably 1 or 2, more preferably 1, radicals R¹⁶;
each R⁸ is independently selected from the group consisting of cyano, azido, nitro, —SCN, —SF₅, C₃-C₈-cycloalkyl, C₃-C₈-halocycloalkyl, C₃-C₈-cycloalkyl substituted by a phenyl group or substituted by a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the phenyl group or the heterocyclic ring may carry one or more, e.g. 1, 2, 3 or 4, preferably 1 or 2, more preferably 1, radicals R¹⁶; —Si(R¹⁴)₂R¹⁵, —OR¹⁰, —OSO₂R¹⁰, —S(O)ₙR¹⁰, —S(O)ₙN(R^{11a})R^{11b}, —N(R^{11a})R^{11b}, —C(═O)N(R^{11a})R^{11b}—C(═S)N(R^{11a})R^{11b}, —C(═O)R¹², —C(═O)OR¹³, phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, radicals R¹⁶;
   or two geminally bound radicals R⁸ together form a group selected from ═O, ═CR¹⁷R¹⁸, ═S(O)ₙR¹⁰, ═S(O)ₙN(R^{11a})R^{11b}, ═NR^{11a}, ═NOR¹⁰ and ═NNR^{11a}R^{11b};
   or two radicals R⁸, together with the carbon atoms to which they are bound, form a 3-, 4-, 5-, 6-, 7- or 8-membered saturated, partially unsaturated or aromatic carbocyclic or heterocyclic ring containing 1, 2 or 3 heteroatoms or heteroatom groups selected from N, O, S, NO, SO and SO₂, as ring members, where the carbocyclic or heterocyclic ring may be substituted by one or more, e.g. 1, 2, 3 or 4, preferably 1 or 2, more preferably 1, radicals R¹⁶;
each R⁹ is independently selected from the group consisting of cyano, azido, nitro, —SCN, —SF₅, C₁-C₆-alkyl, C₁-C₆-haloalkyl, C₁-C₆-alkoxy-C₁-C₆-alkyl, C₁-C₆-haloalkoxy-C₁-C₆-alkyl, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, C₂-C₆-alkenyl, C₂-C₆-haloalkenyl, C₂-C₆-alkynyl, C₂-C₆-haloalkynyl, —Si(R¹⁴)₂R¹⁵, —OR¹⁰, —OSO₂R¹⁰, —S(O)ₙR¹⁰, —S(O)ₙN(R^{11a})R^{11b}, —N(R^{11a})R^{11b}, —C(═O)N(R^{11a})R^{11b}, —C(═S)N(R^{11a})R^{11b}, —C(═O)R¹² —C(═O)OR¹³, phenyl, benzyl, where the phenyl moieties in the two last-mentioned radicals may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, radicals R¹⁶;
   or two geminally bound radicals R⁹ together form a group selected from ═O, ═CR¹⁷R¹⁸, ═S(O)ₙR¹⁰, ═S(O)ₙN(R^{11a})R^{11b}, ═NR^{11a}, ═NOR¹⁰ and ═NNR^{11a}R^{11b};
   or two radicals R⁹, together with the carbon atoms to which they are bound, form a 3-, 4-, 5-, 6-, 7- or 8-membered saturated, partially unsaturated or aromatic carbocyclic or heterocyclic ring containing 1, 2 or 3 heteroatoms or heteroatom groups selected from N, O, S, NO, SO and SO₂, as ring members, where the carbocyclic or heterocyclic ring may be substituted by one or more, e.g. 1, 2, 3 or 4, preferably 1 or 2, more preferably 1, radicals R¹⁶;
each R¹⁰ is independently selected from the group consisting of hydrogen, cyano, C₁-C₆-alkyl which may be partially or fully halogenated and/or may carry one or more, e.g. 1, 2, 3 or 4, preferably 1 or 2, more preferably 1, radicals R²², C₃-C₈-cycloalkyl which may be partially or fully halogenated and/or may carry one or more, e.g. 1, 2, 3 or 4, preferably 1 or 2, more preferably 1, radicals R²³, C₄-C₈-alkylcycloalkyl, C₂-C₆-alkenyl which may be partially or fully halogenated and/or may carry one or more, e.g. 1, 2, 3 or 4, preferably 1 or 2, more preferably 1, radicals R²², C₂-C₆-alkynyl which may be partially or fully halogenated and/or may carry one or more radicals R²², —Si(R¹⁴)₂R¹⁵, —OR¹⁹, —S(O)ₙR¹⁹, —S(O)ₙNR^{11a}R^{11b}, NR^{11a}R^{11b}, —N═CR¹⁷R¹⁸, —C(═O)R¹², —C(═O)NR^{11a}R^{11b}, C(═S)NR^{11a}R^{11b}, —C(═O)OR¹³, phenyl, benzyl, where the phenyl moieties in the two last-mentioned radicals may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, radicals R¹⁶;
   with the proviso that in OR¹⁰, R¹⁰ is not —OR¹⁹;
R^{11a} and R^{11b}, independently of each occurrence and independently of each other, are selected from the group consisting of hydrogen, C₁-C₆-alkyl which may be partially or fully halogenated and/or may carry one or more, e.g. 1, 2, 3 or 4, preferably 1 or 2, more preferably 1, radicals R²², C₃-C₈-cycloalkyl which may be partially or fully halogenated and/or may carry one or more, e.g. 1, 2, 3 or 4, preferably 1 or 2, more preferably 1, radicals R²³, C₃-C₈-cycloalkyl-C₁-C₄-alkyl, where the cycloalkyl moiety in the last-mentioned radical may be partially or fully halogenated and/or may carry one or more, e.g. 1, 2, 3 or 4, preferably 1 or 2, more preferably 1, radicals R²³, C₂-C₆-alkenyl which may be partially or fully halogenated and/or may carry one or more, e.g. 1, 2, 3 or 4, preferably 1 or 2, more preferably 1, radicals R²², C₂-C₆-alkynyl which may be partially or fully halogenated and/or may carry one or more, e.g. 1, 2, 3 or 4, preferably 1 or 2, more preferably 1, radicals R²², OR¹⁹, —S(O)ₙR¹⁹, —S(O)ₙNR^{20a}R^{20b}, —C(═O)R¹², —C(═O)OR¹³, —C(═O)NR^{20a}R^{20b}, —C(═S)R¹², —C(═S)SR¹³, —C(═S)NR^{20a}R^{20b}, —C(═NR^{20a})R¹²; phenyl, benzyl, where the phenyl moieties in the two last-mentioned radicals may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, radicals R¹⁶;
   or
   R^{11a} and R^{11b}, together with the nitrogen atom to which they are bound, form a 3-, 4-, 5-, 6-, 7- or 8-membered saturated, partially unsaturated or aromatic ring, wherein the ring may contain one or two further heteroatoms or heteroatom groups selected from N, O, S, NO, SO and SO₂, as ring members, and/or wherein the ring may be substituted with one or more, e.g. 1, 2, 3 or 4, preferably 1 or 2, more preferably 1, substituents selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, phenyl which may be substituted by 1, 2, 3, 4 or 5, preferably 1 or 2, more preferably 1, radicals R¹⁶, and a 3-, 4-, 5-, 6-, or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁶;
each R¹² is independently selected from the group consisting of hydrogen, halogen, cyano, azido, nitro, —SCN, SF₅, C₁-C₆-alkyl which may be partially or fully halogenated and/or may be substituted by one or more, e.g. 1, 2, 3 or 4, preferably 1 or 2, more preferably 1, radicals R²², C₃-C₈-cycloalkyl which may be partially or fully halogenated and/or may be substituted by one or more, e.g. 1, 2, 3 or 4, preferably 1 or 2, more preferably 1, radicals R²³, C₂-C₆-alkenyl which may be partially or fully halogenated and/or may be substituted by one or more, e.g. 1, 2, 3 or 4, preferably 1 or 2, more preferably 1, radicals R²², C₂-C₆-alkynyl which may be partially or fully halogenated and/or may be substituted by one or more, e.g. 1, 2, 3 or 4, preferably 1 or 2, more preferably 1, radicals R²², —Si(R¹⁴)₂R¹⁵, —OR¹⁹, —OSO₂R¹⁹, —S(O)ₙR¹⁹, —S(O)ₙNR^{20a}R^{20b}, —NR^{20a}R^{20b}, —C(═O)NR^{20a}R^{20b}, —C(═S)NR^{20a}R^{20b}, —C(═O)OR¹³, phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, radicals R¹⁶,
each R¹³ is independently selected from the group consisting of hydrogen, cyano, C₁-C₆-alkyl which may be partially or fully halogenated and/or may be substituted by one or more, e.g. 1, 2, 3 or 4, preferably 1 or 2, more preferably 1, radicals R²², C₃-C₈-cycloalkyl which may be partially or fully halogenated and/or may be substituted by one or more, e.g. 1, 2, 3 or 4, preferably 1 or 2, more preferably 1, radicals R²³, C₂-C₆-alkeny which may be partially or fully halogenated and/or may be substituted by one or more, e.g. 1, 2, 3 or 4, preferably 1 or 2, more preferably 1, radicals R²², C₂-C₆-alkynyl which may be partially or fully halogenated and/or may be substituted by one or more, e.g. 1, 2, 3 or 4, preferably 1 or 2, more preferably 1, radicals R²², —Si(R¹⁴)₂R¹⁵, —S(O)ₙR¹⁹, —S(O)ₙNR^{20a}R^{20b}, —NR^{20a}R^{20b}, —N═CR¹⁷R¹⁸, —C(═O)R¹⁹, —C(═O)NR^{20a}R^{20b}, —C(═S)NR^{20a}R^{20b}, —C(═O)OR²¹, phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1, 2 or 3, more preferably 1 or 2 and in particular 1, radicals R¹⁶;
R¹⁴ and R¹⁵, independently of each other and independently of each occurrence, are selected from the group consisting of halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₄-alkyl, C₁-C₆-haloalkoxy-C₁-C₆-alkyl, phenyl, benzyl, where the phenyl moiety in the two last-mentioned radicals may be substituted by 1, 2, 3, 4 or 5, preferably 1 or 2, more preferably 1, radicals selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- to 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals selected from halogen, cyano, nitro, C₁-C₆-alkyl, C₁-C₆-haloalkyl, C₁-C₆-alkoxy and C₁-C₆-haloalkoxy;
each R¹⁶ is independently selected from the group consisting of halogen, cyano, azido, nitro, SCN, SF₅, C₁-C₁₀-alkyl which may be partially or fully halogenated and/or may be substituted by one or more, e.g. 1, 2, 3 or 4, preferably 1 or 2, more preferably 1, radicals R²², C₃-C₈-cycloalkyl which may be partially or fully halogenated and/or may be substituted by one or more, e.g. 1, 2, 3 or 4, preferably 1 or 2, more preferably 1, radicals R²³, C₂-C₁₀-alkenyl which may be partially or fully halogenated and/or may be substituted by one or more, e.g. 1, 2, 3 or 4, preferably 1 or 2, more preferably 1, radicals R²², C₂-C₁₀-alkynyl which may be partially or fully halogenated and/or may be substituted by one or more, e.g. 1, 2, 3 or 4, preferably 1 or 2, more preferably 1, radicals R²², Si(R¹⁴)₂R¹⁵, —OR¹⁹, —OS(O)ₙR¹⁹, —S(O)ₙR¹⁹, —S(O)ₙNR^{20a}R^{20b}, —NR^{20a}R^{20b}, —C(═O)R¹⁹, —C(═O)OR²¹, —C(═NR^{20a})R¹⁹, —C(═O)NR^{20a}R^{20b}, —C(═S)NR^{20a}R^{20b}, phenyl which may be substituted by 1, 2, 3, 4 or 5, preferably 1 or 2, more preferably 1, radicals selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals selected from halogen, cyano, nitro, C₁-C₆-alkyl, C₁-C₆-haloalkyl, C₁-C₆-alkoxy and C₁-C₆-haloalkoxy; or two radicals R¹⁶ bound geminally on one ring atom of a saturated or partially unsaturated ring may form together a group ═O, ═CR¹⁷R¹⁸; ═S(O)ₙR¹⁹; ═S(O)ₙNR^{20a}R^{20b}, ═NR^{20a}, ═NOR¹⁹ or ═NNR^{20a},
   or
   two radicals R¹⁶ bound on adjacent atoms form a bridge group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR^{20a}—, —CH₂CH═N—, —CH═CH—NR^{20a}—, —OCH═N—, —SCH═N— and thus form together with the atoms to which they are bound to a 5-membered or 6-membered saturated, partially unsaturated or aromatic carbocyclic or heterocyclic ring, wherein the ring may optionally be substituted with one or two substituents selected from ═O, OH, CH₃, OCH₃, halogen, halomethyl and halomethoxy;
R¹⁷ and R¹⁸, independently of each other and independently of each occurrence, are selected from hydrogen, C₁-C₄-alkyl, C₁-C₆-cycloalkyl, C₁-C₄-alkoxy-C₁-C₄-alkyl, phenyl and benzyl;
R¹⁹ is selected from the group consisting of hydrogen, halogen, cyano, azido, nitro, OH, SH, SCN, SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl, C₁-C₆-alkyl, C₂-C₆-alkenyl, C₂-C₆-alkynyl, C₃-C₈-cycloalkyl, wherein the four last-mentioned aliphatic and cycloaliphatic radicals may be partially or fully halogenated and/or wherein in the four last-mentioned aliphatic and cycloaliphatic radicals one or two CH₂ groups may be replaced by a CO group and/or wherein the four last-mentioned aliphatic and cycloaliphatic radicals may carry 1 or 2 radicals selected from C₁-C₄-alkoxy, phenyl, benzyl, pyridyl and phenoxy, wherein the four last-mentioned radicals may in turn be partially or fully halogenated and/or carry 1, 2 or 3 substituents selected from C₁-C₆-alkyl, C₁-C₆-haloalkyl, C₁-C₆-alkoxy, C₁-C₆-haloalkoxy, C₁-C₆-alkoxycarbonyl, C₁-C₆-alkylamino and di-(C₁-C₆-alkyl)-amino;
   with the proviso that R¹⁹ in OR¹⁹ is not halogen, OH, C₁-C₆-alkoxy or C₁-C₆-haloalkoxy;
R^{20a} and R^{20b}, independently of each other and independently of each occurrence, are selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl, C₁-C₆-alkyl, C₂-C₆-alkenyl, C₂-C₆-alkynyl, C₃-C₈-cycloalkyl, wherein the four last-mentioned aliphatic and cycloaliphatic radicals may be partially or fully halogenated and/or wherein in the four last-mentioned aliphatic and cycloaliphatic radicals one or two CH₂ groups may be replaced by a CO group and/or wherein the four last-mentioned aliphatic and cycloaliphatic radicals may carry 1 or 2 radicals selected from C₁-C₄-alkoxy, phenyl, benzyl, pyridyl, and phenoxy, wherein the four last mentioned radicals may in turn be partially or fully halogenated and/or carry 1, 2 or 3 substituents selected from C₁-C₆-alkyl, C₁-C₆-haloalkyl, C₁-C₆-alkoxy, C₁-C₆-haloalkoxy and C₁-C₆-alkoxycarbonyl, or
   R^{20a} and R^{20b}, together with the nitrogen atom to which they are bound, form a 3-, 4-, 5-, 5-, 6- or 7-membered saturated, partially unsaturated or completely unsaturated heterocyclic ring, wherein the ring may contain 1 or 2 further heteroatoms or heteroatom groups selected from N, O, S, NO, SO and SO₂, as ring members, and/or wherein the ring may be substituted with one or more, e.g. 1, 2, 3 or 4, preferably 1 or 2, more preferably 1, substituents selected from halogen, C₁-C₄-haloalkyl, C₁-C₄-alkoxy and C₁-C₄-haloalkoxy;
each R²¹ is independently selected from the group consisting of hydrogen, cyano, C₁-C₆-alkyl, C₂-C₆-alkenyl, C₂-C₆-alkynyl, C₃-C₈-cycloalkyl, wherein the four last-mentioned aliphatic and cycloaliphatic radicals may be partially or fully halogenated and/or wherein in the four last-mentioned aliphatic and cycloaliphatic radicals one or two CH₂ groups may be replaced by a CO group and/or wherein the four last-mentioned aliphatic and cycloaliphatic radicals may carry 1 or 2 radicals selected from C₁-C₄-alkoxy, phenyl, benzyl, pyridyl, and phenoxy, wherein the four last-mentioned radicals may in turn be partially or fully halogenated and/or carry 1, 2 or 3 substituents selected from C₁-C₆-alkyl, C₁-C₆-haloalkyl, C₁-C₆-alkoxy, C₁-C₆-haloalkoxy and C₁-C₆-alkoxycarbonyl;
each R²² is independently is selected from the group consisting of cyano; azido, nitro, —SCN, SF₅, C₃-C₈-cycloalkyl, C₃-C₈-halocycloalkyl, —Si(R¹⁴)₂R¹⁵, —OR¹⁹, —OSO₂R¹⁹, —S(O)ₙR¹⁹, —S(O)ₙN(R^{20a})R^{20b}, —N(R^{20a})R^{20b}, —C(═O)N(R^{20a})R^{20b}, —C(═S)N(R^{20a})R^{20b}, —C(═O)OR²¹, phenyl which may be substituted by 1, 2, 3, 4 or 5, preferably 1 or 2, more preferably 1, radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals independently selected from halogen, cyano, nitro, C₁-C₆-alkyl, C₁-C₆-haloalkyl, C₁-C₆-alkoxy and C₁-C₆-haloalkoxy;
each R²³ is independently is selected from the group consisting of cyano; azido, nitro, —SCN, SF₅, C₁-C₆-alkyl, C₁-C₆-haloalkyl, C₁-C₆-alkoxy-C₁-C₆-alkyl, C₁-C₆-haloalkoxy-C₁-C₆-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, —Si(R¹⁴)₂R¹⁵, —OR¹⁹, —OSO₂R¹⁹, —S(O)ₙR¹⁹, —S(O)ₙN(R^{20a})R^{20b}, —N(R^{20a})R^{20b}, —C(═O)N(R^{20a})R^{20b}, —C(═S)N(R^{20a})R^{20b}, —C(═O)OR²¹, phenyl, benzyl, where the phenyl moieties in the two last-mentioned radicals may be substituted by 1, 2, 3, 4 or 5, preferably 1 or 2, more preferably 1, radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals independently selected from halogen, cyano, nitro, C₁-C₆-alkyl, C₁-C₆-haloalkyl, C₁-C₆-alkoxy and C₁-C₆-haloalkoxy;
n is 0, 1 or 2;
p is 0, 1, 2, 3 or 4; and
q is 0, 1, 2, 3, 4 or 5;
and the stereoisomers and agriculturally or veterinarily acceptable salts thereof.

In one embodiment, the invention relates to compounds I, however except for following compounds:
compounds of formula I, wherein
X is O, NH, NCH₃ or CH₂;
A¹, A², A³ and A⁴ are CH, or one of A¹, A², A³ and A⁴ is N and the other 3 variables are CH, or A¹ and A² are N and A³ and A⁴ are CH;
p is 0 or 1, where in case that p is 1 the radical R² is bound in the position of A²;
R² is selected from methyl, ethyl, F, Cl, Br, methoxy, nitro, CN and CF₃
R³ is F;
(R⁴)_{q} is 3,5-Cl₂, relative to the 1-position of the attachment point of the phenyl ring to the remainder of the molecule; and
B—R¹ is selected from NH₂, NHNH₂, NHCH₃, NHCOCH₃, NHCOCF₃, NHCO-phenyl, NHCOO—C(CH₃)₃, N(CH₃)NH₂, N(CH₃)COCF₃, NR^{a}NR^{b}C(O)—R^{c} and NR^{a}NR^{b}C(S)R^{c}, wherein
   R^{a} is selected from H, CH₃, COCH₃ and COO—C(CH₃)₃;
   R^{b} is selected from H, CH₃ and COCH₃; and
   R^{c} is selected from C₁-C₆-alkyl which can carry 1 substituent selected from methoxy, CN, nitro, N(CH₃)₂, COOCH₃, cyclopropyl, phenyl, pyridyl, thienyl, thiazolyl and thiadiazolyl; C₁-C₄-haloalkyl, C₂-C₄-alkenyl, C₂-C₄-alkynyl, C₃-C₆-cycloalkyl, C₁-C₃-alkoxy, phenyl which may carry 1 substituent selected from nitro and methoxy; phenoxy, pyridyl, thienyl, thiazolyl, thiadiazolyl, styryl, 1-pyrrolidinyl, NHCH₃, N(CH₃)₂, NHCH₂CH₃, and NH-cyclohexyl;
and
   compounds of formula I, wherein
X is O;
A¹, A², A³ and A⁴ are CH, or A³ is N and A¹ A² and A⁴ are CH;
p is 0, 1 or 2, where in case that p is 2, one substituent R² is bound to the position of A²;
R² is selected from F, Cl, Br, I, OH, SH, NH₂, NHCH₃, N(CH₃)₂, nitro, CN, methyl, CF₃ and methoxy;
R³ is F;
(R⁴)_{q} is 3,5-Cl₂, relative to the 1-position of the attachment point of the phenyl ring to the remainder of the molecule;
B is O, NR^{5b}, CR⁶R⁷ or CO; wherein
   R^{5b} is selected from H, methyl, ethyl, CF₃, cyclopropyl, cyclohexyl, phenyl, benzyl, CH═CH₂ and C≡CH; and
   R⁶ and R⁷ are independently selected from H, C₁-C₃-alkyl, CF₃, cyclopropyl and phenyl; and
R¹ is H or NR^{11a}R^{11b}, wherein
   R^{11a} is H; and
      R^{11b} is selected from H, C₁-C₄-alkylcarbonyl, where the alkyl group may carry 1 substituent selected from methoxy, CN and N(CH₃)₂; trifluoromethylcarbonyl, 2,2,2-trifluoroethylcarbonyl, cyclopropylcarbonyl, cyclopropylmethylcarbonyl and phenylcarbonyl.

The present invention also provides an agricultural composition comprising at least one compound of the formula I as defined herein and/or an agriculturally acceptable salt thereof and at least one liquid or solid carrier.

The present invention also provides a veterinary composition comprising at least one compound of the formula I as defined herein and/or a veterinarily acceptable salt thereof and at least one liquid or solid carrier.

The present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The present invention also relates to plant propagation material, in particular seed, comprising at least one compound of formula I and/or an agriculturally acceptable salt thereof as defined herein.

The present invention further relates to a method for treating or protecting an animal from infestation or infection by parasites which comprises bringing the animal in contact with a parasiticidally effective amount of a compound of the formula I or a veterinarily acceptable salt thereof as defined herein. Bringing the animal in contact with the compound I, its salt or the veterinary composition of the invention means applying or administering it to the animal.

The term “steroisomers” encompasses both optical isomers, such as enantiomers or diastereomers, the latter existing due to more than one center of chirality in the molecule, as well as geometrical isomers (cis/trans isomers).

Depending on the substitution pattern, the compounds of the formula I may have one or more centers of chirality, in which case they are present as mixtures of enantiomers or diastereomers. One center of chirality is the carbon ring atom of the azoline ring carrying radical —CF₂R³. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

The compounds of the present invention may be amorphous or may exist in one or more different crystalline states (polymorphs) which may have a different macroscopic properties such as stability or show different biological properties such as activities. The present invention includes both amorphous and crystalline compounds of the formula I, mixtures of different crystalline states of the respective compound I, as well as amorphous or crystalline salts thereof.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally acceptable salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH⁴⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzi-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

By the term “veterinarily acceptable salts” is meant salts of those cations or anions which are known and accepted in the art for the formation of salts for veterinary use. Suitable acid addition salts, e.g. formed by compounds of formula I containing a basic nitrogen atom, e.g. an amino group, include salts with inorganic acids, for example hydrochlorids, sulphates, phosphates, and nitrates and salts of organic acids for example acetic acid, maleic acid, dimaleic acid, fumaric acid, difumaric acid, methane sulfenic acid, methane sulfonic acid, and succinic acid.

The term “invertebrate pest” as used herein encompasses animal populations, such as insects, arachnids and nematodes, which may attack plants, thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” as used herein includes all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “plants” comprises any types of plants including “non-cultivated plants” and in particular “cultivated plants”.

The term “non-cultivated plants” refers to any wild type species or related species or related genera of a cultivated plant.

The term “cultivated plants” as used herein includes plants which have been modified by breeding, mutagenesis or genetic engineering. Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transitional modification of protein(s) (oligo- or polypeptides) poly for example by glycosylation or polymer additions such as prenylated, acetylated or farnesylated moieties or PEG moieties (e.g. as disclosed in Biotechnol Prog. 2001 July-August; 17(4):720-8, Protein Eng Des Sel. 2004 January; 17(1):57-66, Nat. Protoc. 2007; 2(5):1225-35, Curr. Opin. Chem. Biol. 2006 October; 10(5):487-91. Epub 2006 Aug. 28, Biomaterials. 2001 March; 22(5):405-17, Bioconjug Chem. 2005 January-February; 16(1):113-21).

The term “cultivated plants” as used herein further includes plants that have been rendered tolerant to applications of specific classes of herbicides, such as hydroxy-phenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A-0242236, EP-A-242246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e.g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” as used herein further includes plants that are by the use of recombinant DNA techniques capable to synthesize one or more insecticidal proteins, especially those known from the bacterial genus bacillus, particularly from bacillus thuringiensis, such as ä-endotoxins, e.g. CryIA(b), CryIA(c), CryIF, CryLF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are dis-closed, for example, in EP-A 374 753, WO 93/007278, WO 95/34656, EP-A 427 529, EP-A 451 878, WO 03/018810 und WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods insects, particularly to beetles (Coleoptera), flies (Diptera), and butterflies and moths (Lepidoptera) and to plant parasitic nematodes (Nematoda).

The term “cultivated plants” as used herein further includes plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato Solanum bulbocastanum) or T4-lyso-zym (e.g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” as used herein further includes plants that are by the use of recombinant DNA techniques capable to synthesize one or more proteins to increase the productivity (e.g. bio mass production, grain yield, starch content, oil content or protein content), tolerance to drought, salinity or other growth-limiting environ-mental factors or tolerance to pests and fungal, bacterial or viral pathogens of those plants.

The term “cultivated plants” as used herein further includes plants that contain by the use of recombinant DNA techniques a modified amount of substances of content or new substances of content, specifically to improve human or animal nutrition, for ex-ample oil crops that produce health-promoting long-chain omega-3 fatty acids or unsaturated omega-9 fatty acids (e.g. Nexera® rape).

The term “cultivated plants” as used herein further includes plants that contain by the use of recombinant DNA techniques a modified amount of substances of content or new substances of content, specifically to improve raw material production, for example potatoes that produce increased amounts of amylopectin (e.g. Amflora® potato).

The organic moieties mentioned in the above definitions of the variables are—like the term halogen—collective terms for individual listings of the individual group members.

The prefix Cₙ-Cₘ indicates in each case the possible number of carbon atoms in the group.

The term halogen denotes in each case fluorine, bromine, chlorine or iodine, in particular fluorine, chlorine or bromine.

The term “C₁-C₁₀-alkyl” as used herein and in the alkyl moieties of alkoxy, alkylthio, alkylsulfinyl, alkylsulfonyl, alkylcarbonyl, alkoxycarbonyl and the like refers to saturated straight-chain or branched hydrocarbon radicals having 1 to 2 (“C₁-C₂-alkyl”), 1 to 4 (“C₁-C₄-alkyl”), 1 to 6 (“C₁-C₆-alkyl”), 1 to 8 (“C₁-C₈-alkyl”) or 1 to 10 (“C₁-C₁₀-alkyl”) carbon atoms. C₁-C₂-Alkyl is methyl or ethyl. C₁-C₄-Alkyl is additionally propyl, isopropyl, butyl, 1-methylpropyl(sec-butyl), 2-methylpropyl(isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl,
1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl, 2-ethylhexyl and positional isomers thereof. C₁-C₁₀-Alkyl is additionally also, for example, nonyl, decyl and positional isomers thereof.

The term “C₁-C₁₀-haloalkyl” as used herein, which is also expressed as “C₁-C₁₀-alkyl which is partially or fully halogenated”, refers to straight-chain or branched alkyl groups having 1 to 2 (“C₁-C₂-haloalkyl”), 1 to 4 (“C₁-C₄-haloalkyl”), 1 to 6 (“C₁-C₆-haloalkyl”), 1 to 8 (“C₁-C₈-haloalkyl”) or 1 to 10 (“C₁-C₁₀-haloalkyl”) carbon atoms (as mentioned above), where some or all of the hydrogen atoms in these groups are replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl.

“Halomethyl” is methyl in which 1, 2 or 3 of the hydrogen atoms are replaced by halogen atoms. Examples are bromomethyl, chloromethyl, fluoromethyl, dichloromethyl, trichloromethyl, difluoromethyl, trifluoromethyl, chlorofluoromethyl, dichlorofluoromethyl, chlorodifluoromethyl and the like.

The term “C₂-C₁₀-alkenyl” as used herein and in the alkenyl moiety of alkenyloxy and the like refers to monounsaturated straight-chain or branched hydrocarbon radicals having 2 to 4 (“C₂-C₄-alkenyl”), 2 to 6 (“C₂-C₆-alkenyl”), 2 to 8 (“C₂-C₈-alkenyl”), 3 to 8 (“C₃-C₈-alkenyl”), 2 to 10 (“C₂-C₁₀-alkenyl”) or 3 to 10 (“C₃-C₁₀-alkenyl”) carbon atoms and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “C₂-C₁₀-haloalkenyl” as used herein, which is also expressed as “C₁-C₁₀-alkenyl which is partially or fully halogenated”, and the haloalkenyl moieties in haloalkenyloxy, haloalkenylcarbonyl and the like refers to unsaturated straight-chain or branched hydrocarbon radicals having 2 to 4 (“C₂-C₄-haloalkenyl”), 2 to 6 (“C₂-C₆-haloalkenyl”), 2 to 8 (“C₂-C₆-haloalkenyl”) or 2 to 10 (“C₂-C₁₀-haloalkenyl”) carbon atoms and a double bond in any position (as mentioned above), where some or all of the hydrogen atoms in these groups are replaced by halogen atoms as mentioned above, in particular fluorine, chlorine and bromine, for example chlorovinyl, chloroallyl and the like.

The term “C₂-C₁₀-alkynyl” as used herein and the alkynyl moieties in alkynyloxy, alkynylcarbonyl and the like refers to straight-chain or branched hydrocarbon groups having 2 to 4 (“C₂-C₄-alkynyl”), 2 to 6 (“C₂-C₆-alkynyl”), 2 to 8 (“C₂-C₈-alkynyl”), 3 to 8 (“C₃-C₈-alkynyl”), 2 to 10 (“C₂-C₁₀-alkynyl”) or 3 to 10 (“C₃-C₈-alkynyl”) carbon atoms and one or two triple bonds in any position, for example C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “C₂-C₁₀-haloalkynyl” as used herein, which is also expressed as “C₁-C₁₀-alkynyl which is partially or fully halogenated”, and the haloalkynyl moieties in haloalkynyloxy, haloalkynylcarbonyl and the like refers to unsaturated straight-chain or branched hydrocarbon radicals having 2 to 4 (“C₂-C₄-haloalkynyl”), 3 to 4 (“C₃-C₄-haloalkynyl”), 2 to 6 (“C₂-C₆-haloalkynyl”), 3 to 6 (“C₃-C₆-haloalkynyl”), 2 to 8 (“C₂-C₈-haloalkynyl”), 3 to 8 (“C₃-C₈-haloalkynyl”), 2 to 10 (“C₂-C₁₀-haloalkynyl”) or 3 to 10 (“C₃-C₁₀-haloalkynyl”) carbon atoms and one or two triple bonds in any position (as mentioned above), where some or all of the hydrogen atoms in these groups are replaced by halogen atoms as mentioned above, in particular fluorine, chlorine and bromine;

The term “C₃-C₈-cycloalkyl” as used herein refers to mono- or bi- or polycyclic saturated hydrocarbon radicals having 3 to 8, in particular 3 to 6 carbon atoms (“C₃-C₆-cycloalkyl”). Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl.

The term “C₃-C₈-halocycloalkyl” as used herein, which is also expressed as “C₃-C₈-cycloalkyl which is partially or fully halogenated”, and the halocycloalkyl moieties in halocycloalkoxy, halocycloalkylcarbonyl and the like refers to mono- or bi- or polycyclic saturated hydrocarbon groups having 3 to 8 (“C₃-C₈-halocycloalkyl”) or preferably 3 to 6 (“C₃-C₆-halocycloalkyl”) carbon ring members (as mentioned above) in which some or all of the hydrogen atoms are replaced by halogen atoms as mentioned above, in particular fluorine, chlorine and bromine.

The term “C₃-C₈-cycloalkyl-C₁-C₄-alkyl” refers to a C₃-C₈-cycloalkyl group as defined above which is bound to the remainder of the molecule via a C₁-C₄-alkyl group, as defined above. Examples are cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, and the like.

The term “C₃-C₈-halocycloalkyl-C₁-C₄-alkyl” refers to a C₃-C₈-halocycloalkyl group as defined above which is bound to the remainder of the molecule via a C₁-C₄-alkyl group, as defined above.

The term “C₁-C₂-alkoxy” is a C₁-C₂-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is methoxy or ethoxy. C₁-C₄-Alkoxy is additionally, for example, n-propoxy, 1-methylethoxy(isopropoxy), butoxy, 1-methylpropoxy(sec-butoxy), 2-methylpropoxy(isobutoxy) or 1,1-dimethylethoxy(tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. The term “C₁-C₁₀-haloalkoxy” is a C₁-C₁₀-haloalkyl group, as defined above, attached via an oxygen atom. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₂-alkylthio” is a C₁-C₂-alkyl group, as defined above, attached via a sulfur atom. The term “C₁-C₄-alkylthio” is a C₁-C₄-alkyl group, as defined above, attached via a sulfur atom. The term “C₁-C₆-alkylthio” is a C₁-C₆-alkyl group, as defined above, attached via a sulfur atom. The term “C₁-C₁₀-alkylthio” is a C₁-C₁₀-alkyl group, as defined above, attached via a sulfur atom. C₁-C₂-Alkylthio is methylthio or ethylthio. C₁-C₄-Alkylthio is additionally, for example, n-propylthio, 1-methylethylthio(isopropylthio), butylthio, 1-methylpropylthio(sec-butylthio), 2-methylpropylthio(isobutylthio) or 1,1-dimethylethylthio(tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “C₁-C₂-haloalkylthio” is a C₁-C₂-haloalkyl group, as defined above, attached via a sulfur atom. The term “C₁-C₄-haloalkylthio” is a C₁-C₄-haloalkyl group, as defined above, attached via a sulfur atom. The term “C₁-C₆-haloalkylthio” is a C₁-C₆-haloalkyl group, as defined above, attached via a sulfur atom. The term “C₁-C₁₀-haloalkylthio” is a C₁-C₁₀-haloalkyl group, as defined above, attached via a sulfur atom. C₁-C₂-Haloalkylthio is, for example, SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₄-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The term “C₁-C₂-alkylsulfinyl” is a C₁-C₂-alkyl group, as defined above, attached via a sulfinyl [S(O)] group. The term “C₁-C₄-alkylsulfinyl” is a C₁-C₄-alkyl group, as defined above, attached via a sulfinyl [S(O)] group. The term “C₁-C₆-alkylsulfinyl” is a C₁-C₆-alkyl group, as defined above, attached via a sulfinyl [S(O)] group. The term “C₁-C₁₀-alkylsulfinyl” is a C₁-C₁₀-alkyl group, as defined above, attached via a sulfinyl [S(O)] group. C₁-C₂-Alkylsulfinyl is methylsulfinyl or ethylsulfinyl. C₁-C₄-Alkylsulfinyl is additionally, for example, n-propylsulfinyl, 1-methylethylsulfinyl(isopropylsulfinyl), butylsulfinyl, 1-methylpropylsulfinyl(sec-butylsulfinyl), 2-methylpropylsulfinyl(isobutylsulfinyl) or 1,1-dimethylethylsulfinyl(tert-butylsulfinyl). C₁-C₆-Alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl. C₁-C₈-Alkylsulfinyl is additionally, for example, heptylsulfinyl, octylsulfinyl, 2-ethylhexylsulfinyl and positional isomers thereof. C₁-C₁₀-Alkylsulfinyl is additionally, for example, nonylsulfinyl, decylsulfinyl and positional isomers thereof.

The term “C₁-C₂-haloalkylsulfinyl” is a C₁-C₂-haloalkyl group, as defined above, attached via a sulfinyl [S(O)] group. The term “C₁-C₄-haloalkylsulfinyl” is a C₁-C₄-haloalkyl group, as defined above, attached via a sulfinyl [S(O)] group. The term “C₁-C₆-haloalkylsulfinyl” is a C₁-C₆-haloalkyl group, as defined above, attached via a sulfinyl [S(O)] group. The term “C₁-C₁₀-haloalkylsulfinyl” is a C₁-C₁₀-haloalkyl group, as defined above, attached via a sulfinyl [S(O)] group. C₁-C₂-Haloalkylsulfinyl is, for example, S(O)CH₂F, S(O)CHF₂, S(O)CF₃, S(O)CH₂Cl, S(O)CHCl₂, S(O)CCl₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl or S(O)C₂F₅. C₁-C₄-Haloalkylsulfinyl is additionally, for example, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, S(O)CH₂—C₂F₅, S(O)CF₂—C₂F₅, 1-(CH₂F)-2-fluoroethylsulfinyl, 1-(CH₂Cl)-2-chloroethylsulfinyl, 1-(CH₂Br)-2-bromoethylsulfinyl, 4-fluorobutylsulfinyl, 4-chlorobutylsulfinyl, 4-bromobutylsulfinyl or nonafluorobutylsulfinyl. C₁-C₆-Haloalkylsulfinyl is additionally, for example, 5-fluoropentylsulfinyl, 5-chloropentylsulfinyl, 5-brompentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl or dodecafluorohexylsulfinyl.

The term “C₁-C₂-alkylsulfonyl” is a C₁-C₂-alkyl group, as defined above, attached via a sulfonyl [S(O)₂] group. The term “C₁-C₄-alkylsulfonyl” is a C₁-C₄-alkyl group, as defined above, attached via a sulfonyl [S(O)₂] group. The term “C₁-C₆-alkylsulfonyl” is a C₁-C₆-alkyl group, as defined above, attached via a sulfonyl [S(O)₂] group. The term “C₁-C₁₀-alkylsulfonyl” is a C₁-C₁₀-alkyl group, as defined above, attached via a sulfonyl [S(O)₂] group. C₁-C₂-Alkylsulfonyl is methylsulfonyl or ethylsulfonyl. C₁-C₄-Alkylsulfonyl is additionally, for example, n-propylsulfonyl, 1-methylethylsulfonyl(isopropylsulfonyl), butylsulfonyl, 1-methylpropylsulfonyl(sec-butylsulfonyl), 2-methylpropylsulfonyl(isobutylsulfonyl) or 1,1-dimethylethylsulfonyl(tert-butylsulfonyl). C₁-C₆-Alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl. C₁-C₈-Alkylsulfonyl is additionally, for example, heptylsulfonyl, octylsulfonyl, 2-ethylhexylsulfonyl and positional isomers thereof. C₁-C₁₀-Alkylsulfonyl is additionally, for example, nonylsulfonyl, decylsulfonyl and positional isomers thereof.

The term “C₁-C₂-haloalkylsulfonyl” is a C₁-C₂-haloalkyl group, as defined above, attached via a sulfonyl [S(O)₂] group. The term “C₁-C₄-haloalkylsulfonyl” is a C₁-C₄-haloalkyl group, as defined above, attached via a sulfonyl [S(O)₂] group. The term “C₁-C₆-haloalkylsulfonyl” is a C₁-C₆-haloalkyl group, as defined above, attached via a sulfonyl [S(O)₂] group. The term “C₁-C₁₀-haloalkylsulfonyl” is a C₁-C₁₀-haloalkyl group, as defined above, attached via a sulfonyl [S(O)₂] group. C₁-C₂-Haloalkylsulfonyl is, for example, S(O)₂CH₂F, S(O)₂CHF₂, S(O)₂CF₃, S(O)₂CH₂Cl, S(O)₂CHCl₂, S(O)₂CCl₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl or S(O)₂C₂F₅. C₁-C₄-Haloalkylsulfonyl is additionally, for example, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, S(O)₂CH₂—C₂F₅, S(O)₂CF₂—C₂F₅, 1-(CH₂F)-2-fluoroethylsulfonyl, 1-(CH₂Cl)-2-chloroethylsulfonyl, 1-(CH₂Br)-2-bromoethylsulfonyl, 4-fluorobutylsulfonyl, 4-chlorobutylsulfonyl, 4-bromobutylsulfonyl or nonafluorobutylsulfonyl. C₁-C₆-Haloalkylsulfonyl is additionally, for example, 5-fluoropentylsulfonyl, 5-chloropentylsulfonyl, 5-brompentylsulfonyl, 5-iodopentylsulfonyl, undecafluoropentylsulfonyl, 6-fluorohexylsulfonyl, 6-chlorohexylsulfonyl, 6-bromohexylsulfonyl, 6-iodohexylsulfonyl or dodecafluorohexylsulfonyl.

The term “3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members” as used herein refers to monocyclic radicals, the monocyclic radicals being saturated, partially unsaturated or aromatic. The heterocyclic radical may be attached to the remainder of the molecule via a carbon ring member or via a nitrogen ring member.

Examples of 3-, 4-, 5-, 6- or 7-membered saturated heterocyclyl include:

Oxiranyl, aziridinyl, azetidinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-pyrazolidinyl, 4-pyrazolidinyl, 5-pyrazolidinyl, 2-imidazolidinyl, 4-imidazolidinyl, 2-oxazolidinyl, 4-oxazolidinyl, 5-oxazolidinyl, 3-isoxazolidinyl, 4-isoxazolidinyl, 5-isoxazolidinyl, 2-thiazolidinyl, 4-thiazolidinyl, 5-thiazolidinyl, 3-isothiazolidinyl, 4-isothiazolidinyl, 5-isothiazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2-tetrahydropyranyl, 4-tetrahydropyranyl, 1,3-dioxan-5-yl, 1,4-dioxan-2-yl, 2-piperidinyl, 3-piperidinyl, 4-piperidinyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl, hexahydroazepin-1-, -2-, -3- or -4-yl, hexahydrooxepinyl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of 3-, 4-, 5-, 6- or 7-membered partially unsaturated heterocyclyl include:

2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro-[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

3-, 4-, 5-, 6- or 7-membered aromatic heterocyclyl is 5- or 6-membered aromatic heterocyclyl (hetaryl). Examples are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

A 5-, 6-, 7- or 8-membered completely unsaturated heterocyclic ring containing 1, 2 or 3 heteroatoms selected from O, S and N as ring members contains the maximum number of possible double bonds in the ring. In the case of 5- or 6-membered rings, these are heteroaromatic. In the case of 7- and 8-membered rings, these cannot be aromatic, but can be homoaromatic (7-membered ring, 3 double bonds) or can have 4 double bonds (8-membered ring).

C₂-C₇-alkylene is divalent branched or preferably unbranched saturated aliphatic chain having 2 to 7 carbon atoms, for example —CH₂CH₂, —CH(CH₃)—, —CH₂CH₂CH₂—, —CH(CH₃)CH₂—, —CH₂CH(CH₃)—, —CH₂CH₂CH₂CH₂—, —CH₂CH₂CH₂CH₂CH₂—, —CH₂CH₂CH₂CH₂CH₂CH₂—, and —CH₂CH₂CH₂CH₂CH₂CH₂CH₂—.

The remarks made below concerning preferred embodiments of the variables of the compounds of formula I, especially with respect to their substituents W, X, A¹, A², A³, A⁴, B, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, n, p and q, the features of the use and method according to the invention and of the composition of the invention are valid both on their own and, in particular, in every possible combination with each other.

R² is a substituent on the ring containing A¹, A², A³ and A⁴ as ring members.

As a matter of course, the p radicals R² replace a hydrogen atom on a carbon ring atom. For instance, if A¹, A², A³ or A⁴ is defined to be CH and if this position is to be substituted by a radical R², then A¹, A², A³ or A⁴ is of course C—R². If there is more than one radical R², these can be the same or different.

Preferably, at most three and more preferably at most two of A¹, A², A³ and A⁴ are N. If two of A¹, A², A³ and A⁴ are N, it is preferred that A¹ and A² are N and A³ and A⁴ are CH. More preferably, at most one of A¹, A², A³ and A⁴ is N. In one embodiment, A¹, A², A³ and A⁴ are CH. In an alternative embodiment, A¹, A³ and A⁴ are CH and A² is N. In an alternative embodiment, A², A³ and A⁴ are CH and A¹ is N. In an alternative embodiment, A¹, A² and A⁴ are CH and A³ is N. In an alternative embodiment, A¹, A² and A³ are CH and A⁴ is N. In particular, A¹, A², A³ and A⁴ are CH or A¹, A³ and A⁴ are CH and A² is N.

In a preferred embodiment, the ring comprising the groups A¹, A², A³ and A⁴ as ring members carries 0, 1 or 2 and more preferably 0 or 1 substituent R². In other words, p is preferably 0, 1 or 2 and more preferably 0 or 1. In case p is 1, R² is preferably not bound to the position of A². Particularly preferably, p is 0.

As a matter of course, the q radicals R⁴ replace a hydrogen atom on a carbon ring atom. If there is more than one radical R⁴, these can be the same or different.

q is preferably 0, 1, 2 or 3, more preferably 1, 2 or 3, even more preferably 1 or 2 and specifically 2. If q is 3, the three substituents R⁴ are preferably bound in the positions 3, 4 and 5 or 2, 4 and 6, relative to the 1-position of the attachment point of the phenyl ring to the azoline ring. If q is 2, the two substituents R⁴ are preferably bound in the 3- and 5-positions, relative to the 1-position of the attachment point of the phenyl ring to the azoline ring. If q is 1, the substituent R⁴ is preferably bound in the 3-position, relative to the 1-position of the attachment point of the phenyl ring to the azoline ring.

X is preferably O.

W is preferably O.

B is preferably selected from O, S, NR^{5b}, CR⁶R⁷ and C(═W).

Alternatively, B is preferably selected from SO.

Preferably, R¹ is selected from —NR^{11a}R^{11b}, —OR¹⁰, —S(O)ₙR¹⁰, —S(O)ₙNR^{11a}R^{11b}, —C(═O)R¹², —C(═O)OR¹³, —C(═O)NR^{11a}R^{11b}b, —C(═S)R¹², —C(═S)OR¹³, —C(═S)SR¹³, —C(═S)NR^{11a}R^{11b}, —C(═NR^{11a})R¹², —Si(R¹⁴)₂R¹⁵, phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2 and in particular 1, radicals R¹⁶;

where R¹⁰, R^{11a}, R^{11b}, R¹², R¹³, R¹⁴, R¹⁵, R¹⁶ and n have one of the general meanings given above or, in particular, have one of the preferred meanings given below;
or
R¹ forms together with R^{5b} and the nitrogen atom to which they are bound a 5- or 6-membered saturated, partially unsaturated or aromatic heterocyclic ring which may comprise 1 or 2 further heteroatoms or heteroatom groups selected from N, O, S, NO, SO and SO₂, as ring members, where the heterocyclic ring may be substituted by one or more radicals R¹⁶;
or
in case B is CR⁶R⁷ and R⁶ and R⁷ form together a group ═NR^{11a}, ═NOR¹⁰ or ═NNR^{11a}R^{11b}, R¹ forms together with R¹⁰ or R^{11a} and the atoms to which they are bound a 5- or 6-membered saturated, partially unsaturated or aromatic heterocyclic ring which may comprise 1 or 2 further heteroatoms or heteroatom groups selected from N, O, S, NO, SO and SO₂, as ring members, where the heterocyclic ring may be substituted by one or more radicals R¹⁶;
where R^{11b} and R¹⁶ have one of the general meanings given above or, in particular, have one of the preferred meanings given below.

More preferably, R¹ is selected from —NR^{11a}R^{11b}, —OR¹⁰, —S(O)ₙR¹⁰, —S(O)ₙNR^{11a}R^{11b}, —C(═O)R¹², —C(═O)OR¹³, —C(═O)NR^{11a}R^{11b}, —C(═S)R¹², —C(═S)OR¹³, —C(═S)SR¹³, —C(═S)NR^{11a}R^{11b}, —C(═NR^{11a})R¹², —Si(R¹⁴)₂R¹⁵, phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2 and in particular 1, radicals R¹⁶;

where R¹⁰, R^{11a}, R^{11b}, R¹², R¹³, R¹⁴, R¹⁵, R¹⁶ and n have one of the general meanings given above or, in particular, have one of the preferred meanings given below;
or
R¹ forms together with R^{5b} and the nitrogen atom to which they are bound a 5- or 6-membered saturated, partially unsaturated or aromatic heterocyclic ring which may comprise 1 or 2 further heteroatoms or heteroatom groups selected from N, O, S, NO, SO and SO₂, as ring members, where the heterocyclic ring may be substituted by one or more radicals R¹⁶;
or
in case B is CR⁶R⁷ and R⁶ and R⁷ form together a group ═NR^{11a}, ═NOR¹⁰ or ═NNR^{11a}R^{11b}, R¹ forms together with R¹⁰ or R^{11a} and the atoms to which they are bound a 5- or 6-membered saturated, partially unsaturated or aromatic heterocyclic ring which may comprise 1 or 2 further heteroatoms or heteroatom groups selected from N, O, S, NO, SO and SO₂, as ring members, where the heterocyclic ring may be substituted by one or more, preferably 1, 2 or 3, more preferably 1 or 2 and in particular 1, radicals R¹⁶;
where R^{11b} and R¹⁶ have one of the general meanings given above or, in particular, have one of the preferred meanings given below.

Even more preferably, R¹ is selected from —NR^{11a}R^{11b}, —OR¹⁰ and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more radicals R¹⁶;

where R¹⁰, R^{11a}, R^{11b} and R¹⁶ have one of the general meanings given above or, in particular, have one of the preferred meanings given below;
or
R¹ forms together with R^{5b} and the nitrogen atom to which they are bound a 5- or 6-membered partially unsaturated or aromatic heterocyclic ring which may comprise 1 or 2 further heteroatoms or heteroatom groups selected from N, O, S, NO, SO and SO₂, as ring members, where the heterocyclic ring may be substituted by one or more radicals R¹⁶, the moiety —B—R¹ thus resulting in an N-bound 5- or 6-membered partially unsaturated or aromatic heterocyclic ring which may comprise, apart the nitrogen ring atom via which it is bound to the remainder of the molecule, 1 or 2 further heteroatoms or heteroatom groups selected from N, O, S, NO, SO and SO₂, as ring members, where the heterocyclic ring may be substituted by one or more radicals R¹⁶;
or
in case B is CR⁶R⁷ and R⁶ and R⁷ form together a group ═NR^{11a}, ═NOR¹⁰ or ═NNR^{11a}R^{11b}, R¹ forms together with R¹⁰ or R^{11a} and the atoms to which they are bound a 5- or 6-membered partially unsaturated or aromatic heterocyclic ring which may comprise 1 further heteroatom or heteroatom group selected from N, O, S, NO, SO and SO₂, as ring member, where the heterocyclic ring may be substituted by one or more radicals R¹⁶, the moiety —B—R¹ thus resulting in a C-bound 5- or 6-membered partially unsaturated or aromatic heterocyclic ring which may comprise, apart the heteroatom(s) stemming from the groups ═NR^{11a}, ═NOR¹⁰ or ═NNR^{11a}R^{11b}, 1 further heteroatom or heteroatom group selected from N, O, S, NO, SO and SO₂, as ring member, where the heterocyclic ring may be substituted by one or more radicals R¹⁶;
where R^{11b} is hydrogen, C₁-C₄-alkyl or C₁-C₄-haloalkyl and R¹⁶ has one of the general meanings given above or, in particular, has one of the preferred meanings given below.

In case B is O, S or NR^{5b}, R¹ is preferably a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2 and in particular 1, radicals R¹⁶; or in case B is NR^{5b}, R¹ forms together with R^{5b} and the nitrogen atom to which they are bound a 5- or 6-membered aromatic heterocyclic ring which may comprise 1 or 2 further heteroatoms selected from N, O and S as ring members, where the heterocyclic ring may be substituted by one or more radicals R¹⁶;

where R^{5b} and R¹⁶ have one of the general meanings given above or, in particular, have one of the preferred meanings given below.

In case B is O, S or NR^{5b}, R¹ is more preferably a 5- or 6-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more radicals R¹⁶; or in case B is NR^{5b}, R¹ forms together with R^{5b} and the nitrogen atom to which they are bound a 5- or 6-membered aromatic heterocyclic ring which may comprise 1 or 2 further heteroatoms selected from N, O and S as ring members, where the heterocyclic ring may be substituted by one or more, preferably 1, 2 or 3, more preferably 1 or 2 and in particular 1, radicals R¹⁶, the moiety —B—R¹ thus resulting in an N-bound 5- or 6-membered partially unsaturated or aromatic heterocyclic ring which may comprise, apart the nitrogen ring atom via which it is bound to the remainder of the molecule, 1 or 2 further heteroatoms or heteroatom groups selected from N, O, S, NO, SO and SO₂, as ring members, where the heterocyclic ring may be substituted by one or more, preferably 1, 2 or 3, more preferably 1 or 2 and in particular 1, radicals R¹⁶;

where R^{5b} and R¹⁶ have one of the general meanings given above or, in particular, have one of the preferred meanings given below.

In case B is CR⁶R⁷, C(═W) or NR^{5c}—C(═W), R¹ is preferably selected from —NR^{11a}R^{11b} and —OR¹⁰; or, in case B is CR⁶R⁷ and R⁶ and R⁷ form together a group ═NR^{11a}, ═NOR¹⁰ or ═NNR^{11a}R^{11b}, R¹ is —NR^{11a}R^{11b}, or R¹ forms together with R¹⁰ or R^{11a} and the atoms to which they are bound a 5- or 6-membered aromatic heterocyclic ring which may comprise 1 further heteroatom selected from N, O and S as ring member, where the heterocyclic ring may be substituted by one or more, preferably 1, 2 or 3, more preferably 1 or 2 and in particular 1, radicals R¹⁶, the moiety —B—R¹ thus resulting in a C-bound, 5- or 6-membered partially unsaturated or aromatic heterocyclic ring which may comprise, apart the heteroatom(s) stemming from the groups ═NR^{11a}, ═NOR¹⁰ or ═NNR^{11a}R^{11b}, 1 further heteroatom or heteroatom group selected from N, O, S, NO, SO and SO₂, as ring member, where the heterocyclic ring may be substituted by one or more, preferably 1, 2 or 3, more preferably 1 or 2 and in particular 1, radicals R¹⁶;

where R^{5c}, R¹⁰, R^{11a}, R^{11b} and R¹⁶ have one of the general meanings given above or, in particular, have one of the preferred meanings given below.

In the above definition of R¹, R¹⁰ in the group —OR¹⁰ is preferably selected from hydrogen, C₁-C₁₀-alkyl which may be partially or fully halogenated and/or may carry one substituent R²², and is more preferably hydrogen or C₁-C₄-alkyl, specifically hydrogen.

In the above definition of R¹, R^{11a} in the group —NR^{11a}R^{11b} is preferably selected from hydrogen, C₁-C₁₀-alkyl which may be partially or fully halogenated and/or may carry one substituent R²²; and —C(═O)R¹², where R¹² has one of the general meanings given above and is preferably selected from hydrogen, C₁-C₆-alkyl, C₁-C₄-haloalkyl and C₁-C₄-alkyl (preferably methyl or ethyl, more preferably methyl) which carries one substituent R²², where R²² has one of the general meanings given above or, in particular, one of the preferred meanings given below; and R^{11b} is selected from hydrogen and C₁-C₄-alkyl.

In the above definition of R¹⁰ and R^{11a}, R²² is preferably selected from cyano, C₃-C₈-cycloalkyl, C₃-C₈-halocycloalkyl, —OR¹⁹, —OSO₂R¹⁹, —S(O)ₙR¹⁹, —S(O)ₙN(R^{20a})R^{20b}, —N(R^{20a})R^{20b}, —C(═O)N(R^{20a})R^{20b}, —C(═S)N(R^{20a})R^{20b}, —C(═O)OR²¹, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy; and preferably from C₁-C₄-alkylthio, C₁-C₄-haloalkylthio, phenyl and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the phenyl ring or the heterocyclic ring may be substituted by one or more, preferably 1, 2 or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy;

where R¹⁹, R^{20a}, R^{20b}, R²¹ and n have one of the general meanings given above or, in particular, one of the preferred meanings given below.

More preferably, in the above definition of R¹⁰ and R^{11a}, R²² is selected from cyano, C₃-C₈-cycloalkyl, C₃-C₈-halocycloalkyl, —OR¹⁹, —OSO₂R¹⁹, —S(O)ₙR¹⁹, —S(O)ₙN(R^{20a})R^{20b}, —N(R^{20a})R^{20b}, —C(═O)N(R^{20a})R^{20b}, —C(═S)N(R^{20a})R^{20b}, —C(═O)OR²¹, phenyl which may be substituted by 1, 2, 3, 4 or 5, preferably 1, 2 or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy; and preferably from C₁-C₄-alkylthio, C₁-C₄-haloalkylthio, phenyl and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the phenyl ring or the heterocyclic ring may be substituted by one or more, preferably 1, 2 or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy;

where R¹⁹, R^{20a}, R^{20b}, R²¹ and n have one of the general meanings given above or, in particular, one of the preferred meanings given below.

Even more preferably, in the above definition of R¹⁰ and R^{11a}, R²² is —S(O)ₙR¹⁹, where R¹⁹ and n have one of the general meanings given above or, in particular, one of the preferred meanings given below. However, in this case, n is more preferably O and R¹⁹ is more preferably C₁-C₄-alkyl or C₁-C₄-haloalkyl and is specifically methyl or trifluoromethyl.

Alternatively preferably, R¹ is H or C₁-C₄-haloalkyl.

Alternatively preferably, R¹ is C(═O)R¹², C(═O)NR^{11a}R^{11b} or SO₂C₁-C₄-alkyl, where R¹² is C₁-C₄-alkyl, R^{11a} is H and R^{11b} is C₁-C₄-alkyl.

Preferably, each R² is independently selected from the group consisting of halogen, cyano, azido, nitro, —SCN, —SF₅, C₁-C₆-alkyl which may be partially or fully halogenated and/or may be substituted by one or more, preferably 1, 2 or 3, more preferably 1 or 2 and in particular 1, radicals R⁸, C₃-C₈-cycloalkyl which may be partially or fully halogenated and/or may be substituted by one or more, preferably 1, 2 or 3, more preferably 1 or 2 and in particular 1, radicals R⁹, C₂-C₆-alkenyl which may be partially or fully halogenated and/or may be substituted by one or more, preferably 1, 2 or 3, more preferably 1 or 2 and in particular 1, radicals R⁸, C₂-C₆-alkynyl which may be partially or fully halogenated and/or may be substituted by one or more, preferably 1, 2 or 3, more preferably 1 or 2 and in particular 1, radicals R⁸, —Si(R¹⁴)₂R¹⁵, —C(═O)R¹², —C(═O)OR¹³, —C(═NR^{11a})R¹², —C(═S)R¹², phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2 and in particular 1, radicals R¹⁶, where R⁸, R⁹, R^{11a}, R¹², R¹³, R¹⁴, R¹⁵ and R¹⁶ have one of the general meanings given above or, in particular, one of the preferred meanings given below.

More preferably, each R² is independently selected from the group consisting of halogen, cyano, nitro, C₁-C₆-alkyl which may be partially or fully halogenated and/or may be substituted by one or more, preferably 1, 2 or 3, more preferably 1 or 2 and in particular 1, radicals R⁸, —C(═O)R¹², —C(═O)OR¹³, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2 and in particular 1, radicals R¹⁶,

where R⁸, R¹², R¹³ and R¹⁶ have one of the general meanings given above or, in particular, one of the preferred meanings given below.

R³ is preferably hydrogen or halogen, more preferably halogen and is in particular fluorine.

Preferably, each R⁴ is independently selected from the group consisting of halogen, cyano, nitro, —SCN, —SF₅, C₁-C₆-alkyl, C₁-C₆-alkyl which may be partially or fully halogenated and/or may be substituted by one or more, preferably 1, 2 or 3, more preferably 1 or 2 and in particular 1, radicals R⁸, C₃-C₈-cycloalkyl which may be partially or fully halogenated and/or may be substituted by one or more, preferably 1, 2 or 3, more preferably 1 or 2 and in particular 1, radicals R⁹, C₂-C₆-alkenyl which may be partially or fully halogenated and/or may be substituted by one or more, preferably 1, 2 or 3, more preferably 1 or 2 and in particular 1, radicals R⁸, C₂-C₆-alkynyl which may be partially or fully halogenated and/or may be substituted by one or more, preferably 1, 2 or 3, more preferably 1 or 2 and in particular 1, radicals R⁸, —Si(R¹⁴)₂R¹⁵, —OR¹⁰, —OS(O)ₙR¹⁰, —S(O)ₙR¹⁰, —NR^{11a}R^{11b}, —N(R^{11a})C(═O)R¹², —C(═O)R¹², —C(═O)OR¹³, —C(═NR^{11a})R¹² —C(═S)R¹², phenyl which may be substituted by 1, 2, 3, 4 or 5, preferably 1, 2 or 3, more preferably 1 or 2 and in particular 1, radicals R¹⁶; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, preferably 1, 2 or 3, more preferably 1 or 2 and in particular 1, radicals R¹⁶,

where R⁸, R⁹, R¹⁰, R^{11a}, R^{11b}, R¹², R¹³, R¹⁴, R¹⁵ and R¹⁶ and n have one of the general meanings given above or, in particular, one of the preferred meanings given below.

More preferably, each R⁴ is independently selected from halogen, cyano, nitro, hydroxyl, C₁-C₄-alkyl, C₁-C₄-haloalkyl, C₁-C₄-alkoxy and C₁-C₄-haloalkoxy, even more preferably from halogen and C₁-C₄-haloalkyl and is preferably halogen, specifically chlorine, or CF₃.

R^{5a} is preferably hydrogen or methyl and more preferably hydrogen.

Preferably, R^{5b} and R^{5c}, independently of each other, are selected from the group consisting of hydrogen, C₁-C₆-alkyl, C₁-C₆-haloalkyl, OH, C₁-C₄-alkoxy, C₁-C₄-haloalkoxy, C₁-C₆-alkylcarbonyl, C₁-C₆-haloalkylcarbonyl, C₁-C₄-alkoxycarbonyl, C₁-C₄-haloalkoxycarbonyl and benzyl, more preferably from hydrogen and C₁-C₄-alkyl, and are in particular hydrogen.

R⁶ and R⁷, when not forming together a group ═CR¹⁷R¹⁸; ═S(O)ₙR¹⁰; ═S(O)ₙNR^{11a}R^{11b}, ═NR^{11a}, ═NOR¹⁰ or ═NNR^{11a}R^{11b}, independently of each other are preferably selected from the group consisting of hydrogen, C₁-C₄-alkyl, C₁-C₄-haloalkyl, OH, C₁-C₄-alkoxy and C₁-C₄-haloalkoxy and are preferably hydrogen.

R⁸ is preferably selected from the group consisting of cyano, azido, nitro, —SCN, SF₅, C₃-C₈-cycloalkyl which may be substituted by phenyl, C₃-C₈-halocycloalkyl, —Si(R¹⁴)₂R¹⁵, —OR¹⁰, —OSO₂R¹⁰, —S(O)ₙR¹⁰, —S(O)ₙN(R^{11a})R^{11b}, —N(R^{11a})R^{11b}, —C(═O)N(R^{11a})R^{11b}—C(═S)N(R^{11a})R^{11b}, —C(═O)OR¹³, phenyl which may be substituted by 1, 2, 3, 4 or 5, preferably 1, 2 or 3, more preferably 1 or 2 and in particular 1, radicals R¹⁶, and a 3-, 4-5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. by 1, 2, 3, 4 or 5, preferably 1, 2 or 3, more preferably by 1 or 2 and in particular 1, radicals R¹⁶; or two geminally bound radicals R⁸ together form a group selected from ═CR¹⁷R¹⁸, ═S(O)ₙR¹⁰, ═S(O)ₙN(R^{11a})R^{11b}, ═NR^{11a}, ═NOR¹⁰ and ═NNR^{11a}; or two radicals R⁸, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members,

where R¹⁰, R^{11a}, R^{11b}, R¹³, R¹⁴, R¹⁵, R¹⁶, R¹⁷ and R¹⁸ have one of the meanings given above or in particular one of the preferred meanings given below.

R⁸ is more preferably selected from the group consisting of cyano, C₃-C₈-cycloalkyl which may be substituted by phenyl, C₃-C₈-halocycloalkyl, —OR¹⁰, —SR¹⁰, —C(═O)N(R^{11a})R^{11b}, —N(R^{11a})R^{11b}, phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. by 1, 2, 3, 4 or 5, preferably 1, 2 or 3, more preferably by 1 or 2 and in particular 1, radicals R¹⁶;

where R¹⁰, R^{11a}, R^{11b} and R¹⁶ have one of the meanings given above or in particular one of the preferred meanings given below.

R⁸ is even more preferably selected from the group consisting of cyano, C₃-C₆-cycloalkyl which may be substituted by phenyl, C₃-C₆-halocycloalkyl, C₁-C₄-alkoxy, C₁-C₄-haloalkoxy, C₁-C₄-alkylthio, C₁-C₄-haloalkylthio, —C(═O)N(R^{11a})R^{11b}, —N(R^{11a})R^{11b}, phenyl which may be substituted by 1, 2, 3, 4 or 5, preferably 1, 2 or 3, more preferably 1 or 2 and in particular 1, radicals R¹⁶, and a 5- or 6-membered heteroaromatic ring containing 1, 2 or 3 heteroatoms selected from N, O and S, as ring members, where the heteroaromatic ring may be substituted by one or more, e.g. by 1, 2, 3, 4 or 5, preferably 1, 2 or 3, more preferably by 1 or 2 and in particular 1, radicals R¹⁶;

where R^{11a}, R^{11b} and R¹⁶ have one of the meanings given above or in particular one of the preferred meanings given below.

R⁸ is in particular selected from the group consisting of C₃-C₆-cycloalkyl which may be substituted by phenyl, C₁-C₄-alkylthio, C₁-C₄-haloalkylthio, phenyl which may be substituted by 1, 2, 3, 4 or 5, preferably 1, 2 or 3, more preferably 1 or 2 and in particular 1, radicals R¹⁶, —C(═O)N(R^{11a})R^{11b}, —N(R^{11a})R^{11b}, and a 5- or 6-membered heteroaromatic ring containing 1, 2 or 3 heteroatoms selected from N, O and S, as ring members, where the heteroaromatic ring may be substituted by one or more, e.g. by 1, 2, 3, 4 or 5, preferably 1, 2 or 3, more preferably by 1 or 2 and in particular 1, radicals R¹⁶;

where R^{11a}, R^{11b} and R¹⁶ have one of the meanings given above or in particular one of the preferred meanings given below.

R⁹ is preferably selected from the group consisting of halogen, cyano, azido, nitro, —SCN, SF₅, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹⁵, —OR¹⁰, —OSO₂R¹⁰, —S(O)ₙR¹⁰, —S(O)ₙN(R^{11a})R^{11b}, —N(R^{11a})R^{11b} —C(═O)N(R^{11a})R^{11b}, —C(═S)N(R^{11a})R^{11b}, —C(═O)OR¹³, phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁶;

or two geminally bound radicals R⁹ together form a group selected from ═CR¹⁷R¹⁸, ═S(O)ₙR¹⁰, ═S(O)ₙN(R^{11aa})R^{11b}, ═NR^{11a}, ═NOR¹⁰ and ═NNR^{11a};
or two radicals R⁹, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members,
where R¹⁰, R^{11a}, R^{11b}, R¹³, R¹⁴, R¹⁵, R¹⁶, R¹⁷ and R¹⁸ have one of the meanings given above or in particular one of the preferred meanings given below.

R⁹ is more preferably selected from the group consisting of halogen, cyano, C₁-C₆-alkyl, C₁-C₆-haloalkyl, C₁-C₆-alkoxy-C₁-C₆-alkyl, —OR¹⁰, —OSO₂R¹⁰, —S(O)ₙR¹⁰, —S(O)ₙN(R^{11a})R^{11b}, —N(R^{11a})R^{11b}, —C(═O)N(R^{11a})R^{11b}, —C(═S)N(R^{11a})R^{11b}, —C(═O)OR¹³ phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. by 1, 2, 3, 4 or 5, preferably 1, 2 or 3, more preferably by 1 or 2 and in particular 1, radicals R¹⁶;

where R¹⁰, R^{11a}, R^{11b}, R¹³ and R¹⁶ have one of the meanings given above or in particular one of the preferred meanings given below.

R⁹ is even more preferably selected from the group consisting of halogen, C₁-C₄-alkyl, C₁-C₃-haloalkyl, C₁-C₄-alkoxy and C₁-C₃-haloalkoxy. In particular, R⁹ is selected from halogen, C₁-C₄-alkyl and C₁-C₃-haloalkyl.

Preferably, each R¹⁰ is independently selected from the group consisting of hydrogen, C₁-C₆-alkyl which may be partially or fully halogenated and/or may carry one or more, preferably 1, 2 or 3, more preferably 1 or 2 and in particular 1, radicals R²², C₃-C₈-cycloalkyl which may be partially or fully halogenated and/or may carry one or more, preferably 1, 2 or 3, more preferably 1 or 2 and in particular 1, radicals R²³, C₂-C₆-alkenyl which may be partially or fully halogenated and/or may carry one or more, preferably 1, 2 or 3, more preferably 1 or 2 and in particular 1, radicals R²², C₂-C₆-alkynyl which may be partially or fully halogenated and/or may carry one or more, preferably 1, 2 or 3, more preferably 1 or 2 and in particular 1, radicals R²², phenyl, benzyl, where the phenyl moieties in the two last-mentioned radicals may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3, 4 or 5, preferably 1, 2 or 3, more preferably 1 or 2 and in particular 1, radicals R¹⁶;

where R¹⁶, R²² and R²³ have one of the meanings given above or in particular one of the preferred meanings given below.

More preferably, each R¹⁰ is independently selected from the group consisting of hydrogen, C₁-C₆-alkyl which may be partially or fully halogenated and/or may carry one or more, preferably 1, 2 or 3, more preferably 1 or 2 and in particular 1, radicals R²², phenyl, benzyl, where the phenyl moieties in the two last-mentioned radicals may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3, 4 or 5, preferably 1, 2 or 3, more preferably 1 or 2 and in particular 1, radicals R¹⁶;

where R¹⁶ and R²² have one of the meanings given above or in particular one of the preferred meanings given below.

In particular, each R¹⁰ is independently selected from the group consisting of hydrogen and C₁-C₆-alkyl which may be partially or fully halogenated and/or may carry one or more, preferably 1, 2 or 3, more preferably 1 or 2 and in particular 1, radicals R²², where R²² has one of the meanings given above or in particular one of the preferred meanings given below.

R^{11a} and R^{11b}, independently of each occurrence and independently of each other, are preferably selected from the group consisting of hydrogen, C₁-C₆-alkyl which may be partially or fully halogenated and/or may carry one or more, preferably 1, 2 or 3, more preferably 1 or 2 and in particular 1, radicals R²², C₃-C₈-cycloalkyl which may be partially or fully halogenated and/or may carry one or more radicals R²³, C₃-C₈-cycloalkyl-C₁-C₄-alkyl, C₃-C₈-halocycloalkyl-C₁-C₄-alkyl, where the cycloalkyl moieties in the two last-mentioned radicals may be partially or fully halogenated and/or may carry one or more, preferably 1, 2 or 3, more preferably 1 or 2 and in particular 1, radicals R²³, —C(═O)R¹², —C(═O)OR¹³, —C(═O)NR^{20a}R^{20b}, —C(═S)R¹², —C(═S)SR¹³, —C(═S)NR^{20a}R^{20b}, —C(═NR^{20a})R¹²; phenyl, benzyl, where the phenyl moieties in the two last-mentioned radicals may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3, 4 or 5, preferably 1, 2 or 3, more preferably 1 or 2 and in particular 1, radicals R¹⁶;

where R¹², R¹³, R¹⁶, R^{20a}, R^{20b}, R²² and R²³ have one of the meanings given above or in particular one of the preferred meanings given below.

More preferably, R^{11a}, independently of each occurrence, is selected from the group consisting of hydrogen, C₁-C₆-alkyl which may be partially or fully halogenated and/or may carry one or more, preferably 1, 2 or 3, more preferably 1 or 2 and in particular 1, radicals R²², and —C(═O)R¹²,

where R¹² and R²² have one of the meanings given above or in particular one of the preferred meanings given below;
and R^{11b}, independently of each occurrence, is selected from the group consisting of hydrogen and C₁-C₄-alkyl.

R¹² is preferably selected from hydrogen, C₁-C₆-alkyl which may be partially or fully halogenated and/or may be substituted by one or more, preferably 1, 2 or 3, more preferably 1 or 2 and in particular 1, radicals R²², C₃-C₈-cycloalkyl which may be partially or fully halogenated and/or may be substituted by one or more, preferably 1, 2 or 3, more preferably 1 or 2 and in particular 1, radicals R²³, C₂-C₆-alkenyl which may be partially or fully halogenated and/or may be substituted by one or more, preferably 1, 2 or 3, more preferably 1 or 2 and in particular 1, radicals R²², C₂-C₆-alkynyl which may be partially or fully halogenated and/or may be substituted by one or more, preferably 1, 2 or 3, more preferably 1 or 2 and in particular 1, radicals R²², —NR^{20a}R^{20b}, phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3, 4 or 5, preferably 1, 2 or 3, more preferably 1 or 2 and in particular 1, radicals R¹⁶,

where R¹⁶, R^{20a}, R^{20b}, R²² and R²³ have one of the meanings given above or in particular one of the preferred meanings given below.

R¹² is more preferably selected from C₁-C₆-alkyl which may be partially or fully halogenated and/or may be substituted by one or more, preferably 1, 2 or 3, more preferably 1 or 2 and in particular 1, radicals R²², phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3, 4 or 5, preferably 1, 2 or 3, more preferably 1 or 2 and in particular 1, radicals R¹⁶,

where R¹⁶ and R²² have one of the meanings given above or in particular one of the preferred meanings given below.

R¹² is in particular selected from C₁-C₆-alkyl which may be partially or fully halogenated and/or may be substituted by one or more, preferably 1, 2 or 3, more preferably 1 or 2 and in particular 1, radicals R²², where R²² has one of the meanings given above or in particular one of the preferred meanings given below.

R¹³ is preferably selected from hydrogen, C₁-C₆-alkyl which may be partially or fully halogenated and/or may be substituted by one or more, preferably 1, 2 or 3, more preferably 1 or 2 and in particular 1, radicals R²², C₃-C₈-cycloalkyl which may be partially or fully halogenated and/or may be substituted by one or more, preferably 1, 2 or 3, more preferably 1 or 2 and in particular 1, radicals R²³, C₂-C₆-alkenyl which may be partially or fully halogenated and/or may be substituted by one or more, preferably 1, 2 or 3, more preferably 1 or 2 and in particular 1, radicals R²², C₂-C₆-alkynyl which may be partially or fully halogenated and/or may be substituted by one or more, preferably 1, 2 or 3, more preferably 1 or 2 and in particular 1, radicals R²², —NR^{20a}R^{20b}, phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3, 4 or 5, preferably 1, 2 or 3, more preferably 1 or 2 and in particular 1, radicals R¹⁶,

where R¹⁶, R^{20a}, R^{20b}, R²² and R²³ have one of the meanings given above or in particular one of the preferred meanings given below.

R¹³ is more preferably selected from hydrogen, C₁-C₆-alkyl which may be partially or fully halogenated and/or may be substituted by one or more, preferably 1, 2 or 3, more preferably 1 or 2 and in particular 1, radicals R²², phenyl which may be substituted by 1, 2, 3, 4 or 5, preferably 1, 2 or 3, more preferably 1 or 2 and in particular 1, radicals R¹⁶, and a 3-, 4-, 5-, 6- or 7-membered saturated, partially unsaturated or aromatic heterocyclic ring comprising 1, 2 or 3 heteroatoms or heteroatom groups selected from N, O, S, NO, SO and SO₂, as ring members, where the heterocyclic ring may be substituted by one or more, e.g. 1, 2, 3, 4 or 5, preferably 1, 2 or 3, more preferably 1 or 2 and in particular 1, radicals R¹⁶,

where R¹⁶ and R²² have one of the meanings given above or in particular one of the preferred meanings given below.

R¹³ is in particular selected from hydrogen and C₁-C₆-alkyl which may be partially or fully halogenated and/or may be substituted by one or more, preferably 1, 2 or 3, more preferably 1 or 2 and in particular 1, radicals R²², where R²² has one of the meanings given above or in particular one of the preferred meanings given below.

Preferably, R¹⁴ and R¹⁵ are, independently of each other and independently of each occurrence, selected from C₁-C₄-alkyl and are in particular methyl.

Preferably, each R¹⁶ is independently selected from the group consisting of halogen, cyano, C₁-C₆-alkyl which may be partially or fully halogenated and/or may be substituted by one or more, e.g. 1, 2, 3 or 4, preferably 1, 2 or 3, more preferably 1 or 2 and in particular 1, radicals R²², C₃-C₈-cycloalkyl which may be partially or fully halogenated and/or may be substituted by one or more, e.g. 1, 2, 3 or 4, preferably 1, 2 or 3, more preferably 1 or 2 and in particular 1, radicals R²³, —OR¹⁹, —OS(O)ₙR¹⁹, —S(O)ₙR¹⁹, —S(O)ₙN(R^{20a})R^{20b}, —N(R^{20a})R^{20b}, —C(═O)R¹⁹, —C(═O)OR²¹, —C(═O)N(R^{20a})R¹⁹, phenyl which may be substituted by 1, 2, 3, 4 or 5, preferably 1, 2 or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more, e.g. 1, 2, 3 or 4, preferably 1 or 2, more preferably 1, radicals independently selected from halogen, cyano, nitro, C₁-C₆-alkyl, C₁-C₆-haloalkyl, C₁-C₆-alkoxy and C₁-C₆-haloalkoxy;

or two radicals R¹⁶ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, and —O(CH₂)O—, thus forming, together with the atoms to which they are bound, a 5- or 6-membered ring, where the hydrogen atoms of the above groups may be replaced by one or more, e.g. 1, 2, 3 or 4, preferably 1 or 2, more preferably 1, substituents selected from halogen, methyl, halomethyl, hydroxyl, methoxy and halomethoxy or one or more, e.g. 1 or 2, CH₂ groups of the above groups may be replaced by a C═O group,
where R¹⁹, R^{20a}, R^{20b}, R²¹, R²² and R²³ have one of the general meanings given above or in particular one of the preferred meanings given below.

More preferably, each R¹⁶ is independently selected from the group consisting of halogen, cyano, C₁-C₆-alkyl which may be partially or fully halogenated and/or may be substituted by one or more, e.g. 1, 2, 3 or 4, preferably 1 or 2, more preferably 1, radicals R²², —OR¹⁹, —N(R^{20a})R^{20b}, —C(═O)R¹⁹, —C(═O)OR¹²¹, —C(═O)N(R^{20a})R^{20b}, phenyl which may be substituted by 1, 2, 3, 4 or 5, preferably 1, 2 or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more, e.g. 1, 2, 3 or 4, preferably 1 or 2, more preferably 1, radicals independently selected from halogen, cyano, nitro, C₁-C₆-alkyl, C₁-C₆-haloalkyl, C₁-C₆-alkoxy and C₁-C₆-haloalkoxy;

where R¹⁹, R^{20a}, R^{20b}, R²¹ and R²² have one of the general meanings given above or in particular one of the preferred meanings given below.

Even more preferably, each R¹⁶ is independently selected from the group consisting of halogen, C₁-C₄-alkyl, C₁-C₄-haloalkyl, C₁-C₄-alkoxy and C₁-C₄-haloalkoxy. In particular, each R¹⁶ is independently selected from the group consisting of halogen, C₁-C₄-alkyl and C₁-C₄-haloalkyl.

Preferably, R¹⁷ and R¹⁸ are, independently of each other and independently of each occurrence, selected from the group consisting of hydrogen, halogen, C₁-C₆-alkyl and C₁-C₆-haloalkyl. More preferably, R¹⁷ and R¹⁸ are, independently of each other and independently of each occurrence, selected from the group consisting of hydrogen, halogen and C₁-C₆-alkyl and in particular from the group consisting of hydrogen and halogen. Specifically, they are hydrogen.

Preferably, each R¹⁹ is independently selected from the group consisting of hydrogen, C₁-C₆-alkyl, C₁-C₆-haloalkyl, C₃-C₈-cycloalkyl, C₃-C₈-halocycloalkyl, phenyl and benzyl. More preferably, each R¹⁹ is independently selected from the group consisting of hydrogen, C₁-C₆-alkyl, C₁-C₆-haloalkyl and phenyl and is in particular C₁-C₄-alkyl or C₁-C₃-haloalkyl.

Preferably, R^{20a} and R^{20b} are independently of each other and independently of each occurrence selected from the group consisting of hydrogen, C₁-C₆-alkyl, C₂-C₆-alkenyl, C₂-C₆-alkynyl, C₃-C₈-cycloalkyl, wherein the four last-mentioned aliphatic and cycloaliphatic radicals may be partially or fully halogenated and/or wherein in the four last-mentioned aliphatic and cycloaliphatic radicals one or two CH₂ groups may be replaced by a CO group and/or wherein the four last-mentioned aliphatic and cycloaliphatic radicals may carry 1 or 2 radicals selected from C₁-C₄-alkoxy, phenyl, benzyl, pyridyl, and phenoxy, wherein the four last mentioned radicals may in turn be partially or fully halogenated and/or carry 1, 2 or 3 substituents selected from C₁-C₆-alkyl, C₁-C₆-haloalkyl, C₁-C₆-alkoxy, C₁-C₆-haloalkoxy and C₁-C₆-alkoxycarbonyl.

More preferably, R^{20a} and R^{20b} are independently of each other and independently of each occurrence selected from the group consisting of hydrogen, C₁-C₆-alkyl, phenyl and benzyl.

Preferably, each R²¹ is independently selected from the group consisting of hydrogen, C₁-C₆-alkyl, C₂-C₆-alkenyl, C₂-C₆-alkynyl, C₃-C₈-cycloalkyl, wherein the four last-mentioned aliphatic and cycloaliphatic radicals may be partially or fully halogenated and/or wherein in the four last-mentioned aliphatic and cycloaliphatic radicals one or two CH₂ groups may be replaced by a CO group and/or wherein the four last-mentioned aliphatic and cycloaliphatic radicals may carry 1 or 2 radicals selected from C₁-C₄-alkoxy, phenyl, benzyl, pyridyl, and phenoxy, wherein the four last-mentioned radicals may in turn be partially or fully halogenated and/or carry 1, 2 or 3 substituents selected from C₁-C₆-alkyl, C₁-C₆-haloalkyl, C₁-C₆-alkoxy, C₁-C₆-haloalkoxy and C₁-C₆-alkoxycarbonyl.

More preferably, each R²¹ is independently selected from the group consisting of hydrogen, C₁-C₆-alkyl, phenyl and benzyl.

Preferably, each R²² is independently is selected from the group consisting of cyano; C₃-C₈-cycloalkyl, C₃-C₈-halocycloalkyl, —OR¹⁹, —OSO₂R¹⁹, —S(O)ₙR¹⁹, —S(O)ₙN(R^{20a})R^{20b}, —N(R^{20a})R^{20b}, —C(═O)N(R^{20a})R^{20b}, —C(═S)N(R^{20a})R^{20b}, —C(═O)OR²¹, phenyl which may be substituted by 1, 2, 3, 4 or 5, preferably 1, 2 or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3, 4 or 5, preferably 1, 2 or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy;

where R¹⁹, R^{20a}, R^{20b}, R²¹ and n have one of the general meanings given above or, in particular, one of the preferred meanings given above.

More preferably, each R²² is independently selected from —S(O)ₙR¹⁹, phenyl and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the phenyl ring or the heterocyclic ring may be substituted by one or more, e.g. 1, 2, 3, 4 or 5, preferably 1, 2, or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy, where R¹⁹ and n have one of the general meanings given above or, in particular, one of the preferred meanings given above or below. However, in this case, n is more preferably O and R¹⁹ is more preferably C₁-C₄-alkyl or C₁-C₄-haloalkyl and is specifically methyl or trifluoromethyl.

Even more preferably, each R²² is independently selected from C₁-C₄-alkylthio, C₁-C₄-haloalkylthio, phenyl and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the phenyl ring or the heterocyclic ring may be substituted by one or more, e.g. 1, 2, 3, 4 or 5, preferably 1, 2, or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy;

Specifically, R²² is C₁-C₄-alkylthio or C₁-C₄-haloalkylthio, and is more specifically methylthio or trifluoromethylthio.

Preferably, each R²³ is independently is selected from the group consisting of cyano; C₁-C₆-alkyl, C₁-C₆-haloalkyl, C₁-C₆-alkoxy-C₁-C₆-alkyl, C₁-C₆-haloalkoxy-C₁-C₆-alkyl, C₂-C₆-alkenyl, C₂-C₆-haloalkenyl, C₂-C₆-alkynyl, C₂-C₆-haloalkynyl, —OR¹⁹, —OSO₂R¹⁹, —S(O)ₙR¹⁹, —S(O)ₙN(R^{20a})R^{20b}, —N(R^{20a})R^{20b}, —C(═O)N(R^{20a})R^{20b}, —C(═S)N(R^{20a})R^{20b}, —C(═O)OR²¹, phenyl, benzyl, where the phenyl moieties in the two last-mentioned radicals may be substituted by 1, 2, 3, 4 or 5, preferably 1, 2, or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3, 4 or 5, preferably 1, 2, or 3, more preferably 1 or 2 and in particular 1, radicals independently selected from halogen, cyano, nitro, C₁-C₆-alkyl, C₁-C₆-haloalkyl, C₁-C₆-alkoxy and C₁-C₆-haloalkoxy;

where R¹⁹, R^{20a}, R^{20b}, R²¹ and n have one of the general meanings given above or, in particular, one of the preferred meanings given above.

More preferably, each R²³ is independently is selected from the group consisting of cyano; C₁-C₆-alkyl and C₁-C₆-haloalkyl.

n is preferably 0 or 2.

One particularly preferred embodiment of the invention refers to compounds of formula I-1

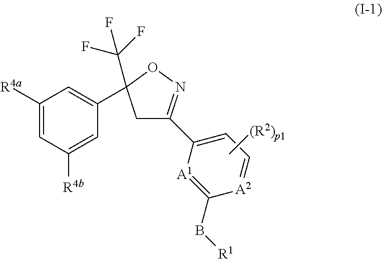

where
A¹ is N or CH, preferably CH;
A² is N or CH;
B, R¹ and R² have one of the general or in particular one of the preferred meanings given above;
R^{4a} and R^{4b} are hydrogen or have one of the general or in particular one of the preferred meanings given above for R⁴, with the proviso that at most one of R^{4a} and R^{4b} is hydrogen; and
p1 is 0 or 1, preferably 0.

In compounds I-1, A¹ is preferably CH. More preferably, A¹ is CH and p1 is preferably 0.

In particular, the invention relates to compounds of formula I-1.1

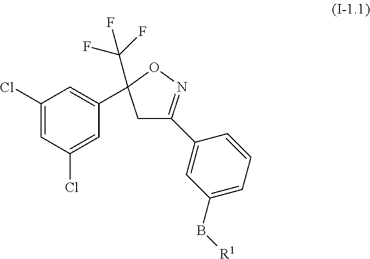

wherein
B is NH; and
R¹ is a 5- or 6-membered saturated, partially unsaturated or aromatic heterocyclic ring containing 1, 2 or 3 heteroatoms selected from N, O and S as ring members;
or
B is NR^{5b}; and
R¹ forms together with R^{5b} and the N-atom to which they are bound an N-bound 5- or 6-membered saturated, partially unsaturated or aromatic heterocyclic ring optionally containing 1 or 2 further heteroatoms selected from N, O and S as ring members, where the heterocyclic ring may carry 1 or 2 substituents R¹⁶; where R¹⁶ is selected from halogen, CN, C₁-C₄-alkyl and oxo;
or
B is CO; and
R¹ is selected from OR¹⁰ and NR^{11a}R^{11b}, where
   R¹⁰, R^{11a} and R^{11b}, independently of each other, are selected from hydrogen, C₁-C₄-alkyl and C₁-C₄-haloalkyl;
or
B is CH₂; and
R¹ is selected from NR^{11a}R^{11b}, OR^{10a}, C(═O)NR^{11a}R^{11b} and SO₂R^{10b}, where
   R^{10a} is selected from C(═O)R¹², C(O)NR^{11a}R^{11b} and C₁-C₄-alkyl which carries 1 radical C(═O)NR^{20a}R^{20b}, where
      R^{20a} and R^{20b} are independently selected from hydrogen and C₁-C₄-alkyl;
   R^{10b} is selected from C₁-C₄-alkyl and C₁-C₄-haloalkyl;
   R^{11a} and R^{11b} are independently selected from hydrogen, C₁-C₄-alkyl which may carry 1 substituent R²², where
      R²² is a 5- or 6-membered saturated, partially unsaturated or aromatic heterocyclic ring containing 1, 2 or 3 heteroatoms selected from N, O and S as ring members; C₁-C₄-haloalkyl and C(═O)R¹²; and
   R¹² is C₁-C₄-alkyl;
or
B is C═N—W¹—R¹⁰, where W¹ is O or NH;
R¹ is H or NH₂; and
R¹⁰ is H, C₁-C₄-alkyl, C(═O)R¹² or C(═O)NR^{11a}R^{11b}; where
   R¹² is C₁-C₄-alkyl; and
   R^{11a} and R^{11b} are independently H or C₁-C₄-alkyl; or
R¹ and R¹⁰, together with the atoms to which they are bound, form a 5- or 6-membered saturated, partially unsaturated or aromatic heterocyclic ring optionally containing 1 further heteroatom selected from N, O and S as ring member, where the heterocyclic ring may carry 1 or 2 substituents R¹⁶; where
   R¹⁶ is selected from halogen, CN and C₁-C₄-alkyl;
or
B is O; and
R¹ is C(═O)R¹², C(═O)NR^{11a}R^{11b} or a 5- or 6-membered saturated, partially unsaturated or aromatic heterocyclic ring containing 1, 2 or 3 heteroatoms selected from N, O and S as ring members, where the heterocyclic ring may carry 1 or 2 substituents R¹⁶; where
   R^{11a} and R^{11b} are independently H or C₁-C₄-alkyl;
   R¹² is C₁-C₄-alkyl; and
   R¹⁶ is selected from halogen, CN and C₁-C₄-alkyl;
or
B is S; and
R¹ is C(═O)R¹²; where
   R¹² is C₁-C₄-alkyl or C₁-C₄-haloalkyl;
or
B is SO; and
R¹ is C₁-C₄-alkyl or C₁-C₄-haloalkyl.

In case that B is CH₂, R¹ is specifically selected from OR^{10a}, C(═O)NR^{11a}R^{11b} and SO₂R^{10b}, where R^{10a}, R^{10b}, R^{11a} and R^{11b} are as defined above.

A specific embodiment of the invention relates to compounds I-1.1, wherein
B is NH; and
R¹ is 2-pyrimidinyl, 2-oxazolidinyl or 2-thiazolidinyl;
or
BR¹ is 1H-[1,2,4]-triazol-1-yl, 4-cyanopyrazol-1-yl or imidazolidin-2-on-1-yl;
or
B is CH₂; and
R¹ is H, NH₂, NH—CO—CH₃, NH—CO—CH₂CH₃, NH—CO—CH₂CH₂CH₃, NH—CO—CH₂—S—CH₃, O—CO—CH₃, O—CO—CH₂CH₃, O—CO—CH₂CH₂CH₃, O—CH₂—CO—N(CH₃)₂, O—CO—NHCH₃, O—CO—NHCH₂CH₃, O—CO—NHCH₃CH₂CH₃, O—CO—NHCH(CH₃)₂, CO—NHCH₃, CO—NHCH₂CH₃, CO—NHCH₃CH₂CH₃, CO—NHCH(CH₃)₂, CO—NHCH₂CF₃, CO—NHCH₂-2-pyridyl, O—CO—N(CH₂CH₃)—CO—NHCH₂CH₃ or SO₂CH₃;
or
B is C═N—OH; and
R¹ is NH₂;
or
B is C═N—OCH₃, C═N—NH—COCH₃ or C═N—NH—CONHCH₃; and
R¹ is H;
or
BR¹ is 5-methyl-[1,2,4]-oxadiazol-3-yl;
or
B is O and
R¹ is N-methylpyrrolidin-2-on-3-yl, COCH₃, CONH—CH₃, CONH—CH₂CH₃ or CONH—CH(CH₃)₂;
or
B is S; and
R¹ is COCH₃;
or
B is SO; and
R¹ is CF₃.

More specifically, in case that B is CH₂, R¹ is O—CO—CH₃, O—CO—CH₂CH₃, O—CO—CH₂CH₂CH₃, O—CH₂—CO—N(CH₃)₂, O—CO—NHCH₃, O—CO—NHCH₂CH₃, O—CO—NHCH₃CH₂CH₃, O—CO—NHCH(CH₃)₂, CO—NHCH₃, CO—NHCH₂CH₃, CO—NHCH₃CH₂CH₃, CO—NHCH(CH₃)₂, CO—NHCH₂CF₃, CO—NHCH₂-2-pyridyl, O—CO—N(CH₂CH₃)—CO—NHCH₂CH₃ or SO₂CH₃.

A further specific embodiment of the invention relates to compounds I-1.2

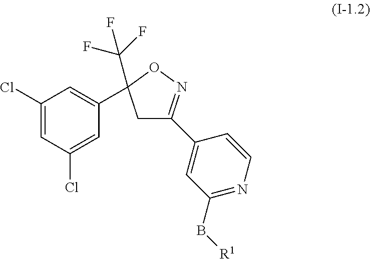

wherein B is CO and R¹ is OH, NH—CH₂CH₂CH₃ or NH—CH₂CF₃.

Examples of preferred compounds are compounds of the following formulae I-1a to I-1ff, where the variables B and R¹ have one of the general or preferred meanings given above, R^{2a} is hydrogen or has one of the general or preferred meanings given above for R² and R^{4a} and R^{4b} are hydrogen or have one of the general or preferred meanings given above for R⁴, where it is however preferred that at lest one of R^{4a} and R^{4b} is not hydrogen. Examples of preferred compounds are the individual compounds compiled in the tables 1 to 128000 below. Moreover, the meanings mentioned below for the individual variables in the tables are per se, independently of the combination in which they are mentioned, a particularly preferred embodiment of the substituents in question.

If in the below tables compounds are disclosed which are disclaimed from the present invention, these compounds are of course not to be understood as belonging to the invention.

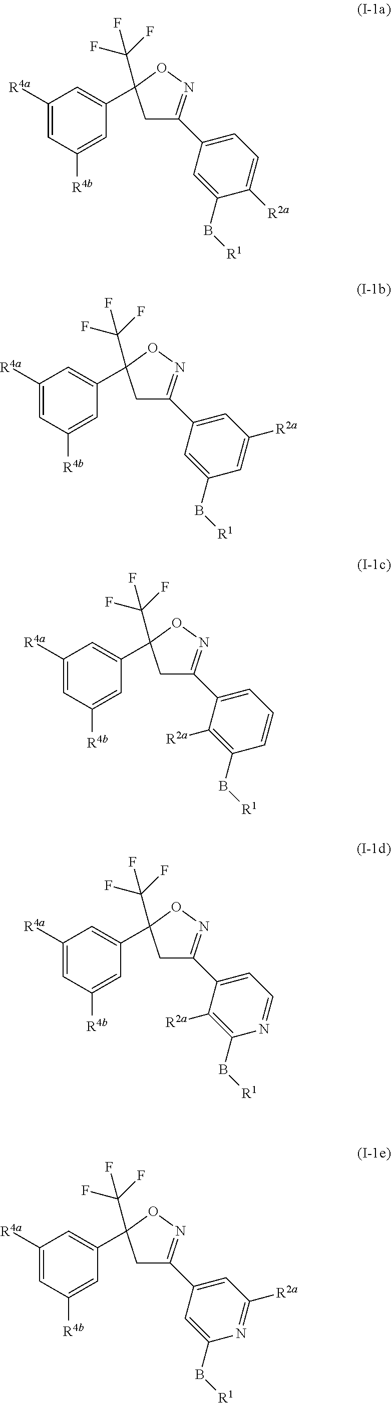

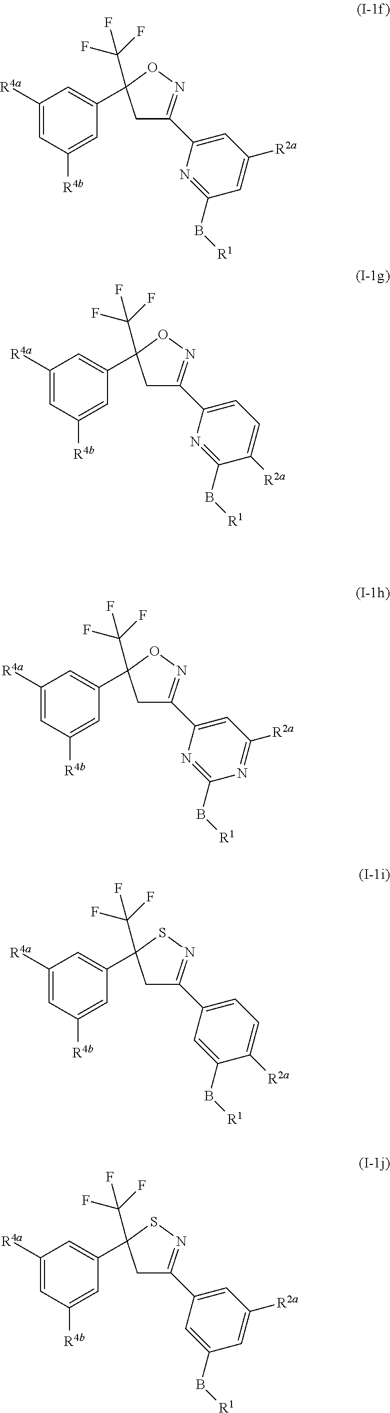

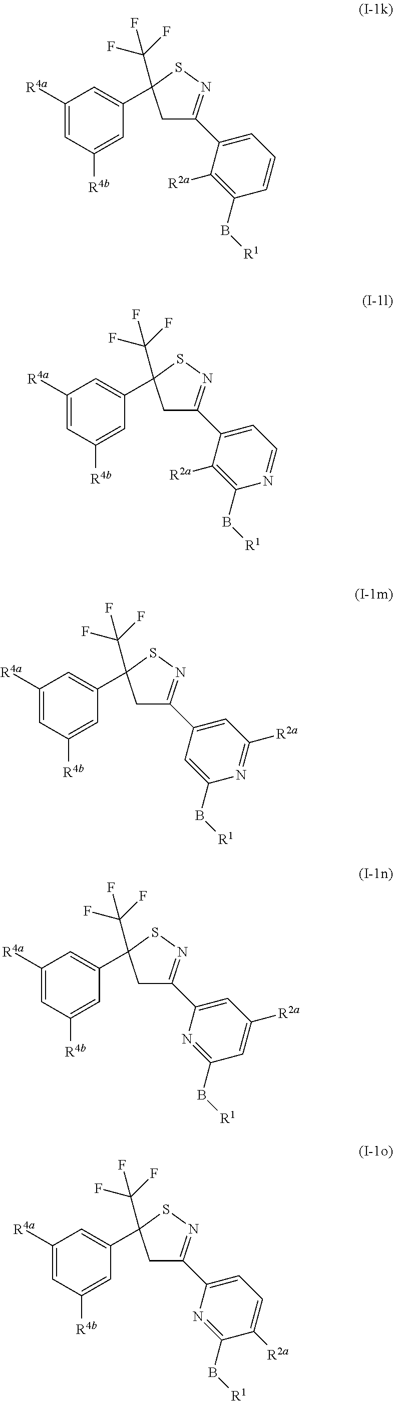

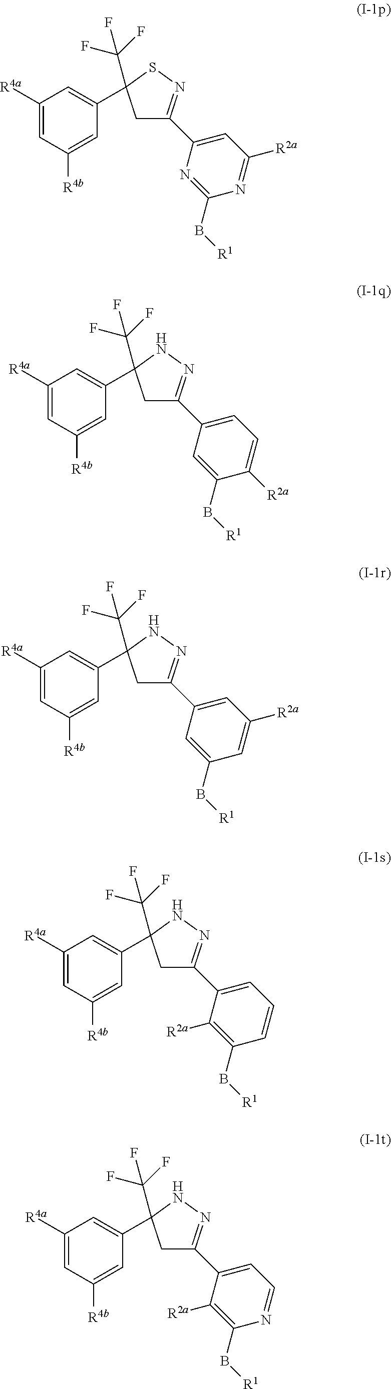

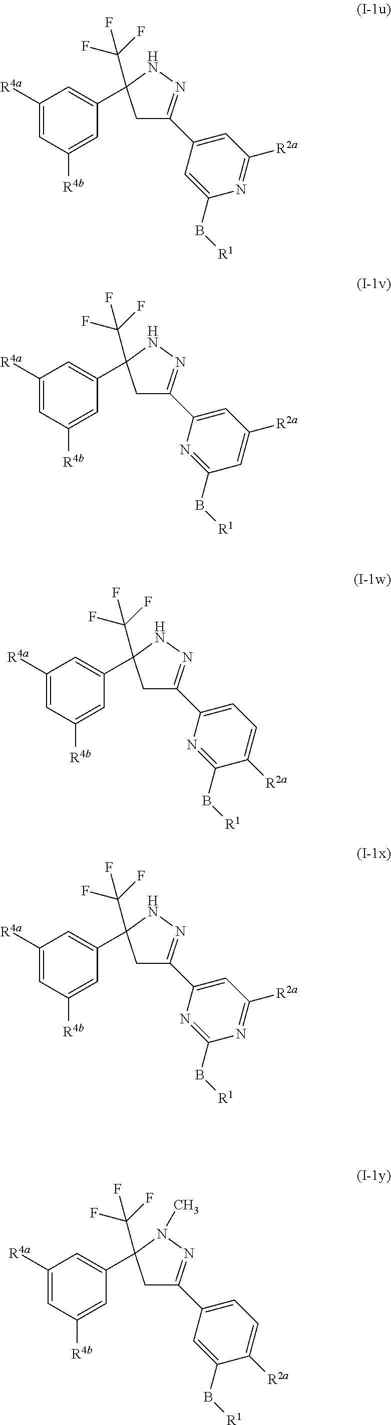

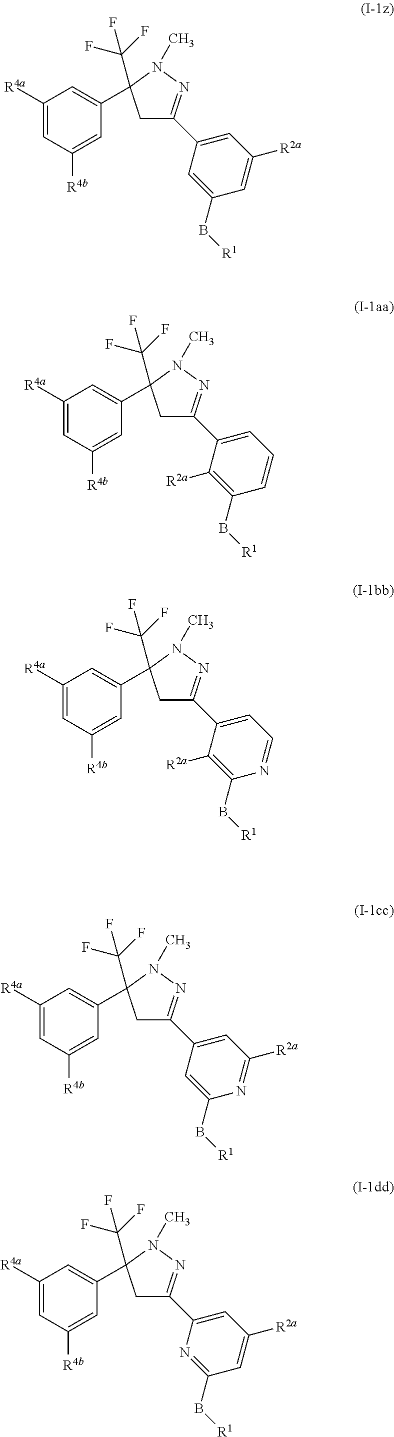

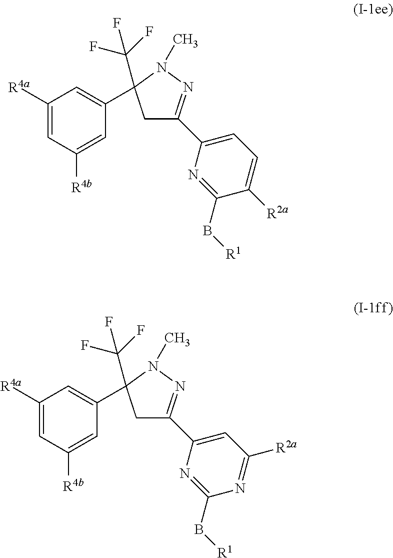

### Table 1

Compounds of the formula I-1a in which R^{4a} is fluorine, R^{4b} is H, R^{2a} is H, B is O and R¹ for a compound corresponds in each case to one row of Table A

### Table 2

Compounds of the formula I-1a in which R^{4a} is chlorine, R^{4b} is H, R^{2a} is H, B is O and R¹ for a compound corresponds in each case to one row of Table A

### Table 3

Compounds of the formula I-1a in which R^{4a} is bromine, R^{4b} is H, R^{2a} is H, B is O and R¹ for a compound corresponds in each case to one row of Table A

### Table 4

Compounds of the formula I-1a in which R^{4a} is methyl, R^{4b} is H, R^{2a} is H, B is O and R¹ for a compound corresponds in each case to one row of Table A

### Table 5

Compounds of the formula I-1a in which R^{4a} is trifluoromethyl, R^{4b} is H, R^{2a} is H, B is O and R¹ for a compound corresponds in each case to one row of Table A

### Table 6

Compounds of the formula I-1a in which R^{4a} and R^{4b} are fluorine, R^{2a} is H, B is O and R¹ for a compound corresponds in each case to one row of Table A

### Table 7

Compounds of the formula I-1a in which R^{4a} and R^{4b} are chlorine, R^{2a} is H, B is O and R¹ for a compound corresponds in each case to one row of Table A

### Table 8

Compounds of the formula I-1a in which R^{4a} and R^{4b} are bromine, R^{2a} is H, B is O and R¹ for a compound corresponds in each case to one row of Table A

### Table 9

Compounds of the formula I-1a in which R^{4a} and R^{4b} are methyl, R^{2a} is H, B is O and R¹ for a compound corresponds in each case to one row of Table A

### Table 10

Compounds of the formula I-1a in which R^{4a} and R^{4b} are trifluoromethyl, R^{2a} is H, B is O and R¹ for a compound corresponds in each case to one row of Table A

### Tables 11 to 20

Compounds of the formula I-1a in which the combination of R^{4a} and R^{4b} is as defined in any of Tables 1 to 10, B is O, R¹ for a compound corresponds in each case to one row of Table A and R^{2a} is fluorine

### Tables 21 to 30

Compounds of the formula I-1a in which the combination of R^{4a} and R^{4b} is as defined in any of Tables 1 to 10, B is O, R¹ for a compound corresponds in each case to one row of Table A and R^{2a} is chlorine

### Tables 31 to 40

Compounds of the formula I-1a in which the combination of R^{4a} and R^{4b} is as defined in any of Tables 1 to 10, B is O, R¹ for a compound corresponds in each case to one row of Table A and R^{2a} is CN

### Tables 41 to 50

Compounds of the formula I-1a in which the combination of R^{4a} and R^{4b} is as defined in any of Tables 1 to 10, B is O, R¹ for a compound corresponds in each case to one row of Table A and R^{2a} is nitro

### Tables 51 to 60

Compounds of the formula I-1a in which the combination of R^{4a} and R^{4b} is as defined in any of Tables 1 to 10, B is O, R¹ for a compound corresponds in each case to one row of Table A and R^{2a} is methyl

### Tables 61 to 70

Compounds of the formula I-1a in which the combination of R^{4a} and R^{4b} is as defined in any of Tables 1 to 10, B is O, R¹ for a compound corresponds in each case to one row of Table A and R^{2a} is ethyl

### Tables 71 to 80

Compounds of the formula I-1a in which the combination of R^{4a} and R^{4b} is as defined in any of Tables 1 to 10, B is O, R¹ for a compound corresponds in each case to one row of Table A and R^{2a} is trifluoromethyl

### Tables 81 to 90

Compounds of the formula I-1a in which the combination of R^{4a} and R^{4b} is as defined in any of Tables 1 to 10, B is O, R¹ for a compound corresponds in each case to one row of Table A and R^{2a} is methoxy

### Tables 91 to 100

Compounds of the formula I-1a in which the combination of R^{4a} and R^{4b} is as defined in any of Tables 1 to 10, B is O, R¹ for a compound corresponds in each case to one row of Table A and R^{2a} is trifluoromethoxy

### Tables 101 to 200

Compounds of the formula I-1a in which the combination of R^{2a}, R^{4a} and R^{4b} is as defined in any of Tables 1 to 100, R¹ for a compound corresponds in each case to one row of Table A and B is S

### Tables 201 to 300

Compounds of the formula I-1a in which the combination of R^{2a}, R^{4a} and R^{4b} is as defined in any of Tables 1 to 100, R¹ for a compound corresponds in each case to one row of Table A and B is SO

### Tables 301 to 400

Compounds of the formula I-1a in which the combination of R^{2a}, R^{4a} and R^{4b} is as defined in any of Tables 1 to 100, R¹ for a compound corresponds in each case to one row of Table B and B is SO

### Tables 401 to 500

Compounds of the formula I-1a in which the combination of R^{2a}, R^{4a} and R^{4b} is as defined in any of Tables 1 to 100, R¹ for a compound corresponds in each case to one row of Table A and B is SO₂

### Tables 501 to 600

Compounds of the formula I-1a in which the combination of R^{2a}, R^{4a} and R^{4b} is as defined in any of Tables 1 to 100, R¹ for a compound corresponds in each case to one row of Table B and B is SO₂

### Tables 601 to 700

Compounds of the formula I-1a in which the combination of R^{2a}, R^{4a} and R^{4b} is as defined in any of Tables 1 to 100, R¹ for a compound corresponds in each case to one row of Table A and B is NH

### Tables 701 to 800

Compounds of the formula I-1a in which the combination of R^{2a}, R^{4a} and R^{4b} is as defined in any of Tables 1 to 100, R¹ for a compound corresponds in each case to one row of Table A and B is NCH₃

### Tables 801 to 900

Compounds of the formula I-1a in which the combination of R^{2a}, R^{4a} and R^{4b} is as defined in any of Tables 1 to 100, R¹ for a compound corresponds in each case to one row of Table A and B is NC(═O)CH₃

### Tables 901 to 1000

Compounds of the formula I-1a in which the combination of R^{2a}, R^{4a} and R^{4b} is as defined in any of Tables 1 to 100, R¹ for a compound corresponds in each case to one row of Table A and B is NC(═O)OCH₃

### Tables 1001 to 1100

Compounds of the formula I-1a in which the combination of R^{2a}, R^{4a} and R^{4b} is as defined in any of Tables 1 to 100, R¹ for a compound corresponds in each case to one row of Table A and B is CH₂

### Tables 1101 to 1200

Compounds of the formula I-1a in which the combination of R^{2a}, R^{4a} and R^{4b} is as defined in any of Tables 1 to 100, R¹ for a compound corresponds in each case to one row of Table B and B is CH₂

### Tables 1201 to 1300

Compounds of the formula I-1a in which the combination of R^{2a}, R^{4a} and R^{4b} is as defined in any of Tables 1 to 100, R¹ for a compound corresponds in each case to one row of Table C and B is CH₂

### Tables 1301 to 1400

Compounds of the formula I-1a in which the combination of R^{2a}, R^{4a} and R^{4b} is as defined in any of Tables 1 to 100, R¹ for a compound corresponds in each case to one row of Table A and B is CH(CH₃)

### Tables 1401 to 1500

Compounds of the formula I-1a in which the combination of R^{2a}, R^{4a} and R^{4b} is as defined in any of Tables 1 to 100, R¹ for a compound corresponds in each case to one row of Table B and B is CH(CH₃)

### Tables 1501 to 1600

Compounds of the formula I-1a in which the combination of R^{2a}, R^{4a} and R^{4b} is as defined in any of Tables 1 to 100, R¹ for a compound corresponds in each case to one row of Table C and B is CH(CH₃)

### Tables 1601 to 1700

Compounds of the formula I-1a in which the combination of R^{2a}, R^{4a} and R^{4b} is as defined in any of Tables 1 to 100, R¹ for a compound corresponds in each case to one row of Table A and B is C(CH₃)₂

### Tables 1701 to 1800

Compounds of the formula I-1a in which the combination of R^{2a}, R^{4a} and R^{4b} is as defined in any of Tables 1 to 100, R¹ for a compound corresponds in each case to one row of Table B and B is C(CH₃)₂

### Tables 1801 to 1900

Compounds of the formula I-1a in which the combination of R^{2a}, R^{4a} and R^{4b} is as defined in any of Tables 1 to 100, R¹ for a compound corresponds in each case to one row of Table C and B is C(CH₃)₂

### Tables 1901 to 2000

Compounds of the formula I-1a in which the combination of R^{2a}, R^{4a} and R^{4b} is as defined in any of Tables 1 to 100, R¹ for a compound corresponds in each case to one row of Table A and B is cyclopropan-1,1-diyl

### Tables 2001 to 2100

Compounds of the formula I-1a in which the combination of R^{2a}, R^{4a} and R^{4b} is as defined in any of Tables 1 to 100, R¹ for a compound corresponds in each case to one row of Table B and B is cyclopropan-1,1-diyl

### Tables 2101 to 2200

Compounds of the formula I-1a in which the combination of R^{2a}, R^{4a} and R^{4b} is as defined in any of Tables 1 to 100, R¹ for a compound corresponds in each case to one row of Table C and B is cyclopropan-1,1-diyl

### Tables 2201 to 2300

Compounds of the formula I-1a in which the combination of R^{2a}, R^{4a} and R^{4b} is as defined in any of Tables 1 to 100, R¹ for a compound corresponds in each case to one row of Table A and B is CO

### Tables 2301 to 2400

Compounds of the formula I-1a in which the combination of R^{2a}, R^{4a} and R^{4b} is as defined in any of Tables 1 to 100, R¹ is NHR^{11a}, wherein R^{11a} for a compound corresponds in each case to one row of Table A and B is CO

### Tables 2401 to 2500

Compounds of the formula I-1a in which the combination of R^{2a}, R^{4a} and R^{4b} is as defined in any of Tables 1 to 100, R¹ is N(CH₃)R^{11a}, wherein R^{11a} for a compound corresponds in each case to one row of Table A and B is CO

### Tables 2501 to 2600

Compounds of the formula I-1a in which the combination of R^{2a}, R^{4a} and R^{4b} is as defined in any of Tables 1 to 100, R¹ is N(COCH₃)R^{11a}, wherein R^{11a} for a compound corresponds in each case to one row of Table A and B is CO

### Tables 2601 to 2700

Compounds of the formula I-1a in which the combination of R^{2a}, R^{4a} and R^{4b} is as defined in any of Tables 1 to 100, R¹ for a compound corresponds in each case to one row of Table A and B is CS

### Tables 2701 to 2800

Compounds of the formula I-1a in which the combination of R^{2a}, R^{4a} and R^{4b} is as defined in any of Tables 1 to 100, R¹ is NHR^{11a}, wherein R^{11a} for a compound corresponds in each case to one row of Table A and B is CS

### Tables 2801 to 2900

Compounds of the formula I-1a in which the combination of R^{2a}, R^{4a} and R^{4b} is as defined in any of Tables 1 to 100, R¹ is N(CH₃)R^{11a}, wherein R^{11a} for a compound corresponds in each case to one row of Table A and B is CS

### Tables 2901 to 3000

Compounds of the formula I-1a in which the combination of R^{2a}, R^{4a} and R^{4b} is as defined in any of Tables 1 to 100, R¹ is N(COCH₃)R^{11a}, wherein R^{11a} for a compound corresponds in each case to one row of Table A and B is CS

### Tables 3001 to 3100

Compounds of the formula I-1a in which the combination of R^{2a}, R^{4a} and R^{4b} is as defined in any of Tables 1 to 100, R¹ for a compound corresponds in each case to one row of Table A and B is NHC(═O)

### Tables 3101 to 3200

Compounds of the formula I-1a in which the combination of R^{2a}, R^{4a} and R^{4b} is as defined in any of Tables 1 to 100, R¹ for a compound corresponds in each case to one row of Table A and B is N(CH₃)C(═O)

### Tables 3201 to 3300

Compounds of the formula I-1a in which the combination of R^{2a}, R^{4a} and R^{4b} is as defined in any of Tables 1 to 100, R¹ for a compound corresponds in each case to one row of Table A and B is N(COCH₃)C(═O)

### Tables 3301 to 3400

Compounds of the formula I-1a in which the combination of R^{2a}, R^{4a} and R^{4b} is as defined in any of Tables 1 to 100, R¹ is H, B is C═N—NHR^{11a} and R^{11a} for a compound corresponds in each case to one row of Table A

### Tables 3401 to 3500

Compounds of the formula I-1a in which the combination of R^{2a}, R^{4a} and R^{4b} is as defined in any of Tables 1 to 100, R¹ is H, B is C═N—N(CH₃)R^{11a} and R^{11a} for a compound corresponds in each case to one row of Table A

### Tables 3501 to 3600

Compounds of the formula I-1a in which the combination of R^{2a}, R^{4a} and R^{4b} is as defined in any of Tables 1 to 100, R¹ is CH₃, B is C═N—NHR^{11a} and R^{11a} for a compound corresponds in each case to one row of Table A

### Tables 3601 to 3700

Compounds of the formula I-1a in which the combination of R^{2a}, R^{4a} and R^{4b} is as defined in any of Tables 1 to 100, R¹ is CH₃, B is C═N—N(CH₃)R^{11a} and R^{11a} for a compound corresponds in each case to one row of Table A

### Tables 3701 to 3800

Compounds of the formula I-1a in which the combination of R^{2a}, R^{4a} and R^{4b} is as defined in any of Tables 1 to 100, R¹ is H, B is C═N—OR¹⁰ and R¹⁰ for a compound corresponds in each case to one row of Table A

### Tables 3801 to 3900

Compounds of the formula I-1a in which the combination of R^{2a}, R^{4a} and R^{4b} is as defined in any of Tables 1 to 100, R¹ is CH₃, B is C═N—OR¹⁰ and R¹⁰ for a compound corresponds in each case to one row of Table A

### Tables 3901 to 4000

Compounds of the formula I-1a in which the combination of R^{2a}, R^{4a} and R^{4b} is as defined in any of Tables 1 to 100 and the combination of B and R¹ for a compound corresponds in each case to one row of Table D

### Tables 4001 to 8000

Compounds of the formula I-1b in which the combination of R^{2a}, R^{4a}, R^{4b} B and R¹ is as defined in any of Tables 1 to 4000

### Tables 8001 to 12000

Compounds of the formula I-1c in which the combination of R^{2a}, R^{4a}, R^{4b} B and R¹ is as defined in any of Tables 1 to 4000

### Tables 12001 to 16000

Compounds of the formula I-1d in which the combination of R^{2a}, R^{4a}, R^{4b} B and R¹ is as defined in any of Tables 1 to 4000

### Tables 16001 to 20000

Compounds of the formula I-1e in which the combination of R^{2a}, R^{4a}, R^{4b} B and R¹ is as defined in any of Tables 1 to 4000

### Tables 20001 to 24000

Compounds of the formula I-1f in which the combination of R^{2a}, R^{4a}, R^{4b} B and R¹ is as defined in any of Tables 1 to 4000

### Tables 24001 to 28000

Compounds of the formula I-1g in which the combination of R^{2a}, R^{4a}, R^{4b} B and R¹ is as defined in any of Tables 1 to 4000

### Tables 28001 to 32000

Compounds of the formula I-1h in which the combination of R^{2a}, R^{4a}, R^{4b} B and R¹ is as defined in any of Tables 1 to 4000

### Tables 32001 to 36000

Compounds of the formula I-1i in which the combination of R^{2a}, R^{4a}, R^{4b} B and R¹ is as defined in any of Tables 1 to 4000

### Tables 36001 to 40000

Compounds of the formula I-1j in which the combination of R^{2a}, R^{4a}, R^{4b} B and R¹ is as defined in any of Tables 1 to 4000

### Tables 40001 to 44000

Compounds of the formula I-1k in which the combination of R^{2a}, R^{4a}, R^{4b} B and R¹ is as defined in any of Tables 1 to 4000

### Tables 44001 to 48000

Compounds of the formula I-1l in which the combination of R^{2a}, R^{4a}, R^{4b} B and R¹ is as defined in any of Tables 1 to 4000

### Tables 48001 to 52000

Compounds of the formula I-1m in which the combination of R^{2a}, R^{4a}, R^{4b} B and R¹ is as defined in any of Tables 1 to 4000

### Tables 52001 to 56000

Compounds of the formula I-1n in which the combination of R^{2a}, R^{4a}, R^{4b} B and R¹ is as defined in any of Tables 1 to 4000

### Tables 56001 to 60000

Compounds of the formula I-1o in which the combination of R^{2a}, R^{4a}, R^{4b} B and R¹ is as defined in any of Tables 1 to 4000

### Tables 60001 to 64000

Compounds of the formula I-1p in which the combination of R^{2a}, R^{4a}, R^{4b} B and R¹ is as defined in any of Tables 1 to 4000

### Tables 64001 to 68000

Compounds of the formula I-1q in which the combination of R^{2a}, R^{4a}, R^{4b} B and R¹ is as defined in any of Tables 1 to 4000

### Tables 68001 to 72000

Compounds of the formula I-1r in which the combination of R^{2a}, R^{4a}, R^{4b} B and R¹ is as defined in any of Tables 1 to 4000

### Tables 72001 to 76000

Compounds of the formula I-1s in which the combination of R^{2a}, R^{4a}, R^{4b} B and R¹ is as defined in any of Tables 1 to 4000

### Tables 76001 to 80000

Compounds of the formula I-1t in which the combination of R^{2a}, R^{4a}, R^{4b} B and R¹ is as defined in any of Tables 1 to 4000

### Tables 80001 to 84000

Compounds of the formula I-1u in which the combination of R^{2a}, R^{4a}, R^{4b} B and R¹ is as defined in any of Tables 1 to 4000

### Tables 84001 to 88000

Compounds of the formula I-1v in which the combination of R^{2a}, R^{4a}, R^{4b} B and R¹ is as defined in any of Tables 1 to 4000

### Tables 88001 to 92000

Compounds of the formula I-1w in which the combination of R^{2a}, R^{4a}, R^{4b} B and R¹ is as defined in any of Tables 1 to 4000

### Tables 92001 to 96000

Compounds of the formula I-1x in which the combination of R^{2a}, R^{4a}, R^{4b} B and R¹ is as defined in any of Tables 1 to 4000

### Tables 96001 to 100000

Compounds of the formula I-1y in which the combination of R^{2a}, R^{4a}, R^{4b} B and R¹ is as defined in any of Tables 1 to 4000

### Tables 100001 to 104000

Compounds of the formula I-1z in which the combination of R^{2a}, R^{4a}, R^{4b} B and R¹ is as defined in any of Tables 1 to 4000

### Tables 104001 to 108000

Compounds of the formula I-1aa in which the combination of R^{2a}, R^{4a}, R^{4b} B and R¹ is as defined in any of Tables 1 to 4000

### Tables 108001 to 112000

Compounds of the formula I-1bb in which the combination of R^{2a}, R^{4a}, R^{4b} B and R¹ is as defined in any of Tables 1 to 4000

### Tables 112001 to 116000

Compounds of the formula I-1cc in which the combination of R^{2a}, R^{4a}, R^{4b} B and R¹ is as defined in any of Tables 1 to 4000

### Tables 116001 to 120000

Compounds of the formula I-1dd in which the combination of R^{2a}, R^{4a}, R^{4b} B and R¹ is as defined in any of Tables 1 to 4000

### Tables 120001 to 124000

Compounds of the formula I-1ee in which the combination of R^{2a}, R^{4a}, R^{4b} B and R¹ is as defined in any of Tables 1 to 4000

### Tables 124001 to 128000

Compounds of the formula I-1ff in which the combination of R^{2a}, R^{4a}, R^{4b} B and R¹ is as defined in any of Tables 1 to 4000

TABLE A

| No. | R¹/R¹⁰/R^{11a} |
|---|---|
| A-1. | methyl |
| A-2. | ethyl |
| A-3. | propyl |
| A-4. | isopropyl |
| A-5. | n-butyl |
| A-6. | sec-butyl |
| A-7. | isobutyl |
| A-8. | tert-butyl |
| A-9. | pentyl |
| A-10. | hexyl |
| A-11. | CHF₂ |
| A-12. | CF₃ |
| A-13. | CH₂CH₂CF₃ |
| A-14. | CH₂CF₂CF₃ |
| A-15. | (CH₂)₃CF₃ |
| A-16. | CH₂-^{c}propyl |
| A-17. | CH(CH₃)-^{c}propyl |
| A-18. | CH₂CN |
| A-19. | CH₂CH₂CN |
| A-20. | CH₂SH |
| A-21. | CH₂SCH₃ |
| A-22. | CH₂SCH₂CH₃ |
| A-23. | CH₂SCF₃ |
| A-24. | CH₂SCH₂CF₃ |
| A-25. | CH₂SCF₂CF₃ |
| A-26. | CH₂CH₂SH |
| A-27. | CH₂CH₂SCH₃ |
| A-28. | CH₂CH₂SCH₂CH₃ |
| A-29. | CH₂CH₂SCF₃ |
| A-30. | CH₂CH₂SCH₂CF₃ |
| A-31. | CH₂CH₂SCF₂CF₃ |
| A-32. | CH₂CH₂CH₂SH |
| A-33. | CH₂CH₂CH₂SCH₃ |
| A-34. | CH₂CH₂CH₂SCH₂CH₃ |
| A-35. | CH₂CH₂CH₂SCF₃ |
| A-36. | CH₂CH₂CH₂SCH₂CF₃ |
| A-37. | CH₂CH₂CH₂SCF₂CF₃ |
| A-38. | CH₂OH |
| A-39. | CH₂OCH₃ |
| A-40. | CH₂OCH₂CH₃ |
| A-41. | CH₂OCF₃ |
| A-42. | CH₂OCH₂CF₃ |
| A-43. | CH₂OCF₂CF₃ |
| A-44. | CH₂CH₂OH |
| A-45. | CH₂CH₂OCH₃ |
| A-46. | CH₂CH₂OCH₂CH₃ |
| A-47. | CH₂CH₂OCF₃ |
| A-48. | CH₂CH₂OCH₂CF₃ |
| A-49. | CH₂CH₂OCF₂CF₃ |
| A-50. | CH₂CH₂CH₂OH |
| A-51. | CH₂CH₂CH₂OCH₃ |
| A-52. | CH₂CH₂CH₂OCH₂CH₃ |
| A-53. | CH₂CH₂CH₂OCF₃ |
| A-54. | CH₂CH₂CH₂OCH₂CF₃ |
| A-55. | CH₂CH₂CH₂OCF₂CF₃ |
| A-56. | cyclopropyl |
| A-57. | 2-fluorocyclopropyl |
| A-58. | 2,2-difluorocyclopropyl |
| A-59. | cyclopentyl |
| A-60. | cyclohexyl |
| A-61. | phenyl |
| A-62. | 2-fluorophenyl |
| A-63. | 3-fluorophenyl |
| A-64. | 4-fluorophenyl |
| A-65. | 2,3-difluorophenyl |
| A-66. | 2,4-difluorophenyl |
| A-67. | 2,5-difluorophenyl |
| A-68. | 2,6-difluorophenyl |
| A-69. | 3,4-difluorophenyl |
| A-70. | 3,5-difluorophenyl |
| A-71. | 2-chlorophenyl |
| A-72. | 3-chlorophenyl |
| A-73. | 4-chlorophenyl |
| A-74. | 2,3-dichlorophenyl |
| A-75. | 2,4-dichlorophenyl |
| A-76. | 2,5-dichlorophenyl |
| A-77. | 2,6-dichlorophenyl |
| A-78. | 3,4-dichlorophenyl |
| A-79. | 3,5-dichlorophenyl |
| A-80. | 2-bromophenyl |
| A-81. | 3-bromophenyl |
| A-82. | 4-bromophenyl |
| A-83. | 2-cyanophenyl |
| A-84. | 3-cyanophenyl |
| A-85. | 4-cyanophenyl |
| A-86. | 2-nitrophenyl |
| A-87. | 3-nitrophenyl |
| A-88. | 4-nitrophenyl |
| A-89. | 2-methylphenyl |
| A-90. | 3-methylphenyl |
| A-91. | 4-methylphenyl |
| A-92. | 2,3-dimethylphenyl |
| A-93. | 2,4-dimethylphenyl |
| A-94. | 2,5-dimethylphenyl |
| A-95. | 2,6-dimethylphenyl |
| A-96. | 3,4-dimethylphenyl |
| A-97. | 3,5-dimethylphenyl |
| A-98. | 2-trifluoromethylphenyl |
| A-99. | 3-trifluoromethylphenyl |
| A-100. | 4-trifluoromethylphenyl |
| A-101. | 2,3-di-(trifluoromethyl)-phenyl |
| A-102. | 2,4-di-(trifluoromethyl)-phenyl |
| A-103. | 2,5-di-(trifluoromethyl)-phenyl |
| A-104. | 2,6-di-(trifluoromethyl)-phenyl |
| A-105. | 3,4-di-(trifluoromethyl)-phenyl |
| A-106. | 3,5-di-(trifluoromethyl)-phenyl |
| A-107. | 2-methoxyphenyl |
| A-108. | 3-methoxyphenyl |
| A-109. | 4-methoxyphenyl |
| A-110. | 2,3-dimethoxyphenyl |
| A-111. | 2,4-dimethoxyphenyl |
| A-112. | 2,5-dimethoxyphenyl |
| A-113. | 2,6-dimethoxyphenyl |
| A-114. | 3,4-dimethoxyphenyl |
| A-115. | 3,5-dimethoxyphenyl |
| A-116. | 2-trifluoromethoxyphenyl |
| A-117. | 3-trifluoromethoxyphenyl |
| A-118. | 4-trifluoromethoxyphenyl |
| A-119. | 2,3-di-(trifluoromethoxy)-phenyl |
| A-120. | 2,4-di-(trifluoromethoxy)-phenyl |
| A-121. | 2,5-di-(trifluoromethoxy)-phenyl |
| A-122. | 2,6-di-(trifluoromethoxy)-phenyl |
| A-123. | 3,4-di-(trifluoromethoxy)-phenyl |
| A-124. | 3,5-di-(trifluoromethoxy)-phenyl |
| A-125. | benzyl |
| A-126. | 2-fluorobenzyl |
| A-127. | 3-fluorobenzyl |
| A-128. | 4-fluorobenzyl |
| A-129. | 2,3-difluorobenzyl |
| A-130. | 2,4-difluorobenzyl |
| A-131. | 2,5-difluorobenzyl |
| A-132. | 2,6-difluorobenzyl |
| A-133. | 3,4-difluorobenzyl |
| A-134. | 3,5-difluorobenzyl |
| A-135. | 2-chlorobenzyl |
| A-136. | 3-chlorobenzyl |
| A-137. | 4-chlorobenzyl |
| A-138. | 2,3-dichlorobenzyl |
| A-139. | 2,4-dichlorobenzyl |
| A-140. | 2,5-dichlorobenzyl |
| A-141. | 2,6-dichlorobenzyl |
| A-142. | 3,4-dichlorobenzyl |
| A-143. | 3,5-dichlorobenzyl |
| A-144. | 2-bromobenzyl |
| A-145. | 3-bromobenzyl |
| A-146. | 4-bromobenzyl |
| A-147. | 2-cyanobenzyl |
| A-148. | 3-cyanobenzyl |
| A-149. | 4-cyanobenzyl |
| A-150. | 2-nitrobenzyl |
| A-151. | 3-nitrobenzyl |
| A-152. | 4-nitrobenzyl |
| A-153. | 2-methylbenzyl |
| A-154. | 3-methylbenzyl |
| A-155. | 4-methylbenzyl |
| A-156. | 2,3-dimethylbenzyl |
| A-157. | 2,4-dimethylbenzyl |
| A-158. | 2,5-dimethylbenzyl |
| A-159. | 2,6-dimethylbenzyl |
| A-160. | 3,4-dimethylbenzyl |
| A-161. | 3,5-dimethylbenzyl |
| A-162. | 2-trifluoromethylbenzyl |
| A-163. | 3-trifluoromethylbenzyl |
| A-164. | 4-trifluoromethylbenzyl |
| A-165. | 2,3-di-(trifluoromethyl)-benzyl |
| A-166. | 2,4-di-(trifluoromethyl)-benzyl |
| A-167. | 2,5-di-(trifluoromethyl)-benzyl |
| A-168. | 2,6-di-(trifluoromethyl)-benzyl |
| A-169. | 3,4-di-(trifluoromethyl)-benzyl |
| A-170. | 3,5-di-(trifluoromethyl)-benzyl |
| A-171. | 2-methoxybenzyl |
| A-172. | 3-methoxybenzyl |
| A-173. | 4-methoxybenzyl |
| A-174. | 2,3-dimethoxybenzyl |
| A-175. | 2,4-dimethoxybenzyl |
| A-176. | 2,5-dimethoxybenzyl |
| A-177. | 2,6-dimethoxybenzyl |
| A-178. | 3,4-dimethoxybenzyl |
| A-179. | 3,5-dimethoxybenzyl |
| A-180. | 2-trifluoromethoxybenzyl |
| A-181. | 3-trifluoromethoxybenzyl |
| A-182. | 4-trifluoromethoxybenzyl |
| A-183. | 2,3-di-(trifluoromethoxy)-benzyl |
| A-184. | 2,4-di-(trifluoromethoxy)-benzyl |
| A-185. | 2,5-di-(trifluoromethoxy)-benzyl |
| A-186. | 2,6-di-(trifluoromethoxy)-benzyl |
| A-187. | 3,4-di-(trifluoromethoxy)-benzyl |
| A-188. | 3,5-di-(trifluoromethoxy)-benzyl |
| A-189. | pyrrolidin-1-yl |
| A-190. | pyrrolidin-2-yl |
| A-191. | pyrrolidin-3-yl |
| A-192. | 1-methyl-pyrrolidin-2-yl |
| A-193. | 1-methyl-pyrrolidin-3-yl |
| A-194. | pyrrolidin-2-on-1-yl |
| A-195. | pyrrolidin-2-on-3-yl |
| A-196. | pyrrolidin-2-on-4-yl |
| A-197. | pyrrolidin-2-on-5-yl |
| A-198. | 1-methyl-pyrrolidin-2-on-3-yl |
| A-199. | 1-methyl-pyrrolidin-2-on-4-yl |
| A-200. | 1-methyl-pyrrolidin-2-on-5-yl |
| A-201. | 2-chloro-pyrrolidin-1-yl |
| A-202. | 3-chloro-pyrrolidin-1-yl |
| A-203. | 2-bromo-pyrrolidin-1-yl |
| A-204. | 3-bromo-pyrrolidin-1-yl |
| A-205. | 2-cyano-pyrrolidin-1-yl |
| A-206. | 3-cyano-pyrrolidin-1-yl |
| A-207. | 2-nitro-pyrrolidin-1-yl |
| A-208. | 3-nitro-pyrrolidin-1-yl |
| A-209. | 2-methyl-pyrrolidin-1-yl |
| A-210. | 3-methyl-pyrrolidin-1-yl |
| A-211. | 2-trifluoromethyl-pyrrolidin-1-yl |
| A-212. | 3-trifluoromethyl-pyrrolidin-1-yl |
| A-213. | 2-methoxy-pyrrolidin-1-yl |
| A-214. | 3-methoxy-pyrrolidin-1-yl |
| A-215. | 2-trifluoromethoxy-pyrrolidin-1-yl |
| A-216. | 3-trifluoromethoxy-pyrrolidin-1-yl |
| A-217. | 3-chloro-pyrrolidin-2-yl |
| A-218. | 4-chloro-pyrrolidin-2-yl |
| A-219. | 5-chloro-pyrrolidin-2-yl |
| A-220. | 3-bromo-pyrrolidin-2-yl |
| A-221. | 4-bromo-pyrrolidin-2-yl |
| A-222. | 5-bromo-pyrrolidin-2-yl |
| A-223. | 3-cyano-pyrrolidin-2-yl |
| A-224. | 4-cyano-pyrrolidin-2-yl |
| A-225. | 5-cyano-pyrrolidin-2-yl |
| A-226. | 3-nitro-pyrrolidin-2-yl |
| A-227. | 4-nitro-pyrrolidin-2-yl |
| A-228. | 5-nitro-pyrrolidin-2-yl |
| A-229. | 3-methyl-pyrrolidin-2-yl |
| A-230. | 4-methyl-pyrrolidin-2-yl |
| A-231. | 5-methyl-pyrrolidin-2-yl |
| A-232. | 3-trifluoromethyl-pyrrolidin-2-yl |
| A-233. | 4-trifluoromethyl-pyrrolidin-2-yl |
| A-234. | 5-trifluoromethyl-pyrrolidin-2-yl |
| A-235. | 3-methoxy-pyrrolidin-2-yl |
| A-236. | 4-methoxy-pyrrolidin-2-yl |
| A-237. | 5-methoxy-pyrrolidin-2-yl |
| A-238. | 3-trifluoromethoxy-pyrrolidin-2-yl |
| A-239. | 4-trifluoromethoxy-pyrrolidin-2-yl |
| A-240. | 5-trifluoromethoxy-pyrrolidin-2-yl |
| A-241. | 2-chloro-pyrrolidin-3-yl |
| A-242. | 4-chloro-pyrrolidin-3-yl |
| A-243. | 5-chloro-pyrrolidin-3-yl |
| A-244. | 2-bromo-pyrrolidin-3-yl |
| A-245. | 4-bromo-pyrrolidin-3-yl |
| A-246. | 5-bromo-pyrrolidin-3-yl |
| A-247. | 2-cyano-pyrrolidin-3-yl |
| A-248. | 4-cyano-pyrrolidin-3-yl |
| A-249. | 5-cyano-pyrrolidin-3-yl |
| A-250. | 2-nitro-pyrrolidin-3-yl |
| A-251. | 4-nitro-pyrrolidin-3-yl |
| A-252. | 5-nitro-pyrrolidin-3-yl |
| A-253. | 2-methyl-pyrrolidin-3-yl |
| A-254. | 4-methyl-pyrrolidin-3-yl |
| A-255. | 5-methyl-pyrrolidin-3-yl |
| A-256. | 2-trifluoromethyl-pyrrolidin-3-yl |
| A-257. | 4-trifluoromethyl-pyrrolidin-3-yl |
| A-258. | 5-trifluoromethyl-pyrrolidin-3-yl |
| A-259. | 2-methoxy-pyrrolidin-3-yl |
| A-260. | 4-methoxy-pyrrolidin-3-yl |
| A-261. | 5-methoxy-pyrrolidin-3-yl |
| A-262. | 2-trifluoromethoxy-pyrrolidin-3-yl |
| A-263. | 4-trifluoromethoxy-pyrrolidin-3-yl |
| A-264. | 5-trifluoromethoxy-pyrrolidin-3-yl |
| A-265. | 3-chloro-1-methyl-pyrrolidin-2-yl |
| A-266. | 4-chloro-1-methyl-pyrrolidin-2-yl |
| A-267. | 5-chloro-1-methyl-pyrrolidin-2-yl |
| A-268. | 3-bromo-1-methyl-pyrrolidin-2-yl |
| A-269. | 4-bromo-1-methyl-pyrrolidin-2-yl |
| A-270. | 5-bromo-1-methyl-pyrrolidin-2-yl |
| A-271. | 3-cyano-1-methyl-pyrrolidin-2-yl |
| A-272. | 4-cyano-1-methyl-pyrrolidin-2-yl |
| A-273. | 5-cyano-1-methyl-pyrrolidin-2-yl |
| A-274. | 3-nitro-1-methyl-pyrrolidin-2-yl |
| A-275. | 4-nitro-1-methyl-pyrrolidin-2-yl |
| A-276. | 5-nitro-1-methyl-pyrrolidin-2-yl |
| A-277. | 3-methyl-1-methyl-pyrrolidin-2-yl |
| A-278. | 4-methyl-1-methyl-pyrrolidin-2-yl |
| A-279. | 5-methyl-1-methyl-pyrrolidin-2-yl |
| A-280. | 3-trifluoromethyl-1-methyl-pyrrolidin-2-yl |
| A-281. | 4-trifluoromethyl-1-methyl-pyrrolidin-2-yl |
| A-282. | 5-trifluoromethyl-1-methyl-pyrrolidin-2-yl |
| A-283. | 3-methoxy-1-methyl-pyrrolidin-2-yl |
| A-284. | 4-methoxy-1-methyl-pyrrolidin-2-yl |
| A-285. | 5-methoxy-1-methyl-pyrrolidin-2-yl |
| A-286. | 3-trifluoromethoxy-1-methyl-pyrrolidin-2-yl |
| A-287. | 4-trifluoromethoxy-1-methyl-pyrrolidin-2-yl |
| A-288. | 5-trifluoromethoxy-1-methyl-pyrrolidin-2-yl |
| A-289. | 2-chloro-1-methyl-pyrrolidin-3-yl |
| A-290. | 4-chloro-1-methyl-pyrrolidin-3-yl |
| A-291. | 5-chloro-1-methyl-pyrrolidin-3-yl |
| A-292. | 2-bromo-1-methyl-pyrrolidin-3-yl |
| A-293. | 4-bromo-1-methyl-pyrrolidin-3-yl |
| A-294. | 5-bromo-1-methyl-pyrrolidin-3-yl |
| A-295. | 2-cyano-1-methyl-pyrrolidin-3-yl |
| A-296. | 4-cyano-1-methyl-pyrrolidin-3-yl |
| A-297. | 5-cyano-1-methyl-pyrrolidin-3-yl |
| A-298. | 2-nitro-1-methyl-pyrrolidin-3-yl |
| A-299. | 4-nitro-1-methyl-pyrrolidin-3-yl |
| A-300. | 5-nitro-1-methyl-pyrrolidin-3-yl |
| A-301. | 2-methyl-1-methyl-pyrrolidin-3-yl |
| A-302. | 4-methyl-1-methyl-pyrrolidin-3-yl |
| A-303. | 5-methyl-1-methyl-pyrrolidin-3-yl |
| A-304. | 2-trifluoromethyl-1-methyl-pyrrolidin-3-yl |
| A-305. | 4-trifluoromethyl-1-methyl-pyrrolidin-3-yl |
| A-306. | 5-trifluoromethyl-1-methyl-pyrrolidin-3-yl |
| A-307. | 2-methoxy-1-methyl-pyrrolidin-3-yl |
| A-308. | 4-methoxy-1-methyl-pyrrolidin-3-yl |
| A-309. | 5-methoxy-1-methyl-pyrrolidin-3-yl |
| A-310. | 2-trifluoromethoxy-1-methyl-pyrrolidin-3-yl |
| A-311. | 4-trifluoromethoxy-1-methyl-pyrrolidin-3-yl |
| A-312. | 5-trifluoromethoxy-1-methyl-pyrrolidin-3-yl |
| A-313. | 3-chloro-pyrrolidin-2-on-1-yl |
| A-314. | 4-chloro-pyrrolidin-2-on-1-yl |
| A-315. | 5-chloro-pyrrolidin-2-on-1-yl |
| A-316. | 3-bromo-pyrrolidin-2-on-1-yl |
| A-317. | 4-bromo-pyrrolidin-2-on-1-yl |
| A-318. | 5-bromo-pyrrolidin-2-on-1-yl |
| A-319. | 3-cyano-pyrrolidin-2-on-1-yl |
| A-320. | 4-cyano-pyrrolidin-2-on-1-yl |
| A-321. | 5-cyano-pyrrolidin-2-on-1-yl |
| A-322. | 3-nitro-pyrrolidin-2-on-1-yl |
| A-323. | 4-nitro-pyrrolidin-2-on-1-yl |
| A-324. | 5-nitro-pyrrolidin-2-on-1-yl |
| A-325. | 3-methyl-pyrrolidin-2-on-1-yl |
| A-326. | 4-methyl-pyrrolidin-2-on-1-yl |
| A-327. | 5-methyl-pyrrolidin-2-on-1-yl |
| A-328. | 3-trifluoromethyl-pyrrolidin-2-on-1-yl |
| A-329. | 4-trifluoromethyl-pyrrolidin-2-on-1-yl |
| A-330. | 5-trifluoromethyl-pyrrolidin-2-on-1-yl |
| A-331. | 3-methoxy-pyrrolidin-2-on-1-yl |
| A-332. | 4-methoxy-pyrrolidin-2-on-1-yl |
| A-333. | 5-methoxy-pyrrolidin-2-on-1-yl |
| A-334. | 3-trifluoromethoxy-pyrrolidin-2-on-1-yl |
| A-335. | 4-trifluoromethoxy-pyrrolidin-2-on-1-yl |
| A-336. | 5-trifluoromethoxy-pyrrolidin-2-on-1-yl |
| A-337. | 4-chloro-pyrrolidin-2-on-3-yl |
| A-338. | 5-chloro-pyrrolidin-2-on-3-yl |
| A-339. | 4-bromo-pyrrolidin-2-on-3-yl |
| A-340. | 5-bromo-pyrrolidin-2-on-3-yl |
| A-341. | 4-cyano-pyrrolidin-2-on-3-yl |
| A-342. | 5-cyano-pyrrolidin-2-on-3-yl |
| A-343. | 4-nitro-pyrrolidin-2-on-3-yl |
| A-344. | 5-nitro-pyrrolidin-2-on-3-yl |
| A-345. | 4-methyl-pyrrolidin-2-on-3-yl |
| A-346. | 5-methyl-pyrrolidin-2-on-3-yl |
| A-347. | 4-trifluoromethyl-pyrrolidin-2-on-3-yl |
| A-348. | 5-trifluoromethyl-pyrrolidin-2-on-3-yl |
| A-349. | 4-methoxy-pyrrolidin-2-on-3-yl |
| A-350. | 5-methoxy-pyrrolidin-2-on-3-yl |
| A-351. | 4-trifluoromethoxy-pyrrolidin-2-on-3-yl |
| A-352. | 5-trifluoromethoxy-pyrrolidin-2-on-3-yl |
| A-353. | 3-chloro-pyrrolidin-2-on-4-yl |
| A-354. | 5-chloro-pyrrolidin-2-on-4-yl |
| A-355. | 3-bromo-pyrrolidin-2-on-4-yl |
| A-356. | 5-bromo-pyrrolidin-2-on-4-yl |
| A-357. | 3-cyano-pyrrolidin-2-on-4-yl |
| A-358. | 5-cyano-pyrrolidin-2-on-4-yl |
| A-359. | 3-nitro-pyrrolidin-2-on-4-yl |
| A-360. | 5-nitro-pyrrolidin-2-on-4-yl |
| A-361. | 3-methyl-pyrrolidin-2-on-4-yl |
| A-362. | 5-methyl-pyrrolidin-2-on-4-yl |
| A-363. | 3-trifluoromethyl-pyrrolidin-2-on-4-yl |
| A-364. | 5-trifluoromethyl-pyrrolidin-2-on-4-yl |
| A-365. | 3-methoxy-pyrrolidin-2-on-4-yl |
| A-366. | 5-methoxy-pyrrolidin-2-on-4-yl |
| A-367. | 3-trifluoromethoxy-pyrrolidin-2-on-4-yl |
| A-368. | 5-trifluoromethoxy-pyrrolidin-2-on-4-yl |
| A-369. | 3-chloro-pyrrolidin-2-on-5-yl |
| A-370. | 4-chloro-pyrrolidin-2-on-5-yl |
| A-371. | 3-bromo-pyrrolidin-2-on-5-yl |
| A-372. | 4-bromo-pyrrolidin-2-on-5-yl |
| A-373. | 3-cyano-pyrrolidin-2-on-5-yl |
| A-374. | 4-cyano-pyrrolidin-2-on-5-yl |
| A-375. | 3-nitro-pyrrolidin-2-on-5-yl |
| A-376. | 4-nitro-pyrrolidin-2-on-5-yl |
| A-377. | 3-methyl-pyrrolidin-2-on-5-yl |
| A-378. | 4-methyl-pyrrolidin-2-on-5-yl |
| A-379. | 3-trifluoromethyl-pyrrolidin-2-on-5-yl |
| A-380. | 4-trifluoromethyl-pyrrolidin-2-on-5-yl |
| A-381. | 3-methoxy-pyrrolidin-2-on-5-yl |
| A-382. | 4-methoxy-pyrrolidin-2-on-5-yl |
| A-383. | 3-trifluoromethoxy-pyrrolidin-2-on-5-yl |
| A-384. | 4-trifluoromethoxy-pyrrolidin-2-on-5-yl |
| A-385. | 4-chloro-1-methyl-pyrrolidin-2-on-3-yl |
| A-386. | 5-chloro-1-methyl-pyrrolidin-2-on-3-yl |
| A-387. | 4-bromo-1-methyl-pyrrolidin-2-on-3-yl |
| A-388. | 5-bromo-1-methyl-pyrrolidin-2-on-3-yl |
| A-389. | 4-cyano-1-methyl-pyrrolidin-2-on-3-yl |
| A-390. | 5-cyano-1-methyl-pyrrolidin-2-on-3-yl |
| A-391. | 4-nitro-1-methyl-pyrrolidin-2-on-3-yl |
| A-392. | 5-nitro-1-methyl-pyrrolidin-2-on-3-yl |
| A-393. | 4-methyl-1-methyl-pyrrolidin-2-on-3-yl |
| A-394. | 5-methyl-1-methyl-pyrrolidin-2-on-3-yl |
| A-395. | 4-trifluoromethyl-1-methyl-pyrrolidin-2-on-3-yl |
| A-396. | 5-trifluoromethyl-1-methyl-pyrrolidin-2-on-3-yl |
| A-397. | 4-methoxy-1-methyl-pyrrolidin-2-on-3-yl |
| A-398. | 5-methoxy-1-methyl-pyrrolidin-2-on-3-yl |
| A-399. | 4-trifluoromethoxy-1-methyl-pyrrolidin-2-on-3-yl |
| A-400. | 5-trifluoromethoxy-1-methyl-pyrrolidin-2-on-3-yl |
| A-401. | 3-chloro-1-methyl-pyrrolidin-2-on-4-yl |
| A-402. | 5-chloro-1-methyl-pyrrolidin-2-on-4-yl |
| A-403. | 3-bromo-1-methyl-pyrrolidin-2-on-4-yl |
| A-404. | 5-bromo-1-methyl-pyrrolidin-2-on-4-yl |
| A-405. | 3-cyano-1-methyl-pyrrolidin-2-on-4-yl |
| A-406. | 5-cyano-1-methyl-pyrrolidin-2-on-4-yl |
| A-407. | 3-nitro-1-methyl-pyrrolidin-2-on-4-yl |
| A-408. | 5-nitro-1-methyl-pyrrolidin-2-on-4-yl |
| A-409. | 3-methyl-1-methyl-pyrrolidin-2-on-4-yl |
| A-410. | 5-methyl-1-methyl-pyrrolidin-2-on-4-yl |
| A-411. | 3-trifluoromethyl-1-methyl-pyrrolidin-2-on-4-yl |
| A-412. | 5-trifluoromethyl-1-methyl-pyrrolidin-2-on-4-yl |
| A-413. | 3-methoxy-1-methyl-pyrrolidin-2-on-4-yl |
| A-414. | 5-methoxy-1-methyl-pyrrolidin-2-on-4-yl |
| A-415. | 3-trifluoromethoxy-1-methyl-pyrrolidin-2-on-4-yl |
| A-416. | 5-trifluoromethoxy-1-methyl-pyrrolidin-2-on-4-yl |
| A-417. | 3-chloro-1-methyl-pyrrolidin-2-on-5-yl |
| A-418. | 4-chloro-1-methyl-pyrrolidin-2-on-5-yl |
| A-419. | 3-bromo-1-methyl-pyrrolidin-2-on-5-yl |
| A-420. | 4-bromo-1-methyl-pyrrolidin-2-on-5-yl |
| A-421. | 3-cyano-1-methyl-pyrrolidin-2-on-5-yl |
| A-422. | 4-cyano-1-methyl-pyrrolidin-2-on-5-yl |
| A-423. | 3-nitro-1-methyl-pyrrolidin-2-on-5-yl |
| A-424. | 4-nitro-1-methyl-pyrrolidin-2-on-5-yl |
| A-425. | 3-methyl-1-methyl-pyrrolidin-2-on-5-yl |
| A-426. | 4-methyl-1-methyl-pyrrolidin-2-on-5-yl |
| A-427. | 3-trifluoromethyl-1-methyl-pyrrolidin-2-on-5-yl |
| A-428. | 5-trifluoromethyl-1-methyl-pyrrolidin-2-on-5-yl |
| A-429. | 3-methoxy-1-methyl-pyrrolidin-2-on-5-yl |
| A-430. | 5-methoxy-1-methyl-pyrrolidin-2-on-5-yl |
| A-431. | 3-trifluoromethoxy-1-methyl-pyrrolidin-2-on-5-yl |
| A-432. | 5-trifluoromethoxy-1-methyl-pyrrolidin-2-on-5-yl |
| A-433. | tetrahydrofuran-2-yl |
| A-434. | tetrahydrofuran-3-yl |
| A-435. | tetrahydrofuran-2-on-3-yl |
| A-436. | tetrahydrofuran-2-on-4-yl |
| A-437. | tetrahydrofuran-2-on-5-yl |
| A-438. | 3-chloro-tetrahydrofuran-2-yl |
| A-439. | 4-chloro-tetrahydrofuran-2-yl |
| A-440. | 5-chloro-tetrahydrofuran-2-yl |
| A-441. | 3-bromo-tetrahydrofuran-2-yl |
| A-442. | 4-bromo-tetrahydrofuran-2-yl |
| A-443. | 5-bromo-tetrahydrofuran-2-yl |
| A-444. | 3-cyano-tetrahydrofuran-2-yl |
| A-445. | 4-cyano-tetrahydrofuran-2-yl |
| A-446. | 5-cyano-tetrahydrofuran-2-yl |
| A-447. | 3-nitro-tetrahydrofuran-2-yl |
| A-448. | 4-nitro-tetrahydrofuran-2-yl |
| A-449. | 5-nitro-tetrahydrofuran-2-yl |
| A-450. | 3-methyl-tetrahydrofuran-2-yl |
| A-451. | 4-methyl-tetrahydrofuran-2-yl |
| A-452. | 5-methyl-tetrahydrofuran-2-yl |
| A-453. | 3-trifluoromethyl-tetrahydrofuran-2-yl |
| A-454. | 4-trifluoromethyl-tetrahydrofuran-2-yl |
| A-455. | 5-trifluoromethyl-tetrahydrofuran-2-yl |
| A-456. | 3-methoxy-tetrahydrofuran-2-yl |
| A-457. | 4-methoxy-tetrahydrofuran-2-yl |
| A-458. | 5-methoxy-tetrahydrofuran-2-yl |
| A-459. | 3-trifluoromethoxy-tetrahydrofuran-2-yl |
| A-460. | 4-trifluoromethoxy-tetrahydrofuran-2-yl |
| A-461. | 5-trifluoromethoxy-tetrahydrofuran-2-yl |
| A-462. | 2-chloro-tetrahydrofuran-3-yl |
| A-463. | 4-chloro-tetrahydrofuran-3-yl |
| A-464. | 5-chloro-tetrahydrofuran-3-yl |
| A-465. | 2-bromo-tetrahydrofuran-3-yl |
| A-466. | 4-bromo-tetrahydrofuran-3-yl |
| A-467. | 5-bromo-tetrahydrofuran-3-yl |
| A-468. | 2-cyano-tetrahydrofuran-3-yl |
| A-469. | 4-cyano-tetrahydrofuran-3-yl |
| A-470. | 5-cyano-tetrahydrofuran-3-yl |
| A-471. | 2-nitro-tetrahydrofuran-3-yl |
| A-472. | 4-nitro-tetrahydrofuran-3-yl |
| A-473. | 5-nitro-tetrahydrofuran-3-yl |
| A-474. | 2-methyl-tetrahydrofuran-3-yl |
| A-475. | 4-methyl-tetrahydrofuran-3-yl |
| A-476. | 5-methyl-tetrahydrofuran-3-yl |
| A-477. | 2-trifluoromethyl-tetrahydrofuran-3-yl |
| A-478. | 4-trifluoromethyl-tetrahydrofuran-3-yl |
| A-479. | 5-trifluoromethyl-tetrahydrofuran-3-yl |
| A-480. | 2-methoxy-tetrahydrofuran-3-yl |
| A-481. | 4-methoxy-tetrahydrofuran-3-yl |
| A-482. | 5-methoxy-tetrahydrofuran-3-yl |
| A-483. | 2-trifluoromethoxy-tetrahydrofuran-3-yl |
| A-484. | 4-trifluoromethoxy-tetrahydrofuran-3-yl |
| A-485. | 5-trifluoromethoxy-tetrahydrofuran-3-yl |
| A-486. | 3-chloro-tetrahydrofuran-2-on-1-yl |
| A-487. | 4-chloro-tetrahydrofuran-2-on-1-yl |
| A-488. | 5-chloro-tetrahydrofuran-2-on-1-yl |
| A-489. | 3-bromo-tetrahydrofuran-2-on-1-yl |
| A-490. | 4-bromo-tetrahydrofuran-2-on-1-yl |
| A-491. | 5-bromo-tetrahydrofuran-2-on-1-yl |
| A-492. | 3-cyano-tetrahydrofuran-2-on-1-yl |
| A-493. | 4-cyano-tetrahydrofuran-2-on-1-yl |
| A-494. | 5-cyano-tetrahydrofuran-2-on-1-yl |
| A-495. | 3-nitro-tetrahydrofuran-2-on-1-yl |
| A-496. | 4-nitro-tetrahydrofuran-2-on-1-yl |
| A-497. | 5-nitro-tetrahydrofuran-2-on-1-yl |
| A-498. | 3-methyl-tetrahydrofuran-2-on-1-yl |
| A-499. | 4-methyl-tetrahydrofuran-2-on-1-yl |
| A-500. | 5-methyl-tetrahydrofuran-2-on-1-yl |
| A-501. | 3-trifluoromethyl-tetrahydrofuran-2-on-1-yl |
| A-502. | 4-trifluoromethyl-tetrahydrofuran-2-on-1-yl |
| A-503. | 5-trifluoromethyl-tetrahydrofuran-2-on-1-yl |
| A-504. | 3-methoxy-tetrahydrofuran-2-on-1-yl |
| A-505. | 4-methoxy-tetrahydrofuran-2-on-1-yl |
| A-506. | 5-methoxy-tetrahydrofuran-2-on-1-yl |
| A-507. | 3-trifluoromethoxy-tetrahydrofuran-2-on-1-yl |
| A-508. | 4-trifluoromethoxy-tetrahydrofuran-2-on-1-yl |
| A-509. | 5-trifluoromethoxy-tetrahydrofuran-2-on-1-yl |
| A-510. | 4-chloro-tetrahydrofuran-2-on-3-yl |
| A-511. | 5-chloro-tetrahydrofuran-2-on-3-yl |
| A-512. | 4-bromo-tetrahydrofuran-2-on-3-yl |
| A-513. | 5-bromo-tetrahydrofuran-2-on-3-yl |
| A-514. | 4-cyano-tetrahydrofuran-2-on-3-yl |
| A-515. | 5-cyano-tetrahydrofuran-2-on-3-yl |
| A-516. | 4-nitro-tetrahydrofuran-2-on-3-yl |
| A-517. | 5-nitro-tetrahydrofuran-2-on-3-yl |
| A-518. | 4-methyl-tetrahydrofuran-2-on-3-yl |
| A-519. | 5-methyl-tetrahydrofuran-2-on-3-yl |
| A-520. | 4-trifluoromethyl-tetrahydrofuran-2-on-3-yl |
| A-521. | 5-trifluoromethyl-tetrahydrofuran-2-on-3-yl |
| A-522. | 4-methoxy-tetrahydrofuran-2-on-3-yl |
| A-523. | 5-methoxy-tetrahydrofuran-2-on-3-yl |
| A-524. | 4-trifluoromethoxy-tetrahydrofuran-2-on-3-yl |
| A-525. | 5-trifluoromethoxy-tetrahydrofuran-2-on-3-yl |
| A-526. | 3-chloro-tetrahydrofuran-2-on-4-yl |
| A-527. | 5-chloro-tetrahydrofuran-2-on-4-yl |
| A-528. | 3-bromo-tetrahydrofuran-2-on-4-yl |
| A-529. | 5-bromo-tetrahydrofuran-2-on-4-yl |
| A-530. | 3-cyano-tetrahydrofuran-2-on-4-yl |
| A-531. | 5-cyano-tetrahydrofuran-2-on-4-yl |
| A-532. | 3-nitro-tetrahydrofuran-2-on-4-yl |
| A-533. | 5-nitro-tetrahydrofuran-2-on-4-yl |
| A-534. | 3-methyl-tetrahydrofuran-2-on-4-yl |
| A-535. | 5-methyl-tetrahydrofuran-2-on-4-yl |
| A-536. | 3-trifluoromethyl-tetrahydrofuran-2-on-4-yl |
| A-537. | 5-trifluoromethyl-tetrahydrofuran-2-on-4-yl |
| A-538. | 3-methoxy-tetrahydrofuran-2-on-4-yl |
| A-539. | 5-methoxy-tetrahydrofuran-2-on-4-yl |
| A-540. | 3-trifluoromethoxy-tetrahydrofuran-2-on-4-yl |
| A-541. | 5-trifluoromethoxy-tetrahydrofuran-2-on-4-yl |
| A-542. | 3-chloro-tetrahydrofuran-2-on-5-yl |
| A-543. | 4-chloro-tetrahydrofuran-2-on-5-yl |
| A-544. | 3-bromo-tetrahydrofuran-2-on-5-yl |
| A-545. | 4-bromo-tetrahydrofuran-2-on-5-yl |
| A-546. | 3-cyano-tetrahydrofuran-2-on-5-yl |
| A-547. | 4-cyano-tetrahydrofuran-2-on-5-yl |
| A-548. | 3-nitro-tetrahydrofuran-2-on-5-yl |
| A-549. | 4-nitro-tetrahydrofuran-2-on-5-yl |
| A-550. | 3-methyl-tetrahydrofuran-2-on-5-yl |
| A-551. | 4-methyl-tetrahydrofuran-2-on-5-yl |
| A-552. | 3-trifluoromethyl-tetrahydrofuran-2-on-5-yl |
| A-553. | 4-trifluoromethyl-tetrahydrofuran-2-on-5-yl |
| A-554. | 3-methoxy-tetrahydrofuran-2-on-5-yl |
| A-555. | 4-methoxy-tetrahydrofuran-2-on-5-yl |
| A-556. | 3-trifluoromethoxy-tetrahydrofuran-2-on-5-yl |
| A-557. | 4-trifluoromethoxy-tetrahydrofuran-2-on-5-yl |
| A-558. | pyrazolidin-1-yl |
| A-559. | pyrazolidin-3-yl |
| A-560. | pyrazolidin-4-yl |
| A-561. | 1-methyl-pyrazolidin-3-yl |
| A-562. | 1-methyl-pyrazolidin-4-yl |
| A-563. | 1-methyl-pyrazolidin-5-yl |
| A-564. | 3-chloro-pyrazolidin-1-yl |
| A-565. | 4-chloro-pyrazolidin-1-yl |
| A-566. | 5-chloro-pyrazolidin-1-yl |
| A-567. | 3-bromo-pyrazolidin-1-yl |
| A-568. | 4-bromo-pyrazolidin-1-yl |
| A-569. | 5-bromo-pyrazolidin-1-yl |
| A-570. | 3-cyano-pyrazolidin-1-yl |
| A-571. | 4-cyano-pyrazolidin-1-yl |
| A-572. | 5-cyano-pyrazolidin-1-yl |
| A-573. | 3-nitro-pyrazolidin-1-yl |
| A-574. | 4-nitro-pyrazolidin-1-yl |
| A-575. | 5-nitro-pyrazolidin-1-yl |
| A-576. | 3-methyl-pyrazolidin-1-yl |
| A-577. | 3-methyl-pyrazolidin-1-yl |
| A-578. | 4-methyl-pyrazolidin-1-yl |
| A-579. | 3-trifluoromethyl-pyrazolidin-1-yl |
| A-580. | 4-trifluoromethyl-pyrazolidin-1-yl |
| A-581. | 5-trifluoromethyl-pyrazolidin-1-yl |
| A-582. | 3-methoxy-pyrazolidin-1-yl |
| A-583. | 4-methoxy-pyrazolidin-1-yl |
| A-584. | 5-methoxy-pyrazolidin-1-yl |
| A-585. | 3-trifluoromethoxy-pyrazolidin-1-yl |
| A-586. | 4-trifluoromethoxy-pyrazolidin-1-yl |
| A-587. | 5-trifluoromethoxy-pyrazolidin-1-yl |
| A-588. | 4-chloro-pyrazolidin-3-yl |
| A-589. | 5-chloro-pyrazolidin-3-yl |
| A-590. | 4-bromo-pyrazolidin-3-yl |
| A-591. | 5-bromo-pyrazolidin-3-yl |
| A-592. | 4-cyano-pyrazolidin-3-yl |
| A-593. | 5-cyano-pyrazolidin-3-yl |
| A-594. | 4-nitro-pyrazolidin-3-yl |
| A-595. | 5-nitro-pyrazolidin-3-yl |
| A-596. | 4-methyl-pyrazolidin-3-yl |
| A-597. | 5-methyl-pyrazolidin-3-yl |
| A-598. | 4-trifluoromethyl-pyrazolidin-3-yl |
| A-599. | 5-trifluoromethyl-pyrazolidin-3-yl |
| A-600. | 4-methoxy-pyrazolidin-3-yl |
| A-601. | 5-methoxy-pyrazolidin-3-yl |
| A-602. | 4-trifluoromethoxy-pyrazolidin-3-yl |
| A-603. | 5-trifluoromethoxy-pyrazolidin-3-yl |
| A-604. | 3-chloro-pyrazolidin-4-yl |
| A-605. | 5-chloro-pyrazolidin-4-yl |
| A-606. | 3-bromo-pyrazolidin-4-yl |
| A-607. | 5-bromo-pyrazolidin-4-yl |
| A-608. | 3-cyano-pyrazolidin-4-yl |
| A-609. | 5-cyano-pyrazolidin-4-yl |
| A-610. | 3-nitro-pyrazolidin-4-yl |
| A-611. | 5-nitro-pyrazolidin-4-yl |
| A-612. | 3-methyl-pyrazolidin-4-yl |
| A-613. | 5-methyl-pyrazolidin-4-yl |
| A-614. | 3-trifluoromethyl-pyrazolidin-4-yl |
| A-615. | 5-trifluoromethyl-pyrazolidin-4-yl |
| A-616. | 3-methoxy-pyrazolidin-4-yl |
| A-617. | 5-methoxy-pyrazolidin-4-yl |
| A-618. | 3-trifluoromethoxy-pyrazolidin-4-yl |
| A-619. | 5-trifluoromethoxy-pyrazolidin-4-yl |
| A-620. | 3-chloro-pyrazolidin-5-yl |
| A-621. | 4-chloro-pyrazolidin-5-yl |
| A-622. | 3-bromo-pyrazolidin-5-yl |
| A-623. | 4-bromo-pyrazolidin-5-yl |
| A-624. | 3-cyano-pyrazolidin-5-yl |
| A-625. | 4-cyano-pyrazolidin-5-yl |
| A-626. | 3-nitro-pyrazolidin-5-yl |
| A-627. | 4-nitro-pyrazolidin-5-yl |
| A-628. | 3-methyl-pyrazolidin-5-yl |
| A-629. | 4-methyl-pyrazolidin-5-yl |
| A-630. | 3-trifluoromethyl-pyrazolidin-5-yl |
| A-631. | 4-trifluoromethyl-pyrazolidin-5-yl |
| A-632. | 3-methoxy-pyrazolidin-5-yl |
| A-633. | 4-methoxy-pyrazolidin-5-yl |
| A-634. | 3-trifluoromethoxy-pyrazolidin-5-yl |
| A-635. | 4-trifluoromethoxy-pyrazolidin-5-yl |
| A-636. | 3-chloro-1-methyl-pyrazolidin-2-yl |
| A-637. | 4-chloro-1-methyl-pyrazolidin-2-yl |
| A-638. | 5-chloro-1-methyl-pyrazolidin-2-yl |
| A-639. | 3-bromo-1-methyl-pyrazolidin-2-yl |
| A-640. | 4-bromo-1-methyl-pyrazolidin-2-yl |
| A-641. | 5-bromo-1-methyl-pyrazolidin-2-yl |
| A-642. | 3-cyano-1-methyl-pyrazolidin-2-yl |
| A-643. | 4-cyano-1-methyl-pyrazolidin-2-yl |
| A-644. | 5-cyano-1-methyl-pyrazolidin-2-yl |
| A-645. | 3-nitro-1-methyl-pyrazolidin-2-yl |
| A-646. | 4-nitro-1-methyl-pyrazolidin-2-yl |
| A-647. | 5-nitro-1-methyl-pyrazolidin-2-yl |
| A-648. | 3-methyl-1-methyl-pyrazolidin-2-yl |
| A-649. | 4-methyl-1-methyl-pyrazolidin-2-yl |
| A-650. | 5-methyl-1-methyl-pyrazolidin-2-yl |
| A-651. | 3-trifluoromethyl-1-methyl-pyrazolidin-2-yl |
| A-652. | 4-trifluoromethyl-1-methyl-pyrazolidin-2-yl |
| A-653. | 5-trifluoromethyl-1-methyl-pyrazolidin-2-yl |
| A-654. | 3-methoxy-1-methyl-pyrazolidin-2-yl |
| A-655. | 4-methoxy-1-methyl-pyrazolidin-2-yl |
| A-656. | 5-methoxy-1-methyl-pyrazolidin-2-yl |
| A-657. | 3-trifluoromethoxy-1-methyl-pyrazolidin-2-yl |
| A-658. | 4-trifluoromethoxy-1-methyl-pyrazolidin-2-yl |
| A-659. | 5-trifluoromethoxy-1-methyl-pyrazolidin-2-yl |
| A-660. | 4-chloro-1-methyl-pyrazolidin-3-yl |
| A-661. | 5-chloro-1-methyl-pyrazolidin-3-yl |
| A-662. | 4-bromo-1-methyl-pyrazolidin-3-yl |
| A-663. | 5-bromo-1-methyl-pyrazolidin-3-yl |
| A-664. | 4-cyano-1-methyl-pyrazolidin-3-yl |
| A-665. | 5-cyano-1-methyl-pyrazolidin-3-yl |
| A-666. | 4-nitro-1-methyl-pyrazolidin-3-yl |
| A-667. | 5-nitro-1-methyl-pyrazolidin-3-yl |
| A-668. | 4-methyl-1-methyl-pyrazolidin-3-yl |
| A-669. | 5-methyl-1-methyl-pyrazolidin-3-yl |
| A-670. | 4-trifluoromethyl-1-methyl-pyrazolidin-3-yl |
| A-671. | 5-trifluoromethyl-1-methyl-pyrazolidin-3-yl |
| A-672. | 4-methoxy-1-methyl-pyrazolidin-3-yl |
| A-673. | 5-methoxy-1-methyl-pyrazolidin-3-yl |
| A-674. | 4-trifluoromethoxy-1-methyl-pyrazolidin-3-yl |
| A-675. | 5-trifluoromethoxy-1-methyl-pyrazolidin-3-yl |
| A-676. | 3-chloro-1-methyl-pyrazolidin-4-yl |
| A-677. | 5-chloro-1-methyl-pyrazolidin-4-yl |
| A-678. | 3-bromo-1-methyl-pyrazolidin-4-yl |
| A-679. | 5-bromo-1-methyl-pyrazolidin-4-yl |
| A-680. | 3-cyano-1-methyl-pyrazolidin-4-yl |
| A-681. | 5-cyano-1-methyl-pyrazolidin-4-yl |
| A-682. | 3-nitro-1-methyl-pyrazolidin-4-yl |
| A-683. | 5-nitro-1-methyl-pyrazolidin-4-yl |
| A-684. | 3-methyl-1-methyl-pyrazolidin-4-yl |
| A-685. | 5-methyl-1-methyl-pyrazolidin-4-yl |
| A-686. | 3-trifluoromethyl-1-methyl-pyrazolidin-4-yl |
| A-687. | 5-trifluoromethyl-1-methyl-pyrazolidin-4-yl |
| A-688. | 3-methoxy-1-methyl-pyrazolidin-4-yl |
| A-689. | 5-methoxy-1-methyl-pyrazolidin-4-yl |
| A-690. | 3-trifluoromethoxy-1-methyl-pyrazolidin-4-yl |
| A-691. | 5-trifluoromethoxy-1-methyl-pyrazolidin-4-yl |
| A-692. | 3-chloro-1-methyl-pyrazolidin-5-yl |
| A-693. | 4-chloro-1-methyl-pyrazolidin-5-yl |
| A-694. | 3-bromo-1-methyl-pyrazolidin-5-yl |
| A-695. | 4-bromo-1-methyl-pyrazolidin-5-yl |
| A-696. | 3-cyano-1-methyl-pyrazolidin-5-yl |
| A-697. | 4-cyano-1-methyl-pyrazolidin-5-yl |
| A-698. | 3-nitro-1-methyl-pyrazolidin-5-yl |
| A-699. | 4-nitro-1-methyl-pyrazolidin-5-yl |
| A-700. | 3-methyl-1-methyl-pyrazolidin-5-yl |
| A-701. | 4-methyl-1-methyl-pyrazolidin-5-yl |
| A-702. | 3-trifluoromethyl-1-methyl-pyrazolidin-5-yl |
| A-703. | 4-trifluoromethyl-1-methyl-pyrazolidin-5-yl |
| A-704. | 3-methoxy-1-methyl-pyrazolidin-5-yl |
| A-705. | 4-methoxy-1-methyl-pyrazolidin-5-yl |
| A-706. | 3-trifluoromethoxy-1-methyl-pyrazolidin-5-yl |
| A-707. | 4-trifluoromethoxy-1-methyl-pyrazolidin-5-yl |
| A-708. | imidazolidin-1-yl |
| A-709. | imidazolidin-2-yl |
| A-710. | imidazolidin-4-yl |
| A-711. | imidazolidin-5-yl |
| A-712. | 1-methyl-imidazolidin-2-yl |
| A-713. | 1-methyl-imidazolidin-4-yl |
| A-714. | 1-methyl-imidazolidin-5-yl |
| A-715. | imidazolidin-2-on-1-yl |
| A-716. | imidazolidin-2-on-4-yl |
| A-717. | imidazolidin-2-on-5-yl |
| A-718. | 1-methyl-imidazolidin-2-on-4-yl |
| A-719. | 1-methyl-imidazolidin-2-on-5-yl |
| A-720. | 2-chloro-imidazolidin-1-yl |
| A-721. | 4-chloro-imidazolidin-1-yl |
| A-722. | 5-chloro-imidazolidin-1-yl |
| A-723. | 2-bromo-imidazolidin-1-yl |
| A-724. | 4-bromo-imidazolidin-1-yl |
| A-725. | 5-bromo-imidazolidin-1-yl |
| A-726. | 2-cyano-imidazolidin-1-yl |
| A-727. | 4-cyano-imidazolidin-1-yl |
| A-728. | 5-cyano-imidazolidin-1-yl |
| A-729. | 2-nitro-imidazolidin-1-yl |
| A-730. | 4-nitro-imidazolidin-1-yl |
| A-731. | 5-nitro-imidazolidin-1-yl |
| A-732. | 2-methyl-imidazolidin-1-yl |
| A-733. | 4-methyl-imidazolidin-1-yl |
| A-734. | 5-methyl-imidazolidin-1-yl |
| A-735. | 2-trifluoromethyl-imidazolidin-1-yl |
| A-736. | 4-trifluoromethyl-imidazolidin-1-yl |
| A-737. | 5-trifluoromethyl-imidazolidin-1-yl |
| A-738. | 2-methoxy-imidazolidin-1-yl |
| A-739. | 4-methoxy-imidazolidin-1-yl |
| A-740. | 5-methoxy-imidazolidin-1-yl |
| A-741. | 2-trifluoromethoxy-imidazolidin-1-yl |
| A-742. | 4-trifluoromethoxy-imidazolidin-1-yl |
| A-743. | 5-trifluoromethoxy-imidazolidin-1-yl |
| A-744. | 4-chloro-imidazolidin-2-yl |
| A-745. | 5-chloro-imidazolidin-2-yl |
| A-746. | 4-bromo-imidazolidin-2-yl |
| A-747. | 5-bromo-imidazolidin-2-yl |
| A-748. | 4-cyano-imidazolidin-2-yl |
| A-749. | 5-cyano-imidazolidin-2-yl |
| A-750. | 4-nitro-imidazolidin-2-yl |
| A-751. | 5-nitro-imidazolidin-2-yl |
| A-752. | 4-methyl-imidazolidin-2-yl |
| A-753. | 5-methyl-imidazolidin-2-yl |
| A-754. | 4-trifluoromethyl-imidazolidin-2-yl |
| A-755. | 5-trifluoromethyl-imidazolidin-2-yl |
| A-756. | 4-methoxy-imidazolidin-2-yl |
| A-757. | 5-methoxy-imidazolidin-2-yl |
| A-758. | 4-trifluoromethoxy-imidazolidin-2-yl |
| A-759. | 5-trifluoromethoxy-imidazolidin-2-yl |
| A-760. | 2-chloro-imidazolidin-4-yl |
| A-761. | 5-chloro-imidazolidin-4-yl |
| A-762. | 2-bromo-imidazolidin-4-yl |
| A-763. | 5-bromo-imidazolidin-4-yl |
| A-764. | 2-cyano-imidazolidin-4-yl |
| A-765. | 5-cyano-imidazolidin-4-yl |
| A-766. | 2-nitro-imidazolidin-4-yl |
| A-767. | 5-nitro-imidazolidin-4-yl |
| A-768. | 2-methyl-imidazolidin-4-yl |
| A-769. | 5-methyl-imidazolidin-4-yl |
| A-770. | 2-trifluoromethyl-imidazolidin-4-yl |
| A-771. | 5-trifluoromethyl-imidazolidin-4-yl |
| A-772. | 2-methoxy-imidazolidin-4-yl |
| A-773. | 5-methoxy-imidazolidin-4-yl |
| A-774. | 2-trifluoromethoxy-imidazolidin-4-yl |
| A-775. | 5-trifluoromethoxy-imidazolidin-4-yl |
| A-776. | 2-chloro-imidazolidin-5-yl |
| A-777. | 4-chloro-imidazolidin-5-yl |
| A-778. | 2-bromo-imidazolidin-5-yl |
| A-779. | 4-bromo-imidazolidin-5-yl |
| A-780. | 2-cyano-imidazolidin-5-yl |
| A-781. | 4-cyano-imidazolidin-5-yl |
| A-782. | 2-nitro-imidazolidin-5-yl |
| A-783. | 4-nitro-imidazolidin-5-yl |
| A-784. | 2-methyl-imidazolidin-5-yl |
| A-785. | 4-methyl-imidazolidin-5-yl |
| A-786. | 2-trifluoromethyl-imidazolidin-5-yl |
| A-787. | 5-trifluoromethyl-imidazolidin-5-yl |
| A-788. | 2-methoxy-imidazolidin-5-yl |
| A-789. | 5-methoxy-imidazolidin-5-yl |
| A-790. | 2-trifluoromethoxy-imidazolidin-5-yl |
| A-791. | 5-trifluoromethoxy-imidazolidin-5-yl |
| A-792. | 4-chloro-1-methyl-imidazolidin-2-yl |
| A-793. | 5-chloro-1-methyl-imidazolidin-2-yl |
| A-794. | 4-bromo-1-methyl-imidazolidin-2-yl |
| A-795. | 5-bromo-1-methyl-imidazolidin-2-yl |
| A-796. | 4-cyano-1-methyl-imidazolidin-2-yl |
| A-797. | 5-cyano-1-methyl-imidazolidin-2-yl |
| A-798. | 4-nitro-1-methyl-imidazolidin-2-yl |
| A-799. | 5-nitro-1-methyl-imidazolidin-2-yl |
| A-800. | 4-methyl-1-methyl-imidazolidin-2-yl |
| A-801. | 5-methyl-1-methyl-imidazolidin-2-yl |
| A-802. | 4-trifluoromethyl-1-methyl-imidazolidin-2-yl |
| A-803. | 5-trifluoromethyl-1-methyl-imidazolidin-2-yl |
| A-804. | 4-methoxy-1-methyl-imidazolidin-2-yl |
| A-805. | 5-methoxy-1-methyl-imidazolidin-2-yl |
| A-806. | 4-trifluoromethoxy-1-methyl-imidazolidin-2-yl |
| A-807. | 5-trifluoromethoxy-1-methyl-imidazolidin-2-yl |
| A-808. | 2-chloro-1-methyl-imidazolidin-4-yl |
| A-809. | 5-chloro-1-methyl-imidazolidin-4-yl |
| A-810. | 2-bromo-1-methyl-imidazolidin-4-yl |
| A-811. | 5-bromo-1-methyl-imidazolidin-4-yl |
| A-812. | 2-cyano-1-methyl-imidazolidin-4-yl |
| A-813. | 5-cyano-1-methyl-imidazolidin-4-yl |
| A-814. | 2-nitro-1-methyl-imidazolidin-4-yl |
| A-815. | 5-nitro-1-methyl-imidazolidin-4-yl |
| A-816. | 2-methyl-1-methyl-imidazolidin-4-yl |
| A-817. | 5-methyl-1-methyl-imidazolidin-4-yl |
| A-818. | 2-trifluoromethyl-1-methyl-imidazolidin-4-yl |
| A-819. | 5-trifluoromethyl-1-methyl-imidazolidin-4-yl |
| A-820. | 2-methoxy-1-methyl-imidazolidin-4-yl |
| A-821. | 5-methoxy-1-methyl-imidazolidin-4-yl |
| A-822. | 2-trifluoromethoxy-1-methyl-imidazolidin-4-yl |
| A-823. | 5-trifluoromethoxy-1-methyl-imidazolidin-4-yl |
| A-824. | 2-chloro-1-methyl-imidazolidin-5-yl |
| A-825. | 4-chloro-1-methyl-imidazolidin-5-yl |
| A-826. | 2-bromo-1-methyl-imidazolidin-5-yl |
| A-827. | 4-bromo-1-methyl-imidazolidin-5-yl |
| A-828. | 2-cyano-1-methyl-imidazolidin-5-yl |
| A-829. | 4-cyano-1-methyl-imidazolidin-5-yl |
| A-830. | 2-nitro-1-methyl-imidazolidin-5-yl |
| A-831. | 4-nitro-1-methyl-imidazolidin-5-yl |
| A-832. | 2-methyl-1-methyl-imidazolidin-5-yl |
| A-833. | 4-methyl-1-methyl-imidazolidin-5-yl |
| A-834. | 2-trifluoromethyl-1-methyl-imidazolidin-5-yl |
| A-835. | 4-trifluoromethyl-1-methyl-imidazolidin-5-yl |
| A-836. | 2-methoxy-1-methyl-imidazolidin-5-yl |
| A-837. | 4-methoxy-1-methyl-imidazolidin-5-yl |
| A-838. | 2-trifluoromethoxy-1-methyl-imidazolidin-5-yl |
| A-839. | 4-trifluoromethoxy-1-methyl-imidazolidin-5-yl |
| A-840. | 4-chloro-imidazolidin-2-on-1-yl |
| A-841. | 5-chloro-imidazolidin-2-on-1-yl |
| A-842. | 4-bromo-imidazolidin-2-on-1-yl |
| A-843. | 5-bromo-imidazolidin-2-on-1-yl |
| A-844. | 4-cyano-imidazolidin-2-on-1-yl |
| A-845. | 5-cyano-imidazolidin-2-on-1-yl |
| A-846. | 4-nitro-imidazolidin-2-on-1-yl |
| A-847. | 5-nitro-imidazolidin-2-on-1-yl |
| A-848. | 4-methyl-imidazolidin-2-on-1-yl |
| A-849. | 5-methyl-imidazolidin-2-on-1-yl |
| A-850. | 4-trifluoromethyl-imidazolidin-2-on-1-yl |
| A-851. | 5-trifluoromethyl-imidazolidin-2-on-1-yl |
| A-852. | 4-methoxy-imidazolidin-2-on-1-yl |
| A-853. | 5-methoxy-imidazolidin-2-on-1-yl |
| A-854. | 4-trifluoromethoxy-imidazolidin-2-on-1-yl |
| A-855. | 5-trifluoromethoxy-imidazolidin-2-on-1-yl |
| A-856. | 5-chloro-imidazolidin-2-on-4-yl |
| A-857. | 5-bromo-imidazolidin-2-on-4-yl |
| A-858. | 5-cyano-imidazolidin-2-on-4-yl |
| A-859. | 5-nitro-imidazolidin-2-on-4-yl |
| A-860. | 5-methyl-imidazolidin-2-on-4-yl |
| A-861. | 5-trifluoromethyl-imidazolidin-2-on-4-yl |
| A-862. | 5-methoxy-imidazolidin-2-on-4-yl |
| A-863. | 5-trifluoromethoxy-imidazolidin-2-on-4-yl |
| A-864. | 4-chloro-imidazolidin-2-on-5-yl |
| A-865. | 4-bromo-imidazolidin-2-on-5-yl |
| A-866. | 4-cyano-imidazolidin-2-on-5-yl |
| A-867. | 4-nitro-imidazolidin-2-on-5-yl |
| A-868. | 4-methyl-imidazolidin-2-on-5-yl |
| A-869. | 4-trifluoromethyl-imidazolidin-2-on-5-yl |
| A-870. | 4-methoxy-imidazolidin-2-on-5-yl |
| A-871. | 4-trifluoromethoxy-imidazolidin-2-on-5-yl |
| A-872. | 5-chloro-1-methyl-imidazolidin-2-on-4-yl |
| A-873. | 5-bromo-1-methyl-imidazolidin-2-on-4-yl |
| A-874. | 5-cyano-1-methyl-imidazolidin-2-on-4-yl |
| A-875. | 5-nitro-1-methyl-imidazolidin-2-on-4-yl |
| A-876. | 5-methyl-1-methyl-imidazolidin-2-on-4-yl |
| A-877. | 5-trifluoromethyl-1-methyl-imidazolidin-2-on-4-yl |
| A-878. | 5-methoxy-1-methyl-imidazolidin-2-on-4-yl |
| A-879. | 5-trifluoromethoxy-1-methyl-imidazolidin-2-on-4-yl |
| A-880. | 4-chloro-1-methyl-imidazolidin-2-on-5-yl |
| A-881. | 4-bromo-1-methyl-imidazolidin-2-on-5-yl |
| A-882. | 4-cyano-1-methyl-imidazolidin-2-on-5-yl |
| A-883. | 4-nitro-1-methyl-imidazolidin-2-on-5-yl |
| A-884. | 4-methyl-1-methyl-imidazolidin-2-on-5-yl |
| A-885. | 4-trifluoromethyl-1-methyl-imidazolidin-2-on-5-yl |
| A-886. | 4-methoxy-1-methyl-imidazolidin-2-on-5-yl |
| A-887. | 4-trifluoromethoxy-1-methyl-imidazolidin-2-on-5-yl |
| A-888. | oxazolidin-2-yl |
| A-889. | oxazolidin-4-yl |
| A-890. | oxazolidin-5-yl |
| A-891. | 4-chloro-oxazolidin-2-yl |
| A-892. | 5-chloro-oxazolidin-2-yl |
| A-893. | 4-bromo-oxazolidin-2-yl |
| A-894. | 5-bromo-oxazolidin-2-yl |
| A-895. | 4-cyano-oxazolidin-2-yl |
| A-896. | 5-cyano-oxazolidin-2-yl |
| A-897. | 4-nitro-oxazolidin-2-yl |
| A-898. | 5-nitro-oxazolidin-2-yl |
| A-899. | 4-methyl-oxazolidin-2-yl |
| A-900. | 5-methyl-oxazolidin-2-yl |
| A-901. | 4-trifluromethyl-oxazolidin-2-yl |
| A-902. | 5-trifluromethyl-oxazolidin-2-yl |
| A-903. | 4-methoxy-oxazolidin-2-yl |
| A-904. | 5-methoxy-oxazolidin-2-yl |
| A-905. | 4-trifluoromethoxy-oxazolidin-2-yl |
| A-906. | 5-trifluoromethoxy-oxazolidin-2-yl |
| A-907. | 2-chloro-oxazolidin-4-yl |
| A-908. | 5-chloro-oxazolidin-4-yl |
| A-909. | 2-bromo-oxazolidin-4-yl |
| A-910. | 5-bromo-oxazolidin-4-yl |
| A-911. | 2-cyano-oxazolidin-4-yl |
| A-912. | 5-cyano-oxazolidin-4-yl |
| A-913. | 2-nitro-oxazolidin-4-yl |
| A-914. | 5-nitro-oxazolidin-4-yl |
| A-915. | 2-methyl-oxazolidin-4-yl |
| A-916. | 5-methyl-oxazolidin-4-yl |
| A-917. | 2-trifluoromethyl-oxazolidin-4-yl |
| A-918. | 5-trifluoromethyl-oxazolidin-4-yl |
| A-919. | 2-methoxy-oxazolidin-4-yl |
| A-920. | 5-methoxy-oxazolidin-4-yl |
| A-921. | 2-trifluoromethoxy-oxazolidin-4-yl |
| A-922. | 5-trifluoromethoxy-oxazolidin-4-yl |
| A-923. | 2-chloro-oxazolidin-5-yl |
| A-924. | 4-chloro-oxazolidin-5-yl |
| A-925. | 2-bromo-oxazolidin-5-yl |
| A-926. | 4-bromo-oxazolidin-5-yl |
| A-927. | 2-cyano-oxazolidin-5-yl |
| A-928. | 4-cyano-oxazolidin-5-yl |
| A-929. | 2-nitro-oxazolidin-5-yl |
| A-930. | 4-nitro-oxazolidin-5-yl |
| A-931. | 2-methyl-oxazolidin-5-yl |
| A-932. | 4-methyl-oxazolidin-5-yl |
| A-933. | 2-trifluoromethyl-oxazolidin-5-yl |
| A-934. | 4-trifluoromethyl-oxazolidin-5-yl |
| A-935. | 2-methoxy-oxazolidin-5-yl |
| A-936. | 4-methoxy-oxazolidin-5-yl |
| A-937. | 2-trifluoromethoxy-oxazolidin-5-yl |
| A-938. | 4-trifluoromethoxy-oxazolidin-5-yl |
| A-939. | thiazolidin-2-yl |
| A-940. | thiazolidin-4-yl |
| A-941. | thiazolidin-5-yl |
| A-942. | 4-chloro-thiazolidin-2-yl |
| A-943. | 5-chloro-thiazolidin-2-yl |
| A-944. | 4-bromo-thiazolidin-2-yl |
| A-945. | 5-bromo-thiazolidin-2-yl |
| A-946. | 4-cyano-thiazolidin-2-yl |
| A-947. | 5-cyano-thiazolidin-2-yl |
| A-948. | 4-nitro-thiazolidin-2-yl |
| A-949. | 5-nitro-thiazolidin-2-yl |
| A-950. | 4-methyl-thiazolidin-2-yl |
| A-951. | 5-methyl-thiazolidin-2-yl |
| A-952. | 4-trifluoromethyl-thiazolidin-2-yl |
| A-953. | 5-trifluoromethyl-thiazolidin-2-yl |
| A-954. | 4-methoxy-thiazolidin-2-yl |
| A-955. | 5-methoxy-thiazolidin-2-yl |
| A-956. | 4-trifluoromethoxy-thiazolidin-2-yl |
| A-957. | 5-trifluoromethoxy-thiazolidin-2-yl |
| A-958. | 2-chloro-thiazolidin-4-yl |
| A-959. | 5-chloro-thiazolidin-4-yl |
| A-960. | 2-bromo-thiazolidin-4-yl |
| A-961. | 5-bromo-thiazolidin-4-yl |
| A-962. | 2-cyano-thiazolidin-4-yl |
| A-963. | 5-cyano-thiazolidin-4-yl |
| A-964. | 2-nitro-thiazolidin-4-yl |
| A-965. | 5-nitro-thiazolidin-4-yl |
| A-966. | 2-methyl-thiazolidin-4-yl |
| A-967. | 5-methyl-thiazolidin-4-yl |
| A-968. | 2-trifluoromethyl-thiazolidin-4-yl |
| A-969. | 5-trifluoromethyl-thiazolidin-4-yl |
| A-970. | 2-methoxy-thiazolidin-4-yl |
| A-971. | 5-methoxy-thiazolidin-4-yl |
| A-972. | 2-trifluoromethoxy-thiazolidin-4-yl |
| A-973. | 5-trifluoromethoxy-thiazolidin-4-yl |
| A-974. | 2-chloro-thiazolidin-5-yl |
| A-975. | 4-chloro-thiazolidin-5-yl |
| A-976. | 2-bromo-thiazolidin-5-yl |
| A-977. | 4-bromo-thiazolidin-5-yl |
| A-978. | 2-cyano-thiazolidin-5-yl |
| A-979. | 4-cyano-thiazolidin-5-yl |
| A-980. | 2-nitro-thiazolidin-5-yl |
| A-981. | 4-nitro-thiazolidin-5-yl |
| A-982. | 2-methyl-thiazolidin-5-yl |
| A-983. | 4-methyl-thiazolidin-5-yl |
| A-984. | 2-trifluoromethyl-thiazolidin-5-yl |
| A-985. | 4-trifluoromethyl-thiazolidin-5-yl |
| A-986. | 2-methoxy-thiazolidin-5-yl |
| A-987. | 4-methoxy-thiazolidin-5-yl |
| A-988. | 2-trifluoromethoxy-thiazolidin-5-yl |
| A-989. | 4-trifluoromethoxy-thiazolidin-5-yl |
| A-990. | isoxazolidin-3-yl |
| A-991. | isoxazolidin-4-yl |
| A-992. | isoxazolidin-5-yl |
| A-993. | 4-chloro-isoxazolidin-3-yl |
| A-994. | 5-chloro-isoxazolidin-3-yl |
| A-995. | 4-bromo-isoxazolidin-3-yl |
| A-996. | 5-bromo-isoxazolidin-3-yl |
| A-997. | 4-cyano-isoxazolidin-3-yl |
| A-998. | 5-cyano-isoxazolidin-3-yl |
| A-999. | 4-nitro-isoxazolidin-3-yl |
| A-1000. | 5-nitro-isoxazolidin-3-yl |
| A-1001. | 4-methyl-isoxazolidin-3-yl |
| A-1002. | 5-methyl-isoxazolidin-3-yl |
| A-1003. | 4-trifluoromethyl-isoxazolidin-3-yl |
| A-1004. | 5-trifluoromethyl-isoxazolidin-3-yl |
| A-1005. | 4-methoxy-isoxazolidin-3-yl |
| A-1006. | 5-methoxy-isoxazolidin-3-yl |
| A-1007. | 4-trifluoromethoxy-isoxazolidin-3-yl |
| A-1008. | 5-trifluoromethoxy-isoxazolidin-3-yl |
| A-1009. | 3-chloro-isoxazolidin-4-yl |
| A-1010. | 5-chloro-isoxazolidin-4-yl |
| A-1011. | 3-bromo-isoxazolidin-4-yl |
| A-1012. | 5-bromo-isoxazolidin-4-yl |
| A-1013. | 3-cyano-isoxazolidin-4-yl |
| A-1014. | 5-cyano-isoxazolidin-4-yl |
| A-1015. | 3-nitro-isoxazolidin-4-yl |
| A-1016. | 5-nitro-isoxazolidin-4-yl |
| A-1017. | 3-methyl-isoxazolidin-4-yl |
| A-1018. | 5-methyl-isoxazolidin-4-yl |
| A-1019. | 3-trifluoromethyl-isoxazolidin-4-yl |
| A-1020. | 5-trifluoromethyl-isoxazolidin-4-yl |
| A-1021. | 3-methoxy-isoxazolidin-4-yl |
| A-1022. | 5-methoxy-isoxazolidin-4-yl |
| A-1023. | 3-trifluoromethoxy-isoxazolidin-4-yl |
| A-1024. | 5-trifluoromethoxy-isoxazolidin-4-yl |
| A-1025. | 3-chloro-isoxazolidin-5-yl |
| A-1026. | 4-chloro-isoxazolidin-5-yl |
| A-1027. | 3-bromo-isoxazolidin-5-yl |
| A-1028. | 4-bromo-isoxazolidin-5-yl |
| A-1029. | 3-cyano-isoxazolidin-5-yl |
| A-1030. | 4-cyano-isoxazolidin-5-yl |
| A-1031. | 3-nitro-isoxazolidin-5-yl |
| A-1032. | 4-nitro-isoxazolidin-5-yl |
| A-1033. | 3-methyl-isoxazolidin-5-yl |
| A-1034. | 4-methyl-isoxazolidin-5-yl |
| A-1035. | 3-trifluoromethyl-isoxazolidin-5-yl |
| A-1036. | 4-trifluoromethyl-isoxazolidin-5-yl |
| A-1037. | 3-methoxy-isoxazolidin-5-yl |
| A-1038. | 4-methoxy-isoxazolidin-5-yl |
| A-1039. | 3-trifluoromethoxy-isoxazolidin-5-yl |
| A-1040. | 4-trifluoromethoxy-isoxazolidin-5-yl |
| A-1041. | isothiazolidin-3-yl |
| A-1042. | isothiazolidin-4-yl |
| A-1043. | isothiazolidin-5-yl |
| A-1044. | 4-chloro-isothiazolidin-3-yl |
| A-1045. | 5-chloro-isothiazolidin-3-yl |
| A-1046. | 4-bromo-isothiazolidin-3-yl |
| A-1047. | 5-bromo-isothiazolidin-3-yl |
| A-1048. | 4-cyano-isothiazolidin-3-yl |
| A-1049. | 5-cyano-isothiazolidin-3-yl |
| A-1050. | 4-nitro-isothiazolidin-3-yl |
| A-1051. | 5-nitro-isothiazolidin-3-yl |
| A-1052. | 4-methyl-isothiazolidin-3-yl |
| A-1053. | 5-methyl-isothiazolidin-3-yl |
| A-1054. | 4-trifluoromethyl-isothiazolidin-3-yl |
| A-1055. | 5-trifluoromethyl-isothiazolidin-3-yl |
| A-1056. | 4-methoxy-isothiazolidin-3-yl |
| A-1057. | 5-methoxy-isothiazolidin-3-yl |
| A-1058. | 4-trifluoromethoxy-isothiazolidin-3-yl |
| A-1059. | 5-trifluoromethoxy-isothiazolidin-3-yl |
| A-1060. | 3-chloro-isothiazolidin-4-yl |
| A-1061. | 5-chloro-isothiazolidin-4-yl |
| A-1062. | 3-bromo-isothiazolidin-4-yl |
| A-1063. | 5-bromo-isothiazolidin-4-yl |
| A-1064. | 3-cyano-isothiazolidin-4-yl |
| A-1065. | 5-cyano-isothiazolidin-4-yl |
| A-1066. | 3-nitro-isothiazolidin-4-yl |
| A-1067. | 5-nitro-isothiazolidin-4-yl |
| A-1068. | 3-methyl-isothiazolidin-4-yl |
| A-1069. | 5-methyl-isothiazolidin-4-yl |
| A-1070. | 3-trifluoromethyl-isothiazolidin-4-yl |
| A-1071. | 5-trifluoromethyl-isothiazolidin-4-yl |
| A-1072. | 3-methoxy-isothiazolidin-4-yl |
| A-1073. | 5-methoxy-isothiazolidin-4-yl |
| A-1074. | 3-trifluoromethoxy-isothiazolidin-4-yl |
| A-1075. | 5-trifluoromethoxy-isothiazolidin-4-yl |
| A-1076. | 3-chloro-isothiazolidin-5-yl |
| A-1077. | 4-chloro-isothiazolidin-5-yl |
| A-1078. | 3-bromo-isothiazolidin-5-yl |
| A-1079. | 4-bromo-isothiazolidin-5-yl |
| A-1080. | 3-cyano-isothiazolidin-5-yl |
| A-1081. | 4-cyano-isothiazolidin-5-yl |
| A-1082. | 3-nitro-isothiazolidin-5-yl |
| A-1083. | 4-nitro-isothiazolidin-5-yl |
| A-1084. | 3-methyl-isothiazolidin-5-yl |
| A-1085. | 4-methyl-isothiazolidin-5-yl |
| A-1086. | 3-trifluoromethyl-isothiazolidin-5-yl |
| A-1087. | 4-trifluoromethyl-isothiazolidin-5-yl |
| A-1088. | 3-methoxy-isothiazolidin-5-yl |
| A-1089. | 4-methoxy-isothiazolidin-5-yl |
| A-1090. | 3-trifluoromethoxy-isothiazolidin-5-yl |
| A-1091. | 4-trifluoromethoxy-isothiazolidin-5-yl |
| A-1092. | 2,3-dihydro-pyrrol-1-yl |
| A-1093. | 2,3-dihydro-pyrrol-2-yl |
| A-1094. | 2,3-dihydro-pyrrol-3-yl |
| A-1095. | 1-methyl-2,3-dihydro-pyrrol-2-yl |
| A-1096. | 1-methyl-2,3-dihydro-pyrrol-3-yl |
| A-1097. | 2-chloro-2,3-dihydro-pyrrol-1-yl |
| A-1098. | 3-chloro-2,3-dihydro-pyrrol-1-yl |
| A-1099. | 2-bromo-2,3-dihydro-pyrrol-1-yl |
| A-1100. | 3-bromo-2,3-dihydro-pyrrol-1-yl |
| A-1101. | 2-cyano-2,3-dihydro-pyrrol-1-yl |
| A-1102. | 3-cyano-2,3-dihydro-pyrrol-1-yl |
| A-1103. | 2-nitro-2,3-dihydro-pyrrol-1-yl |
| A-1104. | 3-nitro-2,3-dihydro-pyrrol-1-yl |
| A-1105. | 3-chloro-2,3-dihydro-pyrrol-2-yl |
| A-1106. | 4-chloro-2,3-dihydro-pyrrol-2-yl |
| A-1107. | 5-chloro-2,3-dihydro-pyrrol-2-yl |
| A-1108. | 3-bromo-2,3-dihydro-pyrrol-2-yl |
| A-1109. | 4-bromo-2,3-dihydro-pyrrol-2-yl |
| A-1110. | 5-bromo-2,3-dihydro-pyrrol-2-yl |
| A-1111. | 3-cyano-2,3-dihydro-pyrrol-2-yl |
| A-1112. | 4-cyano-2,3-dihydro-pyrrol-2-yl |
| A-1113. | 5-cyano-2,3-dihydro-pyrrol-2-yl |
| A-1114. | 3-nitro-2,3-dihydro-pyrrol-2-yl |
| A-1115. | 4-nitro-2,3-dihydro-pyrrol-2-yl |
| A-1116. | 5-nitro-2,3-dihydro-pyrrol-2-yl |
| A-1117. | 3-methyl-2,3-dihydro-pyrrol-2-yl |
| A-1118. | 4-methyl-2,3-dihydro-pyrrol-2-yl |
| A-1119. | 5-methyl-2,3-dihydro-pyrrol-2-yl |
| A-1120. | 3-trifluoromethyl-2,3-dihydro-pyrrol-2-yl |
| A-1121. | 4-trifluoromethyl-2,3-dihydro-pyrrol-2-yl |
| A-1122. | 5-trifluoromethyl-2,3-dihydro-pyrrol-2-yl |
| A-1123. | 3-methoxy-2,3-dihydro-pyrrol-2-yl |
| A-1124. | 4-methoxy-2,3-dihydro-pyrrol-2-yl |
| A-1125. | 5-methoxy-2,3-dihydro-pyrrol-2-yl |
| A-1126. | 3-trifluoromethoxy-2,3-dihydro-pyrrol-2-yl |
| A-1127. | 4-trifluoromethoxy-2,3-dihydro-pyrrol-2-yl |
| A-1128. | 5-trifluoromethoxy-2,3-dihydro-pyrrol-2-yl |
| A-1129. | 2-chloro-2,3-dihydro-pyrrol-3-yl |
| A-1130. | 4-chloro-2,3-dihydro-pyrrol-3-yl |
| A-1131. | 5-chloro-2,3-dihydro-pyrrol-3-yl |
| A-1132. | 2-bromo-2,3-dihydro-pyrrol-3-yl |
| A-1133. | 4-bromo-2,3-dihydro-pyrrol-3-yl |
| A-1134. | 5-bromo-2,3-dihydro-pyrrol-3-yl |
| A-1135. | 2-cyano-2,3-dihydro-pyrrol-3-yl |
| A-1136. | 4-cyano-2,3-dihydro-pyrrol-3-yl |
| A-1137. | 5-cyano-2,3-dihydro-pyrrol-3-yl |
| A-1138. | 2-nitro-2,3-dihydro-pyrrol-3-yl |
| A-1139. | 4-nitro-2,3-dihydro-pyrrol-3-yl |
| A-1140. | 5-nitro-2,3-dihydro-pyrrol-3-yl |
| A-1141. | 2-methyl-2,3-dihydro-pyrrol-3-yl |
| A-1142. | 4-methyl-2,3-dihydro-pyrrol-3-yl |
| A-1143. | 5-methyl-2,3-dihydro-pyrrol-3-yl |
| A-1144. | 2-trifluoromethyl-2,3-dihydro-pyrrol-3-yl |
| A-1145. | 4-trifluoromethyl-2,3-dihydro-pyrrol-3-yl |
| A-1146. | 5-trifluoromethyl-2,3-dihydro-pyrrol-3-yl |
| A-1147. | 2-methoxy-2,3-dihydro-pyrrol-3-yl |
| A-1148. | 4-methoxy-2,3-dihydro-pyrrol-3-yl |
| A-1149. | 5-methoxy-2,3-dihydro-pyrrol-3-yl |
| A-1150. | 2-trifluoromethoxy-2,3-dihydro-pyrrol-3-yl |
| A-1151. | 4-trifluoromethoxy-2,3-dihydro-pyrrol-3-yl |
| A-1152. | 5-trifluoromethoxy-2,3-dihydro-pyrrol-3-yl |
| A-1153. | 3-chloro-1-methyl-2,3-dihydro-pyrrol-2-yl |
| A-1154. | 4-chloro-1-methyl-2,3-dihydro-pyrrol-2-yl |
| A-1155. | 5-chloro-1-methyl-2,3-dihydro-pyrrol-2-yl |
| A-1156. | 3-bromo-1-methyl-2,3-dihydro-pyrrol-2-yl |
| A-1157. | 4-bromo-1-methyl-2,3-dihydro-pyrrol-2-yl |
| A-1158. | 5-bromo-1-methyl-2,3-dihydro-pyrrol-2-yl |
| A-1159. | 3-cyano-1-methyl-2,3-dihydro-pyrrol-2-yl |
| A-1160. | 4-cyano-1-methyl-2,3-dihydro-pyrrol-2-yl |
| A-1161. | 5-cyano-1-methyl-2,3-dihydro-pyrrol-2-yl |
| A-1162. | 3-nitro-1-methyl-2,3-dihydro-pyrrol-2-yl |
| A-1163. | 4-nitro-1-methyl-2,3-dihydro-pyrrol-2-yl |
| A-1164. | 5-nitro-1-methyl-2,3-dihydro-pyrrol-2-yl |
| A-1165. | 3-methyl-1-methyl-2,3-dihydro-pyrrol-2-yl |
| A-1166. | 4-methyl-1-methyl-2,3-dihydro-pyrrol-2-yl |
| A-1167. | 5-methyl-1-methyl-2,3-dihydro-pyrrol-2-yl |
| A-1168. | 3-triluoromethyl-1-methyl-2,3-dihydro-pyrrol-2-yl |
| A-1169. | 4-triluoromethyl-1-methyl-2,3-dihydro-pyrrol-2-yl |
| A-1170. | 5-triluoromethyl-1-methyl-2,3-dihydro-pyrrol-2-yl |
| A-1171. | 3-methoxy-1-methyl-2,3-dihydro-pyrrol-2-yl |
| A-1172. | 4-methoxy-1-methyl-2,3-dihydro-pyrrol-2-yl |
| A-1173. | 5-methoxy-1-methyl-2,3-dihydro-pyrrol-2-yl |
| A-1174. | 3-trifluoromethoxy-1-methyl-2,3-dihydro-pyrrol-2-yl |
| A-1175. | 4-trifluoromethoxy-1-methyl-2,3-dihydro-pyrrol-2-yl |
| A-1176. | 5-trifluoromethoxy-1-methyl-2,3-dihydro-pyrrol-2-yl |
| A-1177. | 2-chloro-1-methyl-2,3-dihydro-pyrrol-3-yl |
| A-1178. | 4-chloro-1-methyl-2,3-dihydro-pyrrol-3-yl |
| A-1179. | 5-chloro-1-methyl-2,3-dihydro-pyrrol-3-yl |
| A-1180. | 2-bromo-1-methyl-2,3-dihydro-pyrrol-3-yl |
| A-1181. | 4-bromo-1-methyl-2,3-dihydro-pyrrol-3-yl |
| A-1182. | 5-bromo-1-methyl-2,3-dihydro-pyrrol-3-yl |
| A-1183. | 2-cyano-1-methyl-2,3-dihydro-pyrrol-3-yl |
| A-1184. | 4-cyano-1-methyl-2,3-dihydro-pyrrol-3-yl |
| A-1185. | 5-cyano-1-methyl-2,3-dihydro-pyrrol-3-yl |
| A-1186. | 2-nitro-1-methyl-2,3-dihydro-pyrrol-3-yl |
| A-1187. | 4-nitro-1-methyl-2,3-dihydro-pyrrol-3-yl |
| A-1188. | 5-nitro-1-methyl-2,3-dihydro-pyrrol-3-yl |
| A-1189. | 2-methyl-1-methyl-2,3-dihydro-pyrrol-3-yl |
| A-1190. | 4-methyl-1-methyl-2,3-dihydro-pyrrol-3-yl |
| A-1191. | 5-methyl-1-methyl-2,3-dihydro-pyrrol-3-yl |
| A-1192. | 2-triluoromethyl-1-methyl-2,3-dihydro-pyrrol-3-yl |
| A-1193. | 4-triluoromethyl-1-methyl-2,3-dihydro-pyrrol-3-yl |
| A-1194. | 5-triluoromethyl-1-methyl-2,3-dihydro-pyrrol-3-yl |
| A-1195. | 2-methoxy-1-methyl-2,3-dihydro-pyrrol-3-yl |
| A-1196. | 4-methoxy-1-methyl-2,3-dihydro-pyrrol-3-yl |
| A-1197. | 5-methoxy-1-methyl-2,3-dihydro-pyrrol-3-yl |
| A-1198. | 2-trifluoromethoxy-1-methyl-2,3-dihydro-pyrrol-3-yl |
| A-1199. | 4-trifluoromethoxy-1-methyl-2,3-dihydro-pyrrol-3-yl |
| A-1200. | 5-trifluoromethoxy-1-methyl-2,3-dihydro-pyrrol-3-yl |
| A-1201. | 2,5-dihydro-pyrrol-1-yl |
| A-1202. | 2,5-dihydro-pyrrol-2-yl |
| A-1203. | 2,5-dihydro-pyrrol-3-yl |
| A-1204. | 1-methyl-2,5-dihydro-pyrrol-2-yl |
| A-1205. | 1-methyl-2,5-dihydro-pyrrol-3-yl |
| A-1206. | 2-chloro-2,5-dihydro-pyrrol-1-yl |
| A-1207. | 3-chloro-2,5-dihydro-pyrrol-1-yl |
| A-1208. | 2-bromo-2,5-dihydro-pyrrol-1-yl |
| A-1209. | 3-bromo-2,5-dihydro-pyrrol-1-yl |
| A-1210. | 2-cyano-2,5-dihydro-pyrrol-1-yl |
| A-1211. | 3-cyano-2,5-dihydro-pyrrol-1-yl |
| A-1212. | 2-nitro-2,5-dihydro-pyrrol-1-yl |
| A-1213. | 3-nitro-2,5-dihydro-pyrrol-1-yl |
| A-1214. | 2-methyl-2,5-dihydro-pyrrol-1-yl |
| A-1215. | 3-methyl-2,5-dihydro-pyrrol-1-yl |
| A-1216. | 2-trifluoromethyl-2,5-dihydro-pyrrol-1-yl |
| A-1217. | 3-trifluoromethyl-2,5-dihydro-pyrrol-1-yl |
| A-1218. | 2-methoxy-2,5-dihydro-pyrrol-1-yl |
| A-1219. | 3-methoxy-2,5-dihydro-pyrrol-1-yl |
| A-1220. | 2-trifluoromethoxy-2,5-dihydro-pyrrol-1-yl |
| A-1221. | 3-trifluoromethoxy-2,5-dihydro-pyrrol-1-yl |
| A-1222. | 3-chloro-2,5-dihydro-pyrrol-2-yl |
| A-1223. | 4-chloro-2,5-dihydro-pyrrol-2-yl |
| A-1224. | 5-chloro-2,5-dihydro-pyrrol-2-yl |
| A-1225. | 3-bromo-2,5-dihydro-pyrrol-2-yl |
| A-1226. | 4-bromo-2,5-dihydro-pyrrol-2-yl |
| A-1227. | 5-bromo-2,5-dihydro-pyrrol-2-yl |
| A-1228. | 3-cyano-2,5-dihydro-pyrrol-2-yl |
| A-1229. | 4-cyano-2,5-dihydro-pyrrol-2-yl |
| A-1230. | 5-cyano-2,5-dihydro-pyrrol-2-yl |
| A-1231. | 3-nitro-2,5-dihydro-pyrrol-2-yl |
| A-1232. | 4-nitro-2,5-dihydro-pyrrol-2-yl |
| A-1233. | 5-nitro-2,5-dihydro-pyrrol-2-yl |
| A-1234. | 3-methyl-2,5-dihydro-pyrrol-2-yl |
| A-1235. | 4-methyl-2,5-dihydro-pyrrol-2-yl |
| A-1236. | 5-methyl-2,5-dihydro-pyrrol-2-yl |
| A-1237. | 3-trifluoromethyl-2,5-dihydro-pyrrol-2-yl |
| A-1238. | 4-trifluoromethyl-2,5-dihydro-pyrrol-2-yl |
| A-1239. | 5-trifluoromethyl-2,5-dihydro-pyrrol-2-yl |
| A-1240. | 3-methoxy-2,5-dihydro-pyrrol-2-yl |
| A-1241. | 4-methoxy-2,5-dihydro-pyrrol-2-yl |
| A-1242. | 5-methoxy-2,5-dihydro-pyrrol-2-yl |
| A-1243. | 3-trifluoromethoxy-2,5-dihydro-pyrrol-2-yl |
| A-1244. | 4-trifluoromethoxy-2,5-dihydro-pyrrol-2-yl |
| A-1245. | 5-trifluoromethoxy-2,5-dihydro-pyrrol-2-yl |
| A-1246. | 2-chloro-2,5-dihydro-pyrrol-3-yl |
| A-1247. | 4-chloro-2,5-dihydro-pyrrol-3-yl |
| A-1248. | 5-chloro-2,5-dihydro-pyrrol-3-yl |
| A-1249. | 2-bromo-2,5-dihydro-pyrrol-3-yl |
| A-1250. | 4-bromo-2,5-dihydro-pyrrol-3-yl |
| A-1251. | 5-bromo-2,5-dihydro-pyrrol-3-yl |
| A-1252. | 2-cyano-2,5-dihydro-pyrrol-3-yl |
| A-1253. | 4-cyano-2,5-dihydro-pyrrol-3-yl |
| A-1254. | 5-cyano-2,5-dihydro-pyrrol-3-yl |
| A-1255. | 2-nitro-2,5-dihydro-pyrrol-3-yl |
| A-1256. | 4-nitro-2,5-dihydro-pyrrol-3-yl |
| A-1257. | 5-nitro-2,5-dihydro-pyrrol-3-yl |
| A-1258. | 2-methyl-2,5-dihydro-pyrrol-3-yl |
| A-1259. | 4-methyl-2,5-dihydro-pyrrol-3-yl |
| A-1260. | 5-methyl-2,5-dihydro-pyrrol-3-yl |
| A-1261. | 2-trifluoromethyl-2,5-dihydro-pyrrol-3-yl |
| A-1262. | 4-trifluoromethyl-2,5-dihydro-pyrrol-3-yl |
| A-1263. | 5-trifluoromethyl-2,5-dihydro-pyrrol-3-yl |
| A-1264. | 2-methoxy-2,5-dihydro-pyrrol-3-yl |
| A-1265. | 4-methoxy-2,5-dihydro-pyrrol-3-yl |
| A-1266. | 5-methoxy-2,5-dihydro-pyrrol-3-yl |
| A-1267. | 2-trifluoromethoxy-2,5-dihydro-pyrrol-3-yl |
| A-1268. | 4-trifluoromethoxy-2,5-dihydro-pyrrol-3-yl |
| A-1269. | 5-trifluoromethoxy-2,5-dihydro-pyrrol-3-yl |
| A-1270. | 3-chloro-1-methyl-2,5-dihydro-pyrrol-2-yl |
| A-1271. | 4-chloro-1-methyl-2,5-dihydro-pyrrol-2-yl |
| A-1272. | 5-chloro-1-methyl-2,5-dihydro-pyrrol-2-yl |
| A-1273. | 3-bromo-1-methyl-2,5-dihydro-pyrrol-2-yl |
| A-1274. | 4-bromo-1-methyl-2,5-dihydro-pyrrol-2-yl |
| A-1275. | 5-bromo-1-methyl-2,5-dihydro-pyrrol-2-yl |
| A-1276. | 3-cyano-1-methyl-2,5-dihydro-pyrrol-2-yl |
| A-1277. | 4-cyano-1-methyl-2,5-dihydro-pyrrol-2-yl |
| A-1278. | 5-cyano-1-methyl-2,5-dihydro-pyrrol-2-yl |
| A-1279. | 3-nitro-1-methyl-2,5-dihydro-pyrrol-2-yl |
| A-1280. | 4-nitro-1-methyl-2,5-dihydro-pyrrol-2-yl |
| A-1281. | 5-nitro-1-methyl-2,5-dihydro-pyrrol-2-yl |
| A-1282. | 3-methyl-1-methyl-2,5-dihydro-pyrrol-2-yl |
| A-1283. | 4-methyl-1-methyl-2,5-dihydro-pyrrol-2-yl |
| A-1284. | 5-methyl-1-methyl-2,5-dihydro-pyrrol-2-yl |
| A-1285. | 3-trifluoromethyl-1-methyl-2,5-dihydro-pyrrol-2-yl |
| A-1286. | 4-trifluoromethyl-1-methyl-2,5-dihydro-pyrrol-2-yl |
| A-1287. | 5-trifluoromethyl-1-methyl-2,5-dihydro-pyrrol-2-yl |
| A-1288. | 3-methoxy-1-methyl-2,5-dihydro-pyrrol-2-yl |
| A-1289. | 4-methoxy-1-methyl-2,5-dihydro-pyrrol-2-yl |
| A-1290. | 5-methoxy-1-methyl-2,5-dihydro-pyrrol-2-yl |
| A-1291. | 3-trifluoromethoxy-1-methyl-2,5-dihydro-pyrrol-2-yl |
| A-1292. | 4-trifluoromethoxy-1-methyl-2,5-dihydro-pyrrol-2-yl |
| A-1293. | 5-trifluoromethoxy-1-methyl-2,5-dihydro-pyrrol-2-yl |
| A-1294. | 2-chloro-1-methyl-2,5-dihydro-pyrrol-3-yl |
| A-1295. | 4-chloro-1-methyl-2,5-dihydro-pyrrol-3-yl |
| A-1296. | 5-chloro-1-methyl-2,5-dihydro-pyrrol-3-yl |
| A-1297. | 2-bromo-1-methyl-2,5-dihydro-pyrrol-3-yl |
| A-1298. | 4-bromo-1-methyl-2,5-dihydro-pyrrol-3-yl |
| A-1299. | 5-bromo-1-methyl-2,5-dihydro-pyrrol-3-yl |
| A-1300. | 2-cyano-1-methyl-2,5-dihydro-pyrrol-3-yl |
| A-1301. | 4-cyano-1-methyl-2,5-dihydro-pyrrol-3-yl |
| A-1302. | 5-cyano-1-methyl-2,5-dihydro-pyrrol-3-yl |
| A-1303. | 2-nitro-1-methyl-2,5-dihydro-pyrrol-3-yl |
| A-1304. | 4-nitro-1-methyl-2,5-dihydro-pyrrol-3-yl |
| A-1305. | 5-nitro-1-methyl-2,5-dihydro-pyrrol-3-yl |
| A-1306. | 2-methyl-1-methyl-2,5-dihydro-pyrrol-3-yl |
| A-1307. | 4-methyl-1-methyl-2,5-dihydro-pyrrol-3-yl |
| A-1308. | 5-methyl-1-methyl-2,5-dihydro-pyrrol-3-yl |
| A-1309. | 2-trifluoromethyl-1-methyl-2,5-dihydro-pyrrol-3-yl |
| A-1310. | 4-trifluoromethyl-1-methyl-2,5-dihydro-pyrrol-3-yl |
| A-1311. | 5-trifluoromethyl-1-methyl-2,5-dihydro-pyrrol-3-yl |
| A-1312. | 2-methoxy-1-methyl-2,5-dihydro-pyrrol-3-yl |
| A-1313. | 4-methoxy-1-methyl-2,5-dihydro-pyrrol-3-yl |
| A-1314. | 5-methoxy-1-methyl-2,5-dihydro-pyrrol-3-yl |
| A-1315. | 2-trifluoromethoxy-1-methyl-2,5-dihydro-pyrrol-3-yl |
| A-1316. | 4-trifluoromethoxy-1-methyl-2,5-dihydro-pyrrol-3-yl |
| A-1317. | 5-trifluoromethoxy-1-methyl-2,5-dihydro-pyrrol-3-yl |
| A-1318. | 2,3-dihydro-pyrazol-1-yl |
| A-1319. | 2,3-dihydro-pyrazol-3-yl |
| A-1320. | 2,3-dihydro-pyrazol-4-yl |
| A-1321. | 2,3-dihydro-pyrazol-5-yl |
| A-1322. | 1-methyl-2,3-dihydro-pyrazol-3-yl |
| A-1323. | 1-methyl-2,3-dihydro-pyrazol-4-yl |
| A-1324. | 1-methyl-2,3-dihydro-pyrazol-5-yl |
| A-1325. | 3-chloro-2,3-dihydro-pyrazol-1-yl |
| A-1326. | 4-chloro-2,3-dihydro-pyrazol-1-yl |
| A-1327. | 5-chloro-2,3-dihydro-pyrazol-1-yl |
| A-1328. | 3-bromo-2,3-dihydro-pyrazol-1-yl |
| A-1329. | 4-bromo-2,3-dihydro-pyrazol-1-yl |
| A-1330. | 5-bromo-2,3-dihydro-pyrazol-1-yl |
| A-1331. | 3-cyano-2,3-dihydro-pyrazol-1-yl |
| A-1332. | 4-cyano-2,3-dihydro-pyrazol-1-yl |
| A-1333. | 5-cyano-2,3-dihydro-pyrazol-1-yl |
| A-1334. | 3-nitro-2,3-dihydro-pyrazol-1-yl |
| A-1335. | 4-nitro-2,3-dihydro-pyrazol-1-yl |
| A-1336. | 5-nitro-2,3-dihydro-pyrazol-1-yl |
| A-1337. | 3-methyl-2,3-dihydro-pyrazol-1-yl |
| A-1338. | 4-methyl-2,3-dihydro-pyrazol-1-yl |
| A-1339. | 5-methyl-2,3-dihydro-pyrazol-1-yl |
| A-1340. | 3-trifluoromethyl-2,3-dihydro-pyrazol-1-yl |
| A-1341. | 4-trifluoromethyl-2,3-dihydro-pyrazol-1-yl |
| A-1342. | 5-trifluoromethyl-2,3-dihydro-pyrazol-1-yl |
| A-1343. | 3-methoxy-2,3-dihydro-pyrazol-1-yl |
| A-1344. | 4-methoxy-2,3-dihydro-pyrazol-1-yl |
| A-1345. | 5-methoxy-2,3-dihydro-pyrazol-1-yl |
| A-1346. | 3-trifluoromethoxy-2,3-dihydro-pyrazol-1-yl |
| A-1347. | 4-trifluoromethoxy-2,3-dihydro-pyrazol-1-yl |
| A-1348. | 5-trifluoromethoxy-2,3-dihydro-pyrazol-1-yl |
| A-1349. | 4-chloro-2,3-dihydro-pyrazol-3-yl |
| A-1350. | 5-chloro-2,3-dihydro-pyrazol-3-yl |
| A-1351. | 4-bromo-2,3-dihydro-pyrazol-3-yl |
| A-1352. | 5-bromo-2,3-dihydro-pyrazol-3-yl |
| A-1353. | 4-cyano-2,3-dihydro-pyrazol-3-yl |
| A-1354. | 5-cyano-2,3-dihydro-pyrazol-3-yl |
| A-1355. | 4-nitro-2,3-dihydro-pyrazol-3-yl |
| A-1356. | 5-nitro-2,3-dihydro-pyrazol-3-yl |
| A-1357. | 4-methyl-2,3-dihydro-pyrazol-3-yl |
| A-1358. | 5-methyl-2,3-dihydro-pyrazol-3-yl |
| A-1359. | 4-trifluoromethyl-2,3-dihydro-pyrazol-3-yl |
| A-1360. | 5-trifluoromethyl-2,3-dihydro-pyrazol-3-yl |
| A-1361. | 4-methoxy-2,3-dihydro-pyrazol-3-yl |
| A-1362. | 5-methoxy-2,3-dihydro-pyrazol-3-yl |
| A-1363. | 4-trifluoromethoxy-2,3-dihydro-pyrazol-3-yl |
| A-1364. | 5-trifluoromethoxy-2,3-dihydro-pyrazol-3-yl |
| A-1365. | 3-chloro-2,3-dihydro-pyrazol-4-yl |
| A-1366. | 5-chloro-2,3-dihydro-pyrazol-4-yl |
| A-1367. | 3-bromo-2,3-dihydro-pyrazol-4-yl |
| A-1368. | 5-bromo-2,3-dihydro-pyrazol-4-yl |
| A-1369. | 3-cyano-2,3-dihydro-pyrazol-4-yl |
| A-1370. | 5-cyano-2,3-dihydro-pyrazol-4-yl |
| A-1371. | 3-nitro-2,3-dihydro-pyrazol-4-yl |
| A-1372. | 5-nitro-2,3-dihydro-pyrazol-4-yl |
| A-1373. | 3-methyl-2,3-dihydro-pyrazol-4-yl |
| A-1374. | 5-methyl-2,3-dihydro-pyrazol-4-yl |
| A-1375. | 3-trifluoromethyl-2,3-dihydro-pyrazol-4-yl |
| A-1376. | 5-trifluoromethyl-2,3-dihydro-pyrazol-4-yl |
| A-1377. | 3-methoxy-2,3-dihydro-pyrazol-4-yl |
| A-1378. | 5-methoxy-2,3-dihydro-pyrazol-4-yl |
| A-1379. | 3-trifluoromethoxy-2,3-dihydro-pyrazol-4-yl |
| A-1380. | 5-trifluoromethoxy-2,3-dihydro-pyrazol-4-yl |
| A-1381. | 3-chloro-2,3-dihydro-pyrazol-5-yl |
| A-1382. | 4-chloro-2,3-dihydro-pyrazol-5-yl |
| A-1383. | 3-bromo-2,3-dihydro-pyrazol-5-yl |
| A-1384. | 4-bromo-2,3-dihydro-pyrazol-5-yl |
| A-1385. | 3-cyano-2,3-dihydro-pyrazol-5-yl |
| A-1386. | 4-cyano-2,3-dihydro-pyrazol-5-yl |
| A-1387. | 3-nitro-2,3-dihydro-pyrazol-5-yl |
| A-1388. | 4-nitro-2,3-dihydro-pyrazol-5-yl |
| A-1389. | 3-methyl-2,3-dihydro-pyrazol-5-yl |
| A-1390. | 4-methyl-2,3-dihydro-pyrazol-5-yl |
| A-1391. | 3-trifluoromethyl-2,3-dihydro-pyrazol-5-yl |
| A-1392. | 4-trifluoromethyl-2,3-dihydro-pyrazol-5-yl |
| A-1393. | 3-methoxy-2,3-dihydro-pyrazol-5-yl |
| A-1394. | 4-methoxy-2,3-dihydro-pyrazol-5-yl |
| A-1395. | 3-trifluoromethoxy-2,3-dihydro-pyrazol-5-yl |
| A-1396. | 4-trifluoromethoxy-2,3-dihydro-pyrazol-5-yl |
| A-1397. | 3-chloro-1-methyl-2,3-dihydro-pyrazol-2-yl |
| A-1398. | 4-chloro-1-methyl-2,3-dihydro-pyrazol-2-yl |
| A-1399. | 5-chloro-1-methyl-2,3-dihydro-pyrazol-2-yl |
| A-1400. | 3-bromo-1-methyl-2,3-dihydro-pyrazol-2-yl |
| A-1401. | 4-bromo-1-methyl-2,3-dihydro-pyrazol-2-yl |
| A-1402. | 5-bromo-1-methyl-2,3-dihydro-pyrazol-2-yl |
| A-1403. | 3-cyano-1-methyl-2,3-dihydro-pyrazol-2-yl |
| A-1404. | 4-cyano-1-methyl-2,3-dihydro-pyrazol-2-yl |
| A-1405. | 5-cyano-1-methyl-2,3-dihydro-pyrazol-2-yl |
| A-1406. | 3-nitro-1-methyl-2,3-dihydro-pyrazol-2-yl |
| A-1407. | 4-nitro-1-methyl-2,3-dihydro-pyrazol-2-yl |
| A-1408. | 5-nitro-1-methyl-2,3-dihydro-pyrazol-2-yl |
| A-1409. | 3-methyl-1-methyl-2,3-dihydro-pyrazol-2-yl |
| A-1410. | 4-methyl-1-methyl-2,3-dihydro-pyrazol-2-yl |
| A-1411. | 5-methyl-1-methyl-2,3-dihydro-pyrazol-2-yl |
| A-1412. | 3-trifluoromethyl-1-methyl-2,3-dihydro-pyrazol-2-yl |
| A-1413. | 4-trifluoromethyl-1-methyl-2,3-dihydro-pyrazol-2-yl |
| A-1414. | 5-trifluoromethyl-1-methyl-2,3-dihydro-pyrazol-2-yl |
| A-1415. | 3-methoxy-1-methyl-2,3-dihydro-pyrazol-2-yl |
| A-1416. | 4-methoxy-1-methyl-2,3-dihydro-pyrazol-2-yl |
| A-1417. | 5-methoxy-1-methyl-2,3-dihydro-pyrazol-2-yl |
| A-1418. | 3-trifluoromethoxy-1-methyl-2,3-dihydro-pyrazol-2-yl |
| A-1419. | 4-trifluoromethoxy-1-methyl-2,3-dihydro-pyrazol-2-yl |
| A-1420. | 5-trifluoromethoxy-1-methyl-2,3-dihydro-pyrazol-2-yl |
| A-1421. | 4-chloro-1-methyl-2,3-dihydro-pyrazol-3-yl |
| A-1422. | 5-chloro-1-methyl-2,3-dihydro-pyrazol-3-yl |
| A-1423. | 4-bromo-1-methyl-2,3-dihydro-pyrazol-3-yl |
| A-1424. | 5-bromo-1-methyl-2,3-dihydro-pyrazol-3-yl |
| A-1425. | 4-cyano-1-methyl-2,3-dihydro-pyrazol-3-yl |
| A-1426. | 5-cyano-1-methyl-2,3-dihydro-pyrazol-3-yl |
| A-1427. | 4-nitro-1-methyl-2,3-dihydro-pyrazol-3-yl |
| A-1428. | 5-nitro-1-methyl-2,3-dihydro-pyrazol-3-yl |
| A-1429. | 4-methyl-1-methyl-2,3-dihydro-pyrazol-3-yl |
| A-1430. | 5-methyl-1-methyl-2,3-dihydro-pyrazol-3-yl |
| A-1431. | 4-trifluoromethyl-1-methyl-2,3-dihydro-pyrazol-3-yl |
| A-1432. | 5-trifluoromethyl-1-methyl-2,3-dihydro-pyrazol-3-yl |
| A-1433. | 4-methoxy-1-methyl-2,3-dihydro-pyrazol-3-yl |
| A-1434. | 5-methoxy-1-methyl-2,3-dihydro-pyrazol-3-yl |
| A-1435. | 4-trifluoromethoxy-1-methyl-2,3-dihydro-pyrazol-3-yl |
| A-1436. | 5-trifluoromethoxy-1-methyl-2,3-dihydro-pyrazol-3-yl |
| A-1437. | 3-chloro-1-methyl-2,3-dihydro-pyrazol-4-yl |
| A-1438. | 5-chloro-1-methyl-2,3-dihydro-pyrazol-4-yl |
| A-1439. | 3-bromo-1-methyl-2,3-dihydro-pyrazol-4-yl |
| A-1440. | 5-bromo-1-methyl-2,3-dihydro-pyrazol-4-yl |
| A-1441. | 3-cyano-1-methyl-2,3-dihydro-pyrazol-4-yl |
| A-1442. | 5-cyano-1-methyl-2,3-dihydro-pyrazol-4-yl |
| A-1443. | 3-nitro-1-methyl-2,3-dihydro-pyrazol-4-yl |
| A-1444. | 5-nitro-1-methyl-2,3-dihydro-pyrazol-4-yl |
| A-1445. | 3-methyl-1-methyl-2,3-dihydro-pyrazol-4-yl |
| A-1446. | 5-methyl-1-methyl-2,3-dihydro-pyrazol-4-yl |
| A-1447. | 3-trifluoromethyl-1-methyl-2,3-dihydro-pyrazol-4-yl |
| A-1448. | 5-trifluoromethyl-1-methyl-2,3-dihydro-pyrazol-4-yl |
| A-1449. | 3-methoxy-1-methyl-2,3-dihydro-pyrazol-4-yl |
| A-1450. | 5-methoxy-1-methyl-2,3-dihydro-pyrazol-4-yl |
| A-1451. | 3-trifluoromethoxy-1-methyl-2,3-dihydro-pyrazol-4-yl |
| A-1452. | 5-trifluoromethoxy-1-methyl-2,3-dihydro-pyrazol-4-yl |
| A-1453. | 3-chloro-1-methyl-2,3-dihydro-pyrazol-5-yl |
| A-1454. | 4-chloro-1-methyl-2,3-dihydro-pyrazol-5-yl |
| A-1455. | 3-bromo-1-methyl-2,3-dihydro-pyrazol-5-yl |
| A-1456. | 4-bromo-1-methyl-2,3-dihydro-pyrazol-5-yl |
| A-1457. | 3-cyano-1-methyl-2,3-dihydro-pyrazol-5-yl |
| A-1458. | 4-cyano-1-methyl-2,3-dihydro-pyrazol-5-yl |
| A-1459. | 3-nitro-1-methyl-2,3-dihydro-pyrazol-5-yl |
| A-1460. | 4-nitro-1-methyl-2,3-dihydro-pyrazol-5-yl |
| A-1461. | 3-methyl-1-methyl-2,3-dihydro-pyrazol-5-yl |
| A-1462. | 4-methyl-1-methyl-2,3-dihydro-pyrazol-5-yl |
| A-1463. | 3-trifluoromethyl-1-methyl-2,3-dihydro-pyrazol-5-yl |
| A-1464. | 4-trifluoromethyl-1-methyl-2,3-dihydro-pyrazol-5-yl |
| A-1465. | 3-methoxy-1-methyl-2,3-dihydro-pyrazol-5-yl |
| A-1466. | 4-methoxy-1-methyl-2,3-dihydro-pyrazol-5-yl |
| A-1467. | 3-trifluoromethoxy-1-methyl-2,3-dihydro-pyrazol-5-yl |
| A-1468. | 4-trifluoromethoxy-1-methyl-2,3-dihydro-pyrazol-5-yl |
| A-1469. | 2,5-dihydro-pyrazol-1-yl |
| A-1470. | 2,5-dihydro-pyrazol-3-yl |
| A-1471. | 2,5-dihydro-pyrazol-4-yl |
| A-1472. | 2,5-dihydro-pyrazol-5-yl |
| A-1473. | 1-methyl-2,5-dihydro-pyrazol-3-yl |
| A-1474. | 1-methyl-2,5-dihydro-pyrazol-4-yl |
| A-1475. | 1-methyl-2,5-dihydro-pyrazol-5-yl |
| A-1476. | 3-chloro-2,5-dihydro-pyrazol-1-yl |
| A-1477. | 4-chloro-2,5-dihydro-pyrazol-1-yl |
| A-1478. | 5-chloro-2,5-dihydro-pyrazol-1-yl |
| A-1479. | 3-bromo-2,5-dihydro-pyrazol-1-yl |
| A-1480. | 4-bromo-2,5-dihydro-pyrazol-1-yl |
| A-1481. | 5-bromo-2,5-dihydro-pyrazol-1-yl |
| A-1482. | 3-cyano-2,5-dihydro-pyrazol-1-yl |
| A-1483. | 4-cyano-2,5-dihydro-pyrazol-1-yl |
| A-1484. | 5-cyano-2,5-dihydro-pyrazol-1-yl |
| A-1485. | 3-nitro-2,5-dihydro-pyrazol-1-yl |
| A-1486. | 4-nitro-2,5-dihydro-pyrazol-1-yl |
| A-1487. | 5-nitro-2,5-dihydro-pyrazol-1-yl |
| A-1488. | 3-methyl-2,5-dihydro-pyrazol-1-yl |
| A-1489. | 4-methyl-2,5-dihydro-pyrazol-1-yl |
| A-1490. | 5-methyl-2,5-dihydro-pyrazol-1-yl |
| A-1491. | 3-trifluoromethyl-2,5-dihydro-pyrazol-1-yl |
| A-1492. | 4-trifluoromethyl-2,5-dihydro-pyrazol-1-yl |
| A-1493. | 5-trifluoromethyl-2,5-dihydro-pyrazol-1-yl |
| A-1494. | 3-methoxy-2,5-dihydro-pyrazol-1-yl |
| A-1495. | 4-methoxy-2,5-dihydro-pyrazol-1-yl |
| A-1496. | 5-methoxy-2,5-dihydro-pyrazol-1-yl |
| A-1497. | 3-trifluoromethoxy-2,5-dihydro-pyrazol-1-yl |
| A-1498. | 4-trifluoromethoxy-2,5-dihydro-pyrazol-1-yl |
| A-1499. | 5-trifluoromethoxy-2,5-dihydro-pyrazol-1-yl |
| A-1500. | 4-chloro-2,5-dihydro-pyrazol-3-yl |
| A-1501. | 5-chloro-2,5-dihydro-pyrazol-3-yl |
| A-1502. | 4-bromo-2,5-dihydro-pyrazol-3-yl |
| A-1503. | 5-bromo-2,5-dihydro-pyrazol-3-yl |
| A-1504. | 4-cyano-2,5-dihydro-pyrazol-3-yl |
| A-1505. | 5-cyano-2,5-dihydro-pyrazol-3-yl |
| A-1506. | 4-nitro-2,5-dihydro-pyrazol-3-yl |
| A-1507. | 5-nitro-2,5-dihydro-pyrazol-3-yl |
| A-1508. | 4-methyl-2,5-dihydro-pyrazol-3-yl |
| A-1509. | 5-methyl-2,5-dihydro-pyrazol-3-yl |
| A-1510. | 4-trifluoromethyl-2,5-dihydro-pyrazol-3-yl |
| A-1511. | 5-trifluoromethyl-2,5-dihydro-pyrazol-3-yl |
| A-1512. | 4-methoxy-2,5-dihydro-pyrazol-3-yl |
| A-1513. | 5-methoxy-2,5-dihydro-pyrazol-3-yl |
| A-1514. | 4-trifluoromethoxy-2,5-dihydro-pyrazol-3-yl |
| A-1515. | 5-trifluoromethoxy-2,5-dihydro-pyrazol-3-yl |
| A-1516. | 3-chloro-2,5-dihydro-pyrazol-4-yl |
| A-1517. | 5-chloro-2,5-dihydro-pyrazol-4-yl |
| A-1518. | 3-bromo-2,5-dihydro-pyrazol-4-yl |
| A-1519. | 5-bromo-2,5-dihydro-pyrazol-4-yl |
| A-1520. | 3-cyano-2,5-dihydro-pyrazol-4-yl |
| A-1521. | 5-cyano-2,5-dihydro-pyrazol-4-yl |
| A-1522. | 3-nitro-2,5-dihydro-pyrazol-4-yl |
| A-1523. | 5-nitro-2,5-dihydro-pyrazol-4-yl |
| A-1524. | 3-methyl-2,5-dihydro-pyrazol-4-yl |
| A-1525. | 5-methyl-2,5-dihydro-pyrazol-4-yl |
| A-1526. | 3-trifluoromethyl-2,5-dihydro-pyrazol-4-yl |
| A-1527. | 5-trifluoromethyl-2,5-dihydro-pyrazol-4-yl |
| A-1528. | 3-methoxy-2,5-dihydro-pyrazol-4-yl |
| A-1529. | 5-methoxy-2,5-dihydro-pyrazol-4-yl |
| A-1530. | 3-trifluoromethoxy-2,5-dihydro-pyrazol-4-yl |
| A-1531. | 5-trifluoromethoxy-2,5-dihydro-pyrazol-4-yl |
| A-1532. | 3-chloro-2,5-dihydro-pyrazol-5-yl |
| A-1533. | 4-chloro-2,5-dihydro-pyrazol-5-yl |
| A-1534. | 3-bromo-2,5-dihydro-pyrazol-5-yl |
| A-1535. | 4-bromo-2,5-dihydro-pyrazol-5-yl |
| A-1536. | 3-cyano-2,5-dihydro-pyrazol-5-yl |
| A-1537. | 4-cyano-2,5-dihydro-pyrazol-5-yl |
| A-1538. | 3-nitro-2,5-dihydro-pyrazol-5-yl |
| A-1539. | 4-nitro-2,5-dihydro-pyrazol-5-yl |
| A-1540. | 3-methyl-2,5-dihydro-pyrazol-5-yl |
| A-1541. | 4-methyl-2,5-dihydro-pyrazol-5-yl |
| A-1542. | 3-trifluoromethyl-2,5-dihydro-pyrazol-5-yl |
| A-1543. | 4-trifluoromethyl-2,5-dihydro-pyrazol-5-yl |
| A-1544. | 3-methoxy-2,5-dihydro-pyrazol-5-yl |
| A-1545. | 4-methoxy-2,5-dihydro-pyrazol-5-yl |
| A-1546. | 3-trifluoromethoxy-2,5-dihydro-pyrazol-5-yl |
| A-1547. | 4-trifluoromethoxy-2,5-dihydro-pyrazol-5-yl |
| A-1548. | 3-chloro-1-methyl-2,5-dihydro-pyrazol-2-yl |
| A-1549. | 4-chloro-1-methyl-2,5-dihydro-pyrazol-2-yl |
| A-1550. | 5-chloro-1-methyl-2,5-dihydro-pyrazol-2-yl |
| A-1551. | 3-bromo-1-methyl-2,5-dihydro-pyrazol-2-yl |
| A-1552. | 4-bromo-1-methyl-2,5-dihydro-pyrazol-2-yl |
| A-1553. | 5-bromo-1-methyl-2,5-dihydro-pyrazol-2-yl |
| A-1554. | 3-cyano-1-methyl-2,5-dihydro-pyrazol-2-yl |
| A-1555. | 4-cyano-1-methyl-2,5-dihydro-pyrazol-2-yl |
| A-1556. | 5-cyano-1-methyl-2,5-dihydro-pyrazol-2-yl |
| A-1557. | 3-nitro-1-methyl-2,5-dihydro-pyrazol-2-yl |
| A-1558. | 4-nitro-1-methyl-2,5-dihydro-pyrazol-2-yl |
| A-1559. | 5-nitro-1-methyl-2,5-dihydro-pyrazol-2-yl |
| A-1560. | 3-methyl-1-methyl-2,5-dihydro-pyrazol-2-yl |
| A-1561. | 4-methyl-1-methyl-2,5-dihydro-pyrazol-2-yl |
| A-1562. | 5-methyl-1-methyl-2,5-dihydro-pyrazol-2-yl |
| A-1563. | 3-trifluoromethyl-1-methyl-2,5-dihydro-pyrazol-2-yl |
| A-1564. | 4-trifluoromethyl-1-methyl-2,5-dihydro-pyrazol-2-yl |
| A-1565. | 5-trifluoromethyl-1-methyl-2,5-dihydro-pyrazol-2-yl |
| A-1566. | 3-methoxy-1-methyl-2,5-dihydro-pyrazol-2-yl |
| A-1567. | 4-methoxy-1-methyl-2,5-dihydro-pyrazol-2-yl |
| A-1568. | 5-methoxy-1-methyl-2,5-dihydro-pyrazol-2-yl |
| A-1569. | 3-trifluoromethoxy-1-methyl-2,5-dihydro-pyrazol-2-yl |
| A-1570. | 4-trifluoromethoxy-1-methyl-2,5-dihydro-pyrazol-2-yl |
| A-1571. | 5-trifluoromethoxy-1-methyl-2,5-dihydro-pyrazol-2-yl |
| A-1572. | 4-chloro-1-methyl-2,5-dihydro-pyrazol-3-yl |
| A-1573. | 5-chloro-1-methyl-2,5-dihydro-pyrazol-3-yl |
| A-1574. | 4-bromo-1-methyl-2,5-dihydro-pyrazol-3-yl |
| A-1575. | 5-bromo-1-methyl-2,5-dihydro-pyrazol-3-yl |
| A-1576. | 4-cyano-1-methyl-2,5-dihydro-pyrazol-3-yl |
| A-1577. | 5-cyano-1-methyl-2,5-dihydro-pyrazol-3-yl |
| A-1578. | 4-nitro-1-methyl-2,5-dihydro-pyrazol-3-yl |
| A-1579. | 5-nitro-1-methyl-2,5-dihydro-pyrazol-3-yl |
| A-1580. | 4-methyl-1-methyl-2,5-dihydro-pyrazol-3-yl |
| A-1581. | 5-methyl-1-methyl-2,5-dihydro-pyrazol-3-yl |
| A-1582. | 4-trifluoromethyl-1-methyl-2,5-dihydro-pyrazol-3-yl |
| A-1583. | 5-trifluoromethyl-1-methyl-2,5-dihydro-pyrazol-3-yl |
| A-1584. | 4-methoxy-1-methyl-2,5-dihydro-pyrazol-3-yl |
| A-1585. | 5-methoxy-1-methyl-2,5-dihydro-pyrazol-3-yl |
| A-1586. | 4-trifluoromethoxy-1-methyl-2,5-dihydro-pyrazol-3-yl |
| A-1587. | 5-trifluoromethoxy-1-methyl-2,5-dihydro-pyrazol-3-yl |
| A-1588. | 3-chloro-1-methyl-2,5-dihydro-pyrazol-4-yl |
| A-1589. | 5-chloro-1-methyl-2,5-dihydro-pyrazol-4-yl |
| A-1590. | 3-bromo-1-methyl-2,5-dihydro-pyrazol-4-yl |
| A-1591. | 5-bromo-1-methyl-2,5-dihydro-pyrazol-4-yl |
| A-1592. | 3-cyano-1-methyl-2,5-dihydro-pyrazol-4-yl |
| A-1593. | 5-cyano-1-methyl-2,5-dihydro-pyrazol-4-yl |
| A-1594. | 3-nitro-1-methyl-2,5-dihydro-pyrazol-4-yl |
| A-1595. | 5-nitro-1-methyl-2,5-dihydro-pyrazol-4-yl |
| A-1596. | 3-methyl-1-methyl-2,5-dihydro-pyrazol-4-yl |
| A-1597. | 5-methyl-1-methyl-2,5-dihydro-pyrazol-4-yl |
| A-1598. | 3-trifluoromethyl-1-methyl-2,5-dihydro-pyrazol-4-yl |
| A-1599. | 5-trifluoromethyl-1-methyl-2,5-dihydro-pyrazol-4-yl |
| A-1600. | 3-methoxy-1-methyl-2,5-dihydro-pyrazol-4-yl |
| A-1601. | 5-methoxy-1-methyl-2,5-dihydro-pyrazol-4-yl |
| A-1602. | 3-trifluoromethoxy-1-methyl-2,5-dihydro-pyrazol-4-yl |
| A-1603. | 5-trifluoromethoxy-1-methyl-2,5-dihydro-pyrazol-4-yl |
| A-1604. | 3-chloro-1-methyl-2,5-dihydro-pyrazol-5-yl |
| A-1605. | 4-chloro-1-methyl-2,5-dihydro-pyrazol-5-yl |
| A-1606. | 3-bromo-1-methyl-2,5-dihydro-pyrazol-5-yl |
| A-1607. | 4-bromo-1-methyl-2,5-dihydro-pyrazol-5-yl |
| A-1608. | 3-cyano-1-methyl-2,5-dihydro-pyrazol-5-yl |
| A-1609. | 4-cyano-1-methyl-2,5-dihydro-pyrazol-5-yl |
| A-1610. | 3-nitro-1-methyl-2,5-dihydro-pyrazol-5-yl |
| A-1611. | 4-nitro-1-methyl-2,5-dihydro-pyrazol-5-yl |
| A-1612. | 3-methyl-1-methyl-2,5-dihydro-pyrazol-5-yl |
| A-1613. | 4-methyl-1-methyl-2,5-dihydro-pyrazol-5-yl |
| A-1614. | 3-trifluoromethyl-1-methyl-2,5-dihydro-pyrazol-5-yl |
| A-1615. | 4-trifluoromethyl-1-methyl-2,5-dihydro-pyrazol-5-yl |
| A-1616. | 3-methoxy-1-methyl-2,5-dihydro-pyrazol-5-yl |
| A-1617. | 4-methoxy-1-methyl-2,5-dihydro-pyrazol-5-yl |
| A-1618. | 3-trifluoromethoxy-1-methyl-2,5-dihydro-pyrazol-5-yl |
| A-1619. | 4-trifluoromethoxy-1-methyl-2,5-dihydro-pyrazol-5-yl |
| A-1620. | 4,5-dihydro-pyrazol-1-yl |
| A-1621. | 4,5-dihydro-pyrazol-3-yl |
| A-1622. | 4,5-dihydro-pyrazol-4-yl |
| A-1623. | 4,5-dihydro-pyrazol-5-yl |
| A-1624. | 1-methyl-4,5-dihydro-pyrazol-3-yl |
| A-1625. | 1-methyl-4,5-dihydro-pyrazol-4-yl |
| A-1626. | 1-methyl-4,5-dihydro-pyrazol-5-yl |
| A-1627. | 3-chloro-4,5-dihydro-pyrazol-1-yl |
| A-1628. | 4-chloro-4,5-dihydro-pyrazol-1-yl |
| A-1629. | 5-chloro-4,5-dihydro-pyrazol-1-yl |
| A-1630. | 3-bromo-4,5-dihydro-pyrazol-1-yl |
| A-1631. | 4-bromo-4,5-dihydro-pyrazol-1-yl |
| A-1632. | 5-bromo-4,5-dihydro-pyrazol-1-yl |
| A-1633. | 3-cyano-4,5-dihydro-pyrazol-1-yl |
| A-1634. | 4-cyano-4,5-dihydro-pyrazol-1-yl |
| A-1635. | 5-cyano-4,5-dihydro-pyrazol-1-yl |
| A-1636. | 3-nitro-4,5-dihydro-pyrazol-1-yl |
| A-1637. | 4-nitro-4,5-dihydro-pyrazol-1-yl |
| A-1638. | 5-nitro-4,5-dihydro-pyrazol-1-yl |
| A-1639. | 3-methyl-4,5-dihydro-pyrazol-1-yl |
| A-1640. | 4-methyl-4,5-dihydro-pyrazol-1-yl |
| A-1641. | 5-methyl-4,5-dihydro-pyrazol-1-yl |
| A-1642. | 3-trifluoromethyl-4,5-dihydro-pyrazol-1-yl |
| A-1643. | 4-trifluoromethyl-4,5-dihydro-pyrazol-1-yl |
| A-1644. | 5-trifluoromethyl-4,5-dihydro-pyrazol-1-yl |
| A-1645. | 3-methoxy-4,5-dihydro-pyrazol-1-yl |
| A-1646. | 4-methoxy-4,5-dihydro-pyrazol-1-yl |
| A-1647. | 5-methoxy-4,5-dihydro-pyrazol-1-yl |
| A-1648. | 3-trifluoromethoxy-4,5-dihydro-pyrazol-1-yl |
| A-1649. | 4-trifluoromethoxy-4,5-dihydro-pyrazol-1-yl |
| A-1650. | 5-trifluoromethoxy-4,5-dihydro-pyrazol-1-yl |
| A-1651. | 4-chloro-4,5-dihydro-pyrazol-3-yl |
| A-1652. | 5-chloro-4,5-dihydro-pyrazol-3-yl |
| A-1653. | 4-bromo-4,5-dihydro-pyrazol-3-yl |
| A-1654. | 5-bromo-4,5-dihydro-pyrazol-3-yl |
| A-1655. | 4-cyano-4,5-dihydro-pyrazol-3-yl |
| A-1656. | 5-cyano-4,5-dihydro-pyrazol-3-yl |
| A-1657. | 4-nitro-4,5-dihydro-pyrazol-3-yl |
| A-1658. | 5-nitro-4,5-dihydro-pyrazol-3-yl |
| A-1659. | 4-methyl-4,5-dihydro-pyrazol-3-yl |
| A-1660. | 5-methyl-4,5-dihydro-pyrazol-3-yl |
| A-1661. | 4-trifluoromethyl-4,5-dihydro-pyrazol-3-yl |
| A-1662. | 5-trifluoromethyl-4,5-dihydro-pyrazol-3-yl |
| A-1663. | 4-methoxy-4,5-dihydro-pyrazol-3-yl |
| A-1664. | 5-methoxy-4,5-dihydro-pyrazol-3-yl |
| A-1665. | 4-trifluoromethoxy-4,5-dihydro-pyrazol-3-yl |
| A-1666. | 5-trifluoromethoxy-4,5-dihydro-pyrazol-3-yl |
| A-1667. | 3-chloro-4,5-dihydro-pyrazol-4-yl |
| A-1668. | 5-chloro-4,5-dihydro-pyrazol-4-yl |
| A-1669. | 3-bromo-4,5-dihydro-pyrazol-4-yl |
| A-1670. | 5-bromo-4,5-dihydro-pyrazol-4-yl |
| A-1671. | 3-cyano-4,5-dihydro-pyrazol-4-yl |
| A-1672. | 5-cyano-4,5-dihydro-pyrazol-4-yl |
| A-1673. | 3-nitro-4,5-dihydro-pyrazol-4-yl |
| A-1674. | 5-nitro-4,5-dihydro-pyrazol-4-yl |
| A-1675. | 3-methyl-4,5-dihydro-pyrazol-4-yl |
| A-1676. | 5-methyl-4,5-dihydro-pyrazol-4-yl |
| A-1677. | 3-trifluoromethyl-4,5-dihydro-pyrazol-4-yl |
| A-1678. | 5-trifluoromethyl-4,5-dihydro-pyrazol-4-yl |
| A-1679. | 3-methoxy-4,5-dihydro-pyrazol-4-yl |
| A-1680. | 5-methoxy-4,5-dihydro-pyrazol-4-yl |
| A-1681. | 3-trifluoromethoxy-4,5-dihydro-pyrazol-4-yl |
| A-1682. | 5-trifluoromethoxy-4,5-dihydro-pyrazol-4-yl |
| A-1683. | 3-chloro-4,5-dihydro-pyrazol-5-yl |
| A-1684. | 4-chloro-4,5-dihydro-pyrazol-5-yl |
| A-1685. | 3-bromo-4,5-dihydro-pyrazol-5-yl |
| A-1686. | 4-bromo-4,5-dihydro-pyrazol-5-yl |
| A-1687. | 3-cyano-4,5-dihydro-pyrazol-5-yl |
| A-1688. | 4-cyano-4,5-dihydro-pyrazol-5-yl |
| A-1689. | 3-nitro-4,5-dihydro-pyrazol-5-yl |
| A-1690. | 4-nitro-4,5-dihydro-pyrazol-5-yl |
| A-1691. | 3-methyl-4,5-dihydro-pyrazol-5-yl |
| A-1692. | 4-methyl-4,5-dihydro-pyrazol-5-yl |
| A-1693. | 3-trifluoromethyl-4,5-dihydro-pyrazol-5-yl |
| A-1694. | 4-trifluoromethyl-4,5-dihydro-pyrazol-5-yl |
| A-1695. | 3-methoxy-4,5-dihydro-pyrazol-5-yl |
| A-1696. | 4-methoxy-4,5-dihydro-pyrazol-5-yl |
| A-1697. | 3-trifluoromethoxy-4,5-dihydro-pyrazol-5-yl |
| A-1698. | 4-trifluoromethoxy-4,5-dihydro-pyrazol-5-yl |
| A-1699. | 3-chloro-1-methyl-4,5-dihydro-pyrazol-2-yl |
| A-1700. | 4-chloro-1-methyl-4,5-dihydro-pyrazol-2-yl |
| A-1701. | 5-chloro-1-methyl-4,5-dihydro-pyrazol-2-yl |
| A-1702. | 3-bromo-1-methyl-4,5-dihydro-pyrazol-2-yl |
| A-1703. | 4-bromo-1-methyl-4,5-dihydro-pyrazol-2-yl |
| A-1704. | 5-bromo-1-methyl-4,5-dihydro-pyrazol-2-yl |
| A-1705. | 3-cyano-1-methyl-4,5-dihydro-pyrazol-2-yl |
| A-1706. | 4-cyano-1-methyl-4,5-dihydro-pyrazol-2-yl |
| A-1707. | 5-cyano-1-methyl-4,5-dihydro-pyrazol-2-yl |
| A-1708. | 3-nitro-1-methyl-4,5-dihydro-pyrazol-2-yl |
| A-1709. | 4-nitro-1-methyl-4,5-dihydro-pyrazol-2-yl |
| A-1710. | 5-nitro-1-methyl-4,5-dihydro-pyrazol-2-yl |
| A-1711. | 3-methyl-1-methyl-4,5-dihydro-pyrazol-2-yl |
| A-1712. | 4-methyl-1-methyl-4,5-dihydro-pyrazol-2-yl |
| A-1713. | 5-methyl-1-methyl-4,5-dihydro-pyrazol-2-yl |
| A-1714. | 3-trifluoromethyl-1-methyl-4,5-dihydro-pyrazol-2-yl |
| A-1715. | 4-trifluoromethyl-1-methyl-4,5-dihydro-pyrazol-2-yl |
| A-1716. | 5-trifluoromethyl-1-methyl-4,5-dihydro-pyrazol-2-yl |
| A-1717. | 3-methoxy-1-methyl-4,5-dihydro-pyrazol-2-yl |
| A-1718. | 4-methoxy-1-methyl-4,5-dihydro-pyrazol-2-yl |
| A-1719. | 5-methoxy-1-methyl-4,5-dihydro-pyrazol-2-yl |
| A-1720. | 3-trifluoromethoxy-1-methyl-4,5-dihydro-pyrazol-2-yl |
| A-1721. | 4-trifluoromethoxy-1-methyl-4,5-dihydro-pyrazol-2-yl |
| A-1722. | 5-trifluoromethoxy-1-methyl-4,5-dihydro-pyrazol-2-yl |
| A-1723. | 4-chloro-1-methyl-4,5-dihydro-pyrazol-3-yl |
| A-1724. | 5-chloro-1-methyl-4,5-dihydro-pyrazol-3-yl |
| A-1725. | 4-bromo-1-methyl-4,5-dihydro-pyrazol-3-yl |
| A-1726. | 5-bromo-1-methyl-4,5-dihydro-pyrazol-3-yl |
| A-1727. | 4-cyano-1-methyl-4,5-dihydro-pyrazol-3-yl |
| A-1728. | 5-cyano-1-methyl-4,5-dihydro-pyrazol-3-yl |
| A-1729. | 4-nitro-1-methyl-4,5-dihydro-pyrazol-3-yl |
| A-1730. | 5-nitro-1-methyl-4,5-dihydro-pyrazol-3-yl |
| A-1731. | 4-methyl-1-methyl-4,5-dihydro-pyrazol-3-yl |
| A-1732. | 5-methyl-1-methyl-4,5-dihydro-pyrazol-3-yl |
| A-1733. | 4-trifluoromethyl-1-methyl-4,5-dihydro-pyrazol-3-yl |
| A-1734. | 5-trifluoromethyl-1-methyl-4,5-dihydro-pyrazol-3-yl |
| A-1735. | 4-methoxy-1-methyl-4,5-dihydro-pyrazol-3-yl |
| A-1736. | 5-methoxy-1-methyl-4,5-dihydro-pyrazol-3-yl |
| A-1737. | 4-trifluoromethoxy-1-methyl-4,5-dihydro-pyrazol-3-yl |
| A-1738. | 5-trifluoromethoxy-1-methyl-4,5-dihydro-pyrazol-3-yl |
| A-1739. | 3-chloro-1-methyl-4,5-dihydro-pyrazol-4-yl |
| A-1740. | 5-chloro-1-methyl-4,5-dihydro-pyrazol-4-yl |
| A-1741. | 3-bromo-1-methyl-4,5-dihydro-pyrazol-4-yl |
| A-1742. | 5-bromo-1-methyl-4,5-dihydro-pyrazol-4-yl |
| A-1743. | 3-cyano-1-methyl-4,5-dihydro-pyrazol-4-yl |
| A-1744. | 5-cyano-1-methyl-4,5-dihydro-pyrazol-4-yl |
| A-1745. | 3-nitro-1-methyl-4,5-dihydro-pyrazol-4-yl |
| A-1746. | 5-nitro-1-methyl-4,5-dihydro-pyrazol-4-yl |
| A-1747. | 3-methyl-1-methyl-4,5-dihydro-pyrazol-4-yl |
| A-1748. | 5-methyl-1-methyl-4,5-dihydro-pyrazol-4-yl |
| A-1749. | 3-trifluoromethyl-1-methyl-4,5-dihydro-pyrazol-4-yl |
| A-1750. | 5-trifluoromethyl-1-methyl-4,5-dihydro-pyrazol-4-yl |
| A-1751. | 3-methoxy-1-methyl-4,5-dihydro-pyrazol-4-yl |
| A-1752. | 5-methoxy-1-methyl-4,5-dihydro-pyrazol-4-yl |
| A-1753. | 3-trifluoromethoxy-1-methyl-4,5-dihydro-pyrazol-4-yl |
| A-1754. | 5-trifluoromethoxy-1-methyl-4,5-dihydro-pyrazol-4-yl |
| A-1755. | 3-chloro-1-methyl-4,5-dihydro-pyrazol-5-yl |
| A-1756. | 4-chloro-1-methyl-4,5-dihydro-pyrazol-5-yl |
| A-1757. | 3-bromo-1-methyl-4,5-dihydro-pyrazol-5-yl |
| A-1758. | 4-bromo-1-methyl-4,5-dihydro-pyrazol-5-yl |
| A-1759. | 3-cyano-1-methyl-4,5-dihydro-pyrazol-5-yl |
| A-1760. | 4-cyano-1-methyl-4,5-dihydro-pyrazol-5-yl |
| A-1761. | 3-nitro-1-methyl-4,5-dihydro-pyrazol-5-yl |
| A-1762. | 4-nitro-1-methyl-4,5-dihydro-pyrazol-5-yl |
| A-1763. | 3-methyl-1-methyl-4,5-dihydro-pyrazol-5-yl |
| A-1764. | 4-methyl-1-methyl-4,5-dihydro-pyrazol-5-yl |
| A-1765. | 3-trifluoromethyl-1-methyl-4,5-dihydro-pyrazol-5-yl |
| A-1766. | 4-trifluoromethyl-1-methyl-4,5-dihydro-pyrazol-5-yl |
| A-1767. | 3-methoxy-1-methyl-4,5-dihydro-pyrazol-5-yl |
| A-1768. | 4-methoxy-1-methyl-4,5-dihydro-pyrazol-5-yl |
| A-1769. | 3-trifluoromethoxy-1-methyl-4,5-dihydro-pyrazol-5-yl |
| A-1770. | 4-trifluoromethoxy-1-methyl-4,5-dihydro-pyrazol-5-yl |
| A-1771. | 2,3-dihydro-imidazol-1-yl |
| A-1772. | 2,3-dihydro-imidazol-2-yl |
| A-1773. | 2,3-dihydro-imidazol-4-yl |
| A-1774. | 2,3-dihydro-imidazol-5-yl |
| A-1775. | 1-methyl-2,3-dihydro-imidazol-2-yl |
| A-1776. | 1-methyl-2,3-dihydro-imidazol-4-yl |
| A-1777. | 1-methyl-2,3-dihydro-imidazol-5-yl |
| A-1778. | 2,3-dihydro-imidazol-2-on-1-yl |
| A-1779. | 2,3-dihydro-imidazol-2-on-4-yl |
| A-1780. | 2,3-dihydro-imidazol-2-on-5-yl |
| A-1781. | 1-methyl-2,3-dihydro-imidazol-2-on-4-yl |
| A-1782. | 1-methyl-2,3-dihydro-imidazol-2-on-5-yl |
| A-1783. | 2-chloro-2,3-dihydro-imidazol-1-yl |
| A-1784. | 4-chloro-2,3-dihydro-imidazol-1-yl |
| A-1785. | 5-chloro-2,3-dihydro-imidazol-1-yl |
| A-1786. | 2-bromo-2,3-dihydro-imidazol-1-yl |
| A-1787. | 4-bromo-2,3-dihydro-imidazol-1-yl |
| A-1788. | 5-bromo-2,3-dihydro-imidazol-1-yl |
| A-1789. | 2-cyano-2,3-dihydro-imidazol-1-yl |
| A-1790. | 4-cyano-2,3-dihydro-imidazol-1-yl |
| A-1791. | 5-cyano-2,3-dihydro-imidazol-1-yl |
| A-1792. | 2-nitro-2,3-dihydro-imidazol-1-yl |
| A-1793. | 4-nitro-2,3-dihydro-imidazol-1-yl |
| A-1794. | 5-nitro-2,3-dihydro-imidazol-1-yl |
| A-1795. | 2-methyl-2,3-dihydro-imidazol-1-yl |
| A-1796. | 4-methyl-2,3-dihydro-imidazol-1-yl |
| A-1797. | 5-methyl-2,3-dihydro-imidazol-1-yl |
| A-1798. | 2-trifluoromethyl-2,3-dihydro-imidazol-1-yl |
| A-1799. | 4-trifluoromethyl-2,3-dihydro-imidazol-1-yl |
| A-1800. | 5-trifluoromethyl-2,3-dihydro-imidazol-1-yl |
| A-1801. | 2-methoxy-2,3-dihydro-imidazol-1-yl |
| A-1802. | 4-methoxy-2,3-dihydro-imidazol-1-yl |
| A-1803. | 5-methoxy-2,3-dihydro-imidazol-1-yl |
| A-1804. | 2-trifluoromethoxy-2,3-dihydro-imidazol-1-yl |
| A-1805. | 4-trifluoromethoxy-2,3-dihydro-imidazol-1-yl |
| A-1806. | 5-trifluoromethoxy-2,3-dihydro-imidazol-1-yl |
| A-1807. | 4-chloro-2,3-dihydro-imidazol-2-yl |
| A-1808. | 5-chloro-2,3-dihydro-imidazol-2-yl |
| A-1809. | 4-bromo-2,3-dihydro-imidazol-2-yl |
| A-1810. | 5-bromo-2,3-dihydro-imidazol-2-yl |
| A-1811. | 4-cyano-2,3-dihydro-imidazol-2-yl |
| A-1812. | 5-cyano-2,3-dihydro-imidazol-2-yl |
| A-1813. | 4-nitro-2,3-dihydro-imidazol-2-yl |
| A-1814. | 5-nitro-2,3-dihydro-imidazol-2-yl |
| A-1815. | 4-methyl-2,3-dihydro-imidazol-2-yl |
| A-1816. | 5-methyl-2,3-dihydro-imidazol-2-yl |
| A-1817. | 4-trifluoromethyl-2,3-dihydro-imidazol-2-yl |
| A-1818. | 5-trifluoromethyl-2,3-dihydro-imidazol-2-yl |
| A-1819. | 4-methoxy-2,3-dihydro-imidazol-2-yl |
| A-1820. | 5-methoxy-2,3-dihydro-imidazol-2-yl |
| A-1821. | 4-trifluoromethoxy-2,3-dihydro-imidazol-2-yl |
| A-1822. | 5-trifluoromethoxy-2,3-dihydro-imidazol-2-yl |
| A-1823. | 2-chloro-2,3-dihydro-imidazol-4-yl |
| A-1824. | 5-chloro-2,3-dihydro-imidazol-4-yl |
| A-1825. | 2-bromo-2,3-dihydro-imidazol-4-yl |
| A-1826. | 5-bromo-2,3-dihydro-imidazol-4-yl |
| A-1827. | 2-cyano-2,3-dihydro-imidazol-4-yl |
| A-1828. | 5-cyano-2,3-dihydro-imidazol-4-yl |
| A-1829. | 2-nitro-2,3-dihydro-imidazol-4-yl |
| A-1830. | 5-nitro-2,3-dihydro-imidazol-4-yl |
| A-1831. | 2-methyl-2,3-dihydro-imidazol-4-yl |
| A-1832. | 5-methyl-2,3-dihydro-imidazol-4-yl |
| A-1833. | 2-trifluoromethyl-2,3-dihydro-imidazol-4-yl |
| A-1834. | 5-trifluoromethyl-2,3-dihydro-imidazol-4-yl |
| A-1835. | 2-methoxy-2,3-dihydro-imidazol-4-yl |
| A-1836. | 5-methoxy-2,3-dihydro-imidazol-4-yl |
| A-1837. | 2-trifluoromethoxy-2,3-dihydro-imidazol-4-yl |
| A-1838. | 5-trifluoromethoxy-2,3-dihydro-imidazol-4-yl |
| A-1839. | 2-chloro-2,3-dihydro-imidazol-5-yl |
| A-1840. | 4-chloro-2,3-dihydro-imidazol-5-yl |
| A-1841. | 2-bromo-2,3-dihydro-imidazol-5-yl |
| A-1842. | 4-bromo-2,3-dihydro-imidazol-5-yl |
| A-1843. | 2-cyano-2,3-dihydro-imidazol-5-yl |
| A-1844. | 4-cyano-2,3-dihydro-imidazol-5-yl |
| A-1845. | 2-nitro-2,3-dihydro-imidazol-5-yl |
| A-1846. | 4-nitro-2,3-dihydro-imidazol-5-yl |
| A-1847. | 2-methyl-2,3-dihydro-imidazol-5-yl |
| A-1848. | 4-methyl-2,3-dihydro-imidazol-5-yl |
| A-1849. | 2-trifluoromethyl-2,3-dihydro-imidazol-5-yl |
| A-1850. | 4-trifluoromethyl-2,3-dihydro-imidazol-5-yl |
| A-1851. | 2-methoxy-2,3-dihydro-imidazol-5-yl |
| A-1852. | 4-methoxy-2,3-dihydro-imidazol-5-yl |
| A-1853. | 2-trifluoromethoxy-2,3-dihydro-imidazol-5-yl |
| A-1854. | 4-trifluoromethoxy-2,3-dihydro-imidazol-5-yl |
| A-1855. | 4-chloro-1-methyl-2,3-dihydro-imidazol-2-yl |
| A-1856. | 5-chloro-1-methyl-2,3-dihydro-imidazol-2-yl |
| A-1857. | 4-bromo-1-methyl-2,3-dihydro-imidazol-2-yl |
| A-1858. | 5-bromo-1-methyl-2,3-dihydro-imidazol-2-yl |
| A-1859. | 4-cyano-1-methyl-2,3-dihydro-imidazol-2-yl |
| A-1860. | 5-cyano-1-methyl-2,3-dihydro-imidazol-2-yl |
| A-1861. | 4-nitro-1-methyl-2,3-dihydro-imidazol-2-yl |
| A-1862. | 5-nitro-1-methyl-2,3-dihydro-imidazol-2-yl |
| A-1863. | 4-methyl-1-methyl-2,3-dihydro-imidazol-2-yl |
| A-1864. | 5-methyl-1-methyl-2,3-dihydro-imidazol-2-yl |
| A-1865. | 4-trifluoromethyl-1-methyl-2,3-dihydro-imidazol-2-yl |
| A-1866. | 5-trifluoromethyl-1-methyl-2,3-dihydro-imidazol-2-yl |
| A-1867. | 4-methoxy-1-methyl-2,3-dihydro-imidazol-2-yl |
| A-1868. | 5-methoxy-1-methyl-2,3-dihydro-imidazol-2-yl |
| A-1869. | 4-trifluoromethoxy-1-methyl-2,3-dihydro-imidazol-2-yl |
| A-1870. | 5-trifluoromethoxy-1-methyl-2,3-dihydro-imidazol-2-yl |
| A-1871. | 2-chloro-1-methyl-2,3-dihydro-imidazol-4-yl |
| A-1872. | 5-chloro-1-methyl-2,3-dihydro-imidazol-4-yl |
| A-1873. | 2-bromo-1-methyl-2,3-dihydro-imidazol-4-yl |
| A-1874. | 5-bromo-1-methyl-2,3-dihydro-imidazol-4-yl |
| A-1875. | 2-cyano-1-methyl-2,3-dihydro-imidazol-4-yl |
| A-1876. | 5-cyano-1-methyl-2,3-dihydro-imidazol-4-yl |
| A-1877. | 2-nitro-1-methyl-2,3-dihydro-imidazol-4-yl |
| A-1878. | 5-nitro-1-methyl-2,3-dihydro-imidazol-4-yl |
| A-1879. | 2-methyl-1-methyl-2,3-dihydro-imidazol-4-yl |
| A-1880. | 5-methyl-1-methyl-2,3-dihydro-imidazol-4-yl |
| A-1881. | 2-trifluoromethyl-1-methyl-2,3-dihydro-imidazol-4-yl |
| A-1882. | 5-trifluoromethyl-1-methyl-2,3-dihydro-imidazol-4-yl |
| A-1883. | 2-methoxy-1-methyl-2,3-dihydro-imidazol-4-yl |
| A-1884. | 5-methoxy-1-methyl-2,3-dihydro-imidazol-4-yl |
| A-1885. | 2-trifluoromethoxy-1-methyl-2,3-dihydro-imidazol-4-yl |
| A-1886. | 5-trifluoromethoxy-1-methyl-2,3-dihydro-imidazol-4-yl |
| A-1887. | 2-chloro-1-methyl-2,3-dihydro-imidazol-5-yl |
| A-1888. | 4-chloro-1-methyl-2,3-dihydro-imidazol-5-yl |
| A-1889. | 2-bromo-1-methyl-2,3-dihydro-imidazol-5-yl |
| A-1890. | 4-bromo-1-methyl-2,3-dihydro-imidazol-5-yl |
| A-1891. | 2-cyano-1-methyl-2,3-dihydro-imidazol-5-yl |
| A-1892. | 4-cyano-1-methyl-2,3-dihydro-imidazol-5-yl |
| A-1893. | 2-nitro-1-methyl-2,3-dihydro-imidazol-5-yl |
| A-1894. | 4-nitro-1-methyl-2,3-dihydro-imidazol-5-yl |
| A-1895. | 2-methyl-1-methyl-2,3-dihydro-imidazol-5-yl |
| A-1896. | 4-methyl-1-methyl-2,3-dihydro-imidazol-5-yl |
| A-1897. | 2-trifluoromethyl-1-methyl-2,3-dihydro-imidazol-5-yl |
| A-1898. | 4-trifluoromethyl-1-methyl-2,3-dihydro-imidazol-5-yl |
| A-1899. | 2-methoxy-1-methyl-2,3-dihydro-imidazol-5-yl |
| A-1900. | 4-methoxy-1-methyl-2,3-dihydro-imidazol-5-yl |
| A-1901. | 2-trifluoromethoxy-1-methyl-2,3-dihydro-imidazol-5-yl |
| A-1902. | 4-trifluoromethoxy-1-methyl-2,3-dihydro-imidazol-5-yl |
| A-1903. | 4-chloro-2,3-dihydro-imidazol-2-on-1-yl |
| A-1904. | 5-chloro-2,3-dihydro-imidazol-2-on-1-yl |
| A-1905. | 4-bromo-2,3-dihydro-imidazol-2-on-1-yl |
| A-1906. | 5-bromo-2,3-dihydro-imidazol-2-on-1-yl |
| A-1907. | 4-cyano-2,3-dihydro-imidazol-2-on-1-yl |
| A-1908. | 5-cyano-2,3-dihydro-imidazol-2-on-1-yl |
| A-1909. | 4-nitro-2,3-dihydro-imidazol-2-on-1-yl |
| A-1910. | 5-nitro-2,3-dihydro-imidazol-2-on-1-yl |
| A-1911. | 4-methyl-2,3-dihydro-imidazol-2-on-1-yl |
| A-1912. | 5-methyl-2,3-dihydro-imidazol-2-on-1-yl |
| A-1913. | 4-trifluoromethyl-2,3-dihydro-imidazol-2-on-1-yl |
| A-1914. | 5-trifluoromethyl-2,3-dihydro-imidazol-2-on-1-yl |
| A-1915. | 4-methoxy-2,3-dihydro-imidazol-2-on-1-yl |
| A-1916. | 5-methoxy-2,3-dihydro-imidazol-2-on-1-yl |
| A-1917. | 4-trifluoromethoxy-2,3-dihydro-imidazol-2-on-1-yl |
| A-1918. | 5-trifluoromethoxy-2,3-dihydro-imidazol-2-on-1-yl |
| A-1919. | 5-chloro-2,3-dihydro-imidazol-2-on-4-yl |
| A-1920. | 5-bromo-2,3-dihydro-imidazol-2-on-4-yl |
| A-1921. | 5-cyano-2,3-dihydro-imidazol-2-on-4-yl |
| A-1922. | 5-nitro-2,3-dihydro-imidazol-2-on-4-yl |
| A-1923. | 5-methyl-2,3-dihydro-imidazol-2-on-4-yl |
| A-1924. | 5-trifluoromethyl-2,3-dihydro-imidazol-2-on-4-yl |
| A-1925. | 5-methoxy-2,3-dihydro-imidazol-2-on-4-yl |
| A-1926. | 5-trifluoromethoxy-2,3-dihydro-imidazol-2-on-4-yl |
| A-1927. | 4-chloro-2,3-dihydro-imidazol-2-on-5-yl |
| A-1928. | 4-bromo-2,3-dihydro-imidazol-2-on-5-yl |
| A-1929. | 4-cyano-2,3-dihydro-imidazol-2-on-5-yl |
| A-1930. | 4-nitro-2,3-dihydro-imidazol-2-on-5-yl |
| A-1931. | 4-methyl-2,3-dihydro-imidazol-2-on-5-yl |
| A-1932. | 4-trifluoromethyl-2,3-dihydro-imidazol-2-on-5-yl |
| A-1933. | 4-methoxy-2,3-dihydro-imidazol-2-on-5-yl |
| A-1934. | 4-trifluoromethoxy-2,3-dihydro-imidazol-2-on-5-yl |
| A-1935. | 5-chloro-1-methyl-2,3-dihydro-imidazol-2-on-4-yl |
| A-1936. | 5-bromo-1-methyl-2,3-dihydro-imidazol-2-on-4-yl |
| A-1937. | 5-cyano-1-methyl-2,3-dihydro-imidazol-2-on-4-yl |
| A-1938. | 5-nitro-1-methyl-2,3-dihydro-imidazol-2-on-4-yl |
| A-1939. | 5-methyl-1-methyl-2,3-dihydro-imidazol-2-on-4-yl |
| A-1940. | 5-trifluoromethyl-1-methyl-2,3-dihydro-imidazol-2-on-4-yl |
| A-1941. | 5-methoxy-1-methyl-2,3-dihydro-imidazol-2-on-4-yl |
| A-1942. | 5-trifluoromethoxy-1-methyl-2,3-dihydro-imidazol-2-on- |
| | 4-yl |
| A-1943. | 4-chloro-1-methyl-2,3-dihydro-imidazol-2-on-5-yl |
| A-1944. | 4-bromo-1-methyl-2,3-dihydro-imidazol-2-on-5-yl |
| A-1945. | 4-cyano-1-methyl-2,3-dihydro-imidazol-2-on-5-yl |
| A-1946. | 4-nitro-1-methyl-2,3-dihydro-imidazol-2-on-5-yl |
| A-1947. | 4-methyl-1-methyl-2,3-dihydro-imidazol-2-on-5-yl |
| A-1948. | 4-trifluoromethyl-1-methyl-2,3-dihydro-imidazol-2-on-5-yl |
| A-1949. | 4-methoxy-1-methyl-2,3-dihydro-imidazol-2-on-5-yl |
| A-1950. | 4-trifluoromethoxy-1-methyl-2,3-dihydro-imidazol-2-on- |
| | 5-yl |
| A-1951. | 2,5-dihydro-imidazol-1-yl |
| A-1952. | 2,5-dihydro-imidazol-2-yl |
| A-1953. | 2,5-dihydro-imidazol-4-yl |
| A-1954. | 2,5-dihydro-imidazol-5-yl |
| A-1955. | 1-methyl-2,5-dihydro-imidazol-2-yl |
| A-1956. | 1-methyl-2,5-dihydro-imidazol-4-yl |
| A-1957. | 1-methyl-2,5-dihydro-imidazol-5-yl |
| A-1958. | 2,5-dihydro-imidazol-2-on-1-yl |
| A-1959. | 2,5-dihydro-imidazol-2-on-4-yl |
| A-1960. | 2,5-dihydro-imidazol-2-on-5-yl |
| A-1961. | 1-methyl-2,5-dihydro-imidazol-2-on-4-yl |
| A-1962. | 1-methyl-2,5-dihydro-imidazol-2-on-5-yl |
| A-1963. | 2-chloro-2,5-dihydro-imidazol-1-yl |
| A-1964. | 4-chloro-2,5-dihydro-imidazol-1-yl |
| A-1965. | 5-chloro-2,5-dihydro-imidazol-1-yl |
| A-1966. | 2-bromo-2,5-dihydro-imidazol-1-yl |
| A-1967. | 4-bromo-2,5-dihydro-imidazol-1-yl |
| A-1968. | 5-bromo-2,5-dihydro-imidazol-1-yl |
| A-1969. | 2-cyano-2,5-dihydro-imidazol-1-yl |
| A-1970. | 4-cyano-2,5-dihydro-imidazol-1-yl |
| A-1971. | 5-cyano-2,5-dihydro-imidazol-1-yl |
| A-1972. | 2-nitro-2,5-dihydro-imidazol-1-yl |
| A-1973. | 4-nitro-2,5-dihydro-imidazol-1-yl |
| A-1974. | 5-nitro-2,5-dihydro-imidazol-1-yl |
| A-1975. | 2-methyl-2,5-dihydro-imidazol-1-yl |
| A-1976. | 4-methyl-2,5-dihydro-imidazol-1-yl |
| A-1977. | 5-methyl-2,5-dihydro-imidazol-1-yl |
| A-1978. | 2-trifluoromethyl-2,5-dihydro-imidazol-1-yl |
| A-1979. | 4-trifluoromethyl-2,5-dihydro-imidazol-1-yl |
| A-1980. | 5-trifluoromethyl-2,5-dihydro-imidazol-1-yl |
| A-1981. | 2-methoxy-2,5-dihydro-imidazol-1-yl |
| A-1982. | 4-methoxy-2,5-dihydro-imidazol-1-yl |
| A-1983. | 5-methoxy-2,5-dihydro-imidazol-1-yl |
| A-1984. | 2-trifluoromethoxy-2,5-dihydro-imidazol-1-yl |
| A-1985. | 4-trifluoromethoxy-2,5-dihydro-imidazol-1-yl |
| A-1986. | 5-trifluoromethoxy-2,5-dihydro-imidazol-1-yl |
| A-1987. | 4-chloro-2,5-dihydro-imidazol-2-yl |
| A-1988. | 5-chloro-2,5-dihydro-imidazol-2-yl |
| A-1989. | 4-bromo-2,5-dihydro-imidazol-2-yl |
| A-1990. | 5-bromo-2,5-dihydro-imidazol-2-yl |
| A-1991. | 4-cyano-2,5-dihydro-imidazol-2-yl |
| A-1992. | 5-cyano-2,5-dihydro-imidazol-2-yl |
| A-1993. | 4-nitro-2,5-dihydro-imidazol-2-yl |
| A-1994. | 5-nitro-2,5-dihydro-imidazol-2-yl |
| A-1995. | 4-methyl-2,5-dihydro-imidazol-2-yl |
| A-1996. | 5-methyl-2,5-dihydro-imidazol-2-yl |
| A-1997. | 4-trifluoromethyl-2,5-dihydro-imidazol-2-yl |
| A-1998. | 5-trifluoromethyl-2,5-dihydro-imidazol-2-yl |
| A-1999. | 4-methoxy-2,5-dihydro-imidazol-2-yl |
| A-2000. | 5-methoxy-2,5-dihydro-imidazol-2-yl |
| A-2001. | 4-trifluoromethoxy-2,5-dihydro-imidazol-2-yl |
| A-2002. | 5-trifluoromethoxy-2,5-dihydro-imidazol-2-yl |
| A-2003. | 2-chloro-2,5-dihydro-imidazol-4-yl |
| A-2004. | 5-chloro-2,5-dihydro-imidazol-4-yl |
| A-2005. | 2-bromo-2,5-dihydro-imidazol-4-yl |
| A-2006. | 5-bromo-2,5-dihydro-imidazol-4-yl |
| A-2007. | 2-cyano-2,5-dihydro-imidazol-4-yl |
| A-2008. | 5-cyano-2,5-dihydro-imidazol-4-yl |
| A-2009. | 2-nitro-2,5-dihydro-imidazol-4-yl |
| A-2010. | 5-nitro-2,5-dihydro-imidazol-4-yl |
| A-2011. | 2-methyl-2,5-dihydro-imidazol-4-yl |
| A-2012. | 5-methyl-2,5-dihydro-imidazol-4-yl |
| A-2013. | 2-trifluoromethyl-2,5-dihydro-imidazol-4-yl |
| A-2014. | 5-trifluoromethyl-2,5-dihydro-imidazol-4-yl |
| A-2015. | 2-methoxy-2,5-dihydro-imidazol-4-yl |
| A-2016. | 5-methoxy-2,5-dihydro-imidazol-4-yl |
| A-2017. | 2-trifluoromethoxy-2,5-dihydro-imidazol-4-yl |
| A-2018. | 5-trifluoromethoxy-2,5-dihydro-imidazol-4-yl |
| A-2019. | 2-chloro-2,5-dihydro-imidazol-5-yl |
| A-2020. | 4-chloro-2,5-dihydro-imidazol-5-yl |
| A-2021. | 2-bromo-2,5-dihydro-imidazol-5-yl |
| A-2022. | 4-bromo-2,5-dihydro-imidazol-5-yl |
| A-2023. | 2-cyano-2,5-dihydro-imidazol-5-yl |
| A-2024. | 4-cyano-2,5-dihydro-imidazol-5-yl |
| A-2025. | 2-nitro-2,5-dihydro-imidazol-5-yl |
| A-2026. | 4-nitro-2,5-dihydro-imidazol-5-yl |
| A-2027. | 4-methyl-2,5-dihydro-imidazol-5-yl |
| A-2028. | 5-methyl-2,5-dihydro-imidazol-5-yl |
| A-2029. | 4-trifluoromethyl-2,5-dihydro-imidazol-5-yl |
| A-2030. | 5-trifluoromethyl-2,5-dihydro-imidazol-5-yl |
| A-2031. | 4-methoxy-2,5-dihydro-imidazol-5-yl |
| A-2032. | 5-methoxy-2,5-dihydro-imidazol-5-yl |
| A-2033. | 4-trifluoromethoxy-2,5-dihydro-imidazol-5-yl |
| A-2034. | 5-trifluoromethoxy-2,5-dihydro-imidazol-5-yl |
| A-2035. | 4-chloro-1-methyl-2,5-dihydro-imidazol-2-yl |
| A-2036. | 5-chloro-1-methyl-2,5-dihydro-imidazol-2-yl |
| A-2037. | 4-bromo-1-methyl-2,5-dihydro-imidazol-2-yl |
| A-2038. | 5-bromo-1-methyl-2,5-dihydro-imidazol-2-yl |
| A-2039. | 4-cyano-1-methyl-2,5-dihydro-imidazol-2-yl |
| A-2040. | 5-cyano-1-methyl-2,5-dihydro-imidazol-2-yl |
| A-2041. | 4-nitro-1-methyl-2,5-dihydro-imidazol-2-yl |
| A-2042. | 5-nitro-1-methyl-2,5-dihydro-imidazol-2-yl |
| A-2043. | 4-methyl-1-methyl-2,5-dihydro-imidazol-2-yl |
| A-2044. | 5-methyl-1-methyl-2,5-dihydro-imidazol-2-yl |
| A-2045. | 4-trifluoromethyl-1-methyl-2,5-dihydro-imidazol-2-yl |
| A-2046. | 5-trifluoromethyl-1-methyl-2,5-dihydro-imidazol-2-yl |
| A-2047. | 4-methoxy-1-methyl-2,5-dihydro-imidazol-2-yl |
| A-2048. | 5-methoxy-1-methyl-2,5-dihydro-imidazol-2-yl |
| A-2049. | 4-trifluoromethoxy-1-methyl-2,5-dihydro-imidazol-2-yl |
| A-2050. | 5-trifluoromethoxy-1-methyl-2,5-dihydro-imidazol-2-yl |
| A-2051. | 2-chloro-1-methyl-2,5-dihydro-imidazol-4-yl |
| A-2052. | 5-chloro-1-methyl-2,5-dihydro-imidazol-4-yl |
| A-2053. | 2-bromo-1-methyl-2,5-dihydro-imidazol-4-yl |
| A-2054. | 5-bromo-1-methyl-2,5-dihydro-imidazol-4-yl |
| A-2055. | 2-cyano-1-methyl-2,5-dihydro-imidazol-4-yl |
| A-2056. | 5-cyano-1-methyl-2,5-dihydro-imidazol-4-yl |
| A-2057. | 2-nitro-1-methyl-2,5-dihydro-imidazol-4-yl |
| A-2058. | 5-nitro-1-methyl-2,5-dihydro-imidazol-4-yl |
| A-2059. | 2-methyl-1-methyl-2,5-dihydro-imidazol-4-yl |
| A-2060. | 5-methyl-1-methyl-2,5-dihydro-imidazol-4-yl |
| A-2061. | 2-trifluoromethyl-1-methyl-2,5-dihydro-imidazol-4-yl |
| A-2062. | 5-trifluoromethyl-1-methyl-2,5-dihydro-imidazol-4-yl |
| A-2063. | 2-methoxy-1-methyl-2,5-dihydro-imidazol-4-yl |
| A-2064. | 5-methoxy-1-methyl-2,5-dihydro-imidazol-4-yl |
| A-2065. | 2-trifluoromethoxy-1-methyl-2,5-dihydro-imidazol-4-yl |
| A-2066. | 5-trifluoromethoxy-1-methyl-2,5-dihydro-imidazol-4-yl |
| A-2067. | 2-chloro-1-methyl-2,5-dihydro-imidazol-5-yl |
| A-2068. | 4-chloro-1-methyl-2,5-dihydro-imidazol-5-yl |
| A-2069. | 2-bromo-1-methyl-2,5-dihydro-imidazol-5-yl |
| A-2070. | 4-bromo-1-methyl-2,5-dihydro-imidazol-5-yl |
| A-2071. | 2-cyano-1-methyl-2,5-dihydro-imidazol-5-yl |
| A-2072. | 4-cyano-1-methyl-2,5-dihydro-imidazol-5-yl |
| A-2073. | 2-nitro-1-methyl-2,5-dihydro-imidazol-5-yl |
| A-2074. | 4-nitro-1-methyl-2,5-dihydro-imidazol-5-yl |
| A-2075. | 2-methyl-1-methyl-2,5-dihydro-imidazol-5-yl |
| A-2076. | 4-methyl-1-methyl-2,5-dihydro-imidazol-5-yl |
| A-2077. | 2-trifluoromethyl-1-methyl-2,5-dihydro-imidazol-5-yl |
| A-2078. | 4-trifluoromethyl-1-methyl-2,5-dihydro-imidazol-5-yl |
| A-2079. | 2-methoxy-1-methyl-2,5-dihydro-imidazol-5-yl |
| A-2080. | 4-methoxy-1-methyl-2,5-dihydro-imidazol-5-yl |
| A-2081. | 2-trifluoromethoxy-1-methyl-2,5-dihydro-imidazol-5-yl |
| A-2082. | 4-trifluoromethoxy-1-methyl-2,5-dihydro-imidazol-5-yl |
| A-2083. | 4-chloro-2,5-dihydro-imidazol-2-on-1-yl |
| A-2084. | 5-chloro-2,5-dihydro-imidazol-2-on-1-yl |
| A-2085. | 4-bromo-2,5-dihydro-imidazol-2-on-1-yl |
| A-2086. | 5-bromo-2,5-dihydro-imidazol-2-on-1-yl |
| A-2087. | 4-cyano-2,5-dihydro-imidazol-2-on-1-yl |
| A-2088. | 5-cyano-2,5-dihydro-imidazol-2-on-1-yl |
| A-2089. | 4-nitro-2,5-dihydro-imidazol-2-on-1-yl |
| A-2090. | 5-nitro-2,5-dihydro-imidazol-2-on-1-yl |
| A-2091. | 4-methyl-2,5-dihydro-imidazol-2-on-1-yl |
| A-2092. | 5-methyl-2,5-dihydro-imidazol-2-on-1-yl |
| A-2093. | 4-trifluoromethyl-2,5-dihydro-imidazol-2-on-1-yl |
| A-2094. | 5-trifluoromethyl-2,5-dihydro-imidazol-2-on-1-yl |
| A-2095. | 4-methoxy-2,5-dihydro-imidazol-2-on-1-yl |
| A-2096. | 5-methoxy-2,5-dihydro-imidazol-2-on-1-yl |
| A-2097. | 4-trifluoromethoxy-2,5-dihydro-imidazol-2-on-1-yl |
| A-2098. | 5-trifluoromethoxy-2,5-dihydro-imidazol-2-on-1-yl |
| A-2099. | 5-chloro-2,5-dihydro-imidazol-2-on-4-yl |
| A-2100. | 5-bromo-2,5-dihydro-imidazol-2-on-4-yl |
| A-2101. | 5-cyano-2,5-dihydro-imidazol-2-on-4-yl |
| A-2102. | 5-nitro-2,5-dihydro-imidazol-2-on-4-yl |
| A-2103. | 5-methyl-2,5-dihydro-imidazol-2-on-4-yl |
| A-2104. | 5-trifluoromethyl-2,5-dihydro-imidazol-2-on-4-yl |
| A-2105. | 5-methoxy-2,5-dihydro-imidazol-2-on-4-yl |
| A-2106. | 5-trifluoromethoxy-2,5-dihydro-imidazol-2-on-4-yl |
| A-2107. | 4-chloro-2,5-dihydro-imidazol-2-on-5-yl |
| A-2108. | 4-bromo-2,5-dihydro-imidazol-2-on-5-yl |
| A-2109. | 4-cyano-2,5-dihydro-imidazol-2-on-5-yl |
| A-2110. | 4-nitro-2,5-dihydro-imidazol-2-on-5-yl |
| A-2111. | 4-methyl-2,5-dihydro-imidazol-2-on-5-yl |
| A-2112. | 4-trifluoromethyl-2,5-dihydro-imidazol-2-on-5-yl |
| A-2113. | 4-methoxy-2,5-dihydro-imidazol-2-on-5-yl |
| A-2114. | 4-trifluoromethoxy-2,5-dihydro-imidazol-2-on-5-yl |
| A-2115. | 5-chloro-1-methyl-2,5-dihydro-imidazol-2-on-4-yl |
| A-2116. | 5-bromo-1-methyl-2,5-dihydro-imidazol-2-on-4-yl |
| A-2117. | 5-cyano-1-methyl-2,5-dihydro-imidazol-2-on-4-yl |
| A-2118. | 5-nitro-1-methyl-2,5-dihydro-imidazol-2-on-4-yl |
| A-2119. | 5-methyl-1-methyl-2,5-dihydro-imidazol-2-on-4-yl |
| A-2120. | 5-trifluoromethyl-1-methyl-2,5-dihydro-imidazol-2-on-4-yl |
| A-2121. | 5-methoxy-1-methyl-2,5-dihydro-imidazol-2-on-4-yl |
| A-2122. | 5-trifluoromethoxy-1-methyl-2,5-dihydro-imidazol-2-on- |
| | 4-yl |
| A-2123. | 4-chloro-1-methyl-2,5-dihydro-imidazol-2-on-5-yl |
| A-2124. | 4-bromo-1-methyl-2,5-dihydro-imidazol-2-on-5-yl |
| A-2125. | 4-cyano-1-methyl-2,5-dihydro-imidazol-2-on-5-yl |
| A-2126. | 4-nitro-1-methyl-2,5-dihydro-imidazol-2-on-5-yl |
| A-2127. | 4-methyl-1-methyl-2,5-dihydro-imidazol-2-on-5-yl |
| A-2128. | 4-trifluoromethyl-1-methyl-2,5-dihydro-imidazol-2-on-5-yl |
| A-2129. | 4-methoxy-1-methyl-2,5-dihydro-imidazol-2-on-5-yl |
| A-2130. | 4-trifluoromethoxy-1-methyl-2,5-dihydro-imidazol-2-on- |
| | 5-yl |
| A-2131. | 4,5-dihydro-imidazol-1-yl |
| A-2132. | 4,5-dihydro-imidazol-2-yl |
| A-2133. | 4,5-dihydro-imidazol-4-yl |
| A-2134. | 4,5-dihydro-imidazol-5-yl |
| A-2135. | 1-methyl-4,5-dihydro-imidazol-2-yl |
| A-2136. | 1-methyl-4,5-dihydro-imidazol-4-yl |
| A-2137. | 1-methyl-4,5-dihydro-imidazol-5-yl |
| A-2138. | 2-chloro-4,5-dihydro-imidazol-1-yl |
| A-2139. | 4-chloro-4,5-dihydro-imidazol-1-yl |
| A-2140. | 5-chloro-4,5-dihydro-imidazol-1-yl |
| A-2141. | 2-bromo-4,5-dihydro-imidazol-1-yl |
| A-2142. | 4-bromo-4,5-dihydro-imidazol-1-yl |
| A-2143. | 5-bromo-4,5-dihydro-imidazol-1-yl |
| A-2144. | 2-cyano-4,5-dihydro-imidazol-1-yl |
| A-2145. | 4-cyano-4,5-dihydro-imidazol-1-yl |
| A-2146. | 5-cyano-4,5-dihydro-imidazol-1-yl |
| A-2147. | 2-nitro-4,5-dihydro-imidazol-1-yl |
| A-2148. | 4-nitro-4,5-dihydro-imidazol-1-yl |
| A-2149. | 5-nitro-4,5-dihydro-imidazol-1-yl |
| A-2150. | 2-methyl-4,5-dihydro-imidazol-1-yl |
| A-2151. | 4-methyl-4,5-dihydro-imidazol-1-yl |
| A-2152. | 5-methyl-4,5-dihydro-imidazol-1-yl |
| A-2153. | 2-trifluoromethyl-4,5-dihydro-imidazol-1-yl |
| A-2154. | 4-trifluoromethyl-4,5-dihydro-imidazol-1-yl |
| A-2155. | 5-trifluoromethyl-4,5-dihydro-imidazol-1-yl |
| A-2156. | 2-methoxy-4,5-dihydro-imidazol-1-yl |
| A-2157. | 4-methoxy-4,5-dihydro-imidazol-1-yl |
| A-2158. | 5-methoxy-4,5-dihydro-imidazol-1-yl |
| A-2159. | 2-trifluoromethoxy-4,5-dihydro-imidazol-1-yl |
| A-2160. | 4-trifluoromethoxy-4,5-dihydro-imidazol-1-yl |
| A-2161. | 5-trifluoromethoxy-4,5-dihydro-imidazol-1-yl |
| A-2162. | 4-chloro-4,5-dihydro-imidazol-2-yl |
| A-2163. | 5-chloro-4,5-dihydro-imidazol-2-yl |
| A-2164. | 4-bromo-4,5-dihydro-imidazol-2-yl |
| A-2165. | 5-bromo-4,5-dihydro-imidazol-2-yl |
| A-2166. | 4-cyano-4,5-dihydro-imidazol-2-yl |
| A-2167. | 5-cyano-4,5-dihydro-imidazol-2-yl |
| A-2168. | 4-nitro-4,5-dihydro-imidazol-2-yl |
| A-2169. | 5-nitro-4,5-dihydro-imidazol-2-yl |
| A-2170. | 4-methyl-4,5-dihydro-imidazol-2-yl |
| A-2171. | 5-methyl-4,5-dihydro-imidazol-2-yl |
| A-2172. | 4-trifluoromethyl-4,5-dihydro-imidazol-2-yl |
| A-2173. | 5-trifluoromethyl-4,5-dihydro-imidazol-2-yl |
| A-2174. | 4-methoxy-4,5-dihydro-imidazol-2-yl |
| A-2175. | 5-methoxy-4,5-dihydro-imidazol-2-yl |
| A-2176. | 4-trifluoromethoxy-4,5-dihydro-imidazol-2-yl |
| A-2177. | 5-trifluoromethoxy-4,5-dihydro-imidazol-2-yl |
| A-2178. | 2-chloro-4,5-dihydro-imidazol-4-yl |
| A-2179. | 5-chloro-4,5-dihydro-imidazol-4-yl |
| A-2180. | 2-bromo-4,5-dihydro-imidazol-4-yl |
| A-2181. | 5-bromo-4,5-dihydro-imidazol-4-yl |
| A-2182. | 2-cyano-4,5-dihydro-imidazol-4-yl |
| A-2183. | 5-cyano-4,5-dihydro-imidazol-4-yl |
| A-2184. | 2-nitro-4,5-dihydro-imidazol-4-yl |
| A-2185. | 5-nitro-4,5-dihydro-imidazol-4-yl |
| A-2186. | 2-methyl-4,5-dihydro-imidazol-4-yl |
| A-2187. | 5-methyl-4,5-dihydro-imidazol-4-yl |
| A-2188. | 2-trifluoromethyl-4,5-dihydro-imidazol-4-yl |
| A-2189. | 5-trifluoromethyl-4,5-dihydro-imidazol-4-yl |
| A-2190. | 2-methoxy-4,5-dihydro-imidazol-4-yl |
| A-2191. | 5-methoxy-4,5-dihydro-imidazol-4-yl |
| A-2192. | 2-trifluoromethoxy-4,5-dihydro-imidazol-4-yl |
| A-2193. | 5-trifluoromethoxy-4,5-dihydro-imidazol-4-yl |
| A-2194. | 2-chloro-4,5-dihydro-imidazol-5-yl |
| A-2195. | 4-chloro-4,5-dihydro-imidazol-5-yl |
| A-2196. | 2-bromo-4,5-dihydro-imidazol-5-yl |
| A-2197. | 4-bromo-4,5-dihydro-imidazol-5-yl |
| A-2198. | 2-cyano-4,5-dihydro-imidazol-5-yl |
| A-2199. | 4-cyano-4,5-dihydro-imidazol-5-yl |
| A-2200. | 2-nitro-4,5-dihydro-imidazol-5-yl |
| A-2201. | 4-nitro-4,5-dihydro-imidazol-5-yl |
| A-2202. | 2-methyl-4,5-dihydro-imidazol-5-yl |
| A-2203. | 4-methyl-4,5-dihydro-imidazol-5-yl |
| A-2204. | 2-trifluoromethyl-4,5-dihydro-imidazol-5-yl |
| A-2205. | 4-trifluoromethyl-4,5-dihydro-imidazol-5-yl |
| A-2206. | 2-methoxy-4,5-dihydro-imidazol-5-yl |
| A-2207. | 4-methoxy-4,5-dihydro-imidazol-5-yl |
| A-2208. | 2-trifluoromethoxy-4,5-dihydro-imidazol-5-yl |
| A-2209. | 4-trifluoromethoxy-4,5-dihydro-imidazol-5-yl |
| A-2210. | 4-chloro-1-methyl-4,5-dihydro-imidazol-2-yl |
| A-2211. | 5-chloro-1-methyl-4,5-dihydro-imidazol-2-yl |
| A-2212. | 4-bromo-1-methyl-4,5-dihydro-imidazol-2-yl |
| A-2213. | 5-bromo-1-methyl-4,5-dihydro-imidazol-2-yl |
| A-2214. | 4-cyano-1-methyl-4,5-dihydro-imidazol-2-yl |
| A-2215. | 5-cyano-1-methyl-4,5-dihydro-imidazol-2-yl |
| A-2216. | 4-nitro-1-methyl-4,5-dihydro-imidazol-2-yl |
| A-2217. | 5-nitro-1-methyl-4,5-dihydro-imidazol-2-yl |
| A-2218. | 4-methyl-1-methyl-4,5-dihydro-imidazol-2-yl |
| A-2219. | 5-methyl-1-methyl-4,5-dihydro-imidazol-2-yl |
| A-2220. | 4-trifluoromethyl-1-methyl-4,5-dihydro-imidazol-2-yl |
| A-2221. | 5-trifluoromethyl-1-methyl-4,5-dihydro-imidazol-2-yl |
| A-2222. | 4-methoxy-1-methyl-4,5-dihydro-imidazol-2-yl |
| A-2223. | 5-methoxy-1-methyl-4,5-dihydro-imidazol-2-yl |
| A-2224. | 4-trifluoromethoxy-1-methyl-4,5-dihydro-imidazol-2-yl |
| A-2225. | 5-trifluoromethoxy-1-methyl-4,5-dihydro-imidazol-2-yl |
| A-2226. | 2-chloro-1-methyl-4,5-dihydro-imidazol-4-yl |
| A-2227. | 5-chloro-1-methyl-4,5-dihydro-imidazol-4-yl |
| A-2228. | 2-bromo-1-methyl-4,5-dihydro-imidazol-4-yl |
| A-2229. | 5-bromo-1-methyl-4,5-dihydro-imidazol-4-yl |
| A-2230. | 2-cyano-1-methyl-4,5-dihydro-imidazol-4-yl |
| A-2231. | 5-cyano-1-methyl-4,5-dihydro-imidazol-4-yl |
| A-2232. | 2-nitro-1-methyl-4,5-dihydro-imidazol-4-yl |
| A-2233. | 5-nitro-1-methyl-4,5-dihydro-imidazol-4-yl |
| A-2234. | 2-methyl-1-methyl-4,5-dihydro-imidazol-4-yl |
| A-2235. | 5-methyl-1-methyl-4,5-dihydro-imidazol-4-yl |
| A-2236. | 2-trifluoromethyl-1-methyl-4,5-dihydro-imidazol-4-yl |
| A-2237. | 5-trifluoromethyl-1-methyl-4,5-dihydro-imidazol-4-yl |
| A-2238. | 2-methoxy-1-methyl-4,5-dihydro-imidazol-4-yl |
| A-2239. | 5-methoxy-1-methyl-4,5-dihydro-imidazol-4-yl |
| A-2240. | 2-trifluoromethoxy-1-methyl-4,5-dihydro-imidazol-4-yl |
| A-2241. | 5-trifluoromethoxy-1-methyl-4,5-dihydro-imidazol-4-yl |
| A-2242. | 2-chloro-1-methyl-4,5-dihydro-imidazol-5-yl |
| A-2243. | 4-chloro-1-methyl-4,5-dihydro-imidazol-5-yl |
| A-2244. | 2-bromo-1-methyl-4,5-dihydro-imidazol-5-yl |
| A-2245. | 4-bromo-1-methyl-4,5-dihydro-imidazol-5-yl |
| A-2246. | 2-cyano-1-methyl-4,5-dihydro-imidazol-5-yl |
| A-2247. | 4-cyano-1-methyl-4,5-dihydro-imidazol-5-yl |
| A-2248. | 2-nitro-1-methyl-4,5-dihydro-imidazol-5-yl |
| A-2249. | 4-nitro-1-methyl-4,5-dihydro-imidazol-5-yl |
| A-2250. | 2-methyl-1-methyl-4,5-dihydro-imidazol-5-yl |
| A-2251. | 4-methyl-1-methyl-4,5-dihydro-imidazol-5-yl |
| A-2252. | 2-trifluoromethyl-1-methyl-4,5-dihydro-imidazol-5-yl |
| A-2253. | 4-trifluoromethyl-1-methyl-4,5-dihydro-imidazol-5-yl |
| A-2254. | 2-methoxy-1-methyl-4,5-dihydro-imidazol-5-yl |
| A-2255. | 4-methoxy-1-methyl-4,5-dihydro-imidazol-5-yl |
| A-2256. | 2-trifluoromethoxy-1-methyl-4,5-dihydro-imidazol-5-yl |
| A-2257. | 4-trifluoromethoxy-1-methyl-4,5-dihydro-imidazol-5-yl |
| A-2258. | 2,3-dihydro-oxazol-2-yl |
| A-2259. | 2,3-dihydro-oxazol-3-yl |
| A-2260. | 2,3-dihydro-oxazol-4-yl |
| A-2261. | 2,3-dihydro-oxazol-5-yl |
| A-2262. | 4-chloro-2,3-dihydro-oxazol-2-yl |
| A-2263. | 5-chloro-2,3-dihydro-oxazol-2-yl |
| A-2264. | 4-bromo-2,3-dihydro-oxazol-2-yl |
| A-2265. | 5-bromo-2,3-dihydro-oxazol-2-yl |
| A-2266. | 3-cyano-2,3-dihydro-oxazol-2-yl |
| A-2267. | 4-cyano-2,3-dihydro-oxazol-2-yl |
| A-2268. | 5-cyano-2,3-dihydro-oxazol-2-yl |
| A-2269. | 3-nitro-2,3-dihydro-oxazol-2-yl |
| A-2270. | 4-nitro-2,3-dihydro-oxazol-2-yl |
| A-2271. | 5-nitro-2,3-dihydro-oxazol-2-yl |
| A-2272. | 3-methyl-2,3-dihydro-oxazol-2-yl |
| A-2273. | 4-methyl-2,3-dihydro-oxazol-2-yl |
| A-2274. | 5-methyl-2,3-dihydro-oxazol-2-yl |
| A-2275. | 3-trifluoromethyl-2,3-dihydro-oxazol-2-yl |
| A-2276. | 4-trifluoromethyl-2,3-dihydro-oxazol-2-yl |
| A-2277. | 5-trifluoromethyl-2,3-dihydro-oxazol-2-yl |
| A-2278. | 3-trifluoromethyl-2,3-dihydro-oxazol-2-yl |
| A-2279. | 4-trifluoromethyl-2,3-dihydro-oxazol-2-yl |
| A-2280. | 5-methoxy-2,3-dihydro-oxazol-2-yl |
| A-2281. | 3-trifluoromethoxy-2,3-dihydro-oxazol-2-yl |
| A-2282. | 4-trifluoromethoxy-2,3-dihydro-oxazol-2-yl |
| A-2283. | 5-trifluoromethoxy-2,3-dihydro-oxazol-2-yl |
| A-2284. | 2-chloro-2,3-dihydro-oxazol-3-yl |
| A-2285. | 4-chloro-2,3-dihydro-oxazol-3-yl |
| A-2286. | 5-chloro-2,3-dihydro-oxazol-3-yl |
| A-2287. | 2-bromo-2,3-dihydro-oxazol-3-yl |
| A-2288. | 4-bromo-2,3-dihydro-oxazol-3-yl |
| A-2289. | 5-bromo-2,3-dihydro-oxazol-3-yl |
| A-2290. | 2-cyano-2,3-dihydro-oxazol-3-yl |
| A-2291. | 4-cyano-2,3-dihydro-oxazol-3-yl |
| A-2292. | 5-cyano-2,3-dihydro-oxazol-3-yl |
| A-2293. | 2-nitro-2,3-dihydro-oxazol-3-yl |
| A-2294. | 4-nitro-2,3-dihydro-oxazol-3-yl |
| A-2295. | 5-nitro-2,3-dihydro-oxazol-3-yl |
| A-2296. | 2-methyl-2,3-dihydro-oxazol-3-yl |
| A-2297. | 4-methyl-2,3-dihydro-oxazol-3-yl |
| A-2298. | 5-methyl-2,3-dihydro-oxazol-3-yl |
| A-2299. | 2-trifluoromethyl-2,3-dihydro-oxazol-3-yl |
| A-2300. | 4-trifluoromethyl-2,3-dihydro-oxazol-3-yl |
| A-2301. | 5-trifluoromethyl-2,3-dihydro-oxazol-3-yl |
| A-2302. | 2-methoxy-2,3-dihydro-oxazol-3-yl |
| A-2303. | 4-methoxy-2,3-dihydro-oxazol-3-yl |
| A-2304. | 5-methoxy-2,3-dihydro-oxazol-3-yl |
| A-2305. | 2-trifluoromethoxy-2,3-dihydro-oxazol-3-yl |
| A-2306. | 4-trifluoromethoxy-2,3-dihydro-oxazol-3-yl |
| A-2307. | 5-trifluoromethoxy-2,3-dihydro-oxazol-3-yl |
| A-2308. | 2-chloro-2,3-dihydro-oxazol-4-yl |
| A-2309. | 5-chloro-2,3-dihydro-oxazol-4-yl |
| A-2310. | 2-bromo-2,3-dihydro-oxazol-4-yl |
| A-2311. | 5-bromo-2,3-dihydro-oxazol-4-yl |
| A-2312. | 2-cyano-2,3-dihydro-oxazol-4-yl |
| A-2313. | 3-cyano-2,3-dihydro-oxazol-4-yl |
| A-2314. | 5-cyano-2,3-dihydro-oxazol-4-yl |
| A-2315. | 2-nitro-2,3-dihydro-oxazol-4-yl |
| A-2316. | 3-nitro-2,3-dihydro-oxazol-4-yl |
| A-2317. | 5-nitro-2,3-dihydro-oxazol-4-yl |
| A-2318. | 2-methyl-2,3-dihydro-oxazol-4-yl |
| A-2319. | 3-methyl-2,3-dihydro-oxazol-4-yl |
| A-2320. | 5-methyl-2,3-dihydro-oxazol-4-yl |
| A-2321. | 2-trifluoromethyl-2,3-dihydro-oxazol-4-yl |
| A-2322. | 3-trifluoromethyl-2,3-dihydro-oxazol-4-yl |
| A-2323. | 5-trifluoromethyl-2,3-dihydro-oxazol-4-yl |
| A-2324. | 2-methoxy-2,3-dihydro-oxazol-4-yl |
| A-2325. | 3-methoxy-2,3-dihydro-oxazol-4-yl |
| A-2326. | 5-methoxy-2,3-dihydro-oxazol-4-yl |
| A-2327. | 2-trifluoromethoxy-2,3-dihydro-oxazol-4-yl |
| A-2328. | 3-trifluoromethoxy-2,3-dihydro-oxazol-4-yl |
| A-2329. | 5-trifluoromethoxy-2,3-dihydro-oxazol-4-yl |
| A-2330. | 2-chloro-2,3-dihydro-oxazol-5-yl |
| A-2331. | 4-chloro-2,3-dihydro-oxazol-5-yl |
| A-2332. | 2-bromo-2,3-dihydro-oxazol-5-yl |
| A-2333. | 4-bromo-2,3-dihydro-oxazol-5-yl |
| A-2334. | 2-cyano-2,3-dihydro-oxazol-5-yl |
| A-2335. | 3-cyano-2,3-dihydro-oxazol-5-yl |
| A-2336. | 4-cyano-2,3-dihydro-oxazol-5-yl |
| A-2337. | 2-nitro-2,3-dihydro-oxazol-5-yl |
| A-2338. | 3-nitro-2,3-dihydro-oxazol-5-yl |
| A-2339. | 4-nitro-2,3-dihydro-oxazol-5-yl |
| A-2340. | 2-methyl-2,3-dihydro-oxazol-5-yl |
| A-2341. | 3-methyl-2,3-dihydro-oxazol-5-yl |
| A-2342. | 4-methyl-2,3-dihydro-oxazol-5-yl |
| A-2343. | 2-trifluoromethyl-2,3-dihydro-oxazol-5-yl |
| A-2344. | 3-trifluoromethyl-2,3-dihydro-oxazol-5-yl |
| A-2345. | 4-trifluoromethyl-2,3-dihydro-oxazol-5-yl |
| A-2346. | 2-methoxy-2,3-dihydro-oxazol-5-yl |
| A-2347. | 3-methoxy-2,3-dihydro-oxazol-5-yl |
| A-2348. | 4-methoxy-2,3-dihydro-oxazol-5-yl |
| A-2349. | 2-trifluoromethoxy-2,3-dihydro-oxazol-5-yl |
| A-2350. | 3-trifluoromethoxy-2,3-dihydro-oxazol-5-yl |
| A-2351. | 4-trifluoromethoxy-2,3-dihydro-oxazol-5-yl |
| A-2352. | 2,5-dihydro-oxazol-2-yl |
| A-2353. | 2,5-dihydro-oxazol-4-yl |
| A-2354. | 2,5-dihydro-oxazol-5-yl |
| A-2355. | 4-chloro-2,5-dihydro-oxazol-2-yl |
| A-2356. | 5-chloro-2,5-dihydro-oxazol-2-yl |
| A-2357. | 4-bromo-2,5-dihydro-oxazol-2-yl |
| A-2358. | 5-bromo-2,5-dihydro-oxazol-2-yl |
| A-2359. | 4-cyano-2,5-dihydro-oxazol-2-yl |
| A-2360. | 5-cyano-2,5-dihydro-oxazol-2-yl |
| A-2361. | 4-nitro-2,5-dihydro-oxazol-2-yl |
| A-2362. | 5-nitro-2,5-dihydro-oxazol-2-yl |
| A-2363. | 4-methyl-2,5-dihydro-oxazol-2-yl |
| A-2364. | 5-methyl-2,5-dihydro-oxazol-2-yl |
| A-2365. | 4-trifluoromethyl-2,5-dihydro-oxazol-2-yl |
| A-2366. | 5-trifluoromethyl-2,5-dihydro-oxazol-2-yl |
| A-2367. | 4-methoxy-2,5-dihydro-oxazol-2-yl |
| A-2368. | 5-methoxy-2,5-dihydro-oxazol-2-yl |
| A-2369. | 4-trifluoromethoxy-2,5-dihydro-oxazol-2-yl |
| A-2370. | 5-trifluoromethoxy-2,5-dihydro-oxazol-2-yl |
| A-2371. | 2-chloro-2,5-dihydro-oxazol-4-yl |
| A-2372. | 5-chloro-2,5-dihydro-oxazol-4-yl |
| A-2373. | 2-bromo-2,5-dihydro-oxazol-4-yl |
| A-2374. | 5-bromo-2,5-dihydro-oxazol-4-yl |
| A-2375. | 2-cyano-2,5-dihydro-oxazol-4-yl |
| A-2376. | 5-cyano-2,5-dihydro-oxazol-4-yl |
| A-2377. | 2-nitro-2,5-dihydro-oxazol-4-yl |
| A-2378. | 5-nitro-2,5-dihydro-oxazol-4-yl |
| A-2379. | 2-methyl-2,5-dihydro-oxazol-4-yl |
| A-2380. | 5-methyl-2,5-dihydro-oxazol-4-yl |
| A-2381. | 2-trifluoromethyl-2,5-dihydro-oxazol-4-yl |
| A-2382. | 5-trifluoromethyl-2,5-dihydro-oxazol-4-yl |
| A-2383. | 2-methoxy-2,5-dihydro-oxazol-4-yl |
| A-2384. | 5-methoxy-2,5-dihydro-oxazol-4-yl |
| A-2385. | 2-trifluoromethoxy-2,5-dihydro-oxazol-4-yl |
| A-2386. | 5-trifluoromethoxy-2,5-dihydro-oxazol-4-yl |
| A-2387. | 2-chloro-2,5-dihydro-oxazol-5-yl |
| A-2388. | 4-chloro-2,5-dihydro-oxazol-5-yl |
| A-2389. | 2-bromo-2,5-dihydro-oxazol-5-yl |
| A-2390. | 4-bromo-2,5-dihydro-oxazol-5-yl |
| A-2391. | 2-cyano-2,5-dihydro-oxazol-5-yl |
| A-2392. | 4-cyano-2,5-dihydro-oxazol-5-yl |
| A-2393. | 2-nitro-2,5-dihydro-oxazol-5-yl |
| A-2394. | 4-nitro-2,5-dihydro-oxazol-5-yl |
| A-2395. | 2-methyl-2,5-dihydro-oxazol-5-yl |
| A-2396. | 4-methyl-2,5-dihydro-oxazol-5-yl |
| A-2397. | 2-trifluoromethyl-2,5-dihydro-oxazol-5-yl |
| A-2398. | 4-trifluoromethyl-2,5-dihydro-oxazol-5-yl |
| A-2399. | 2-methoxy-2,5-dihydro-oxazol-5-yl |
| A-2400. | 4-methoxy-2,5-dihydro-oxazol-5-yl |
| A-2401. | 2-trifluoromethoxy-2,5-dihydro-oxazol-5-yl |
| A-2402. | 4-trifluoromethoxy-2,5-dihydro-oxazol-5-yl |
| A-2403. | 4,5-dihydro-oxazol-2-yl |
| A-2404. | 4,5-dihydro-oxazol-4-yl |
| A-2405. | 4,5-dihydro-oxazol-5-yl |
| A-2406. | 4-chloro-4,5-dihydro-oxazol-2-yl |
| A-2407. | 5-chloro-4,5-dihydro-oxazol-2-yl |
| A-2408. | 4-bromo-4,5-dihydro-oxazol-2-yl |
| A-2409. | 5-bromo-4,5-dihydro-oxazol-2-yl |
| A-2410. | 4-cyano-4,5-dihydro-oxazol-2-yl |
| A-2411. | 5-cyano-4,5-dihydro-oxazol-2-yl |
| A-2412. | 4-nitro-4,5-dihydro-oxazol-2-yl |
| A-2413. | 5-nitro-4,5-dihydro-oxazol-2-yl |
| A-2414. | 4-methyl-4,5-dihydro-oxazol-2-yl |
| A-2415. | 5-methyl-4,5-dihydro-oxazol-2-yl |
| A-2416. | 4-trifluoromethyl-4,5-dihydro-oxazol-2-yl |
| A-2417. | 5-trifluoromethyl-4,5-dihydro-oxazol-2-yl |
| A-2418. | 4-methoxy-4,5-dihydro-oxazol-2-yl |
| A-2419. | 5-methoxy-4,5-dihydro-oxazol-2-yl |
| A-2420. | 4-trifluoromethoxy-4,5-dihydro-oxazol-2-yl |
| A-2421. | 5-trifluoromethoxy-4,5-dihydro-oxazol-2-yl |
| A-2422. | 2-chloro-4,5-dihydro-oxazol-4-yl |
| A-2423. | 5-chloro-4,5-dihydro-oxazol-4-yl |
| A-2424. | 2-bromo-4,5-dihydro-oxazol-4-yl |
| A-2425. | 5-bromo-4,5-dihydro-oxazol-4-yl |
| A-2426. | 2-cyano-4,5-dihydro-oxazol-4-yl |
| A-2427. | 5-cyano-4,5-dihydro-oxazol-4-yl |
| A-2428. | 2-nitro-4,5-dihydro-oxazol-4-yl |
| A-2429. | 5-nitro-4,5-dihydro-oxazol-4-yl |
| A-2430. | 2-methyl-4,5-dihydro-oxazol-4-yl |
| A-2431. | 5-methyl-4,5-dihydro-oxazol-4-yl |
| A-2432. | 2-trifluoromethyl-4,5-dihydro-oxazol-4-yl |
| A-2433. | 5-trifluoromethyl-4,5-dihydro-oxazol-4-yl |
| A-2434. | 2-methoxy-4,5-dihydro-oxazol-4-yl |
| A-2435. | 5-methoxy-4,5-dihydro-oxazol-4-yl |
| A-2436. | 2-trifluoromethoxy-4,5-dihydro-oxazol-4-yl |
| A-2437. | 5-trifluoromethoxy-4,5-dihydro-oxazol-4-yl |
| A-2438. | 2-chloro-4,5-dihydro-oxazol-5-yl |
| A-2439. | 4-chloro-4,5-dihydro-oxazol-5-yl |
| A-2440. | 2-bromo-4,5-dihydro-oxazol-5-yl |
| A-2441. | 4-bromo-4,5-dihydro-oxazol-5-yl |
| A-2442. | 2-cyano-4,5-dihydro-oxazol-5-yl |
| A-2443. | 4-cyano-4,5-dihydro-oxazol-5-yl |
| A-2444. | 2-nitro-4,5-dihydro-oxazol-5-yl |
| A-2445. | 4-nitro-4,5-dihydro-oxazol-5-yl |
| A-2446. | 2-methyl-4,5-dihydro-oxazol-5-yl |
| A-2447. | 4-methyl-4,5-dihydro-oxazol-5-yl |
| A-2448. | 2-trifluoromethyl-4,5-dihydro-oxazol-5-yl |
| A-2449. | 4-trifluoromethyl-4,5-dihydro-oxazol-5-yl |
| A-2450. | 2-methoxy-4,5-dihydro-oxazol-5-yl |
| A-2451. | 4-methoxy-4,5-dihydro-oxazol-5-yl |
| A-2452. | 2-trifluoromethoxy-4,5-dihydro-oxazol-5-yl |
| A-2453. | 4-trifluoromethoxy-4,5-dihydro-oxazol-5-yl |
| A-2454. | 2,3-dihydro-thiazol-2-yl |
| A-2455. | 2,3-dihydro-thiazol-3-yl |
| A-2456. | 2,3-dihydro-thiazol-4-yl |
| A-2457. | 2,3-dihydro-thiazol-5-yl |
| A-2458. | 4-chloro-2,3-dihydro-thiazol-2-yl |
| A-2459. | 5-chloro-2,3-dihydro-thiazol-2-yl |
| A-2460. | 4-bromo-2,3-dihydro-thiazol-2-yl |
| A-2461. | 5-bromo-2,3-dihydro-thiazol-2-yl |
| A-2462. | 3-cyano-2,3-dihydro-thiazol-2-yl |
| A-2463. | 4-cyano-2,3-dihydro-thiazol-2-yl |
| A-2464. | 5-cyano-2,3-dihydro-thiazol-2-yl |
| A-2465. | 3-nitro-2,3-dihydro-thiazol-2-yl |
| A-2466. | 4-nitro-2,3-dihydro-thiazol-2-yl |
| A-2467. | 5-nitro-2,3-dihydro-thiazol-2-yl |
| A-2468. | 3-methyl-2,3-dihydro-thiazol-2-yl |
| A-2469. | 4-methyl-2,3-dihydro-thiazol-2-yl |
| A-2470. | 5-methyl-2,3-dihydro-thiazol-2-yl |
| A-2471. | 3-trifluoromethyl-2,3-dihydro-thiazol-2-yl |
| A-2472. | 4-trifluoromethyl-2,3-dihydro-thiazol-2-yl |
| A-2473. | 5-trifluoromethyl-2,3-dihydro-thiazol-2-yl |
| A-2474. | 3-trifluoromethyl-2,3-dihydro-thiazol-2-yl |
| A-2475. | 4-trifluoromethyl-2,3-dihydro-thiazol-2-yl |
| A-2476. | 5-methoxy-2,3-dihydro-thiazol-2-yl |
| A-2477. | 3-trifluoromethoxy-2,3-dihydro-thiazol-2-yl |
| A-2478. | 4-trifluoromethoxy-2,3-dihydro-thiazol-2-yl |
| A-2479. | 5-trifluoromethoxy-2,3-dihydro-thiazol-2-yl |
| A-2480. | 2-chloro-2,3-dihydro-thiazol-3-yl |
| A-2481. | 4-chloro-2,3-dihydro-thiazol-3-yl |
| A-2482. | 5-chloro-2,3-dihydro-thiazol-3-yl |
| A-2483. | 2-bromo-2,3-dihydro-thiazol-3-yl |
| A-2484. | 4-bromo-2,3-dihydro-thiazol-3-yl |
| A-2485. | 5-bromo-2,3-dihydro-thiazol-3-yl |
| A-2486. | 2-cyano-2,3-dihydro-thiazol-3-yl |
| A-2487. | 4-cyano-2,3-dihydro-thiazol-3-yl |
| A-2488. | 5-cyano-2,3-dihydro-thiazol-3-yl |
| A-2489. | 2-nitro-2,3-dihydro-thiazol-3-yl |
| A-2490. | 4-nitro-2,3-dihydro-thiazol-3-yl |
| A-2491. | 5-nitro-2,3-dihydro-thiazol-3-yl |
| A-2492. | 2-methyl-2,3-dihydro-thiazol-3-yl |
| A-2493. | 4-methyl-2,3-dihydro-thiazol-3-yl |
| A-2494. | 5-methyl-2,3-dihydro-thiazol-3-yl |
| A-2495. | 2-trifluoromethyl-2,3-dihydro-thiazol-3-yl |
| A-2496. | 4-trifluoromethyl-2,3-dihydro-thiazol-3-yl |
| A-2497. | 5-trifluoromethyl-2,3-dihydro-thiazol-3-yl |
| A-2498. | 2-methoxy-2,3-dihydro-thiazol-3-yl |
| A-2499. | 4-methoxy-2,3-dihydro-thiazol-3-yl |
| A-2500. | 5-methoxy-2,3-dihydro-thiazol-3-yl |
| A-2501. | 2-trifluoromethoxy-2,3-dihydro-thiazol-3-yl |
| A-2502. | 4-trifluoromethoxy-2,3-dihydro-thiazol-3-yl |
| A-2503. | 5-trifluoromethoxy-2,3-dihydro-thiazol-3-yl |
| A-2504. | 2-chloro-2,3-dihydro-thiazol-4-yl |
| A-2505. | 5-chloro-2,3-dihydro-thiazol-4-yl |
| A-2506. | 2-bromo-2,3-dihydro-thiazol-4-yl |
| A-2507. | 5-bromo-2,3-dihydro-thiazol-4-yl |
| A-2508. | 2-cyano-2,3-dihydro-thiazol-4-yl |
| A-2509. | 3-cyano-2,3-dihydro-thiazol-4-yl |
| A-2510. | 5-cyano-2,3-dihydro-thiazol-4-yl |
| A-2511. | 2-nitro-2,3-dihydro-thiazol-4-yl |
| A-2512. | 3-nitro-2,3-dihydro-thiazol-4-yl |
| A-2513. | 5-nitro-2,3-dihydro-thiazol-4-yl |
| A-2514. | 2-methyl-2,3-dihydro-thiazol-4-yl |
| A-2515. | 3-methyl-2,3-dihydro-thiazol-4-yl |
| A-2516. | 5-methyl-2,3-dihydro-thiazol-4-yl |
| A-2517. | 2-trifluoromethyl-2,3-dihydro-thiazol-4-yl |
| A-2518. | 3-trifluoromethyl-2,3-dihydro-thiazol-4-yl |
| A-2519. | 5-trifluoromethyl-2,3-dihydro-thiazol-4-yl |
| A-2520. | 2-methoxy-2,3-dihydro-thiazol-4-yl |
| A-2521. | 3-methoxy-2,3-dihydro-thiazol-4-yl |
| A-2522. | 5-methoxy-2,3-dihydro-thiazol-4-yl |
| A-2523. | 2-trifluoromethoxy-2,3-dihydro-thiazol-4-yl |
| A-2524. | 3-trifluoromethoxy-2,3-dihydro-thiazol-4-yl |
| A-2525. | 5-trifluoromethoxy-2,3-dihydro-thiazol-4-yl |
| A-2526. | 2-chloro-2,3-dihydro-thiazol-5-yl |
| A-2527. | 4-chloro-2,3-dihydro-thiazol-5-yl |
| A-2528. | 2-bromo-2,3-dihydro-thiazol-5-yl |
| A-2529. | 4-bromo-2,3-dihydro-thiazol-5-yl |
| A-2530. | 2-cyano-2,3-dihydro-thiazol-5-yl |
| A-2531. | 3-cyano-2,3-dihydro-thiazol-5-yl |
| A-2532. | 4-cyano-2,3-dihydro-thiazol-5-yl |
| A-2533. | 2-nitro-2,3-dihydro-thiazol-5-yl |
| A-2534. | 3-nitro-2,3-dihydro-thiazol-5-yl |
| A-2535. | 4-nitro-2,3-dihydro-thiazol-5-yl |
| A-2536. | 2-methyl-2,3-dihydro-thiazol-5-yl |
| A-2537. | 3-methyl-2,3-dihydro-thiazol-5-yl |
| A-2538. | 4-methyl-2,3-dihydro-thiazol-5-yl |
| A-2539. | 2-trifluoromethyl-2,3-dihydro-thiazol-5-yl |
| A-2540. | 3-trifluoromethyl-2,3-dihydro-thiazol-5-yl |
| A-2541. | 4-trifluoromethyl-2,3-dihydro-thiazol-5-yl |
| A-2542. | 2-methoxy-2,3-dihydro-thiazol-5-yl |
| A-2543. | 3-methoxy-2,3-dihydro-thiazol-5-yl |
| A-2544. | 4-methoxy-2,3-dihydro-thiazol-5-yl |
| A-2545. | 2-trifluoromethoxy-2,3-dihydro-thiazol-5-yl |
| A-2546. | 3-trifluoromethoxy-2,3-dihydro-thiazol-5-yl |
| A-2547. | 4-trifluoromethoxy-2,3-dihydro-thiazol-5-yl |
| A-2548. | 2,5-dihydro-thiazol-2-yl |
| A-2549. | 2,5-dihydro-thiazol-4-yl |
| A-2550. | 2,5-dihydro-thiazol-5-yl |
| A-2551. | 4-chloro-2,5-dihydro-thiazol-2-yl |
| A-2552. | 5-chloro-2,5-dihydro-thiazol-2-yl |
| A-2553. | 4-bromo-2,5-dihydro-thiazol-2-yl |
| A-2554. | 5-bromo-2,5-dihydro-thiazol-2-yl |
| A-2555. | 4-cyano-2,5-dihydro-thiazol-2-yl |
| A-2556. | 5-cyano-2,5-dihydro-thiazol-2-yl |
| A-2557. | 4-nitro-2,5-dihydro-thiazol-2-yl |
| A-2558. | 5-nitro-2,5-dihydro-thiazol-2-yl |
| A-2559. | 4-methyl-2,5-dihydro-thiazol-2-yl |
| A-2560. | 5-methyl-2,5-dihydro-thiazol-2-yl |
| A-2561. | 4-trifluoromethyl-2,5-dihydro-thiazol-2-yl |
| A-2562. | 5-trifluoromethyl-2,5-dihydro-thiazol-2-yl |
| A-2563. | 4-methoxy-2,5-dihydro-thiazol-2-yl |
| A-2564. | 5-methoxy-2,5-dihydro-thiazol-2-yl |
| A-2565. | 4-trifluoromethoxy-2,5-dihydro-thiazol-2-yl |
| A-2566. | 5-trifluoromethoxy-2,5-dihydro-thiazol-2-yl |
| A-2567. | 2-chloro-2,5-dihydro-thiazol-4-yl |
| A-2568. | 5-chloro-2,5-dihydro-thiazol-4-yl |
| A-2569. | 2-bromo-2,5-dihydro-thiazol-4-yl |
| A-2570. | 5-bromo-2,5-dihydro-thiazol-4-yl |
| A-2571. | 2-cyano-2,5-dihydro-thiazol-4-yl |
| A-2572. | 5-cyano-2,5-dihydro-thiazol-4-yl |
| A-2573. | 2-nitro-2,5-dihydro-thiazol-4-yl |
| A-2574. | 5-nitro-2,5-dihydro-thiazol-4-yl |
| A-2575. | 2-methyl-2,5-dihydro-thiazol-4-yl |
| A-2576. | 5-methyl-2,5-dihydro-thiazol-4-yl |
| A-2577. | 2-trifluoromethyl-2,5-dihydro-thiazol-4-yl |
| A-2578. | 5-trifluoromethyl-2,5-dihydro-thiazol-4-yl |
| A-2579. | 2-methoxy-2,5-dihydro-thiazol-4-yl |
| A-2580. | 5-methoxy-2,5-dihydro-thiazol-4-yl |
| A-2581. | 2-trifluoromethoxy-2,5-dihydro-thiazol-4-yl |
| A-2582. | 5-trifluoromethoxy-2,5-dihydro-thiazol-4-yl |
| A-2583. | 2-chloro-2,5-dihydro-thiazol-5-yl |
| A-2584. | 4-chloro-2,5-dihydro-thiazol-5-yl |
| A-2585. | 2-bromo-2,5-dihydro-thiazol-5-yl |
| A-2586. | 4-bromo-2,5-dihydro-thiazol-5-yl |
| A-2587. | 2-cyano-2,5-dihydro-thiazol-5-yl |
| A-2588. | 4-cyano-2,5-dihydro-thiazol-5-yl |
| A-2589. | 2-nitro-2,5-dihydro-thiazol-5-yl |
| A-2590. | 4-nitro-2,5-dihydro-thiazol-5-yl |
| A-2591. | 2-methyl-2,5-dihydro-thiazol-5-yl |
| A-2592. | 4-methyl-2,5-dihydro-thiazol-5-yl |
| A-2593. | 2-trifluoromethyl-2,5-dihydro-thiazol-5-yl |
| A-2594. | 4-trifluoromethyl-2,5-dihydro-thiazol-5-yl |
| A-2595. | 2-methoxy-2,5-dihydro-thiazol-5-yl |
| A-2596. | 4-methoxy-2,5-dihydro-thiazol-5-yl |
| A-2597. | 2-trifluoromethoxy-2,5-dihydro-thiazol-5-yl |
| A-2598. | 4-trifluoromethoxy-2,5-dihydro-thiazol-5-yl |
| A-2599. | 4,5-dihydro-thiazol-2-yl |
| A-2600. | 4,5-dihydro-thiazol-4-yl |
| A-2601. | 4,5-dihydro-thiazol-5-yl |
| A-2602. | 4-chloro-4,5-dihydro-thiazol-2-yl |
| A-2603. | 5-chloro-4,5-dihydro-thiazol-2-yl |
| A-2604. | 4-bromo-4,5-dihydro-thiazol-2-yl |
| A-2605. | 5-bromo-4,5-dihydro-thiazol-2-yl |
| A-2606. | 4-cyano-4,5-dihydro-thiazol-2-yl |
| A-2607. | 5-cyano-4,5-dihydro-thiazol-2-yl |
| A-2608. | 4-nitro-4,5-dihydro-thiazol-2-yl |
| A-2609. | 5-nitro-4,5-dihydro-thiazol-2-yl |
| A-2610. | 4-methyl-4,5-dihydro-thiazol-2-yl |
| A-2611. | 5-methyl-4,5-dihydro-thiazol-2-yl |
| A-2612. | 4-trifluoromethyl-4,5-dihydro-thiazol-2-yl |
| A-2613. | 5-trifluoromethyl-4,5-dihydro-thiazol-2-yl |
| A-2614. | 4-methoxy-4,5-dihydro-thiazol-2-yl |
| A-2615. | 5-methoxy-4,5-dihydro-thiazol-2-yl |
| A-2616. | 4-trifluoromethoxy-4,5-dihydro-thiazol-2-yl |
| A-2617. | 5-trifluoromethoxy-4,5-dihydro-thiazol-2-yl |
| A-2618. | 2-chloro-4,5-dihydro-thiazol-4-yl |
| A-2619. | 5-chloro-4,5-dihydro-thiazol-4-yl |
| A-2620. | 2-bromo-4,5-dihydro-thiazol-4-yl |
| A-2621. | 5-bromo-4,5-dihydro-thiazol-4-yl |
| A-2622. | 2-cyano-4,5-dihydro-thiazol-4-yl |
| A-2623. | 5-cyano-4,5-dihydro-thiazol-4-yl |
| A-2624. | 2-nitro-4,5-dihydro-thiazol-4-yl |
| A-2625. | 5-nitro-4,5-dihydro-thiazol-4-yl |
| A-2626. | 2-methyl-4,5-dihydro-thiazol-4-yl |
| A-2627. | 5-methyl-4,5-dihydro-thiazol-4-yl |
| A-2628. | 2-trifluoromethyl-4,5-dihydro-thiazol-4-yl |
| A-2629. | 5-trifluoromethyl-4,5-dihydro-thiazol-4-yl |
| A-2630. | 2-methoxy-4,5-dihydro-thiazol-4-yl |
| A-2631. | 5-methoxy-4,5-dihydro-thiazol-4-yl |
| A-2632. | 2-trifluoromethoxy-4,5-dihydro-thiazol-4-yl |
| A-2633. | 5-trifluoromethoxy-4,5-dihydro-thiazol-4-yl |
| A-2634. | 2-chloro-4,5-dihydro-thiazol-5-yl |
| A-2635. | 4-chloro-4,5-dihydro-thiazol-5-yl |
| A-2636. | 2-bromo-4,5-dihydro-thiazol-5-yl |
| A-2637. | 4-bromo-4,5-dihydro-thiazol-5-yl |
| A-2638. | 2-cyano-4,5-dihydro-thiazol-5-yl |
| A-2639. | 4-cyano-4,5-dihydro-thiazol-5-yl |
| A-2640. | 2-nitro-4,5-dihydro-thiazol-5-yl |
| A-2641. | 4-nitro-4,5-dihydro-thiazol-5-yl |
| A-2642. | 2-methyl-4,5-dihydro-thiazol-5-yl |
| A-2643. | 4-methyl-4,5-dihydro-thiazol-5-yl |
| A-2644. | 2-trifluoromethyl-4,5-dihydro-thiazol-5-yl |
| A-2645. | 4-trifluoromethyl-4,5-dihydro-thiazol-5-yl |
| A-2646. | 2-methoxy-4,5-dihydro-thiazol-5-yl |
| A-2647. | 4-methoxy-4,5-dihydro-thiazol-5-yl |
| A-2648. | 2-trifluoromethoxy-4,5-dihydro-thiazol-5-yl |
| A-2649. | 4-trifluoromethoxy-4,5-dihydro-thiazol-5-yl |
| A-2650. | 2,3-dihydro-isoxazol-2-yl |
| A-2651. | 2,3-dihydro-isoxazol-3-yl |
| A-2652. | 2,3-dihydro-isoxazol-4-yl |
| A-2653. | 2,3-dihydro-isoxazol-5-yl |
| A-2654. | 3-chloro-2,3-dihydro-isoxazol-2-yl |
| A-2655. | 4-chloro-2,3-dihydro-isoxazol-2-yl |
| A-2656. | 5-chloro-2,3-dihydro-isoxazol-2-yl |
| A-2657. | 3-bromo-2,3-dihydro-isoxazol-2-yl |
| A-2658. | 4-bromo-2,3-dihydro-isoxazol-2-yl |
| A-2659. | 5-bromo-2,3-dihydro-isoxazol-2-yl |
| A-2660. | 3-cyano-2,3-dihydro-isoxazol-2-yl |
| A-2661. | 4-cyano-2,3-dihydro-isoxazol-2-yl |
| A-2662. | 5-cyano-2,3-dihydro-isoxazol-2-yl |
| A-2663. | 3-nitro-2,3-dihydro-isoxazol-2-yl |
| A-2664. | 4-nitro-2,3-dihydro-isoxazol-2-yl |
| A-2665. | 5-nitro-2,3-dihydro-isoxazol-2-yl |
| A-2666. | 3-methyl-2,3-dihydro-isoxazol-2-yl |
| A-2667. | 4-methyl-2,3-dihydro-isoxazol-2-yl |
| A-2668. | 5-methyl-2,3-dihydro-isoxazol-2-yl |
| A-2669. | 3-trifluoromethyl-2,3-dihydro-isoxazol-2-yl |
| A-2670. | 4-trifluoromethyl-2,3-dihydro-isoxazol-2-yl |
| A-2671. | 5-trifluoromethyl-2,3-dihydro-isoxazol-2-yl |
| A-2672. | 3-trifluoromethyl-2,3-dihydro-isoxazol-2-yl |
| A-2673. | 4-trifluoromethyl-2,3-dihydro-isoxazol-2-yl |
| A-2674. | 5-methoxy-2,3-dihydro-isoxazol-2-yl |
| A-2675. | 3-trifluoromethoxy-2,3-dihydro-isoxazol-2-yl |
| A-2676. | 4-trifluoromethoxy-2,3-dihydro-isoxazol-2-yl |
| A-2677. | 5-trifluoromethoxy-2,3-dihydro-isoxazol-2-yl |
| A-2678. | 4-chloro-2,3-dihydro-isoxazol-3-yl |
| A-2679. | 5-chloro-2,3-dihydro-isoxazol-3-yl |
| A-2680. | 4-bromo-2,3-dihydro-isoxazol-3-yl |
| A-2681. | 5-bromo-2,3-dihydro-isoxazol-3-yl |
| A-2682. | 2-cyano-2,3-dihydro-isoxazol-3-yl |
| A-2683. | 4-cyano-2,3-dihydro-isoxazol-3-yl |
| A-2684. | 5-cyano-2,3-dihydro-isoxazol-3-yl |
| A-2685. | 2-nitro-2,3-dihydro-isoxazol-3-yl |
| A-2686. | 4-nitro-2,3-dihydro-isoxazol-3-yl |
| A-2687. | 5-nitro-2,3-dihydro-isoxazol-3-yl |
| A-2688. | 2-methyl-2,3-dihydro-isoxazol-3-yl |
| A-2689. | 4-methyl-2,3-dihydro-isoxazol-3-yl |
| A-2690. | 5-methyl-2,3-dihydro-isoxazol-3-yl |
| A-2691. | 2-trifluoromethyl-2,3-dihydro-isoxazol-3-yl |
| A-2692. | 4-trifluoromethyl-2,3-dihydro-isoxazol-3-yl |
| A-2693. | 5-trifluoromethyl-2,3-dihydro-isoxazol-3-yl |
| A-2694. | 2-methoxy-2,3-dihydro-isoxazol-3-yl |
| A-2695. | 4-methoxy-2,3-dihydro-isoxazol-3-yl |
| A-2696. | 5-methoxy-2,3-dihydro-isoxazol-3-yl |
| A-2697. | 2-trifluoromethoxy-2,3-dihydro-isoxazol-3-yl |
| A-2698. | 4-trifluoromethoxy-2,3-dihydro-isoxazol-3-yl |
| A-2699. | 5-trifluoromethoxy-2,3-dihydro-isoxazol-3-yl |
| A-2700. | 3-chloro-2,3-dihydro-isoxazol-4-yl |
| A-2701. | 5-chloro-2,3-dihydro-isoxazol-4-yl |
| A-2702. | 3-bromo-2,3-dihydro-isoxazol-4-yl |
| A-2703. | 5-bromo-2,3-dihydro-isoxazol-4-yl |
| A-2704. | 2-cyano-2,3-dihydro-isoxazol-4-yl |
| A-2705. | 3-cyano-2,3-dihydro-isoxazol-4-yl |
| A-2706. | 5-cyano-2,3-dihydro-isoxazol-4-yl |
| A-2707. | 2-nitro-2,3-dihydro-isoxazol-4-yl |
| A-2708. | 3-nitro-2,3-dihydro-isoxazol-4-yl |
| A-2709. | 5-nitro-2,3-dihydro-isoxazol-4-yl |
| A-2710. | 2-methyl-2,3-dihydro-isoxazol-4-yl |
| A-2711. | 3-methyl-2,3-dihydro-isoxazol-4-yl |
| A-2712. | 5-methyl-2,3-dihydro-isoxazol-4-yl |
| A-2713. | 2-trifluoromethyl-2,3-dihydro-isoxazol-4-yl |
| A-2714. | 3-trifluoromethyl-2,3-dihydro-isoxazol-4-yl |
| A-2715. | 5-trifluoromethyl-2,3-dihydro-isoxazol-4-yl |
| A-2716. | 2-methoxy-2,3-dihydro-isoxazol-4-yl |
| A-2717. | 3-methoxy-2,3-dihydro-isoxazol-4-yl |
| A-2718. | 5-methoxy-2,3-dihydro-isoxazol-4-yl |
| A-2719. | 2-trifluoromethoxy-2,3-dihydro-isoxazol-4-yl |
| A-2720. | 3-trifluoromethoxy-2,3-dihydro-isoxazol-4-yl |
| A-2721. | 5-trifluoromethoxy-2,3-dihydro-isoxazol-4-yl |
| A-2722. | 3-chloro-2,3-dihydro-isoxazol-5-yl |
| A-2723. | 4-chloro-2,3-dihydro-isoxazol-5-yl |
| A-2724. | 3-bromo-2,3-dihydro-isoxazol-5-yl |
| A-2725. | 4-bromo-2,3-dihydro-isoxazol-5-yl |
| A-2726. | 2-cyano-2,3-dihydro-isoxazol-5-yl |
| A-2727. | 3-cyano-2,3-dihydro-isoxazol-5-yl |
| A-2728. | 4-cyano-2,3-dihydro-isoxazol-5-yl |
| A-2729. | 2-nitro-2,3-dihydro-isoxazol-5-yl |
| A-2730. | 3-nitro-2,3-dihydro-isoxazol-5-yl |
| A-2731. | 4-nitro-2,3-dihydro-isoxazol-5-yl |
| A-2732. | 2-methyl-2,3-dihydro-isoxazol-5-yl |
| A-2733. | 3-methyl-2,3-dihydro-isoxazol-5-yl |
| A-2734. | 4-methyl-2,3-dihydro-isoxazol-5-yl |
| A-2735. | 2-trifluoromethyl-2,3-dihydro-isoxazol-5-yl |
| A-2736. | 3-trifluoromethyl-2,3-dihydro-isoxazol-5-yl |
| A-2737. | 4-trifluoromethyl-2,3-dihydro-isoxazol-5-yl |
| A-2738. | 2-methoxy-2,3-dihydro-isoxazol-5-yl |
| A-2739. | 3-methoxy-2,3-dihydro-isoxazol-5-yl |
| A-2740. | 4-methoxy-2,3-dihydro-isoxazol-5-yl |
| A-2741. | 2-trifluoromethoxy-2,3-dihydro-isoxazol-5-yl |
| A-2742. | 3-trifluoromethoxy-2,3-dihydro-isoxazol-5-yl |
| A-2743. | 4-trifluoromethoxy-2,3-dihydro-isoxazol-5-yl |
| A-2744. | 2,5-dihydro-isoxazol-2-yl |
| A-2745. | 2,5-dihydro-isoxazol-3-yl |
| A-2746. | 2,5-dihydro-isoxazol-4-yl |
| A-2747. | 2,5-dihydro-isoxazol-5-yl |
| A-2748. | 3-chloro-2,5-dihydro-isoxazol-2-yl |
| A-2749. | 4-chloro-2,5-dihydro-isoxazol-2-yl |
| A-2750. | 5-chloro-2,5-dihydro-isoxazol-2-yl |
| A-2751. | 3-bromo-2,5-dihydro-isoxazol-2-yl |
| A-2752. | 4-bromo-2,5-dihydro-isoxazol-2-yl |
| A-2753. | 5-bromo-2,5-dihydro-isoxazol-2-yl |
| A-2754. | 3-cyano-2,5-dihydro-isoxazol-2-yl |
| A-2755. | 4-cyano-2,5-dihydro-isoxazol-2-yl |
| A-2756. | 5-cyano-2,5-dihydro-isoxazol-2-yl |
| A-2757. | 3-nitro-2,5-dihydro-isoxazol-2-yl |
| A-2758. | 4-nitro-2,5-dihydro-isoxazol-2-yl |
| A-2759. | 5-nitro-2,5-dihydro-isoxazol-2-yl |
| A-2760. | 3-methyl-2,5-dihydro-isoxazol-2-yl |
| A-2761. | 4-methyl-2,5-dihydro-isoxazol-2-yl |
| A-2762. | 5-methyl-2,5-dihydro-isoxazol-2-yl |
| A-2763. | 3-trifluoromethyl-2,5-dihydro-isoxazol-2-yl |
| A-2764. | 4-trifluoromethyl-2,5-dihydro-isoxazol-2-yl |
| A-2765. | 5-trifluoromethyl-2,5-dihydro-isoxazol-2-yl |
| A-2766. | 4-methoxy-2,5-dihydro-isoxazol-2-yl |
| A-2767. | 4-methoxy-2,5-dihydro-isoxazol-2-yl |
| A-2768. | 5-methoxy-2,5-dihydro-isoxazol-2-yl |
| A-2769. | 3-trifluoromethoxy-2,5-dihydro-isoxazol-2-yl |
| A-2770. | 4-trifluoromethoxy-2,5-dihydro-isoxazol-2-yl |
| A-2771. | 5-trifluoromethoxy-2,5-dihydro-isoxazol-2-yl |
| A-2772. | 3-chloro-2,5-dihydro-isoxazol-4-yl |
| A-2773. | 5-chloro-2,5-dihydro-isoxazol-4-yl |
| A-2774. | 2-bromo-2,5-dihydro-isoxazol-4-yl |
| A-2775. | 5-bromo-2,5-dihydro-isoxazol-4-yl |
| A-2776. | 2-cyano-2,5-dihydro-isoxazol-4-yl |
| A-2777. | 3-cyano-2,5-dihydro-isoxazol-4-yl |
| A-2778. | 5-cyano-2,5-dihydro-isoxazol-4-yl |
| A-2779. | 2-nitro-2,5-dihydro-isoxazol-4-yl |
| A-2780. | 3-nitro-2,5-dihydro-isoxazol-4-yl |
| A-2781. | 5-nitro-2,5-dihydro-isoxazol-4-yl |
| A-2782. | 2-methyl-2,5-dihydro-isoxazol-4-yl |
| A-2783. | 3-methyl-2,5-dihydro-isoxazol-4-yl |
| A-2784. | 5-methyl-2,5-dihydro-isoxazol-4-yl |
| A-2785. | 2-trifluoromethyl-2,5-dihydro-isoxazol-4-yl |
| A-2786. | 3-trifluoromethyl-2,5-dihydro-isoxazol-4-yl |
| A-2787. | 5-trifluoromethyl-2,5-dihydro-isoxazol-4-yl |
| A-2788. | 2-methoxy-2,5-dihydro-isoxazol-4-yl |
| A-2789. | 3-methoxy-2,5-dihydro-isoxazol-4-yl |
| A-2790. | 5-methoxy-2,5-dihydro-isoxazol-4-yl |
| A-2791. | 2-trifluoromethoxy-2,5-dihydro-isoxazol-4-yl |
| A-2792. | 3-trifluoromethoxy-2,5-dihydro-isoxazol-4-yl |
| A-2793. | 5-trifluoromethoxy-2,5-dihydro-isoxazol-4-yl |
| A-2794. | 3-chloro-2,5-dihydro-isoxazol-5-yl |
| A-2795. | 4-chloro-2,5-dihydro-isoxazol-5-yl |
| A-2796. | 3-bromo-2,5-dihydro-isoxazol-5-yl |
| A-2797. | 4-bromo-2,5-dihydro-isoxazol-5-yl |
| A-2798. | 2-cyano-2,5-dihydro-isoxazol-5-yl |
| A-2799. | 3-cyano-2,5-dihydro-isoxazol-5-yl |
| A-2800. | 4-cyano-2,5-dihydro-isoxazol-5-yl |
| A-2801. | 2-nitro-2,5-dihydro-isoxazol-5-yl |
| A-2802. | 3-nitro-2,5-dihydro-isoxazol-5-yl |
| A-2803. | 4-nitro-2,5-dihydro-isoxazol-5-yl |
| A-2804. | 2-methyl-2,5-dihydro-isoxazol-5-yl |
| A-2805. | 3-methyl-2,5-dihydro-isoxazol-5-yl |
| A-2806. | 4-methyl-2,5-dihydro-isoxazol-5-yl |
| A-2807. | 2-trifluoromethyl-2,5-dihydro-isoxazol-5-yl |
| A-2808. | 3-trifluoromethyl-2,5-dihydro-isoxazol-5-yl |
| A-2809. | 4-trifluoromethyl-2,5-dihydro-isoxazol-5-yl |
| A-2810. | 2-methoxy-2,5-dihydro-isoxazol-5-yl |
| A-2811. | 3-methoxy-2,5-dihydro-isoxazol-5-yl |
| A-2812. | 4-methoxy-2,5-dihydro-isoxazol-5-yl |
| A-2813. | 2-trifluoromethoxy-2,5-dihydro-isoxazol-5-yl |
| A-2814. | 3-trifluoromethoxy-2,5-dihydro-isoxazol-5-yl |
| A-2815. | 4-trifluoromethoxy-2,5-dihydro-isoxazol-5-yl |
| A-2816. | 4,5-dihydro-isoxazol-3-yl |
| A-2817. | 4,5-dihydro-isoxazol-4-yl |
| A-2818. | 4,5-dihydro-isoxazol-5-yl |
| A-2819. | 4-chloro-4,5-dihydro-isoxazol-3-yl |
| A-2820. | 5-chloro-4,5-dihydro-isoxazol-3-yl |
| A-2821. | 4-bromo-4,5-dihydro-isoxazol-3-yl |
| A-2822. | 5-bromo-4,5-dihydro-isoxazol-3-yl |
| A-2823. | 4-cyano-4,5-dihydro-isoxazol-3-yl |
| A-2824. | 5-cyano-4,5-dihydro-isoxazol-3-yl |
| A-2825. | 4-nitro-4,5-dihydro-isoxazol-3-yl |
| A-2826. | 5-nitro-4,5-dihydro-isoxazol-3-yl |
| A-2827. | 4-methyl-4,5-dihydro-isoxazol-3-yl |
| A-2828. | 5-methyl-4,5-dihydro-isoxazol-3-yl |
| A-2829. | 4-trifluoromethyl-4,5-dihydro-isoxazol-3-yl |
| A-2830. | 5-trifluoromethyl-4,5-dihydro-isoxazol-3-yl |
| A-2831. | 4-methoxy-4,5-dihydro-isoxazol-3-yl |
| A-2832. | 5-methoxy-4,5-dihydro-isoxazol-3-yl |
| A-2833. | 4-trifluoromethoxy-4,5-dihydro-isoxazol-3-yl |
| A-2834. | 5-trifluoromethoxy-4,5-dihydro-isoxazol-3-yl |
| A-2835. | 3-chloro-4,5-dihydro-isoxazol-4-yl |
| A-2836. | 5-chloro-4,5-dihydro-isoxazol-4-yl |
| A-2837. | 3-bromo-4,5-dihydro-isoxazol-4-yl |
| A-2838. | 5-bromo-4,5-dihydro-isoxazol-4-yl |
| A-2839. | 3-cyano-4,5-dihydro-isoxazol-4-yl |
| A-2840. | 5-cyano-4,5-dihydro-isoxazol-4-yl |
| A-2841. | 3-nitro-4,5-dihydro-isoxazol-4-yl |
| A-2842. | 5-nitro-4,5-dihydro-isoxazol-4-yl |
| A-2843. | 3-methyl-4,5-dihydro-isoxazol-4-yl |
| A-2844. | 5-methyl-4,5-dihydro-isoxazol-4-yl |
| A-2845. | 3-trifluoromethyl-4,5-dihydro-isoxazol-4-yl |
| A-2846. | 5-trifluoromethyl-4,5-dihydro-isoxazol-4-yl |
| A-2847. | 3-methoxy-4,5-dihydro-isoxazol-4-yl |
| A-2848. | 5-methoxy-4,5-dihydro-isoxazol-4-yl |
| A-2849. | 3-trifluoromethoxy-4,5-dihydro-isoxazol-4-yl |
| A-2850. | 5-trifluoromethoxy-4,5-dihydro-isoxazol-4-yl |
| A-2851. | 3-chloro-4,5-dihydro-isoxazol-5-yl |
| A-2852. | 4-chloro-4,5-dihydro-isoxazol-5-yl |
| A-2853. | 3-bromo-4,5-dihydro-isoxazol-5-yl |
| A-2854. | 4-bromo-4,5-dihydro-isoxazol-5-yl |
| A-2855. | 3-cyano-4,5-dihydro-isoxazol-5-yl |
| A-2856. | 4-cyano-4,5-dihydro-isoxazol-5-yl |
| A-2857. | 3-nitro-4,5-dihydro-isoxazol-5-yl |
| A-2858. | 4-nitro-4,5-dihydro-isoxazol-5-yl |
| A-2859. | 3-methyl-4,5-dihydro-isoxazol-5-yl |
| A-2860. | 4-methyl-4,5-dihydro-isoxazol-5-yl |
| A-2861. | 3-trifluoromethyl-4,5-dihydro-isoxazol-5-yl |
| A-2862. | 4-trifluoromethyl-4,5-dihydro-isoxazol-5-yl |
| A-2863. | 3-methoxy-4,5-dihydro-isoxazol-5-yl |
| A-2864. | 4-methoxy-4,5-dihydro-isoxazol-5-yl |
| A-2865. | 3-trifluoromethoxy-4,5-dihydro-isoxazol-5-yl |
| A-2866. | 4-trifluoromethoxy-4,5-dihydro-isoxazol-5-yl |
| A-2867. | 2,3-dihydro-isothiazol-2-yl |
| A-2868. | 2,3-dihydro-isothiazol-3-yl |
| A-2869. | 2,3-dihydro-isothiazol-4-yl |
| A-2870. | 2,3-dihydro-isothiazol-5-yl |
| A-2871. | 3-chloro-2,3-dihydro-isothiazol-2-yl |
| A-2872. | 4-chloro-2,3-dihydro-isothiazol-2-yl |
| A-2873. | 5-chloro-2,3-dihydro-isothiazol-2-yl |
| A-2874. | 3-bromo-2,3-dihydro-isothiazol-2-yl |
| A-2875. | 4-bromo-2,3-dihydro-isothiazol-2-yl |
| A-2876. | 5-bromo-2,3-dihydro-isothiazol-2-yl |
| A-2877. | 3-cyano-2,3-dihydro-isothiazol-2-yl |
| A-2878. | 4-cyano-2,3-dihydro-isothiazol-2-yl |
| A-2879. | 5-cyano-2,3-dihydro-isothiazol-2-yl |
| A-2880. | 3-nitro-2,3-dihydro-isothiazol-2-yl |
| A-2881. | 4-nitro-2,3-dihydro-isothiazol-2-yl |
| A-2882. | 5-nitro-2,3-dihydro-isothiazol-2-yl |
| A-2883. | 3-methyl-2,3-dihydro-isothiazol-2-yl |
| A-2884. | 4-methyl-2,3-dihydro-isothiazol-2-yl |
| A-2885. | 5-methyl-2,3-dihydro-isothiazol-2-yl |
| A-2886. | 3-trifluoromethyl-2,3-dihydro-isothiazol-2-yl |
| A-2887. | 4-trifluoromethyl-2,3-dihydro-isothiazol-2-yl |
| A-2888. | 5-trifluoromethyl-2,3-dihydro-isothiazol-2-yl |
| A-2889. | 3-trifluoromethyl-2,3-dihydro-isothiazol-2-yl |
| A-2890. | 4-trifluoromethyl-2,3-dihydro-isothiazol-2-yl |
| A-2891. | 5-methoxy-2,3-dihydro-isothiazol-2-yl |
| A-2892. | 3-trifluoromethoxy-2,3-dihydro-isothiazol-2-yl |
| A-2893. | 4-trifluoromethoxy-2,3-dihydro-isothiazol-2-yl |
| A-2894. | 5-trifluoromethoxy-2,3-dihydro-isothiazol-2-yl |
| A-2895. | 4-chloro-2,3-dihydro-isothiazol-3-yl |
| A-2896. | 5-chloro-2,3-dihydro-isothiazol-3-yl |
| A-2897. | 4-bromo-2,3-dihydro-isothiazol-3-yl |
| A-2898. | 5-bromo-2,3-dihydro-isothiazol-3-yl |
| A-2899. | 2-cyano-2,3-dihydro-isothiazol-3-yl |
| A-2900. | 4-cyano-2,3-dihydro-isothiazol-3-yl |
| A-2901. | 5-cyano-2,3-dihydro-isothiazol-3-yl |
| A-2902. | 2-nitro-2,3-dihydro-isothiazol-3-yl |
| A-2903. | 4-nitro-2,3-dihydro-isothiazol-3-yl |
| A-2904. | 5-nitro-2,3-dihydro-isothiazol-3-yl |
| A-2905. | 2-methyl-2,3-dihydro-isothiazol-3-yl |
| A-2906. | 4-methyl-2,3-dihydro-isothiazol-3-yl |
| A-2907. | 5-methyl-2,3-dihydro-isothiazol-3-yl |
| A-2908. | 2-trifluoromethyl-2,3-dihydro-isothiazol-3-yl |
| A-2909. | 4-trifluoromethyl-2,3-dihydro-isothiazol-3-yl |
| A-2910. | 5-trifluoromethyl-2,3-dihydro-isothiazol-3-yl |
| A-2911. | 2-methoxy-2,3-dihydro-isothiazol-3-yl |
| A-2912. | 4-methoxy-2,3-dihydro-isothiazol-3-yl |
| A-2913. | 5-methoxy-2,3-dihydro-isothiazol-3-yl |
| A-2914. | 2-trifluoromethoxy-2,3-dihydro-isothiazol-3-yl |
| A-2915. | 4-trifluoromethoxy-2,3-dihydro-isothiazol-3-yl |
| A-2916. | 5-trifluoromethoxy-2,3-dihydro-isothiazol-3-yl |
| A-2917. | 3-chloro-2,3-dihydro-isothiazol-4-yl |
| A-2918. | 5-chloro-2,3-dihydro-isothiazol-4-yl |
| A-2919. | 3-bromo-2,3-dihydro-isothiazol-4-yl |
| A-2920. | 5-bromo-2,3-dihydro-isothiazol-4-yl |
| A-2921. | 2-cyano-2,3-dihydro-isothiazol-4-yl |
| A-2922. | 3-cyano-2,3-dihydro-isothiazol-4-yl |
| A-2923. | 5-cyano-2,3-dihydro-isothiazol-4-yl |
| A-2924. | 2-nitro-2,3-dihydro-isothiazol-4-yl |
| A-2925. | 3-nitro-2,3-dihydro-isothiazol-4-yl |
| A-2926. | 5-nitro-2,3-dihydro-isothiazol-4-yl |
| A-2927. | 2-methyl-2,3-dihydro-isothiazol-4-yl |
| A-2928. | 3-methyl-2,3-dihydro-isothiazol-4-yl |
| A-2929. | 5-methyl-2,3-dihydro-isothiazol-4-yl |
| A-2930. | 2-trifluoromethyl-2,3-dihydro-isothiazol-4-yl |
| A-2931. | 3-trifluoromethyl-2,3-dihydro-isothiazol-4-yl |
| A-2932. | 5-trifluoromethyl-2,3-dihydro-isothiazol-4-yl |
| A-2933. | 2-methoxy-2,3-dihydro-isothiazol-4-yl |
| A-2934. | 3-methoxy-2,3-dihydro-isothiazol-4-yl |
| A-2935. | 5-methoxy-2,3-dihydro-isothiazol-4-yl |
| A-2936. | 2-trifluoromethoxy-2,3-dihydro-isothiazol-4-yl |
| A-2937. | 3-trifluoromethoxy-2,3-dihydro-isothiazol-4-yl |
| A-2938. | 5-trifluoromethoxy-2,3-dihydro-isothiazol-4-yl |
| A-2939. | 3-chloro-2,3-dihydro-isothiazol-5-yl |
| A-2940. | 4-chloro-2,3-dihydro-isothiazol-5-yl |
| A-2941. | 3-bromo-2,3-dihydro-isothiazol-5-yl |
| A-2942. | 4-bromo-2,3-dihydro-isothiazol-5-yl |
| A-2943. | 2-cyano-2,3-dihydro-isothiazol-5-yl |
| A-2944. | 3-cyano-2,3-dihydro-isothiazol-5-yl |
| A-2945. | 4-cyano-2,3-dihydro-isothiazol-5-yl |
| A-2946. | 2-nitro-2,3-dihydro-isothiazol-5-yl |
| A-2947. | 3-nitro-2,3-dihydro-isothiazol-5-yl |
| A-2948. | 4-nitro-2,3-dihydro-isothiazol-5-yl |
| A-2949. | 2-methyl-2,3-dihydro-isothiazol-5-yl |
| A-2950. | 3-methyl-2,3-dihydro-isothiazol-5-yl |
| A-2951. | 4-methyl-2,3-dihydro-isothiazol-5-yl |
| A-2952. | 2-trifluoromethyl-2,3-dihydro-isothiazol-5-yl |
| A-2953. | 3-trifluoromethyl-2,3-dihydro-isothiazol-5-yl |
| A-2954. | 4-trifluoromethyl-2,3-dihydro-isothiazol-5-yl |
| A-2955. | 2-methoxy-2,3-dihydro-isothiazol-5-yl |
| A-2956. | 3-methoxy-2,3-dihydro-isothiazol-5-yl |
| A-2957. | 4-methoxy-2,3-dihydro-isothiazol-5-yl |
| A-2958. | 2-trifluoromethoxy-2,3-dihydro-isothiazol-5-yl |
| A-2959. | 3-trifluoromethoxy-2,3-dihydro-isothiazol-5-yl |
| A-2960. | 4-trifluoromethoxy-2,3-dihydro-isothiazol-5-yl |
| A-2961. | 2,5-dihydro-isothiazol-2-yl |
| A-2962. | 2,5-dihydro-isothiazol-3-yl |
| A-2963. | 2,5-dihydro-isothiazol-4-yl |
| A-2964. | 2,5-dihydro-isothiazol-5-yl |
| A-2965. | 3-chloro-2,5-dihydro-isothiazol-2-yl |
| A-2966. | 4-chloro-2,5-dihydro-isothiazol-2-yl |
| A-2967. | 5-chloro-2,5-dihydro-isothiazol-2-yl |
| A-2968. | 3-bromo-2,5-dihydro-isothiazol-2-yl |
| A-2969. | 4-bromo-2,5-dihydro-isothiazol-2-yl |
| A-2970. | 5-bromo-2,5-dihydro-isothiazol-2-yl |
| A-2971. | 3-cyano-2,5-dihydro-isothiazol-2-yl |
| A-2972. | 4-cyano-2,5-dihydro-isothiazol-2-yl |
| A-2973. | 5-cyano-2,5-dihydro-isothiazol-2-yl |
| A-2974. | 3-nitro-2,5-dihydro-isothiazol-2-yl |
| A-2975. | 4-nitro-2,5-dihydro-isothiazol-2-yl |
| A-2976. | 5-nitro-2,5-dihydro-isothiazol-2-yl |
| A-2977. | 3-methyl-2,5-dihydro-isothiazol-2-yl |
| A-2978. | 4-methyl-2,5-dihydro-isothiazol-2-yl |
| A-2979. | 5-methyl-2,5-dihydro-isothiazol-2-yl |
| A-2980. | 3-trifluoromethyl-2,5-dihydro-isothiazol-2-yl |
| A-2981. | 4-trifluoromethyl-2,5-dihydro-isothiazol-2-yl |
| A-2982. | 5-trifluoromethyl-2,5-dihydro-isothiazol-2-yl |
| A-2983. | 4-methoxy-2,5-dihydro-isothiazol-2-yl |
| A-2984. | 4-methoxy-2,5-dihydro-isothiazol-2-yl |
| A-2985. | 5-methoxy-2,5-dihydro-isothiazol-2-yl |
| A-2986. | 3-trifluoromethoxy-2,5-dihydro-isothiazol-2-yl |
| A-2987. | 4-trifluoromethoxy-2,5-dihydro-isothiazol-2-yl |
| A-2988. | 5-trifluoromethoxy-2,5-dihydro-isothiazol-2-yl |
| A-2989. | 3-chloro-2,5-dihydro-isothiazol-4-yl |
| A-2990. | 5-chloro-2,5-dihydro-isothiazol-4-yl |
| A-2991. | 2-bromo-2,5-dihydro-isothiazol-4-yl |
| A-2992. | 5-bromo-2,5-dihydro-isothiazol-4-yl |
| A-2993. | 2-cyano-2,5-dihydro-isothiazol-4-yl |
| A-2994. | 3-cyano-2,5-dihydro-isothiazol-4-yl |
| A-2995. | 5-cyano-2,5-dihydro-isothiazol-4-yl |
| A-2996. | 2-nitro-2,5-dihydro-isothiazol-4-yl |
| A-2997. | 3-nitro-2,5-dihydro-isothiazol-4-yl |
| A-2998. | 5-nitro-2,5-dihydro-isothiazol-4-yl |
| A-2999. | 2-methyl-2,5-dihydro-isothiazol-4-yl |
| A-3000. | 3-methyl-2,5-dihydro-isothiazol-4-yl |
| A-3001. | 5-methyl-2,5-dihydro-isothiazol-4-yl |
| A-3002. | 2-trifluoromethyl-2,5-dihydro-isothiazol-4-yl |
| A-3003. | 3-trifluoromethyl-2,5-dihydro-isothiazol-4-yl |
| A-3004. | 5-trifluoromethyl-2,5-dihydro-isothiazol-4-yl |
| A-3005. | 2-methoxy-2,5-dihydro-isothiazol-4-yl |
| A-3006. | 3-methoxy-2,5-dihydro-isothiazol-4-yl |
| A-3007. | 5-methoxy-2,5-dihydro-isothiazol-4-yl |
| A-3008. | 2-trifluoromethoxy-2,5-dihydro-isothiazol-4-yl |
| A-3009. | 3-trifluoromethoxy-2,5-dihydro-isothiazol-4-yl |
| A-3010. | 5-trifluoromethoxy-2,5-dihydro-isothiazol-4-yl |
| A-3011. | 3-chloro-2,5-dihydro-isothiazol-5-yl |
| A-3012. | 4-chloro-2,5-dihydro-isothiazol-5-yl |
| A-3013. | 3-bromo-2,5-dihydro-isothiazol-5-yl |
| A-3014. | 4-bromo-2,5-dihydro-isothiazol-5-yl |
| A-3015. | 2-cyano-2,5-dihydro-isothiazol-5-yl |
| A-3016. | 3-cyano-2,5-dihydro-isothiazol-5-yl |
| A-3017. | 4-cyano-2,5-dihydro-isothiazol-5-yl |
| A-3018. | 2-nitro-2,5-dihydro-isothiazol-5-yl |
| A-3019. | 3-nitro-2,5-dihydro-isothiazol-5-yl |
| A-3020. | 4-nitro-2,5-dihydro-isothiazol-5-yl |
| A-3021. | 2-methyl-2,5-dihydro-isothiazol-5-yl |
| A-3022. | 3-methyl-2,5-dihydro-isothiazol-5-yl |
| A-3023. | 4-methyl-2,5-dihydro-isothiazol-5-yl |
| A-3024. | 2-trifluoromethyl-2,5-dihydro-isothiazol-5-yl |
| A-3025. | 3-trifluoromethyl-2,5-dihydro-isothiazol-5-yl |
| A-3026. | 4-trifluoromethyl-2,5-dihydro-isothiazol-5-yl |
| A-3027. | 2-methoxy-2,5-dihydro-isothiazol-5-yl |
| A-3028. | 3-methoxy-2,5-dihydro-isothiazol-5-yl |
| A-3029. | 4-methoxy-2,5-dihydro-isothiazol-5-yl |
| A-3030. | 2-trifluoromethoxy-2,5-dihydro-isothiazol-5-yl |
| A-3031. | 3-trifluoromethoxy-2,5-dihydro-isothiazol-5-yl |
| A-3032. | 4-trifluoromethoxy-2,5-dihydro-isothiazol-5-yl |
| A-3033. | 4,5-dihydro-isothiazol-3-yl |
| A-3034. | 4,5-dihydro-isothiazol-4-yl |
| A-3035. | 4,5-dihydro-isothiazol-5-yl |
| A-3036. | 4-chloro-4,5-dihydro-isothiazol-3-yl |
| A-3037. | 5-chloro-4,5-dihydro-isothiazol-3-yl |
| A-3038. | 4-bromo-4,5-dihydro-isothiazol-3-yl |
| A-3039. | 5-bromo-4,5-dihydro-isothiazol-3-yl |
| A-3040. | 4-cyano-4,5-dihydro-isothiazol-3-yl |
| A-3041. | 5-cyano-4,5-dihydro-isothiazol-3-yl |
| A-3042. | 4-nitro-4,5-dihydro-isothiazol-3-yl |
| A-3043. | 5-nitro-4,5-dihydro-isothiazol-3-yl |
| A-3044. | 4-methyl-4,5-dihydro-isothiazol-3-yl |
| A-3045. | 5-methyl-4,5-dihydro-isothiazol-3-yl |
| A-3046. | 4-trifluoromethyl-4,5-dihydro-isothiazol-3-yl |
| A-3047. | 5-trifluoromethyl-4,5-dihydro-isothiazol-3-yl |
| A-3048. | 4-methoxy-4,5-dihydro-isothiazol-3-yl |
| A-3049. | 5-methoxy-4,5-dihydro-isothiazol-3-yl |
| A-3050. | 4-trifluoromethoxy-4,5-dihydro-isothiazol-3-yl |
| A-3051. | 5-trifluoromethoxy-4,5-dihydro-isothiazol-3-yl |
| A-3052. | 3-chloro-4,5-dihydro-isothiazol-4-yl |
| A-3053. | 5-chloro-4,5-dihydro-isothiazol-4-yl |
| A-3054. | 3-bromo-4,5-dihydro-isothiazol-4-yl |
| A-3055. | 5-bromo-4,5-dihydro-isothiazol-4-yl |
| A-3056. | 3-cyano-4,5-dihydro-isothiazol-4-yl |
| A-3057. | 5-cyano-4,5-dihydro-isothiazol-4-yl |
| A-3058. | 3-nitro-4,5-dihydro-isothiazol-4-yl |
| A-3059. | 5-nitro-4,5-dihydro-isothiazol-4-yl |
| A-3060. | 3-methyl-4,5-dihydro-isothiazol-4-yl |
| A-3061. | 5-methyl-4,5-dihydro-isothiazol-4-yl |
| A-3062. | 3-trifluoromethyl-4,5-dihydro-isothiazol-4-yl |
| A-3063. | 5-trifluoromethyl-4,5-dihydro-isothiazol-4-yl |
| A-3064. | 3-methoxy-4,5-dihydro-isothiazol-4-yl |
| A-3065. | 5-methoxy-4,5-dihydro-isothiazol-4-yl |
| A-3066. | 3-trifluoromethoxy-4,5-dihydro-isothiazol-4-yl |
| A-3067. | 5-trifluoromethoxy-4,5-dihydro-isothiazol-4-yl |
| A-3068. | 3-chloro-4,5-dihydro-isothiazol-5-yl |
| A-3069. | 4-chloro-4,5-dihydro-isothiazol-5-yl |
| A-3070. | 3-bromo-4,5-dihydro-isothiazol-5-yl |
| A-3071. | 4-bromo-4,5-dihydro-isothiazol-5-yl |
| A-3072. | 3-cyano-4,5-dihydro-isothiazol-5-yl |
| A-3073. | 4-cyano-4,5-dihydro-isothiazol-5-yl |
| A-3074. | 3-nitro-4,5-dihydro-isothiazol-5-yl |
| A-3075. | 4-nitro-4,5-dihydro-isothiazol-5-yl |
| A-3076. | 3-methyl-4,5-dihydro-isothiazol-5-yl |
| A-3077. | 4-methyl-4,5-dihydro-isothiazol-5-yl |
| A-3078. | 3-trifluoromethyl-4,5-dihydro-isothiazol-5-yl |
| A-3079. | 4-trifluoromethyl-4,5-dihydro-isothiazol-5-yl |
| A-3080. | 3-methoxy-4,5-dihydro-isothiazol-5-yl |
| A-3081. | 4-methoxy-4,5-dihydro-isothiazol-5-yl |
| A-3082. | 3-trifluoromethoxy-4,5-dihydro-isothiazol-5-yl |
| A-3083. | 4-trifluoromethoxy-4,5-dihydro-isothiazol-5-yl |
| A-3084. | pyrrol-1-yl |
| A-3085. | pyrrol-2-yl |
| A-3086. | pyrrol-3-yl |
| A-3087. | 1-methyl-pyrrol-2-yl |
| A-3088. | 1-methyl-pyrrol-3-yl |
| A-3089. | 2-chloro-pyrrol-1-yl |
| A-3090. | 3-chloro-pyrrol-1-yl |
| A-3091. | 2-bromo-pyrrol-1-yl |
| A-3092. | 3-bromo-pyrrol-1-yl |
| A-3093. | 2-cyano-pyrrol-1-yl |
| A-3094. | 3-cyano-pyrrol-1-yl |
| A-3095. | 2-nitro-pyrrol-1-yl |
| A-3096. | 3-nitro-pyrrol-1-yl |
| A-3097. | 2-methyl-pyrrol-1-yl |
| A-3098. | 3-methyl-pyrrol-1-yl |
| A-3099. | 2-trifluoromethyl-pyrrol-1-yl |
| A-3100. | 3-trifluoromethyl-pyrrol-1-yl |
| A-3101. | 2-methoxy-pyrrol-1-yl |
| A-3102. | 3-methoxy-pyrrol-1-yl |
| A-3103. | 2-trifluoromethoxy-pyrrol-1-yl |
| A-3104. | 3-trifluoromethoxy-pyrrol-1-yl |
| A-3105. | 3-chloro-pyrrol-2-yl |
| A-3106. | 4-chloro-pyrrol-2-yl |
| A-3107. | 5-chloro-pyrrol-2-yl |
| A-3108. | 3-bromo-pyrrol-2-yl |
| A-3109. | 4-bromo-pyrrol-2-yl |
| A-3110. | 5-bromo-pyrrol-2-yl |
| A-3111. | 3-cyano-pyrrol-2-yl |
| A-3112. | 4-cyano-pyrrol-2-yl |
| A-3113. | 5-cyano-pyrrol-2-yl |
| A-3114. | 3-nitro-pyrrol-2-yl |
| A-3115. | 4-nitro-pyrrol-2-yl |
| A-3116. | 5-nitro-pyrrol-2-yl |
| A-3117. | 3-methyl-pyrrol-2-yl |
| A-3118. | 4-methyl-pyrrol-2-yl |
| A-3119. | 5-methyl-pyrrol-2-yl |
| A-3120. | 3-trifluoromethyl-pyrrol-2-yl |
| A-3121. | 4-trifluoromethyl-pyrrol-2-yl |
| A-3122. | 5-trifluoromethyl-pyrrol-2-yl |
| A-3123. | 3-methoxy-pyrrol-2-yl |
| A-3124. | 4-methoxy-pyrrol-2-yl |
| A-3125. | 5-methoxy-pyrrol-2-yl |
| A-3126. | 3-trifluoromethoxy-pyrrol-2-yl |
| A-3127. | 4-trifluoromethoxy-pyrrol-2-yl |
| A-3128. | 5-trifluoromethoxy-pyrrol-2-yl |
| A-3129. | 2-chloro-pyrrol-3-yl |
| A-3130. | 4-chloro-pyrrol-3-yl |
| A-3131. | 5-chloro-pyrrol-3-yl |
| A-3132. | 2-bromo-pyrrol-3-yl |
| A-3133. | 4-bromo-pyrrol-3-yl |
| A-3134. | 5-bromo-pyrrol-3-yl |
| A-3135. | 2-cyano-pyrrol-3-yl |
| A-3136. | 4-cyano-pyrrol-3-ly |
| A-3137. | 5-cyano-pyrrol-3-yl |
| A-3138. | 2-nitro-pyrrol-3-yl |
| A-3139. | 4-nitro-pyrrol-3-yl |
| A-3140. | 5-nitro-pyrrol-3-yl |
| A-3141. | 2-methyl-pyrrol-3-yl |
| A-3142. | 4-methyl-pyrrol-3-yl |
| A-3143. | 5-methyl-pyrrol-3-yl |
| A-3144. | 2-trifluoromethyl-pyrrol-3-yl |
| A-3145. | 4-trifluoromethyl-pyrrol-3-yl |
| A-3146. | 5-trifluoromethyl-pyrrol-3-yl |
| A-3147. | 2-methoxy-pyrrol-3-yl |
| A-3148. | 4-methoxy-pyrrol-3-yl |
| A-3149. | 5-methoxy-pyrrol-3-yl |
| A-3150. | 2-trifluoromethoxy-pyrrol-3-yl |
| A-3151. | 4-trifluoromethoxy-pyrrol-3-yl |
| A-3152. | 5-trifluoromethoxy-pyrrol-3-yl |
| A-3153. | 3-chloro-1-methyl-pyrrol-2-yl |
| A-3154. | 4-chloro-1-methyl-pyrrol-2-yl |
| A-3155. | 5-chloro-1-methyl-pyrrol-2-yl |
| A-3156. | 3-bromo-1-methyl-pyrrol-2-yl |
| A-3157. | 4-bromo-1-methyl-pyrrol-2-yl |
| A-3158. | 5-bromo-1-methyl-pyrrol-2-yl |
| A-3159. | 3-cyano-1-methyl-pyrrol-2-yl |
| A-3160. | 4-cyano-1-methyl-pyrrol-2-yl |
| A-3161. | 5-cyano-1-methyl-pyrrol-2-yl |
| A-3162. | 3-nitro-1-methyl-pyrrol-2-yl |
| A-3163. | 4-nitro-1-methyl-pyrrol-2-yl |
| A-3164. | 5-nitro-1-methyl-pyrrol-2-yl |
| A-3165. | 3-methyl-1-methyl-pyrrol-2-yl |
| A-3166. | 4-methyl-1-methyl-pyrrol-2-yl |
| A-3167. | 5-methyl-1-methyl-pyrrol-2-yl |
| A-3168. | 3-trifluoromethyl-1-methyl-pyrrol-2-yl |
| A-3169. | 4-trifluoromethyl-1-methyl-pyrrol-2-yl |
| A-3170. | 5-trifluoromethyl-1-methyl-pyrrol-2-yl |
| A-3171. | 3-methoxy-1-methyl-pyrrol-2-yl |
| A-3172. | 4-methoxy-1-methyl-pyrrol-2-yl |
| A-3173. | 5-methoxy-1-methyl-pyrrol-2-yl |
| A-3174. | 3-trifluoromethoxy-1-methyl-pyrrol-2-yl |
| A-3175. | 4-trifluoromethoxy-1-methyl-pyrrol-2-yl |
| A-3176. | 5-trifluoromethoxy-1-methyl-pyrrol-2-yl |
| A-3177. | 2-chloro-1-methyl-pyrrol-3-yl |
| A-3178. | 4-chloro-1-methyl-pyrrol-3-yl |
| A-3179. | 5-chloro-1-methyl-pyrrol-3-yl |
| A-3180. | 2-bromo-1-methyl-pyrrol-3-yl |
| A-3181. | 4-bromo-1-methyl-pyrrol-3-yl |
| A-3182. | 5-bromo-1-methyl-pyrrol-3-yl |
| A-3183. | 2-cyano-1-methyl-pyrrol-3-yl |
| A-3184. | 4-cyano-1-methyl-pyrrol-3-ly |
| A-3185. | 5-cyano-1-methyl-pyrrol-3-yl |
| A-3186. | 2-nitro-1-methyl-pyrrol-3-yl |
| A-3187. | 4-nitro-1-methyl-pyrrol-3-yl |
| A-3188. | 5-nitro-1-methyl-pyrrol-3-yl |
| A-3189. | 2-methyl-1-methyl-pyrrol-3-yl |
| A-3190. | 4-methyl-1-methyl-pyrrol-3-yl |
| A-3191. | 5-methyl-1-methyl-pyrrol-3-yl |
| A-3192. | 2-trifluoromethyl-1-methyl-pyrrol-3-yl |
| A-3193. | 4-trifluoromethyl-1-methyl-pyrrol-3-yl |
| A-3194. | 5-trifluoromethyl-1-methyl-pyrrol-3-yl |
| A-3195. | 2-methoxy-1-methyl-pyrrol-3-yl |
| A-3196. | 4-methoxy-1-methyl-pyrrol-3-yl |
| A-3197. | 5-methoxy-1-methyl-pyrrol-3-yl |
| A-3198. | 2-trifluoromethoxy-1-methyl-pyrrol-3-yl |
| A-3199. | 4-trifluoromethoxy-1-methyl-pyrrol-3-yl |
| A-3200. | 5-trifluoromethoxy-1-methyl-pyrrol-3-yl |
| A-3201. | furan-2-yl |
| A-3202. | furan-3-yl |
| A-3203. | 3-chloro-furan-2-yl |
| A-3204. | 4-chloro-furan-2-yl |
| A-3205. | 5-chloro-furan-2-yl |
| A-3206. | 3-bromo-furan-2-yl |
| A-3207. | 4-bromo-furan-2-yl |
| A-3208. | 5-bromo-furan-2-yl |
| A-3209. | 3-cyano-furan-2-yl |
| A-3210. | 4-cyano-furan-2-yl |
| A-3211. | 5-cyano-furan-2-yl |
| A-3212. | 3-nitro-furan-2-yl |
| A-3213. | 4-nitro-furan-2-yl |
| A-3214. | 5-nitro-furan-2-yl |
| A-3215. | 3-methyl-furan-2-yl |
| A-3216. | 4-methyl-furan-2-yl |
| A-3217. | 5-methyl-furan-2-yl |
| A-3218. | 3-trifluoromethyl-furan-2-yl |
| A-3219. | 4-trifluoromethyl-furan-2-yl |
| A-3220. | 5-trifluoromethyl-furan-2-yl |
| A-3221. | 3-methoxy-furan-2-yl |
| A-3222. | 4-methoxy-furan-2-yl |
| A-3223. | 5-methoxy-furan-2-yl |
| A-3224. | 3-trifluoromethoxy-furan-2-yl |
| A-3225. | 4-trifluoromethoxy-furan-2-yl |
| A-3226. | 5-trifluoromethoxy-furan-2-yl |
| A-3227. | 2-chloro-furan-3-yl |
| A-3228. | 4-chloro-furan-3-yl |
| A-3229. | 5-chloro-furan-3-yl |
| A-3230. | 2-bromo-furan-3-yl |
| A-3231. | 4-bromo-furan-3-yl |
| A-3232. | 5-bromo-furan-3-yl |
| A-3233. | 2-cyano-furan-3-yl |
| A-3234. | 4-cyano-furan-3-ly |
| A-3235. | 5-cyano-furan-3-yl |
| A-3236. | 2-nitro-furan-3-yl |
| A-3237. | 4-nitro-furan-3-yl |
| A-3238. | 5-nitro-furan-3-yl |
| A-3239. | 2-methyl-furan-3-yl |
| A-3240. | 4-methyl-furan-3-yl |
| A-3241. | 5-methyl-furan-3-yl |
| A-3242. | 2-trifluoromethyl-furan-3-yl |
| A-3243. | 4-trifluoromethyl-furan-3-yl |
| A-3244. | 5-trifluoromethyl-furan-3-yl |
| A-3245. | 2-methoxy-furan-3-yl |
| A-3246. | 4-methoxy-furan-3-yl |
| A-3247. | 5-methoxy-furan-3-yl |
| A-3248. | 2-trifluoromethoxy-furan-3-yl |
| A-3249. | 4-trifluoromethoxy-furan-3-yl |
| A-3250. | 5-trifluoromethoxy-furan-3-yl |
| A-3251. | thien-2-yl |
| A-3252. | thien-3-yl |
| A-3253. | 3-chloro-thien-2-yl |
| A-3254. | 4-chloro-thien-2-yl |
| A-3255. | 5-chloro-thien-2-yl |
| A-3256. | 3-bromo-thien-2-yl |
| A-3257. | 4-bromo-thien-2-yl |
| A-3258. | 5-bromo-thien-2-yl |
| A-3259. | 3-cyano-thien-2-yl |
| A-3260. | 4-cyano-thien-2-yl |
| A-3261. | 5-cyano-thien-2-yl |
| A-3262. | 3-nitro-thien-2-yl |
| A-3263. | 4-nitro-thien-2-yl |
| A-3264. | 5-nitro-thien-2-yl |
| A-3265. | 3-methyl-thien-2-yl |
| A-3266. | 4-methyl-thien-2-yl |
| A-3267. | 5-methyl-thien-2-yl |
| A-3268. | 3-trifluoromethyl-thien-2-yl |
| A-3269. | 4-trifluoromethyl-thien-2-yl |
| A-3270. | 5-trifluoromethyl-thien-2-yl |
| A-3271. | 3-methoxy-thien-2-yl |
| A-3272. | 4-methoxy-thien-2-yl |
| A-3273. | 5-methoxy-thien-2-yl |
| A-3274. | 3-trifluoromethoxy-thien-2-yl |
| A-3275. | 4-trifluoromethoxy-thien-2-yl |
| A-3276. | 5-trifluoromethoxy-thien-2-yl |
| A-3277. | 2-chloro-thien-3-yl |
| A-3278. | 4-chloro-thien-3-yl |
| A-3279. | 5-chloro-thien-3-yl |
| A-3280. | 2-bromo-thien-3-yl |
| A-3281. | 4-bromo-thien-3-yl |
| A-3282. | 5-bromo-thien-3-yl |
| A-3283. | 2-cyano-thien-3-yl |
| A-3284. | 4-cyano-thien-3-ly |
| A-3285. | 5-cyano-thien-3-yl |
| A-3286. | 2-nitro-thien-3-yl |
| A-3287. | 4-nitro-thien-3-yl |
| A-3288. | 5-nitro-thien-3-yl |
| A-3289. | 2-methyl-thien-3-yl |
| A-3290. | 4-methyl-thien-3-yl |
| A-3291. | 5-methyl-thien-3-yl |
| A-3292. | 2-trifluoromethyl-thien-3-yl |
| A-3293. | 4-trifluoromethyl-thien-3-yl |
| A-3294. | 5-trifluoromethyl-thien-3-yl |
| A-3295. | 2-methoxy-thien-3-yl |
| A-3296. | 4-methoxy-thien-3-yl |
| A-3297. | 5-methoxy-thien-3-yl |
| A-3298. | 2-trifluoromethoxy-thien-3-yl |
| A-3299. | 4-trifluoromethoxy-thien-3-yl |
| A-3300. | 5-trifluoromethoxy-thien-3-yl |
| A-3301. | pyrazol-1-yl |
| A-3302. | pyrazol-3-yl |
| A-3303. | pyrazol-4-yl |
| A-3304. | pyrazol-5-yl |
| A-3305. | 1-methyl-pyrazol-3-yl |
| A-3306. | 1-methyl-pyrazol-4-yl |
| A-3307. | 1-methyl-pyrazol-5-yl |
| A-3308. | 3-chloro-pyrazol-1-yl |
| A-3309. | 4-chloro-pyrazol-1-yl |
| A-3310. | 5-chloro-pyrazol-1-yl |
| A-3311. | 3-bromo-pyrazol-1-yl |
| A-3312. | 4-bromo-pyrazol-1-yl |
| A-3313. | 5-bromo-pyrazol-1-yl |
| A-3314. | 3-cyano-pyrazol-1-yl |
| A-3315. | 4-cyano-pyrazol-1-yl |
| A-3316. | 5-cyano-pyrazol-1-yl |
| A-3317. | 3-nitro-pyrazol-1-yl |
| A-3318. | 4-nitro-pyrazol-1-yl |
| A-3319. | 5-nitro-pyrazol-1-yl |
| A-3320. | 3-methyl-pyrazol-1-yl |
| A-3321. | 4-methyl-pyrazol-1-yl |
| A-3322. | 5-methyl-pyrazol-1-yl |
| A-3323. | 3-trifluoromethyl-pyrazol-1-yl |
| A-3324. | 4-trifluoromethyl-pyrazol-1-yl |
| A-3325. | 5-trifluoromethyl-pyrazol-1-yl |
| A-3326. | 3-methoxy-pyrazol-1-yl |
| A-3327. | 4-methoxy-pyrazol-1-yl |
| A-3328. | 5-methoxy-pyrazol-1-yl |
| A-3329. | 3-trifluoromethoxy-pyrazol-1-yl |
| A-3330. | 4-trifluoromethoxy-pyrazol-1-yl |
| A-3331. | 5-trifluoromethoxy-pyrazol-1-yl |
| A-3332. | 4-chloro-pyrazol-3-yl |
| A-3333. | 5-chloro-pyrazol-3-yl |
| A-3334. | 4-bromo-pyrazol-3-yl |
| A-3335. | 5-bromo-pyrazol-3-yl |
| A-3336. | 4-cyano-pyrazol-3-yl |
| A-3337. | 5-cyano-pyrazol-3-yl |
| A-3338. | 4-nitro-pyrazol-3-yl |
| A-3339. | 5-nitro-pyrazol-3-yl |
| A-3340. | 4-methyl-pyrazol-3-yl |
| A-3341. | 5-methyl-pyrazol-3-yl |
| A-3342. | 4-trifluoromethyl-pyrazol-3-yl |
| A-3343. | 5-trifluoromethyl-pyrazol-3-yl |
| A-3344. | 4-methoxy-pyrazol-3-yl |
| A-3345. | 5-methoxy-pyrazol-3-yl |
| A-3346. | 4-trifluoromethoxy-pyrazol-3-yl |
| A-3347. | 5-trifluoromethoxy-pyrazol-3-yl |
| A-3348. | 3-chloro-pyrazol-4-yl |
| A-3349. | 5-chloro-pyrazol-4-yl |
| A-3350. | 3-bromo-pyrazol-4-yl |
| A-3351. | 5-bromo-pyrazol-4-yl |
| A-3352. | 3-cyano-pyrazol-4-yl |
| A-3353. | 5-cyano-pyrazol-4-yl |
| A-3354. | 3-nitro-pyrazol-4-yl |
| A-3355. | 5-nitro-pyrazol-4-yl |
| A-3356. | 3-methyl-pyrazol-4-yl |
| A-3357. | 5-methyl-pyrazol-4-yl |
| A-3358. | 3-trifluoromethyl-pyrazol-4-yl |
| A-3359. | 5-trifluoromethyl-pyrazol-4-yl |
| A-3360. | 3-methoxy-pyrazol-4-yl |
| A-3361. | 5-methoxy-pyrazol-4-yl |
| A-3362. | 3-trifluoromethoxy-pyrazol-4-yl |
| A-3363. | 5-trifluoromethoxy-pyrazol-4-yl |
| A-3364. | 3-chloro-pyrazol-5-yl |
| A-3365. | 4-chloro-pyrazol-5-yl |
| A-3366. | 3-bromo-pyrazol-5-yl |
| A-3367. | 4-bromo-pyrazol-5-yl |
| A-3368. | 3-cyano-pyrazol-5-yl |
| A-3369. | 4-cyano-pyrazol-5-yl |
| A-3370. | 3-nitro-pyrazol-5-yl |
| A-3371. | 4-nitro-pyrazol-5-yl |
| A-3372. | 3-methyl-pyrazol-5-yl |
| A-3373. | 4-methyl-pyrazol-5-yl |
| A-3374. | 3-trifluoromethyl-pyrazol-5-yl |
| A-3375. | 4-trifluoromethyl-pyrazol-5-yl |
| A-3376. | 3-methoxy-pyrazol-5-yl |
| A-3377. | 4-methoxy-pyrazol-5-yl |
| A-3378. | 3-trifluoromethoxy-pyrazol-5-yl |
| A-3379. | 4-trifluoromethoxy-pyrazol-5-yl |
| A-3380. | 3-chloro-1-methyl-pyrazol-2-yl |
| A-3381. | 4-chloro-1-methyl-pyrazol-2-yl |
| A-3382. | 5-chloro-1-methyl-pyrazol-2-yl |
| A-3383. | 3-bromo-1-methyl-pyrazol-2-yl |
| A-3384. | 4-bromo-1-methyl-pyrazol-2-yl |
| A-3385. | 5-bromo-1-methyl-pyrazol-2-yl |
| A-3386. | 3-cyano-1-methyl-pyrazol-2-yl |
| A-3387. | 4-cyano-1-methyl-pyrazol-2-yl |
| A-3388. | 5-cyano-1-methyl-pyrazol-2-yl |
| A-3389. | 3-nitro-1-methyl-pyrazol-2-yl |
| A-3390. | 4-nitro-1-methyl-pyrazol-2-yl |
| A-3391. | 5-nitro-1-methyl-pyrazol-2-yl |
| A-3392. | 3-methyl-1-methyl-pyrazol-2-yl |
| A-3393. | 4-methyl-1-methyl-pyrazol-2-yl |
| A-3394. | 5-methyl-1-methyl-pyrazol-2-yl |
| A-3395. | 3-trifluoromethyl-1-methyl-pyrazol-2-yl |
| A-3396. | 4-trifluoromethyl-1-methyl-pyrazol-2-yl |
| A-3397. | 5-trifluoromethyl-1-methyl-pyrazol-2-yl |
| A-3398. | 3-methoxy-1-methyl-pyrazol-2-yl |
| A-3399. | 4-methoxy-1-methyl-pyrazol-2-yl |
| A-3400. | 5-methoxy-1-methyl-pyrazol-2-yl |
| A-3401. | 3-trifluoromethoxy-1-methyl-pyrazol-2-yl |
| A-3402. | 4-trifluoromethoxy-1-methyl-pyrazol-2-yl |
| A-3403. | 5-trifluoromethoxy-1-methyl-pyrazol-2-yl |
| A-3404. | 4-chloro-1-methyl-pyrazol-3-yl |
| A-3405. | 5-chloro-1-methyl-pyrazol-3-yl |
| A-3406. | 4-bromo-1-methyl-pyrazol-3-yl |
| A-3407. | 5-bromo-1-methyl-pyrazol-3-yl |
| A-3408. | 4-cyano-1-methyl-pyrazol-3-yl |
| A-3409. | 5-cyano-1-methyl-pyrazol-3-yl |
| A-3410. | 4-nitro-1-methyl-pyrazol-3-yl |
| A-3411. | 5-nitro-1-methyl-pyrazol-3-yl |
| A-3412. | 4-methyl-1-methyl-pyrazol-3-yl |
| A-3413. | 5-methyl-1-methyl-pyrazol-3-yl |
| A-3414. | 4-trifluoromethyl-1-methyl-pyrazol-3-yl |
| A-3415. | 5-trifluoromethyl-1-methyl-pyrazol-3-yl |
| A-3416. | 4-methoxy-1-methyl-pyrazol-3-yl |
| A-3417. | 5-methoxy-1-methyl-pyrazol-3-yl |
| A-3418. | 4-trifluoromethoxy-1-methyl-pyrazol-3-yl |
| A-3419. | 5-trifluoromethoxy-1-methyl-pyrazol-3-yl |
| A-3420. | 3-chloro-1-methyl-pyrazol-4-yl |
| A-3421. | 5-chloro-1-methyl-pyrazol-4-yl |
| A-3422. | 3-bromo-1-methyl-pyrazol-4-yl |
| A-3423. | 5-bromo-1-methyl-pyrazol-4-yl |
| A-3424. | 3-cyano-1-methyl-pyrazol-4-yl |
| A-3425. | 5-cyano-1-methyl-pyrazol-4-yl |
| A-3426. | 3-nitro-1-methyl-pyrazol-4-yl |
| A-3427. | 5-nitro-1-methyl-pyrazol-4-yl |
| A-3428. | 3-methyl-1-methyl-pyrazol-4-yl |
| A-3429. | 5-methyl-1-methyl-pyrazol-4-yl |
| A-3430. | 3-trifluoromethyl-1-methyl-pyrazol-4-yl |
| A-3431. | 5-trifluoromethyl-1-methyl-pyrazol-4-yl |
| A-3432. | 3-methoxy-1-methyl-pyrazol-4-yl |
| A-3433. | 5-methoxy-1-methyl-pyrazol-4-yl |
| A-3434. | 3-trifluoromethoxy-1-methyl-pyrazol-4-yl |
| A-3435. | 5-trifluoromethoxy-1-methyl-pyrazol-4-yl |
| A-3436. | 3-chloro-1-methyl-pyrazol-5-yl |
| A-3437. | 4-chloro-1-methyl-pyrazol-5-yl |
| A-3438. | 3-bromo-1-methyl-pyrazol-5-yl |
| A-3439. | 4-bromo-1-methyl-pyrazol-5-yl |
| A-3440. | 3-cyano-1-methyl-pyrazol-5-yl |
| A-3441. | 4-cyano-1-methyl-pyrazol-5-yl |
| A-3442. | 3-nitro-1-methyl-pyrazol-5-yl |
| A-3443. | 4-nitro-1-methyl-pyrazol-5-yl |
| A-3444. | 3-methyl-1-methyl-pyrazol-5-yl |
| A-3445. | 4-methyl-1-methyl-pyrazol-5-yl |
| A-3446. | 3-trifluoromethyl-1-methyl-pyrazol-5-yl |
| A-3447. | 4-trifluoromethyl-1-methyl-pyrazol-5-yl |
| A-3448. | 3-methoxy-1-methyl-pyrazol-5-yl |
| A-3449. | 4-methoxy-1-methyl-pyrazol-5-yl |
| A-3450. | 3-trifluoromethoxy-1-methyl-pyrazol-5-yl |
| A-3451. | 4-trifluoromethoxy-1-methyl-pyrazol-5-yl |
| A-3452. | imidazol-1-yl |
| A-3453. | imidazol-2-yl |
| A-3454. | imidazol-4-yl |
| A-3455. | imidazol-5-yl |
| A-3456. | 1-methyl-imidazol-2-yl |
| A-3457. | 1-methyl-imidazol-4-yl |
| A-3458. | 1-methyl-imidazol-5-yl |
| A-3459. | 2-chloro-imidazol-1-yl |
| A-3460. | 4-chloro-imidazol-1-yl |
| A-3461. | 5-chloro-imidazol-1-yl |
| A-3462. | 2-bromo-imidazol-1-yl |
| A-3463. | 4-bromo-imidazol-1-yl |
| A-3464. | 5-bromo-imidazol-1-yl |
| A-3465. | 2-cyano-imidazol-1-yl |
| A-3466. | 4-cyano-imidazol-1-yl |
| A-3467. | 5-cyano-imidazol-1-yl |
| A-3468. | 2-nitro-imidazol-1-yl |
| A-3469. | 4-nitro-imidazol-1-yl |
| A-3470. | 5-nitro-imidazol-1-yl |
| A-3471. | 2-methyl-imidazol-1-yl |
| A-3472. | 4-methyl-imidazol-1-yl |
| A-3473. | 5-methyl-imidazol-1-yl |
| A-3474. | 2-trifluoromethyl-imidazol-1-yl |
| A-3475. | 4-trifluoromethyl-imidazol-1-yl |
| A-3476. | 5-trifluoromethyl-imidazol-1-yl |
| A-3477. | 2-methoxy-imidazol-1-yl |
| A-3478. | 4-methoxy-imidazol-1-yl |
| A-3479. | 5-methoxy-imidazol-1-yl |
| A-3480. | 2-trifluoromethoxy-imidazol-1-yl |
| A-3481. | 4-trifluoromethoxy-imidazol-1-yl |
| A-3482. | 5-trifluoromethoxy-imidazol-1-yl |
| A-3483. | 4-chloro-imidazol-2-yl |
| A-3484. | 5-chloro-imidazol-2-yl |
| A-3485. | 4-bromo-imidazol-2-yl |
| A-3486. | 5-bromo-imidazol-2-yl |
| A-3487. | 4-cyano-imidazol-2-yl |
| A-3488. | 5-cyano-imidazol-2-yl |
| A-3489. [symbol] | 4-nitro-imidazol-2-yl |
| A-3490. | 5-nitro-imidazol-2-yl |
| A-3491. | 4-methyl-imidazol-2-yl |
| A-3492. | 5-methyl-imidazol-2-yl |
| A-3493. | 4-trifluoromethyl-imidazol-2-yl |
| A-3494. | 5-trifluoromethyl-imidazol-2-yl |
| A-3495. | 4-methoxy-imidazol-2-yl |
| A-3496. | 5-methoxy-imidazol-2-yl |
| A-3497. | 4-trifluoromethoxy-imidazol-2-yl |
| A-3498. | 5-trifluoromethoxy-imidazol-2-yl |
| A-3499. | 2-chloro-imidazol-4-yl |
| A-3500. | 5-chloro-imidazol-4-yl |
| A-3501. | 2-bromo-imidazol-4-yl |
| A-3502. | 5-bromo-imidazol-4-yl |
| A-3503. | 2-cyano-imidazol-4-yl |
| A-3504. | 5-cyano-imidazol-4-yl |
| A-3505. | 2-nitro-imidazol-4-yl |
| A-3506. | 5-nitro-imidazol-4-yl |
| A-3507. | 2-methyl-imidazol-4-yl |
| A-3508. | 5-methyl-imidazol-4-yl |
| A-3509. | 2-trifluoromethyl-imidazol-4-yl |
| A-3510. | 5-trifluoromethyl-imidazol-4-yl |
| A-3511. | 2-methoxy-imidazol-4-yl |
| A-3512. | 5-methoxy-imidazol-4-yl |
| A-3513. | 2-trifluoromethoxy-imidazol-4-yl |
| A-3514. | 5-trifluoromethoxy-imidazol-4-yl |
| A-3515. | 2-chloro-imidazol-5-yl |
| A-3516. | 4-chloro-imidazol-5-yl |
| A-3517. | 2-bromo-imidazol-5-yl |
| A-3518. | 4-bromo-imidazol-5-yl |
| A-3519. | 2-cyano-imidazol-5-yl |
| A-3520. | 4-cyano-imidazol-5-yl |
| A-3521. | 2-nitro-imidazol-5-yl |
| A-3522. | 4-nitro-imidazol-5-yl |
| A-3523. | 2-methyl-imidazol-5-yl |
| A-3524. | 4-methyl-imidazol-5-yl |
| A-3525. | 2-trifluoromethyl-imidazol-5-yl |
| A-3526. | 4-trifluoromethyl-imidazol-5-yl |
| A-3527. | 2-methoxy-imidazol-5-yl |
| A-3528. | 4-methoxy-imidazol-5-yl |
| A-3529. | 2-trifluoromethoxy-imidazol-5-yl |
| A-3530. | 4-trifluoromethoxy-imidazol-5-yl |
| A-3531. | 4-chloro-1-methyl-imidazol-2-yl |
| A-3532. | 5-chloro-1-methyl-imidazol-2-yl |
| A-3533. | 4-bromo-1-methyl-imidazol-2-yl |
| A-3534. | 5-bromo-1-methyl-imidazol-2-yl |
| A-3535. | 4-cyano-1-methyl-imidazol-2-yl |
| A-3536. | 5-cyano-1-methyl-imidazol-2-yl |
| A-3537. | 4-nitro-1-methyl-imidazol-2-yl |
| A-3538. | 5-nitro-1-methyl-imidazol-2-yl |
| A-3539. | 4-methyl-1-methyl-imidazol-2-yl |
| A-3540. | 5-methyl-1-methyl-imidazol-2-yl |
| A-3541. | 4-trifluoromethyl-1-methyl-imidazol-2-yl |
| A-3542. | 5-trifluoromethyl-1-methyl-imidazol-2-yl |
| A-3543. | 4-methoxy-1-methyl-imidazol-2-yl |
| A-3544. | 5-methoxy-1-methyl-imidazol-2-yl |
| A-3545. | 4-trifluoromethoxy-1-methyl-imidazol-2-yl |
| A-3546. | 5-trifluoromethoxy-1-methyl-imidazol-2-yl |
| A-3547. | 2-chloro-1-methyl-imidazol-4-yl |
| A-3548. | 5-chloro-1-methyl-imidazol-4-yl |
| A-3549. | 2-bromo-1-methyl-imidazol-4-yl |
| A-3550. | 5-bromo-1-methyl-imidazol-4-yl |
| A-3551. | 2-cyano-1-methyl-imidazol-4-yl |
| A-3552. | 5-cyano-1-methyl-imidazol-4-yl |
| A-3553. | 2-nitro-1-methyl-imidazol-4-yl |
| A-3554. | 5-nitro-1-methyl-imidazol-4-yl |
| A-3555. | 2-methyl-1-methyl-imidazol-4-yl |
| A-3556. | 5-methyl-1-methyl-imidazol-4-yl |
| A-3557. | 2-trifluoromethyl-1-methyl-imidazol-4-yl |
| A-3558. | 5-trifluoromethyl-1-methyl-imidazol-4-yl |
| A-3559. | 2-methoxy-1-methyl-imidazol-4-yl |
| A-3560. | 5-methoxy-1-methyl-imidazol-4-yl |
| A-3561. | 2-trifluoromethoxy-1-methyl-imidazol-4-yl |
| A-3562. | 5-trifluoromethoxy-1-methyl-imidazol-4-yl |
| A-3563. | 2-chloro-1-methyl-imidazol-5-yl |
| A-3564. | 4-chloro-1-methyl-imidazol-5-yl |
| A-3565. | 2-bromo-1-methyl-imidazol-5-yl |
| A-3566. | 4-bromo-1-methyl-imidazol-5-yl |
| A-3567. | 2-cyano-1-methyl-imidazol-5-yl |
| A-3568. | 4-cyano-1-methyl-imidazol-5-yl |
| A-3569. | 2-nitro-1-methyl-imidazol-5-yl |
| A-3570. | 4-nitro-1-methyl-imidazol-5-yl |
| A-3571. | 2-methyl-1-methyl-imidazol-5-yl |
| A-3572. | 4-methyl-1-methyl-imidazol-5-yl |
| A-3573. | 2-trifluoromethyl-1-methyl-imidazol-5-yl |
| A-3574. | 4-trifluoromethyl-1-methyl-imidazol-5-yl |
| A-3575. | 2-methoxy-1-methyl-imidazol-5-yl |
| A-3576. | 4-methoxy-1-methyl-imidazol-5-yl |
| A-3577. | 2-trifluoromethoxy-1-methyl-imidazol-5-yl |
| A-3578. | 4-trifluoromethoxy-1-methyl-imidazol-5-yl |
| A-3579. | oxazol-2-yl |
| A-3580. | oxazol-4-yl |
| A-3581. | oxazol-5-yl |
| A-3582. | 4-chloro-oxazol-2-yl |
| A-3583. | 5-chloro-oxazol-2-yl |
| A-3584. | 4-bromo-oxazol-2-yl |
| A-3585. | 5-bromo-oxazol-2-yl |
| A-3586. | 4-cyano-oxazol-2-yl |
| A-3587. | 5-cyano-oxazol-2-yl |
| A-3588. | 4-nitro-oxazol-2-yl |
| A-3589. | 5-nitro-oxazol-2-yl |
| A-3590. | 4-methyl-oxazol-2-yl |
| A-3591. | 5-methyl-oxazol-2-yl |
| A-3592. | 4-trifluoromethyl-oxazol-2-yl |
| A-3593. | 5-trifluoromethyl-oxazol-2-yl |
| A-3594. | 4-methoxy-oxazol-2-yl |
| A-3595. | 5-methoxy-oxazol-2-yl |
| A-3596. | 4-trifluoromethoxy-oxazol-2-yl |
| A-3597. | 5-trifluoromethoxy-oxazol-2-yl |
| A-3598. | 2-chloro-oxazol-4-yl |
| A-3599. | 5-chloro-oxazol-4-yl |
| A-3600. | 2-bromo-oxazol-4-yl |
| A-3601. | 5-bromo-oxazol-4-yl |
| A-3602. | 2-cyano-oxazol-4-yl |
| A-3603. | 5-cyano-oxazol-4-yl |
| A-3604. | 2-nitro-oxazol-4-yl |
| A-3605. | 5-nitro-oxazol-4-yl |
| A-3606. | 2-methyl-oxazol-4-yl |
| A-3607. | 5-methyl-oxazol-4-yl |
| A-3608. | 2-trifluoromethyl-oxazol-4-yl |
| A-3609. | 5-trifluoromethyl-oxazol-4-yl |
| A-3610. | 2-methoxy-oxazol-4-yl |
| A-3611. | 5-methoxy-oxazol-4-yl |
| A-3612. | 2-trifluoromethoxy-oxazol-4-yl |
| A-3613. | 5-trifluoromethoxy-oxazol-4-yl |
| A-3614. | 2-chloro-oxazol-5-yl |
| A-3615. | 4-chloro-oxazol-5-yl |
| A-3616. | 2-bromo-oxazol-5-yl |
| A-3617. | 4-bromo-oxazol-5-yl |
| A-3618. | 2-cyano-oxazol-5-yl |
| A-3619. | 4-cyano-oxazol-5-yl |
| A-3620. | 2-nitro-oxazol-5-yl |
| A-3621. | 4-nitro-oxazol-5-yl |
| A-3622. | 2-methyl-oxazol-5-yl |
| A-3623. | 4-methyl-oxazol-5-yl |
| A-3624. | 2-trifluoromethyl-oxazol-5-yl |
| A-3625. | 4-trifluoromethyl-oxazol-5-yl |
| A-3626. | 2-methoxy-oxazol-5-yl |
| A-3627. | 4-methoxy-oxazol-5-yl |
| A-3628. | 2-trifluoromethoxy-oxazol-5-yl |
| A-3629. | 4-trifluoromethoxy-oxazol-5-yl |
| A-3630. | thiazol-2-yl |
| A-3631. | thiazol-4-yl |
| A-3632. | thiazol-5-yl |
| A-3633. | 4-chloro-thiazol-2-yl |
| A-3634. | 5-chloro-thiazol-2-yl |
| A-3635. | 4-bromo-thiazol-2-yl |
| A-3636. | 5-bromo-thiazol-2-yl |
| A-3637. | 4-cyano-thiazol-2-yl |
| A-3638. | 5-cyano-thiazol-2-yl |
| A-3639. | 4-nitro-thiazol-2-yl |
| A-3640. | 5-nitro-thiazol-2-yl |
| A-3641. | 4-methyl-thiazol-2-yl |
| A-3642. | 5-methyl-thiazol-2-yl |
| A-3643. | 4-trifluoromethyl-thiazol-2-yl |
| A-3644. | 5-trifluoromethyl-thiazol-2-yl |
| A-3645. | 4-methoxy-thiazol-2-yl |
| A-3646. | 5-methoxy-thiazol-2-yl |
| A-3647. | 4-trifluoromethoxy-thiazol-2-yl |
| A-3648. | 5-trifluoromethoxy-thiazol-2-yl |
| A-3649. | 2-chloro-thiazol-4-yl |
| A-3650. | 5-chloro-thiazol-4-yl |
| A-3651. | 2-bromo-thiazol-4-yl |
| A-3652. | 5-bromo-thiazol-4-yl |
| A-3653. | 2-cyano-thiazol-4-yl |
| A-3654. | 5-cyano-thiazol-4-yl |
| A-3655. | 2-nitro-thiazol-4-yl |
| A-3656. | 5-nitro-thiazol-4-yl |
| A-3657. | 2-methyl-thiazol-4-yl |
| A-3658. | 5-methyl-thiazol-4-yl |
| A-3659. | 2-trifluoromethyl-thiazol-4-yl |
| A-3660. | 5-trifluoromethyl-thiazol-4-yl |
| A-3661. | 2-methoxy-thiazol-4-yl |
| A-3662. | 5-methoxy-thiazol-4-yl |
| A-3663. | 2-trifluoromethoxy-thiazol-4-yl |
| A-3664. | 5-trifluoromethoxy-thiazol-4-yl |
| A-3665. | 2-chloro-thiazol-5-yl |
| A-3666. | 4-chloro-thiazol-5-yl |
| A-3667. | 2-bromo-thiazol-5-yl |
| A-3668. | 4-bromo-thiazol-5-yl |
| A-3669. | 2-cyano-thiazol-5-yl |
| A-3670. | 4-cyano-thiazol-5-yl |
| A-3671. | 2-nitro-thiazol-5-yl |
| A-3672. | 4-nitro-thiazol-5-yl |
| A-3673. | 2-methyl-thiazol-5-yl |
| A-3674. | 4-methyl-thiazol-5-yl |
| A-3675. | 2-trifluoromethyl-thiazol-5-yl |
| A-3676. | 4-trifluoromethyl-thiazol-5-yl |
| A-3677. | 2-methoxy-thiazol-5-yl |
| A-3678. | 4-methoxy-thiazol-5-yl |
| A-3679. | 2-trifluoromethoxy-thiazol-5-yl |
| A-3680. | 4-trifluoromethoxy-thiazol-5-yl |
| A-3681. | isoxazol-3-yl |
| A-3682. | isoxazol-4-yl |
| A-3683. | isoxazol-5-yl |
| A-3684. | 4-chloro-isoxazol-3-yl |
| A-3685. | 5-chloro-isoxazol-3-yl |
| A-3686. | 4-bromo-isoxazol-3-yl |
| A-3687. | 5-bromo-isoxazol-3-yl |
| A-3688. | 4-cyano-isoxazol-3-yl |
| A-3689. | 5-cyano-isoxazol-3-yl |
| A-3690. [symbol] | 4-nitro-isoxazol-3-yl |
| A-3691. | 5-nitro-isoxazol-3-yl |
| A-3692. | 4-methyl-isoxazol-3-yl |
| A-3693. | 5-methyl-isoxazol-3-yl |
| A-3694. | 4-trifluoromethyl-isoxazol-3-yl |
| A-3695. | 5-trifluoromethyl-isoxazol-3-yl |
| A-3696. | 4-methoxy-isoxazol-3-yl |
| A-3697. | 5-methoxy-isoxazol-3-yl |
| A-3698. | 4-trifluoromethoxy-isoxazol-3-yl |
| A-3699. | 5-trifluoromethoxy-isoxazol-3-yl |
| A-3700. | 3-chloro-isoxazol-4-yl |
| A-3701. | 5-chloro-isoxazol-4-yl |
| A-3702. | 3-bromo-isoxazol-4-yl |
| A-3703. | 5-bromo-isoxazol-4-yl |
| A-3704. | 3-cyano-isoxazol-4-yl |
| A-3705. | 5-cyano-isoxazol-4-yl |
| A-3706. | 3-nitro-isoxazol-4-yl |
| A-3707. | 5-nitro-isoxazol-4-yl |
| A-3708. | 3-methyl-isoxazol-4-yl |
| A-3709. | 5-methyl-isoxazol-4-yl |
| A-3710. | 3-trifluoromethyl-isoxazol-4-yl |
| A-3711. | 5-trifluoromethyl-isoxazol-4-yl |
| A-3712. | 3-methoxy-isoxazol-4-yl |
| A-3713. | 5-methoxy-isoxazol-4-yl |
| A-3714. | 3-trifluoromethoxy-isoxazol-4-yl |
| A-3715. | 5-trifluoromethoxy-isoxazol-4-yl |
| A-3716. | 3-chloro-isoxazol-5-yl |
| A-3717. | 4-chloro-isoxazol-5-yl |
| A-3718. | 3-bromo-isoxazol-5-yl |
| A-3719. | 4-bromo-isoxazol-5-yl |
| A-3720. | 3-cyano-isoxazol-5-yl |
| A-3721. | 4-cyano-isoxazol-5-yl |
| A-3722. | 3-nitro-isoxazol-5-yl |
| A-3723. | 4-nitro-isoxazol-5-yl |
| A-3724. | 3-methyl-isoxazol-5-yl |
| A-3725. | 4-methyl-isoxazol-5-yl |
| A-3726. | 3-trifluoromethyl-isoxazol-5-yl |
| A-3727. | 4-trifluoromethyl-isoxazol-5-yl |
| A-3728. | 3-methoxy-isoxazol-5-yl |
| A-3729. | 4-methoxy-isoxazol-5-yl |
| A-3730. | 3-trifluoromethoxy-isoxazol-5-yl |
| A-3731. | 4-trifluoromethoxy-isoxazol-5-yl |
| A-3732. | isothiazol-3-yl |
| A-3733. | isothiazol-4-yl |
| A-3734. | isothiazol-5-yl |
| A-3735. | 4-chloro-isothiazol-3-yl |
| A-3736. | 5-chloro-isothiazol-3-yl |
| A-3737. | 4-bromo-isothiazol-3-yl |
| A-3738. | 5-bromo-isothiazol-3-yl |
| A-3739. | 4-cyano-isothiazol-3-yl |
| A-3740. | 5-cyano-isothiazol-3-yl |
| A-3741. $$ | 4-nitro-isothiazol-3-yl |
| A-3742. | 5-nitro-isothiazol-3-yl |
| A-3743. | 4-methyl-isothiazol-3-yl |
| A-3744. | 5-methyl-isothiazol-3-yl |
| A-3745. | 4-trifluoromethyl-isothiazol-3-yl |
| A-3746. | 5-trifluoromethyl-isothiazol-3-yl |
| A-3747. | 4-methoxy-isothiazol-3-yl |
| A-3748. | 5-methoxy-isothiazol-3-yl |
| A-3749. | 4-trifluoromethoxy-isothiazol-3-yl |
| A-3750. | 5-trifluoromethoxy-isothiazol-3-yl |
| A-3751. | 3-chloro-isothiazol-4-yl |
| A-3752. | 5-chloro-isothiazol-4-yl |
| A-3753. | 3-bromo-isothiazol-4-yl |
| A-3754. | 5-bromo-isothiazol-4-yl |
| A-3755. | 3-cyano-isothiazol-4-yl |
| A-3756. | 5-cyano-isothiazol-4-yl |
| A-3757. | 3-nitro-isothiazol-4-yl |
| A-3758. | 5-nitro-isothiazol-4-yl |
| A-3759. | 3-methyl-isothiazol-4-yl |
| A-3760. | 5-methyl-isothiazol-4-yl |
| A-3761. | 3-trifluoromethyl-isothiazol-4-yl |
| A-3762. | 5-trifluoromethyl-isothiazol-4-yl |
| A-3763. | 3-methoxy-isothiazol-4-yl |
| A-3764. | 5-methoxy-isothiazol-4-yl |
| A-3765. | 3-trifluoromethoxy-isothiazol-4-yl |
| A-3766. | 5-trifluoromethoxy-isothiazol-4-yl |
| A-3767. | 3-chloro-isothiazol-5-yl |
| A-3768. | 4-chloro-isothiazol-5-yl |
| A-3769. | 3-bromo-isothiazol-5-yl |
| A-3770. | 4-bromo-isothiazol-5-yl |
| A-3771. | 3-cyano-isothiazol-5-yl |
| A-3772. | 4-cyano-isothiazol-5-yl |
| A-3773. | 3-nitro-isothiazol-5-yl |
| A-3774. | 4-nitro-isothiazol-5-yl |
| A-3775. | 3-methyl-isothiazol-5-yl |
| A-3776. | 4-methyl-isothiazol-5-yl |
| A-3777. | 3-trifluoromethyl-isothiazol-5-yl |
| A-3778. | 4-trifluoromethyl-isothiazol-5-yl |
| A-3779. | 3-methoxy-isothiazol-5-yl |
| A-3780. | 4-methoxy-isothiazol-5-yl |
| A-3781. | 3-trifluoromethoxy-isothiazol-5-yl |
| A-3782. | 4-trifluoromethoxy-isothiazol-5-yl |
| A-3783. | piperidin-1-yl |
| A-3784. | piperidin-2-yl |
| A-3785. | piperidin-3-yl |
| A-3786. | piperidin-4-yl |
| A-3787. | 1-methyl-piperidin-2-yl |
| A-3788. | 1-methyl-piperidin-3-yl |
| A-3789. | 1-methyl-piperidin-4-yl |
| A-3790. | 2-chloro-piperidin-1-yl |
| A-3791. | 3-chloro-piperidin-1-yl |
| A-3792. | 4-chloro-piperidin-1-yl |
| A-3793. | 2-bromo-piperidin-1-yl |
| A-3794. | 3-bromo-piperidin-1-yl |
| A-3795. | 4-bromo-piperidin-1-yl |
| A-3796. | 2-cyano-piperidin-1-yl |
| A-3797. | 3-cyano-piperidin-1-yl |
| A-3798. | 4-cyano-piperidin-1-yl |
| A-3799. | 2-nitro-piperidin-1-yl |
| A-3800. | 3-nitro-piperidin-1-yl |
| A-3801. | 4-nitro-piperidin-1-yl |
| A-3802. | 2-methyl-piperidin-1-yl |
| A-3803. | 3-methyl-piperidin-1-yl |
| A-3804. | 4-methyl-piperidin-1-yl |
| A-3805. | 2-trifluoromethyl-piperidin-1-yl |
| A-3806. | 3-trifluoromethyl-piperidin-1-yl |
| A-3807. | 4-trifluoromethyl-piperidin-1-yl |
| A-3808. | 2-methoxy-piperidin-1-yl |
| A-3809. | 3-methoxy-piperidin-1-yl |
| A-3810. | 4-methoxy-piperidin-1-yl |
| A-3811. | 2-trifluoromethoxy-piperidin-1-yl |
| A-3812. | 3-trifluoromethoxy-piperidin-1-yl |
| A-3813. | 4-trifluoromethoxy-piperidin-1-yl |
| A-3814. | 3-chloro-piperidin-2-yl |
| A-3815. | 4-chloro-piperidin-2-yl |
| A-3816. | 3-bromo-piperidin-2-yl |
| A-3817. | 4-bromo-piperidin-2-yl |
| A-3818. | 3-cyano-piperidin-2-yl |
| A-3819. | 4-cyano-piperidin-2-yl |
| A-3820. | 3-nitro-piperidin-2-yl |
| A-3821. | 4-nitro-piperidin-2-yl |
| A-3822. | 3-methyl-piperidin-2-yl |
| A-3823. | 4-methyl-piperidin-2-yl |
| A-3824. | 3-trifluoromethyl-piperidin-2-yl |
| A-3825. | 4-trifluoromethyl-piperidin-2-yl |
| A-3826. | 3-methoxy-piperidin-2-yl |
| A-3827. | 4-methoxy-piperidin-2-yl |
| A-3828. | 3-trifluoromethoxy-piperidin-2-yl |
| A-3829. | 4-trifluoromethoxy-piperidin-2-yl |
| A-3830. | 2-chloro-piperidin-3-yl |
| A-3831. | 4-chloro-piperidin-3-yl |
| A-3832. | 2-bromo-piperidin-3-yl |
| A-3833. | 4-bromo-piperidin-3-yl |
| A-3834. | 2-cyano-piperidin-3-yl |
| A-3835. | 4-cyano-piperidin-3-yl |
| A-3836. | 2-nitro-piperidin-3-yl |
| A-3837. | 4-nitro-piperidin-3-yl |
| A-3838. | 2-methyl-piperidin-3-yl |
| A-3839. | 4-methyl-piperidin-3-yl |
| A-3840. | 2-trifluoromethyl-piperidin-3-yl |
| A-3841. | 4-trifluoromethyl-piperidin-3-yl |
| A-3842. | 2-methoxy-piperidin-3-yl |
| A-3843. | 4-methoxy-piperidin-3-yl |
| A-3844. | 2-trifluoromethoxy-piperidin-3-yl |
| A-3845. | 4-trifluoromethoxy-piperidin-3-yl |
| A-3846. | 2-chloro-piperidin-4-yl |
| A-3847. | 3-chloro-piperidin-4-yl |
| A-3848. | 2-bromo-piperidin-4-yl |
| A-3849. | 3-bromo-piperidin-4-yl |
| A-3850. | 2-cyano-piperidin-4-yl |
| A-3851. | 3-cyano-piperidin-4-yl |
| A-3852. | 2-nitro-piperidin-4-yl |
| A-3853. | 3-nitro-piperidin-4-yl |
| A-3854. | 2-methyl-piperidin-4-yl |
| A-3855. | 3-methyl-piperidin-4-yl |
| A-3856. | 2-trifluoromethyl-piperidin-4-yl |
| A-3857. | 3-trifluoromethyl-piperidin-4-yl |
| A-3858. | 2-methoxy-piperidin-4-yl |
| A-3859. | 3-methoxy-piperidin-4-yl |
| A-3860. | 2-trifluoromethoxy-piperidin-4-yl |
| A-3861. | 3-trifluoromethoxy-piperidin-4-yl |
| A-3862. | 3-chloro-1-methyl-piperidin-2-yl |
| A-3863. | 4-chloro-1-methyl-piperidin-2-yl |
| A-3864. | 5-chloro-1-methyl-piperidin-2-yl |
| A-3865. | 6-chloro-1-methyl-piperidin-2-yl |
| A-3866. | 3-bromo-1-methyl-piperidin-2-yl |
| A-3867. | 4-bromo-1-methyl-piperidin-2-yl |
| A-3868. | 5-bromo-1-methyl-piperidin-2-yl |
| A-3869. | 6-bromo-1-methyl-piperidin-2-yl |
| A-3870. | 3-cyano-1-methyl-piperidin-2-yl |
| A-3871. | 4-cyano-1-methyl-piperidin-2-yl |
| A-3872. | 5-cyano-1-methyl-piperidin-2-yl |
| A-3873. | 6-cyano-1-methyl-piperidin-2-yl |
| A-3874. | 3-nitro-1-methyl-piperidin-2-yl |
| A-3875. | 4-nitro-1-methyl-piperidin-2-yl |
| A-3876. | 5-nitro-1-methyl-piperidin-2-yl |
| A-3877. | 6-nitro-1-methyl-piperidin-2-yl |
| A-3878. | 3-methyl-1-methyl-piperidin-2-yl |
| A-3879. | 4-methyl-1-methyl-piperidin-2-yl |
| A-3880. | 5-methyl-1-methyl-piperidin-2-yl |
| A-3881. | 5-methyl-1-methyl-piperidin-2-yl |
| A-3882. | 3-trifluoromethyl-1-methyl-piperidin-2-yl |
| A-3883. | 4-trifluoromethyl-1-methyl-piperidin-2-yl |
| A-3884. | 5-trifluoromethyl-1-methyl-piperidin-2-yl |
| A-3885. | 6-trifluoromethyl-1-methyl-piperidin-2-yl |
| A-3886. | 3-methoxy-1-methyl-piperidin-2-yl |
| A-3887. | 4-methoxy-1-methyl-piperidin-2-yl |
| A-3888. | 5-methoxy-1-methyl-piperidin-2-yl |
| A-3889. | 6-methoxy-1-methyl-piperidin-2-yl |
| A-3890. | 3-trifluoromethoxy-1-methyl-piperidin-2-yl |
| A-3891. | 4-trifluoromethoxy-1-methyl-piperidin-2-yl |
| A-3892. | 5-trifluoromethoxy-1-methyl-piperidin-2-yl |
| A-3893. | 6-trifluoromethoxy-1-methyl-piperidin-2-yl |
| A-3894. | 2-chloro-1-methyl-piperidin-3-yl |
| A-3895. | 4-chloro-1-methyl-piperidin-3-yl |
| A-3896. | 5-chloro-1-methyl-piperidin-3-yl |
| A-3897. | 6-chloro-1-methyl-piperidin-3-yl |
| A-3898. | 2-bromo-1-methyl-piperidin-3-yl |
| A-3899. | 4-bromo-1-methyl-piperidin-3-yl |
| A-3900. | 5-bromo-1-methyl-piperidin-3-yl |
| A-3901. | 6-bromo-1-methyl-piperidin-3-yl |
| A-3902. | 2-cyano-1-methyl-piperidin-3-yl |
| A-3903. | 4-cyano-1-methyl-piperidin-3-yl |
| A-3904. | 5-cyano-1-methyl-piperidin-3-yl |
| A-3905. | 6-cyano-1-methyl-piperidin-3-yl |
| A-3906. | 2-nitro-1-methyl-piperidin-3-yl |
| A-3907. | 4-nitro-1-methyl-piperidin-3-yl |
| A-3908. | 5-nitro-1-methyl-piperidin-3-yl |
| A-3909. | 6-nitro-1-methyl-piperidin-3-yl |
| A-3910. | 2-methyl-1-methyl-piperidin-3-yl |
| A-3911. | 4-methyl-1-methyl-piperidin-3-yl |
| A-3912. | 5-methyl-1-methyl-piperidin-3-yl |
| A-3913. | 5-methyl-1-methyl-piperidin-3-yl |
| A-3914. | 2-trifluoromethyl-1-methyl-piperidin-3-yl |
| A-3915. | 4-trifluoromethyl-1-methyl-piperidin-3-yl |
| A-3916. | 5-trifluoromethyl-1-methyl-piperidin-3-yl |
| A-3917. | 6-trifluoromethyl-1-methyl-piperidin-3-yl |
| A-3918. | 2-methoxy-1-methyl-piperidin-3-yl |
| A-3919. | 4-methoxy-1-methyl-piperidin-3-yl |
| A-3920. | 5-methoxy-1-methyl-piperidin-3-yl |
| A-3921. | 6-methoxy-1-methyl-piperidin-3-yl |
| A-3922. | 2-trifluoromethoxy-1-methyl-piperidin-3-yl |
| A-3923. | 4-trifluoromethoxy-1-methyl-piperidin-3-yl |
| A-3924. | 5-trifluoromethoxy-1-methyl-piperidin-3-yl |
| A-3925. | 6-trifluoromethoxy-1-methyl-piperidin-3-yl |
| A-3926. | 2-chloro-1-methyl-piperidin-4-yl |
| A-3927. | 3-chloro-1-methyl-piperidin-4-yl |
| A-3928. | 5-chloro-1-methyl-piperidin-4-yl |
| A-3929. | 6-chloro-1-methyl-piperidin-4-yl |
| A-3930. | 2-bromo-1-methyl-piperidin-4-yl |
| A-3931. | 3-bromo-1-methyl-piperidin-4-yl |
| A-3932. | 5-bromo-1-methyl-piperidin-4-yl |
| A-3933. | 6-bromo-1-methyl-piperidin-4-yl |
| A-3934. | 2-cyano-1-methyl-piperidin-4-yl |
| A-3935. | 3-cyano-1-methyl-piperidin-4-yl |
| A-3936. | 5-cyano-1-methyl-piperidin-4-yl |
| A-3937. | 6-cyano-1-methyl-piperidin-4-yl |
| A-3938. | 2-nitro-1-methyl-piperidin-4-yl |
| A-3939. | 3-nitro-1-methyl-piperidin-4-yl |
| A-3940. | 5-nitro-1-methyl-piperidin-4-yl |
| A-3941. | 6-nitro-1-methyl-piperidin-4-yl |
| A-3942. | 2-methyl-1-methyl-piperidin-4-yl |
| A-3943. | 3-methyl-1-methyl-piperidin-4-yl |
| A-3944. | 5-methyl-1-methyl-piperidin-4-yl |
| A-3945. | 5-methyl-1-methyl-piperidin-4-yl |
| A-3946. | 2-trifluoromethyl-1-methyl-piperidin-4-yl |
| A-3947. | 3-trifluoromethyl-1-methyl-piperidin-4-yl |
| A-3948. | 5-trifluoromethyl-1-methyl-piperidin-4-yl |
| A-3949. | 6-trifluoromethyl-1-methyl-piperidin-4-yl |
| A-3950. | 2-methoxy-1-methyl-piperidin-4-yl |
| A-3951. | 3-methoxy-1-methyl-piperidin-4-yl |
| A-3952. | 5-methoxy-1-methyl-piperidin-4-yl |
| A-3953. | 6-methoxy-1-methyl-piperidin-4-yl |
| A-3954. | 2-trifluoromethoxy-1-methyl-piperidin-4-yl |
| A-3955. | 3-trifluoromethoxy-1-methyl-piperidin-4-yl |
| A-3956. | 5-trifluoromethoxy-1-methyl-piperidin-4-yl |
| A-3957. | 6-trifluoromethoxy-1-methyl-piperidin-4-yl |
| A-3958. | morpholin-2-yl |
| A-3959. | morpholin-3-yl |
| A-3960. | morpholin-4-yl |
| A-3961. | 4-methyl-morpholin-2-yl |
| A-3962. | 4-methyl-morpholin-3-yl |
| A-3963. | 3-chloro-morpholin-2-yl |
| A-3964. | 5-chloro-morpholin-2-yl |
| A-3965. | 6-chloro-morpholin-2-yl |
| A-3966. | 3-bromo-morpholin-2-yl |
| A-3967. | 5-bromo-morpholin-2-yl |
| A-3968. | 6-bromo-morpholin-2-yl |
| A-3969. | 3-cyano-morpholin-2-yl |
| A-3970. | 5-cyano-morpholin-2-yl |
| A-3971. | 6-cyano-morpholin-2-yl |
| A-3972. | 3-nitro-morpholin-2-yl |
| A-3973. | 5-nitro-morpholin-2-yl |
| A-3974. | 6-nitro-morpholin-2-yl |
| A-3975. | 3-methyl-morpholin-2-yl |
| A-3976. | 5-methyl-morpholin-2-yl |
| A-3977. | 6-methyl-morpholin-2-yl |
| A-3978. | 3-trifluoromethyl-morpholin-2-yl |
| A-3979. | 5-trifluoromethyl-morpholin-2-yl |
| A-3980. | 6-trifluoromethyl-morpholin-2-yl |
| A-3981. | 3-methoxy-morpholin-2-yl |
| A-3982. | 5-methoxy-morpholin-2-yl |
| A-3983. | 6-methoxy-morpholin-2-yl |
| A-3984. | 3-trifluoromethoxy-morpholin-2-yl |
| A-3985. | 5-trifluoromethoxy-morpholin-2-yl |
| A-3986. | 6-trifluoromethoxy-morpholin-2-yl |
| A-3987. | 2-chloro-morpholin-3-yl |
| A-3988. | 5-chloro-morpholin-3-yl |
| A-3989. | 6-chloro-morpholin-3-yl |
| A-3990. | 2-bromo-morpholin-3-yl |
| A-3991. | 5-bromo-morpholin-3-yl |
| A-3992. | 6-bromo-morpholin-3-yl |
| A-3993. | 2-cyano-morpholin-3-yl |
| A-3994. | 5-cyano-morpholin-3-yl |
| A-3995. | 6-cyano-morpholin-3-yl |
| A-3996. | 2-nitro-morpholin-3-yl |
| A-3997. | 5-nitro-morpholin-3-yl |
| A-3998. | 6-nitro-morpholin-3-yl |
| A-3999. | 2-methyl-morpholin-3-yl |
| A-4000. | 5-methyl-morpholin-3-yl |
| A-4001. | 6-methyl-morpholin-3-yl |
| A-4002. | 2-trifluoromethyl-morpholin-3-yl |
| A-4003. | 5-trifluoromethyl-morpholin-3-yl |
| A-4004. | 6-trifluoromethyl-morpholin-3-yl |
| A-4005. | 2-methoxy-morpholin-3-yl |
| A-4006. | 5-methoxy-morpholin-3-yl |
| A-4007. | 6-methoxy-morpholin-3-yl |
| A-4008. | 2-trifluoromethoxy-morpholin-3-yl |
| A-4009. | 5-trifluoromethoxy-morpholin-3-yl |
| A-4010. | 6-trifluoromethoxy-morpholin-3-yl |
| A-4011. | 2-chloro-morpholin-4-yl |
| A-4012. | 3-chloro-morpholin-4-yl |
| A-4013. | 2-bromo-morpholin-4-yl |
| A-4014. | 3-bromo-morpholin-4-yl |
| A-4015. | 2-cyano-morpholin-4-yl |
| A-4016. | 3-cyano-morpholin-4-yl |
| A-4017. | 2-nitro-morpholin-4-yl |
| A-4018. | 3-nitro-morpholin-4-yl |
| A-4019. | 2-methyl-morpholin-4-yl |
| A-4020. | 3-methyl-morpholin-4-yl |
| A-4021. | 2-trifluoromethyl-morpholin-4-yl |
| A-4022. | 3-trifluoromethyl-morpholin-4-yl |
| A-4023. | 2-methoxy-morpholin-4-yl |
| A-4024. | 3-methoxy-morpholin-4-yl |
| A-4025. | 2-trifluoromethoxy-morpholin-4-yl |
| A-4026. | 3-trifluoromethoxy-morpholin-4-yl |
| A-4027. | 3-chloro-4-methyl-morpholin-2-yl |
| A-4028. | 5-chloro-4-methyl-morpholin-2-yl |
| A-4029. | 6-chloro-4-methyl-morpholin-2-yl |
| A-4030. | 3-bromo-4-methyl-morpholin-2-yl |
| A-4031. | 5-bromo-4-methyl-morpholin-2-yl |
| A-4032. | 6-bromo-4-methyl-morpholin-2-yl |
| A-4033. | 3-cyano-4-methyl-morpholin-2-yl |
| A-4034. | 5-cyano-4-methyl-morpholin-2-yl |
| A-4035. | 6-cyano-4-methyl-morpholin-2-yl |
| A-4036. | 3-nitro-4-methyl-morpholin-2-yl |
| A-4037. | 5-nitro-4-methyl-morpholin-2-yl |
| A-4038. | 6-nitro-4-methyl-morpholin-2-yl |
| A-4039. | 3-methyl-4-methyl-morpholin-2-yl |
| A-4040. | 5-methyl-4-methyl-morpholin-2-yl |
| A-4041. | 6-methyl-4-methyl-morpholin-2-yl |
| A-4042. | 3-trifluoromethyl-4-methyl-morpholin-2-yl |
| A-4043. | 5-trifluoromethyl-4-methyl-morpholin-2-yl |
| A-4044. | 6-trifluoromethyl-4-methyl-morpholin-2-yl |
| A-4045. | 3-methoxy-4-methyl-morpholin-2-yl |
| A-4046. | 5-methoxy-4-methyl-morpholin-2-yl |
| A-4047. | 6-methoxy-4-methyl-morpholin-2-yl |
| A-4048. | 3-trifluoromethoxy-4-methyl-morpholin-2-yl |
| A-4049. | 5-trifluoromethoxy-4-methyl-morpholin-2-yl |
| A-4050. | 6-trifluoromethoxy-4-methyl-morpholin-2-yl |
| A-4051. | 2-chloro-4-methyl-morpholin-3-yl |
| A-4052. | 5-chloro-4-methyl-morpholin-3-yl |
| A-4053. | 6-chloro-4-methyl-morpholin-3-yl |
| A-4054. | 2-bromo-4-methyl-morpholin-3-yl |
| A-4055. | 5-bromo-4-methyl-morpholin-3-yl |
| A-4056. | 6-bromo-4-methyl-morpholin-3-yl |
| A-4057. | 2-cyano-4-methyl-morpholin-3-yl |
| A-4058. | 5-cyano-4-methyl-morpholin-3-yl |
| A-4059. | 6-cyano-4-methyl-morpholin-3-yl |
| A-4060. | 2-nitro-4-methyl-morpholin-3-yl |
| A-4061. | 5-nitro-4-methyl-morpholin-3-yl |
| A-4062. | 6-nitro-4-methyl-morpholin-3-yl |
| A-4063. | 2-methyl-4-methyl-morpholin-3-yl |
| A-4064. | 5-methyl-4-methyl-morpholin-3-yl |
| A-4065. | 6-methyl-4-methyl-morpholin-3-yl |
| A-4066. | 2-trifluoromethyl-4-methyl-morpholin-3-yl |
| A-4067. | 5-trifluoromethyl-4-methyl-morpholin-3-yl |
| A-4068. | 6-trifluoromethyl-4-methyl-morpholin-3-yl |
| A-4069. | 2-methoxy-4-methyl-morpholin-3-yl |
| A-4070. | 5-methoxy-4-methyl-morpholin-3-yl |
| A-4071. | 6-methoxy-4-methyl-morpholin-3-yl |
| A-4072. | 2-trifluoromethoxy-4-methyl-morpholin-3-yl |
| A-4073. | 5-trifluoromethoxy-4-methyl-morpholin-3-yl |
| A-4074. | 6-trifluoromethoxy-4-methyl-morpholin-3-yl |
| A-4075. | piperazin-1-yl |
| A-4076. | piperazin-2-yl |
| A-4077. | 4-methyl-piperazin-1-yl |
| A-4078. | 4-methyl-piperazin-2-yl |
| A-4079. | 4-methyl-piperazin-3-yl |
| A-4080. | 2-chloro-piperazin-1-yl |
| A-4081. | 3-chloro-piperazin-1-yl |
| A-4082. | 2-bromo-piperazin-1-yl |
| A-4083. | 3-bromo-piperazin-1-yl |
| A-4084. | 2-cyano-piperazin-1-yl |
| A-4085. | 3-cyano-piperazin-1-yl |
| A-4086. | 2-nitro-piperazin-1-yl |
| A-4087. | 3-nitro-piperazin-1-yl |
| A-4088. | 2-methyl-piperazin-1-yl |
| A-4089. | 3-methyl-piperazin-1-yl |
| A-4090. | 2-trifluoromethyl-piperazin-1-yl |
| A-4091. | 3-trifluoromethyl-piperazin-1-yl |
| A-4092. | 2-methoxy-piperazin-1-yl |
| A-4093. | 3-methoxy-piperazin-1-yl |
| A-4094. | 2-trifluoromethoxy-piperazin-1-yl |
| A-4095. | 3-trifluoromethoxy-piperazin-1-yl |
| A-4096. | 3-chloro-piperazin-2-yl |
| A-4097. | 5-chloro-piperazin-2-yl |
| A-4098. | 6-chloro-piperazin-2-yl |
| A-4099. | 3-bromo-piperazin-2-yl |
| A-4100. | 5-bromo-piperazin-2-yl |
| A-4101. | 6-bromo-piperazin-2-yl |
| A-4102. | 3-cyano-piperazin-2-yl |
| A-4103. | 5-cyano-piperazin-2-yl |
| A-4104. | 6-cyano-piperazin-2-yl |
| A-4105. | 3-nitro-piperazin-2-yl |
| A-4106. | 5-nitro-piperazin-2-yl |
| A-4107. | 6-nitro-piperazin-2-yl |
| A-4108. | 3-methyl-piperazin-2-yl |
| A-4109. | 5-methyl-piperazin-2-yl |
| A-4110. | 6-methyl-piperazin-2-yl |
| A-4111. | 3-trifluoromethyl-piperazin-2-yl |
| A-4112. | 5-trifluoromethyl-piperazin-2-yl |
| A-4113. | 6-trifluoromethyl-piperazin-2-yl |
| A-4114. | 3-methoxy-piperazin-2-yl |
| A-4115. | 5-methoxy-piperazin-2-yl |
| A-4116. | 6-methoxy-piperazin-2-yl |
| A-4117. | 3-trifluoromethoxy-piperazin-2-yl |
| A-4118. | 5-trifluoromethoxy-piperazin-2-yl |
| A-4119. | 6-trifluoromethoxy-piperazin-2-yl |
| A-4120. | 2-chloro-piperazin-3-yl |
| A-4121. | 5-chloro-piperazin-3-yl |
| A-4122. | 6-chloro-piperazin-3-yl |
| A-4123. | 2-bromo-piperazin-3-yl |
| A-4124. | 5-bromo-piperazin-3-yl |
| A-4125. | 6-bromo-piperazin-3-yl |
| A-4126. | 2-cyano-piperazin-3-yl |
| A-4127. | 5-cyano-piperazin-3-yl |
| A-4128. | 6-cyano-piperazin-3-yl |
| A-4129. | 2-nitro-piperazin-3-yl |
| A-4130. | 5-nitro-piperazin-3-yl |
| A-4131. | 6-nitro-piperazin-3-yl |
| A-4132. | 2-methyl-piperazin-3-yl |
| A-4133. | 5-methyl-piperazin-3-yl |
| A-4134. | 6-methyl-piperazin-3-yl |
| A-4135. | 2-trifluoromethyl-piperazin-3-yl |
| A-4136. | 5-trifluoromethyl-piperazin-3-yl |
| A-4137. | 6-trifluoromethyl-piperazin-3-yl |
| A-4138. | 2-methoxy-piperazin-3-yl |
| A-4139. | 5-methoxy-piperazin-3-yl |
| A-4140. | 6-methoxy-piperazin-3-yl |
| A-4141. | 2-trifluoromethoxy-piperazin-3-yl |
| A-4142. | 5-trifluoromethoxy-piperazin-3-yl |
| A-4143. | 6-trifluoromethoxy-piperazin-3-yl |
| A-4144. | 2-chloro-4-methyl-piperazin-1-yl |
| A-4145. | 3-chloro-4-methyl-piperazin-1-yl |
| A-4146. | 2-bromo-4-methyl-piperazin-1-yl |
| A-4147. | 3-bromo-4-methyl-piperazin-1-yl |
| A-4148. | 2-cyano-4-methyl-piperazin-1-yl |
| A-4149. | 3-cyano-4-methyl-piperazin-1-yl |
| A-4150. | 2-nitro-4-methyl-piperazin-1-yl |
| A-4151. | 3-nitro-4-methyl-piperazin-1-yl |
| A-4152. | 2-methyl-4-methyl-piperazin-1-yl |
| A-4153. | 3-methyl-4-methyl-piperazin-1-yl |
| A-4154. | 2-trifluoromethyl-4-methyl-piperazin-1-yl |
| A-4155. | 3-trifluoromethyl-4-methyl-piperazin-1-yl |
| A-4156. | 2-methoxy-4-methyl-piperazin-1-yl |
| A-4157. | 3-methoxy-4-methyl-piperazin-1-yl |
| A-4158. | 2-trifluoromethoxy-4-methyl-piperazin-1-yl |
| A-4159. | 3-trifluoromethoxy-4-methyl-piperazin-1-yl |
| A-4160. | 3-chloro-4-methyl-piperazin-2-yl |
| A-4161. | 5-chloro-4-methyl-piperazin-2-yl |
| A-4162. | 6-chloro-4-methyl-piperazin-2-yl |
| A-4163. | 3-bromo-4-methyl-piperazin-2-yl |
| A-4164. | 5-bromo-4-methyl-piperazin-2-yl |
| A-4165. | 6-bromo-4-methyl-piperazin-2-yl |
| A-4166. | 3-cyano-4-methyl-piperazin-2-yl |
| A-4167. | 5-cyano-4-methyl-piperazin-2-yl |
| A-4168. | 6-cyano-4-methyl-piperazin-2-yl |
| A-4169. | 3-nitro-4-methyl-piperazin-2-yl |
| A-4170. | 5-nitro-4-methyl-piperazin-2-yl |
| A-4171. | 6-nitro-4-methyl-piperazin-2-yl |
| A-4172. | 3-methyl-4-methyl-piperazin-2-yl |
| A-4173. | 5-methyl-4-methyl-piperazin-2-yl |
| A-4174. | 6-methyl-4-methyl-piperazin-2-yl |
| A-4175. | 3-trifluoromethyl-4-methyl-piperazin-2-yl |
| A-4176. | 5-trifluoromethyl-4-methyl-piperazin-2-yl |
| A-4177. | 6-trifluoromethyl-4-methyl-piperazin-2-yl |
| A-4178. | 3-methoxy-4-methyl-piperazin-2-yl |
| A-4179. | 5-methoxy-4-methyl-piperazin-2-yl |
| A-4180. | 6-methoxy-4-methyl-piperazin-2-yl |
| A-4181. | 3-trifluoromethoxy-4-methyl-piperazin-2-yl |
| A-4182. | 5-trifluoromethoxy-4-methyl-piperazin-2-yl |
| A-4183. | 6-trifluoromethoxy-4-methyl-piperazin-2-yl |
| A-4184. | 2-chloro-4-methyl-piperazin-3-yl |
| A-4185. | 5-chloro-4-methyl-piperazin-3-yl |
| A-4186. | 6-chloro-4-methyl-piperazin-3-yl |
| A-4187. | 2-bromo-4-methyl-piperazin-3-yl |
| A-4188. | 5-bromo-4-methyl-piperazin-3-yl |
| A-4189. | 6-bromo-4-methyl-piperazin-3-yl |
| A-4190. | 2-cyano-4-methyl-piperazin-3-yl |
| A-4191. | 5-cyano-4-methyl-piperazin-3-yl |
| A-4192. | 6-cyano4-methyl-piperazin-3-yl |
| A-4193. | 2-nitro-4-methyl-piperazin-3-yl |
| A-4194. | 5-nitro-4-methyl-piperazin-3-yl |
| A-4195. | 6-nitro-4-methyl-piperazin-3-yl |
| A-4196. | 2-methyl-4-methyl-piperazin-3-yl |
| A-4197. | 5-methyl-4-methyl-piperazin-3-yl |
| A-4198. | 6-methyl-4-methyl-piperazin-3-yl |
| A-4199. | 2-trifluoromethyl-4-methyl-piperazin-3-yl |
| A-4200. | 5-trifluoromethyl-4-methyl-piperazin-3-yl |
| A-4201. | 6-trifluoromethyl-4-methyl-piperazin-3-yl |
| A-4202. | 2-methoxy-4-methyl-piperazin-3-yl |
| A-4203. | 5-methoxy-4-methyl-piperazin-3-yl |
| A-4204. | 6-methoxy-4-methyl-piperazin-3-yl |
| A-4205. | 2-trifluoromethoxy-4-methyl-piperazin-3-yl |
| A-4206. | 5-trifluoromethoxy-4-methyl-piperazin-3-yl |
| A-4207. | 6-trifluoromethoxy-4-methyl-piperazin-3-yl |
| A-4208. | pyridin-2-yl |
| A-4209. | pyridin-3-yl |
| A-4210. | pyridin-4-yl |
| A-4211. | 3-chloro-pyridin-2-yl |
| A-4212. | 4-chloro-pyridin-2-yl |
| A-4213. | 5-chloro-pyridin-2-yl |
| A-4214. | 6-chloro-pyridin-2-yl |
| A-4215. | 3-bromo-pyridin-2-yl |
| A-4216. | 4-bromo-pyridin-2-yl |
| A-4217. | 5-bromo-pyridin-2-yl |
| A-4218. | 6-bromo-pyridin-2-yl |
| A-4219. | 3-cyano-pyridin-2-yl |
| A-4220. | 4-cyano-pyridin-2-yl |
| A-4221. | 5-cyano-pyridin-2-yl |
| A-4222. | 6-cyano-pyridin-2-yl |
| A-4223. | 3-nitro-pyridin-2-yl |
| A-4224. | 4-nitro-pyridin-2-yl |
| A-4225. | 5-nitro-pyridin-2-yl |
| A-4226. | 6-nitro-pyridin-2-yl |
| A-4227. | 3-methyl-pyridin-2-yl |
| A-4228. | 4-methyl-pyridin-2-yl |
| A-4229. | 5-methyl-pyridin-2-yl |
| A-4230. | 6-methyl-pyridin-2-yl |
| A-4231. | 3-trifluoromethyl-pyridin-2-yl |
| A-4232. | 4-trifluoromethyl-pyridin-2-yl |
| A-4233. | 5-trifluoromethyl-pyridin-2-yl |
| A-4234. | 6-trifluoromethyl-pyridin-2-yl |
| A-4235. | 3-methoxy-pyridin-2-yl |
| A-4236. | 4-methoxy-pyridin-2-yl |
| A-4237. | 5-methoxy-pyridin-2-yl |
| A-4238. | 6-methoxy-pyridin-2-yl |
| A-4239. | 3-trifluoromethoxy-pyridin-2-yl |
| A-4240. | 4-trifluoromethoxy-pyridin-2-yl |
| A-4241. | 5-trifluoromethoxy-pyridin-2-yl |
| A-4242. | 6-trifluoromethoxy-pyridin-2-yl |
| A-4243. | 2-chloro-pyridin-3-yl |
| A-4244. | 4-chloro-pyridin-3-yl |
| A-4245. | 5-chloro-pyridin-3-yl |
| A-4246. | 6-chloro-pyridin-3-yl |
| A-4247. | 2-bromo-pyridin-3-yl |
| A-4248. | 4-bromo-pyridin-3-yl |
| A-4249. | 5-bromo-pyridin-3-yl |
| A-4250. | 6-bromo-pyridin-3-yl |
| A-4251. | 2-cyano-pyridin-3-yl |
| A-4252. | 4-cyano-pyridin-3-yl |
| A-4253. | 5-cyano-pyridin-3-yl |
| A-4254. | 6-cyano-pyridin-3-yl |
| A-4255. | 2-nitro-pyridin-3-yl |
| A-4256. | 4-nitro-pyridin-3-yl |
| A-4257. | 5-nitro-pyridin-3-yl |
| A-4258. | 6-nitro-pyridin-3-yl |
| A-4259. | 2-methyl-pyridin-3-yl |
| A-4260. | 4-methyl-pyridin-3-yl |
| A-4261. | 5-methyl-pyridin-3-yl |
| A-4262. | 6-methyl-pyridin-3-yl |
| A-4263. | 2-trifluoromethyl-pyridin-3-yl |
| A-4264. | 4-trifluoromethyl-pyridin-3-yl |
| A-4265. | 5-trifluoromethyl-pyridin-3-yl |
| A-4266. | 6-trifluoromethyl-pyridin-3-yl |
| A-4267. | 2-methoxy-pyridin-3-yl |
| A-4268. | 4-methoxy-pyridin-3-yl |
| A-4269. | 5-methoxy-pyridin-3-yl |
| A-4270. | 6-methoxy-pyridin-3-yl |
| A-4271. | 2-trifluoromethoxy-pyridin-3-yl |
| A-4272. | 4-trifluoromethoxy-pyridin-3-yl |
| A-4273. | 5-trifluoromethoxy-pyridin-3-yl |
| A-4274. | 6-trifluoromethoxy-pyridin-3-yl |
| A-4275. | 2-chloro-pyridin-4-yl |
| A-4276. | 3-chloro-pyridin-4-yl |
| A-4277. | 2-bromo-pyridin-4-yl |
| A-4278. | 3-bromo-pyridin-4-yl |
| A-4279. | 2-cyano-pyridin-4-yl |
| A-4280. | 3-cyano-pyridin-4-yl |
| A-4281. | 2-nitro-pyridin-4-yl |
| A-4282. | 3-nitro-pyridin-4-yl |
| A-4283. | 2-methyl-pyridin-4-yl |
| A-4284. | 3-methyl-pyridin-4-yl |
| A-4285. | 2-trifluoromethyl-pyridin-4-yl |
| A-4286. | 3-trifluoromethyl-pyridin-4-yl |
| A-4287. | 2-methoxy-pyridin-4-yl |
| A-4288. | 3-methoxy-pyridin-4-yl |
| A-4289. | 2-trifluoromethoxy-pyridin-4-yl |
| A-4290. | 3-trifluoromethoxy-pyridin-4-yl |
| A-4291. | pyrimidin-2-yl |
| A-4292. | pyrimidin-4-yl |
| A-4293. | pyrimidin-5-yl |
| A-4294. | 4-chloro-pyrimidin-2-yl |
| A-4295. | 5-chloro-pyrimidin-2-yl |
| A-4296. | 4-bromo-pyrimidin-2-yl |
| A-4297. | 5-bromo-pyrimidin-2-yl |
| A-4298. | 4-cyano-pyrimidin-2-yl |
| A-4299. | 5-cyano-pyrimidin-2-yl |
| A-4300. | 4-nitro-pyrimidin-2-yl |
| A-4301. | 5-nitro-pyrimidin-2-yl |
| A-4302. | 4-methyl-pyrimidin-2-yl |
| A-4303. | 5-methyl-pyrimidin-2-yl |
| A-4304. | 4-trifluoromethyl-pyrimidin-2-yl |
| A-4305. | 5-trifluoromethyl-pyrimidin-2-yl |
| A-4306. | 4-methoxy-pyrimidin-2-yl |
| A-4307. | 5-methoxy-pyrimidin-2-yl |
| A-4308. | 4-trifluoromethoxy-pyrimidin-2-yl |
| A-4309. | 5-trifluoromethoxy-pyrimidin-2-yl |
| A-4310. | 2-chloro-pyrimidin-4-yl |
| A-4311. | 5-chloro-pyrimidin-4-yl |
| A-4312. | 6-chloro-pyrimidin-4-yl |
| A-4313. | 2-bromo-pyrimidin-4-yl |
| A-4314. | 5-bromo-pyrimidin-4-yl |
| A-4315. | 6-bromo-pyrimidin-4-yl |
| A-4316. | 2-cyano-pyrimidin-4-yl |
| A-4317. | 5-cyano-pyrimidin-4-yl |
| A-4318. | 6-cyano-pyrimidin-4-yl |
| A-4319. | 2-nitro-pyrimidin-4-yl |
| A-4320. | 5-nitro-pyrimidin-4-yl |
| A-4321. | 6-nitro-pyrimidin-4-yl |
| A-4322. | 2-methyl-pyrimidin-4-yl |
| A-4323. | 5-methyl-pyrimidin-4-yl |
| A-4324. | 6-methyl-pyrimidin-4-yl |
| A-4325. | 2-trifluoromethyl-pyrimidin-4-yl |
| A-4326. | 5-trifluoromethyl-pyrimidin-4-yl |
| A-4327. | 6-trifluoromethyl-pyrimidin-4-yl |
| A-4328. | 2-methoxy-pyrimidin-4-yl |
| A-4329. | 5-methoxy-pyrimidin-4-yl |
| A-4330. | 6-methoxy-pyrimidin-4-yl |
| A-4331. | 2-trifluoromethoxy-pyrimidin-4-yl |
| A-4332. | 5-trifluoromethoxy-pyrimidin-4-yl |
| A-4333. | 6-trifluoromethoxy-pyrimidin-4-yl |
| A-4334. | 2-chloro-pyrimidin-5-yl |
| A-4335. | 4-chloro-pyrimidin-5-yl |
| A-4336. | 2-bromo-pyrimidin-5-yl |
| A-4337. | 4-bromo-pyrimidin-5-yl |
| A-4338. | 2-cyano-pyrimidin-5-yl |
| A-4339. | 4-cyano-pyrimidin-5-yl |
| A-4340. | 2-nitro-pyrimidin-5-yl |
| A-4341. | 4-nitro-pyrimidin-5-yl |
| A-4342. | 2-methyl-pyrimidin-5-yl |
| A-4343. | 4-methyl-pyrimidin-5-yl |
| A-4344. | 2-trifluoromethyl-pyrimidin-5-yl |
| A-4345. | 4-trifluoromethyl-pyrimidin-5-yl |
| A-4346. | 2-methoxy-pyrimidin-5-yl |
| A-4347. | 4-methoxy-pyrimidin-5-yl |
| A-4348. | 2-trifluoromethoxy-pyrimidin-5-yl |
| A-4349. | 4-trifluoromethoxy-pyrimidin-5-yl |

[symbol]

indicates data missing or illegible when filed

TABLE B

| No. | R¹ |
|---|---|
| B-1. | NH₂ |
| B-2. | N(H)CH₃ |
| B-3. | N(CH₃)₂ |
| B-4. | N(H)CH₂CH₃ |
| B-5. | N(CH₂CH₃)₂ |
| B-6. | N(H)CH₂CH₂CH₃ |
| B-7. | N(CH₂CH₂CH₃)₂ |
| B-8. | N(H)CH(CH₃)₂ |
| B-9. | N(H)CH₂CH₂CH₂CH₃ |
| B-10. | N(CH₂CH₂CH₂CH₃)₂ |
| B-11. | N(H)CH₂CH(CH₃)CH₃ |
| B-12. | N(H)CH₂CH(CH₃)₂ |
| B-13. | N(H)C(CH₃)₃ |
| B-14. | N(H)(CH₂)₄CH₃ |
| B-15. | N[(CH₂)₄CH₃]₂ |
| B-16. | N(H)(CH₂)₅CH₃ |
| B-17. | N[(CH₂)₅CH₃]₂ |
| B-18. | N(H)CH₂CN |
| B-19. | N(H)CH₂CH₂CN |
| B-20. | N(H)CH₂CH₂CH₂CN |
| B-21. | N(H)CH₂CH₂OH |
| B-22. | N(H)CH₂CH₂OCH₃ |
| B-23. | N(CH₂CH₂OH)₂ |
| B-24. | N(CH₂CH₂OCH₃)₂ |
| B-25. | N(H)CF₃ |
| B-26. | N(CF₃)₂ |
| B-27. | N(H)CH₂CF₃ |
| B-28. | N(H)CF₂CF₃ |
| B-29. | N(CH₂CF₃)₂ |
| B-30. | N(CF₂CF₃)₂ |
| B-31. | N(H)CH₂CH₂CF₃ |
| B-32. | N(H)CH₂CF₂CH₃ |
| B-33. | N(H)CH₂CF₂CF₃ |
| B-34. | N(H)CF₂CH₂CH₃ |
| B-35. | N(H)CF₂CF₂CF₃ |
| B-36. | N(CH₂CH₂CF₃)₂ |
| B-37. | N(CH₂CF₂CH₃)₂ |
| B-38. | N(CH₂CF₂CF₃)₂ |
| B-39. | N(CF₂CH₂CH₃)₂ |
| B-40. | N(CF₂CF₂CF₃)₂ |
| B-41. | N(H)CH(CF₃)₂ |
| B-42. | N(H)CH₂-^{c}propyl |
| B-43. | N(H)-benzyl |
| B-44. | N(H)—CH₂SH |
| B-45. | N(H)—CH₂SCH₃ |
| B-46. | N(H)—CH₂SCH₂CH₃ |
| B-47. | N(H)—CH₂SCF₃ |
| B-48. | N(H)—CH₂SCH₂CF₃ |
| B-49. | N(H)—CH₂SCF₂CF₃ |
| B-50. | N(H)—CH₂CH₂SH |
| B-51. | N(H)—CH₂CH₂SCH₃ |
| B-52. | N(H)—CH₂CH₂SCH₂CH₃ |
| B-53. | N(H)—CH₂CH₂SCF₃ |
| B-54. | N(H)—CH₂CH₂SCH₂CF₃ |
| B-55. | N(H)—CH₂CH₂SCF₂CF₃ |
| B-56. | N(H)—CH₂CH₂CH₂SH |
| B-57. | N(H)—CH₂CH₂CH₂SCH₃ |
| B-58. | N(H)—CH₂CH₂CH₂SCH₂CH₃ |
| B-59. | N(H)—CH₂CH₂CH₂SCF₃ |
| B-60. | N(H)—CH₂CH₂CH₂SCH₂CF₃ |
| B-61. | N(H)—CH₂CH₂CH₂SCF₂CF₃ |
| B-62. | OH |
| B-63. | OCH₃ |
| B-64. | OCH₂CH₃ |
| B-65. | OCH₂CH₂CH₃ |
| B-66. | OCH(CH₃)₂ |
| B-67. | OCH₂CH₂CH₂CH₃ |
| B-68. | OCH₂CH(CH₃)CH₃ |
| B-69. | OCH₂CH(CH₃)₂ |
| B-70. | OC(CH₃)₃ |
| B-71. | O(CH₂)₄CH₃ |
| B-72. | O(CH₂)₅CH₃ |
| B-73. | OCH₂CN |
| B-74. | OCH₂CH₂CN |
| B-75. | OCH₂CH₂CH₂CN |
| B-76. | OCH₂CH₂OH |
| B-77. | OCH₂CH₂OCH₃ |
| B-78. | OCF₃ |
| B-79. | OCH₂CF₃ |
| B-80. | OCF₂CF₃ |
| B-81. | OCH₂CH₂CF₃ |
| B-82. | OCH₂CF₂CH₃ |
| B-83. | OCH₂CF₂CF₃ |
| B-84. | OCF₂CH₂CH₃ |
| B-85. | OCF₂CF₂CF₃ |
| B-86. | OCH₂-^{c}propyl |
| B-87. | O-benzyl |
| B-88. | OCH₂SH |
| B-89. | OCH₂SCH₃ |
| B-90. | OCH₂SCH₂CH₃ |
| B-91. | OCH₂SCF₃ |
| B-92. | OCH₂SCH₂CF₃ |
| B-93. | OCH₂SCF₂CF₃ |
| B-94. | OCH₂CH₂SH |
| B-95. | OCH₂CH₂SCH₃ |
| B-96. | OCH₂CH₂SCH₂CH₃ |
| B-97. | OCH₂CH₂SCF₃ |
| B-98. | OCH₂CH₂SCH₂CF₃ |
| B-99. | OCH₂CH₂SCF₂CF₃ |
| B-100. | OCH₂CH₂CH₂SH |
| B-101. | OCH₂CH₂CH₂SCH₃ |
| B-102. | OCH₂CH₂CH₂SCH₂CH₃ |
| B-103. | OCH₂CH₂CH₂SCF₃ |
| B-104. | OCH₂CH₂CH₂SCH₂CF₃ |
| B-105. | OCH₂CH₂CH₂SCF₂CF₃ |
| B-106. | OCH₂OH |
| B-107. | OCH₂OCH₃ |
| B-108. | OCH₂OCH₂CH₃ |
| B-109. | OCH₂OCF₃ |
| B-110. | OCH₂OCH₂CF₃ |
| B-111. | OCH₂OCF₂CF₃ |
| B-112. | OCH₂CH₂OH |
| B-113. | OCH₂CH₂OCH₃ |
| B-114. | OCH₂CH₂OCH₂CH₃ |
| B-115. | OCH₂CH₂OCF₃ |
| B-116. | OCH₂CH₂OCH₂CF₃ |
| B-117. | OCH₂CH₂OCF₂CF₃ |
| B-118. | OCH₂CH₂CH₂OH |
| B-119. | OCH₂CH₂CH₂OCH₃ |
| B-120. | OCH₂CH₂CH₂OCH₂CH₃ |
| B-121. | OCH₂CH₂CH₂OCF₃ |
| B-122. | OCH₂CH₂CH₂OCH₂CF₃ |
| B-123. | OCH₂CH₂CH₂OCF₂CF₃ |

TABLE C

| No. | R¹ |
|---|---|
| C-1. | N(H)—CO—CH₃ |
| C-2. | N(H)—CO—CH₂CH₃ |
| C-3. | N(H)—CO—CH₂CH₂CH₃ |
| C-4. | N(H)—CO—CH(CH₃)₂ |
| C-5. | N(H)—CO—CH₂CH₂CH₂CH₃ |
| C-6. | N(H)—CO—CH₂CH(CH₃)CH₃ |
| C-7. | N(H)—CO—CH₂CH(CH₃)₂ |
| C-8. | N(H)—CO—C(CH₃)3 |
| C-9. | N(H)—CO—(CH₂)₄CH₃ |
| C-10. | N(H)—CO—(CH₂)₅CH₃ |
| C-11. | N(H)—CO—CH₂CN |
| C-12. | N(H)—CO—CH₂CH₂CN |
| C-13. | N(H)—CO—CH₂CH₂CH₂CN |
| C-14. | N(H)—CO—CF₃ |
| C-15. | N(H)—CO—CH₂CF₃ |
| C-16. | N(H)—CO—CF₂CF₃ |
| C-17. | N(H)—CO—CH₂CH₂CF₃ |
| C-18. | N(H)—CO—CH₂CF₂CH₃ |
| C-19. | N(H)—CO—CH₂CF₂CF₃ |
| C-20. | N(H)—CO—CF₂CH₂CH₃ |
| C-21. | N(H)—CO—CF₂CF₂CF₃ |
| C-22. | N(H)—CO—CH(CF₃)₂ |
| C-23. | N(H)—CO—CH₂-^{c}propyl |
| C-24. | N(H)—CO-benzyl |
| C-25. | N(H)—CO—CH₂SH |
| C-26. | N(H)—CO—CH₂SCH₃ |
| C-27. | N(H)—CO—CH₂SCH₂CH₃ |
| C-28. | N(H)—CO—CH₂SCF₃ |
| C-29. | N(H)—CO—CH₂SCH₂CF₃ |
| C-30. | N(H)—CO—CH₂SCF₂CF₃ |
| C-31. | N(H)—CO—CH₂CH₂SH |
| C-32. | N(H)—CO—CH₂CH₂SCH₃ |
| C-33. | N(H)—CO—CH₂CH₂SCH₂CH₃ |
| C-34. | N(H)—CO—CH₂CH₂SCF₃ |
| C-35. | N(H)—CO—CH₂CH₂SCH₂CF₃ |
| C-36. | N(H)—CO—CH₂CH₂SCF₂CF₃ |
| C-37. | N(H)—CO—CH₂CH₂CH₂SH |
| C-38. | N(H)—CO—CH₂CH₂CH₂SCH₃ |
| C-39. | N(H)—CO—CH₂CH₂CH₂SCH₂CH₃ |
| C-40. | N(H)—CO—CH₂CH₂CH₂SCF₃ |
| C-41. | N(H)—CO—CH₂CH₂CH₂SCH₂CF₃ |
| C-42. | N(H)—CO—CH₂CH₂CH₂SCF₂CF₃ |
| C-43. | N(H)—CO—CH₂OH |
| C-44. | N(H)—CO—CH₂OCH₃ |
| C-45. | N(H)—CO—CH₂OCH₂CH₃ |
| C-46. | N(H)—CO—CH₂OCF₃ |
| C-47. | N(H)—CO—CH₂OCH₂CF₃ |
| C-48. | N(H)—CO—CH₂OCF₂CF₃ |
| C-49. | N(H)—CO—CH₂CH₂OH |
| C-50. | N(H)—CO—CH₂CH₂OCH₃ |
| C-51. | N(H)—CO—CH₂CH₂OCH₂CH₃ |
| C-52. | N(H)—CO—CH₂CH₂OCF₃ |
| C-53. | N(H)—CO—CH₂CH₂OCH₂CF₃ |
| C-54. | N(H)—CO—CH₂CH₂OCF₂CF₃ |
| C-55. | N(H)—CO—CH₂CH₂CH₂OH |
| C-56. | N(H)—CO—CH₂CH₂CH₂OCH₃ |
| C-57. | N(H)—CO—CH₂CH₂CH₂OCH₂CH₃ |
| C-58. | N(H)—CO—CH₂CH₂CH₂OCF₃ |
| C-59. | N(H)—CO—CH₂CH₂CH₂OCH₂CF₃ |
| C-60. | N(H)—CO—CH₂CH₂CH₂OCF₂CF₃ |
| C-61. | CO—NH₂ |
| C-62. | CO—N(H)CH₃ |
| C-63. | CO—N(CH₃)₂ |
| C-64. | CO—N(H)CH₂CH₃ |
| C-65. | CO—N(CH₂CH₃)₂ |
| C-66. | CO—N(H)CH₂CH₂CH₃ |
| C-67. | CO—N(CH₂CH₂CH₃)₂ |
| C-68. | CO—N(H)CH(CH₃)₂ |
| C-69. | CO—N(H)CH₂CH₂CH₂CH₃ |
| C-70. | CO—N(CH₂CH₂CH₂CH₃)₂ |
| C-71. | CO—N(H)CH₂CH(CH₃)CH₃ |
| C-72. | CO—N(H)CH₂CH(CH₃)₂ |
| C-73. | CO—N(H)C(CH₃)3 |
| C-74. | CO—N(H)(CH₂)₄CH₃ |
| C-75. | CO—N[(CH₂)₄CH₃]₂ |
| C-76. | CO—N(H)(CH₂)₅CH₃ |
| C-77. | CO—N[(CH₂)₅CH₃]₂ |
| C-78. | CO—N(H)CH₂CN |
| C-79. | CO—N(H)CH₂CH₂CN |
| C-80. | CO—N(H)CH₂CH₂CH₂CN |
| C-81. | CO—N(H)CF₃ |
| C-82. | CO—N(CF₃)₂ |
| C-83. | CO—N(H)CH₂CF₃ |
| C-84. | CO—N(H)CF₂CF₃ |
| C-85. | CO—N(CH₂CF₃)₂ |
| C-86. | CO—N(CF₂CF₃)₂ |
| C-87. | CO—N(H)CH₂CH₂CF₃ |
| C-88. | CO—N(H)CH₂CF₂CH₃ |
| C-89. | CO—N(H)CH₂CF₂CF₃ |
| C-90. | CO—N(H)CF₂CH₂CH₃ |
| C-91. | CO—N(H)CF₂CF₂CF₃ |
| C-92. | CO—N(CH₂CH₂CF₃)₂ |
| C-93. | CO—N(CH₂CF₂CH₃)₂ |
| C-94. | CO—N(CH₂CF₂CF₃)₂ |
| C-95. | CO—N(CF₂CH₂CH₃)₂ |
| C-96. | CO—N(CF₂CF₂CF₃)₂ |
| C-97. | CO—N(H)CH(CF₃)₂ |
| C-98. | CO—N(H)CH₂-^{c}propyl |
| C-99. | CO—N(H)-benzyl |
| C-100. | CO—N(H)—CH₂SH |
| C-101. | CO—N(H)—CH₂SCH₃ |
| C-102. | CO—N(H)—CH₂SCH₂CH₃ |
| C-103. | CO—N(H)—CH₂SCF₃ |
| C-104. | CO—N(H)—CH₂SCH₂CF₃ |
| C-105. | CO—N(H)—CH₂SCF₂CF₃ |
| C-106. | CO—N(H)—CH₂CH₂SH |
| C-107. | CO—N(H)—CH₂CH₂SCH₃ |
| C-108. | CO—N(H)—CH₂CH₂SCH₂CH₃ |
| C-109. | CO—N(H)—CH₂CH₂SCF₃ |
| C-110. | CO—N(H)—CH₂CH₂SCH₂CF₃ |
| C-111. | CO—N(H)—CH₂CH₂SCF₂CF₃ |
| C-112. | CO—N(H)—CH₂CH₂CH₂SH |
| C-113. | CO—N(H)—CH₂CH₂CH₂SCH₃ |
| C-114. | CO—N(H)—CH₂CH₂CH₂SCH₂CH₃ |
| C-115. | CO—N(H)—CH₂CH₂CH₂SCF₃ |
| C-116. | CO—N(H)—CH₂CH₂CH₂SCH₂CF₃ |
| C-117. | CO—N(H)—CH₂CH₂CH₂SCF₂CF₃ |
| C-118. | CO—N(H)—CH₂OH |
| C-119. | CO—N(H)—CH₂OCH₃ |
| C-120. | CO—N(H)—CH₂OCH₂CH₃ |
| C-121. | CO—N(H)—CH₂OCF₃ |
| C-122. | CO—N(H)—CH₂OCH₂CF₃ |
| C-123. | CO—N(H)—CH₂OCF₂CF₃ |
| C-124. | CO—N(H)—CH₂CH₂OH |
| C-125. | CO—N(H)—CH₂CH₂OCH₃ |
| C-126. | CO—N(CH₂CH₂OH)₂ |
| C-127. | CO—N(CH₂CH₂OCH₃)₂ |
| C-128. | CO—N(H)—CH₂CH₂OCH₂CH₃ |
| C-129. | CO—N(H)—CH₂CH₂OCF₃ |
| C-130. | CO—N(H)—CH₂CH₂OCH₂CF₃ |
| C-131. | CO—N(H)—CH₂CH₂OCF₂CF₃ |
| C-132. | CO—N(H)—CH₂CH₂CH₂OH |
| C-133. | CO—N(H)—CH₂CH₂CH₂OCH₃ |
| C-134. | CO—N(H)—CH₂CH₂CH₂OCH₂CH₃ |
| C-135. | CO—N(H)—CH₂CH₂CH₂OCF₃ |
| C-136. | CO—N(H)—CH₂CH₂CH₂OCH₂CF₃ |
| C-137. | CO—N(H)—CH₂CH₂CH₂OCF₂CF₃ |

TABLE D

| No. | B-R¹ |
|---|---|
| D-1. | pyrrolidin-1-yl |
| D-2. | pyrrolidin-2-on-1-yl |
| D-3. | 2-chloro-pyrrolidin-1-yl |
| D-4. | 3-chloro-pyrrolidin-1-yl |
| D-5. | 2-bromo-pyrrolidin-1-yl |
| D-6. | 3-bromo-pyrrolidin-1-yl |
| D-7. | 2-cyano-pyrrolidin-1-yl |
| D-8. | 3-cyano-pyrrolidin-1-yl |
| D-9. | 2-nitro-pyrrolidin-1-yl |
| D-10. | 3-nitro-pyrrolidin-1-yl |
| D-11. | 2-methyl-pyrrolidin-1-yl |
| D-12. | 3-methyl-pyrrolidin-1-yl |
| D-13. | 2-trifluoromethyl-pyrrolidin-1-yl |
| D-14. | 3-trifluoromethyl-pyrrolidin-1-yl |
| D-15. | 2-methoxy-pyrrolidin-1-yl |
| D-16. | 3-methoxy-pyrrolidin-1-yl |
| D-17. | 2-trifluoromethoxy-pyrrolidin-1-yl |
| D-18. | 3-trifluoromethoxy-pyrrolidin-1-yl |
| D-19. | 3-chloro-pyrrolidin-2-on-1-yl |
| D-20. | 4-chloro-pyrrolidin-2-on-1-yl |
| D-21. | 5-chloro-pyrrolidin-2-on-1-yl |
| D-22. | 3-bromo-pyrrolidin-2-on-1-yl |
| D-23. | 4-bromo-pyrrolidin-2-on-1-yl |
| D-24. | 5-bromo-pyrrolidin-2-on-1-yl |
| D-25. | 3-cyano-pyrrolidin-2-on-1-yl |
| D-26. | 4-cyano-pyrrolidin-2-on-1-yl |
| D-27. | 5-cyano-pyrrolidin-2-on-1-yl |
| D-28. | 3-nitro-pyrrolidin-2-on-1-yl |
| D-29. | 4-nitro-pyrrolidin-2-on-1-yl |
| D-30. | 5-nitro-pyrrolidin-2-on-1-yl |
| D-31. | 3-methyl-pyrrolidin-2-on-1-yl |
| D-32. | 4-methyl-pyrrolidin-2-on-1-yl |
| D-33. | 5-methyl-pyrrolidin-2-on-1-yl |
| D-34. | pyrazolidin-1-yl |
| D-35. | 3-chloro-pyrazolidin-1-yl |
| D-36. | 4-chloro-pyrazolidin-1-yl |
| D-37. | 5-chloro-pyrazolidin-1-yl |
| D-38. | 3-bromo-pyrazolidin-1-yl |
| D-39. | 4-bromo-pyrazolidin-1-yl |
| D-40. | 5-bromo-pyrazolidin-1-yl |
| D-41. | 3-cyano-pyrazolidin-1-yl |
| D-42. | 4-cyano-pyrazolidin-1-yl |
| D-43. | 5-cyano-pyrazolidin-1-yl |
| D-44. | 3-nitro-pyrazolidin-1-yl |
| D-45. | 4-nitro-pyrazolidin-1-yl |
| D-46. | 5-nitro-pyrazolidin-1-yl |
| D-47. | 3-methyl-pyrazolidin-1-yl |
| D-48. | 3-methyl-pyrazolidin-1-yl |
| D-49. | 4-methyl-pyrazolidin-1-yl |
| D-50. | 3-trifluoromethyl-pyrazolidin-1-yl |
| D-51. | 4-trifluoromethyl-pyrazolidin-1-yl |
| D-52. | 5-trifluoromethyl-pyrazolidin-1-yl |
| D-53. | 3-methoxy-pyrazolidin-1-yl |
| D-54. | 4-methoxy-pyrazolidin-1-yl |
| D-55. | 5-methoxy-pyrazolidin-1-yl |
| D-56. | 3-trifluoromethoxy-pyrazolidin-1-yl |
| D-57. | 4-trifluoromethoxy-pyrazolidin-1-yl |
| D-58. | 5-trifluoromethoxy-pyrazolidin-1-yl |
| D-59. | imidazolidin-1-yl |
| D-60. | imidazolidin-2-on-1-yl |
| D-61. | 2-chloro-imidazolidin-1-yl |
| D-62. | 4-chloro-imidazolidin-1-yl |
| D-63. | 5-chloro-imidazolidin-1-yl |
| D-64. | 2-bromo-imidazolidin-1-yl |
| D-65. | 4-bromo-imidazolidin-1-yl |
| D-66. | 5-bromo-imidazolidin-1-yl |
| D-67. | 2-cyano-imidazolidin-1-yl |
| D-68. | 4-cyano-imidazolidin-1-yl |
| D-69. | 5-cyano-imidazolidin-1-yl |
| D-70. | 2-nitro-imidazolidin-1-yl |
| D-71. | 4-nitro-imidazolidin-1-yl |
| D-72. | 5-nitro-imidazolidin-1-yl |
| D-73. | 2-methyl-imidazolidin-l-yl |
| D-74. | 4-methyl-imidazolidin-1-yl |
| D-75. | 5-methyl-imidazolidin-1-yl |
| D-76. | 2-trifluoromethyl-imidazolidin-1-yl |
| D-77. | 4-trifluoromethyl-imidazolidin-l-yl |
| D-78. | 5-trifluoromethyl-imidazolidin-1-yl |
| D-79. | 2-methoxy-imidazolidin-1-yl |
| D-80. | 4-methoxy-imidazolidin-1-yl |
| D-81. | 5-methoxy-imidazolidin-1-yl |
| D-82. | 2-trifluoromethoxy-imidazolidin-1-yl |
| D-83. | 4-trifluoromethoxy-imidazolidin-1-yl |
| D-84. | 5-trifluoromethoxy-imidazolidin-1-yl |
| D-85. | 4-chloro-imidazolidin-2-on-1-yl |
| D-86. | 5-chloro-imidazolidin-2-on-1-yl |
| D-87. | 4-bromo-imidazolidin-2-on-1-yl |
| D-88. | 5-bromo-imidazolidin-2-on-1-yl |
| D-89. | 4-cyano-imidazolidin-2-on-1-yl |
| D-90. | 5-cyano-imidazolidin-2-on-1-yl |
| D-91. | 4-nitro-imidazolidin-2-on-1-yl |
| D-92. | 5-nitro-imidazolidin-2-on-1-yl |
| D-93. | 4-methyl-imidazolidin-2-on-1-yl |
| D-94. | 5-methyl-imidazolidin-2-on-1-yl |
| D-95. | 4-trifluoromethyl-imidazolidin-2-on-1-yl |
| D-96. | 5-trifluoromethyl-imidazolidin-2-on-1-yl |
| D-97. | 4-methoxy-imidazolidin-2-on-1-yl |
| D-98. | 5-methoxy-imidazolidin-2-on-1-yl |
| D-99. | 4-trifluoromethoxy-imidazolidin-2-on-1-yl |
| D-100. | 5-trifluoromethoxy-imidazolidin-2-on-1-yl |
| D-101. | 2,3-dihydro-pyrrol-1-yl |
| D-102. | 2-chloro-2,3-dihydro-pyrrol-1-yl |
| D-103. | 3-chloro-2,3-dihydro-pyrrol-1-yl |
| D-104. | 2-bromo-2,3-dihydro-pyrrol-1-yl |
| D-105. | 3-bromo-2,3-dihydro-pyrrol-1-yl |
| D-106. | 2-cyano-2,3-dihydro-pyrrol-1-yl |
| D-107. | 3-cyano-2,3-dihydro-pyrrol-1-yl |
| D-108. | 2-nitro-2,3-dihydro-pyrrol-1-yl |
| D-109. | 3-nitro-2,3-dihydro-pyrrol-1-yl |
| D-110. | 2,5-dihydro-pyrrol-1-yl |
| D-111. | 2-chloro-2,5-dihydro-pyrrol-1-yl |
| D-112. | 3-chloro-2,5-dihydro-pyrrol-1-yl |
| D-113. | 2-bromo-2,5-dihydro-pyrrol-1-yl |
| D-114. | 3-bromo-2,5-dihydro-pyrrol-1-yl |
| D-115. | 2-cyano-2,5-dihydro-pyrrol-1-yl |
| D-116. | 3-cyano-2,5-dihydro-pyrrol-1-yl |
| D-117. | 2-nitro-2,5-dihydro-pyrrol-1-yl |
| D-118. | 3-nitro-2,5-dihydro-pyrrol-1-yl |
| D-119. | 2-methyl-2,5-dihydro-pyrrol-1-yl |
| D-120. | 3-methyl-2,5-dihydro-pyrrol-1-yl |
| D-121. | 2-trifluoromethyl-2,5-dihydro-pyrrol-1-yl |
| D-122. | 3-trifluoromethyl-2,5-dihydro-pyrrol-1-yl |
| D-123. | 2-methoxy-2,5-dihydro-pyrrol-1-yl |
| D-124. | 3-methoxy-2,5-dihydro-pyrrol-1-yl |
| D-125. | 2-trifluoromethoxy-2,5-dihydro-pyrrol-1-yl |
| D-126. | 3-trifluoromethoxy-2,5-dihydro-pyrrol-1-yl |
| D-127. | 2,3-dihydro-pyrazol-1-yl |
| D-128. | 3-chloro-2,3-dihydro-pyrazol-1-yl |
| D-129. | 4-chloro-2,3-dihydro-pyrazol-1-yl |
| D-130. | 5-chloro-2,3-dihydro-pyrazol-1-yl |
| D-131. | 3-bromo-2,3-dihydro-pyrazol-1-yl |
| D-132. | 4-bromo-2,3-dihydro-pyrazol-1-yl |
| D-133. | 5-bromo-2,3-dihydro-pyrazol-1-yl |
| D-134. | 3-cyano-2,3-dihydro-pyrazol-1-yl |
| D-135. | 4-cyano-2,3-dihydro-pyrazol-1-yl |
| D-136. | 5-cyano-2,3-dihydro-pyrazol-1-yl |
| D-137. | 3-nitro-2,3-dihydro-pyrazol-1-yl |
| D-138. | 4-nitro-2,3-dihydro-pyrazol-1-yl |
| D-139. | 5-nitro-2,3-dihydro-pyrazol-1-yl |
| D-140. | 3-methyl-2,3-dihydro-pyrazol-1-yl |
| D-141. | 4-methyl-2,3-dihydro-pyrazol-1-yl |
| D-142. | 5-methyl-2,3-dihydro-pyrazol-1-yl |
| D-143. | 3-trifluoromethyl-2,3-dihydro-pyrazol-1-yl |
| D-144. | 4-trifluoromethyl-2,3-dihydro-pyrazol-1-yl |
| D-145. | 5-trifluoronnethyl-2,3-dihydro-pyrazol-1-yl |
| D-146. | 3-methoxy-2,3-dihydro-pyrazol-1-yl |
| D-147. | 4-methoxy-2,3-dihydro-pyrazol-1-yl |
| D-148. y | 5-methoxy-2,3-dihydro-pyrazol-1-yl |
| D-149. | 3-trifluoromethoxy-2,3-dihydro-pyrazol-1-yl |
| D-150. | 4-trifluoromethoxy-2,3-dihydro-pyrazol-1-yl |
| D-151. | 5-trifluoromethoxy-2,3-dihydro-pyrazol-1-yl |
| D-152. | 2,5-dihydro-pyrazol-1-yl |
| D-153. | 3-chloro-2,5-dihydro-pyrazol-1-yl |
| D-154. | 4-chloro-2,5-dihydro-pyrazol-1-yl |
| D-155. | 5-chloro-2,5-dihydro-pyrazol-1-yl |
| D-156. | 3-bromo-2,5-dihydro-pyrazol-1-yl |
| D-157. | 4-bromo-2,5-dihydro-pyrazol-1-yl |
| D-158. | 5-bromo-2,5-dihydro-pyrazol-1-yl |
| D-159. | 3-cyano-2,5-dihydro-pyrazol-1-yl |
| D-160. | 4-cyano-2,5-dihydro-pyrazol-1-yl |
| D-161. | 5-cyano-2,5-dihydro-pyrazol-1-yl |
| D-162. | 3-nitro-2,5-dihydro-pyrazol-1-yl |
| D-163. | 4-nitro-2,5-dihydro-pyrazol-1-yl |
| D-164. | 5-nitro-2,5-dihydro-pyrazol-1-yl |
| D-165. | 3-methyl-2,5-dihydro-pyrazol-1-yl |
| D-166. | 4-methyl-2,5-dihydro-pyrazol-1-yl |
| D-167. | 5-methyl-2,5-dihydro-pyrazol-1-yl |
| D-168. | 3-trifluoromethyl-2,5-dihydro-pyrazol-1-yl |
| D-169. | 4-trifluoromethyl-2,5-dihydro-pyrazol-1-yl |
| D-170. | 5-trifluoromethyl-2,5-dihydro-pyrazol-1-yl |
| D-171. | 3-methoxy-2,5-dihydro-pyrazol-1-yl |
| D-172. | 4-methoxy-2,5-dihydro-pyrazol-1-yl |
| D-173. | 5-methoxy-2,5-dihydro-pyrazol-1-yl |
| D-174. | 3-trifluoromethoxy-2,5-dihydro-pyrazol-1-yl |
| D-175. | 4-trifluoromethoxy-2,5-dihydro-pyrazol-1-yl |
| D-176. | 5-trifluoromethoxy-2,5-dihydro-pyrazol-1-yl |
| D-177. | 4,5-dihydro-pyrazol-1-yl |
| D-178. | 3-chloro-4,5-dihydro-pyrazol-1-yl |
| D-179. | 4-chloro-4,5-dihydro-pyrazol-1-yl |
| D-180. | 5-chloro-4,5-dihydro-pyrazol-1-yl |
| D-181. | 3-bromo-4,5-dihydro-pyrazol-1-yl |
| D-182. | 4-bromo-4,5-dihydro-pyrazol-1-yl |
| D-183. | 5-bromo-4,5-dihydro-pyrazol-1-yl |
| D-184. | 3-cyano-4,5-dihydro-pyrazol-1-yl |
| D-185. | 4-cyano-4,5-dihydro-pyrazol-1-yl |
| D-186. | 5-cyano-4,5-dihydro-pyrazol-1-yl |
| D-187. | 3-nitro-4,5-dihydro-pyrazol-1-yl |
| D-188. | 4-nitro-4,5-dihydro-pyrazol-1-yl |
| D-189. | 5-nitro-4,5-dihydro-pyrazol-1-yl |
| D-190. | 3-methyl-4,5-dihydro-pyrazol-1-yl |
| D-191. | 4-methyl-4,5-dihydro-pyrazol-1-yl |
| D-192. | 5-methyl-4,5-dihydro-pyrazol-1-yl |
| D-193. | 3-trifluoromethyl-4,5-dihydro-pyrazol-1-yl |
| D-194. | 4-trifluoromethyl-4,5-dihydro-pyrazol-1-yl |
| D-195. | 5-trifluoromethyl-4,5-dihydro-pyrazol-1-yl |
| D-196. | 3-methoxy-4,5-dihydro-pyrazol-1-yl |
| D-197. | 4-methoxy-4,5-dihydro-pyrazol-1-yl |
| D-198. | 5-methoxy-4,5-dihydro-pyrazol-1-yl |
| D-199. | 2,3-dihydro-imidazol-1-yl |
| D-200. | 2,3-dihydro-imidazol-2-on-1-yl |
| D-201. | 2-chloro-2,3-dihydro-imidazol-1-yl |
| D-202. | 4-chloro-2,3-dihydro-imidazol-1-yl |
| D-203. | 5-chloro-2,3-dihydro-imidazol-1-yl |
| D-204. | 2-bromo-2,3-dihydro-imidazol-1-yl |
| D-205. | 4-bromo-2,3-dihydro-imidazol-1-yl |
| D-206. | 5-bromo-2,3-dihydro-imidazol-1-yl |
| D-207. | 2-cyano-2,3-dihydro-imidazol-1-yl |
| D-208. | 4-cyano-2,3-dihydro-imidazol-1-yl |
| D-209. | 5-cyano-2,3-dihydro-imidazol-1-yl |
| D-210. | 2-nitro-2,3-dihydro-imidazol-1-yl |
| D-211. | 4-nitro-2,3-dihydro-imidazol-1-yl |
| D-212. | 5-nitro-2,3-dihydro-imidazol-1-yl |
| D-213. | 2-methyl-2,3-dihydro-imidazol-1-yl |
| D-214. | 4-methyl-2,3-dihydro-imidazol-1-yl |
| D-215. | 5-methyl-2,3-dihydro-imidazol-1-yl |
| D-216. | 2-trifluoromethyl-2,3-dihydro-imidazol-1-yl |
| D-217. | 4-trifluoromethyl-2,3-dihydro-imidazol-1-yl |
| D-218. | 5-trifluoromethyl-2,3-dihydro-imidazol-1-yl |
| D-219. | 2-methoxy-2,3-dihydro-imidazol-1-yl |
| D-220. | 4-methoxy-2,3-dihydro-imidazol-1-yl |
| D-221. | 5-methoxy-2,3-dihydro-imidazol-1-yl |
| D-222. | 2-trifluoromethoxy-2,3-dihydro-imidazol-1-yl |
| D-223. | 4-trifluoromethoxy-2,3-dihydro-imidazol-1-yl |
| D-224. | 5-trifluoromethoxy-2,3-dihydro-imidazol-1-yl |
| D-225. | 5-chloro-2,3-dihydro-imidazol-2-on-1-yl |
| D-226. | 4-bromo-2,3-dihydro-imidazol-2-on-1-yl |
| D-227. | 5-bromo-2,3-dihydro-imidazol-2-on-1-yl |
| D-228. | 4-cyano-2,3-dihydro-imidazol-2-on-1-yl |
| D-229. | 5-cyano-2,3-dihydro-imidazol-2-on-1-yl |
| D-230. | 4-nitro-2,3-dihydro-imidazol-2-on-1-yl |
| D-231. | 5-nitro-2,3-dihydro-imidazol-2-on-1-yl |
| D-232. | 4-methyl-2,3-dihydro-imidazol-2-on-1-yl |
| D-233. | 5-methyl-2,3-dihydro-imidazol-2-on-1-yl |
| D-234. | 4-trifluoromethyl-2,3-dihydro-imidazol-2-on-1-yl |
| D-235. | 5-trifluoromethyl-2,3-dihydro-imidazol-2-on-1-yl |
| D-236. | 4-methoxy-2,3-dihydro-imidazol-2-on-1-yl |
| D-237. | 5-methoxy-2,3-dihydro-imidazol-2-on-1-yl |
| D-238. | 4-trifluoromethoxy-2,3-dihydro-imidazol-2-on-1-yl |
| D-239. | 5-trifluoromethoxy-2,3-dihydro-imidazol-2-on-1-yl |
| D-240. | 2,5-dihydro-imidazol-1-yl |
| D-241. | 2,5-dihydro-imidazol-2-on-1-yl |
| D-242. | 2-chloro-2,5-dihydro-imidazol-1-yl |
| D-243. | 4-chloro-2,5-dihydro-imidazol-1-yl |
| D-244. | 5-chloro-2,5-dihydro-imidazol-1-yl |
| D-245. | 2-bromo-2,5-dihydro-imidazol-1-yl |
| D-246. | 4-bromo-2,5-dihydro-imidazol-1-yl |
| D-247. | 5-bromo-2,5-dihydro-imidazol-1-yl |
| D-248. | 2-cyano-2,5-dihydro-imidazol-1-yl |
| D-249. | 4-cyano-2,5-dihydro-imidazol-1-yl |
| D-250. | 5-cyano-2,5-dihydro-imidazol-1-yl |
| D-251. | 2-nitro-2,5-dihydro-imidazol-1-yl |
| D-252. | 4-nitro-2,5-dihydro-imidazol-1-yl |
| D-253. | 5-nitro-2,5-dihydro-imidazol-1-yl |
| D-254. | 2-methyl-2,5-dihydro-imidazol-1-yl |
| D-255. | 4-methyl-2,5-dihydro-imidazol-1-yl |
| D-256. | 5-methyl-2,5-dihydro-imidazol-1-yl |
| D-257. | 2-trifluoromethyl-2,5-dihydro-imidazol-1-yl |
| D-258. | 4-trifluoromethyl-2,5-dihydro-imidazol-1-yl |
| D-259. | 5-trifluoromethyl-2,5-dihydro-imidazol-1-yl |
| D-260. | 2-methoxy-2,5-dihydro-imidazol-1-yl |
| D-261. | 4-methoxy-2,5-dihydro-imidazol-1-yl |
| D-262. | 5-methoxy-2,5-dihydro-imidazol-1-yl |
| D-263. | 2-trifluoromethoxy-2,5-dihydro-imidazol-1-yl |
| D-264. | 4-trifluoromethoxy-2,5-dihydro-imidazol-1-yl |
| D-265. | 5-trifluoromethoxy-2,5-dihydro-imidazol-1-yl |
| D-266. | 4-chloro-2,5-dihydro-imidazol-2-on-1-yl |
| D-267. | 5-chloro-2,5-dihydro-imidazol-2-on-1-yl |
| D-268. | 4-bromo-2,5-dihydro-imidazol-2-on-1-yl |
| D-269. | 5-bromo-2,5-dihydro-imidazol-2-on-1-yl |
| D-270. | 4-cyano-2,5-dihydro-imidazol-2-on-1-yl |
| D-271. | 5-cyano-2,5-dihydro-imidazol-2-on-1-yl |
| D-272. | 4-nitro-2,5-dihydro-imidazol-2-on-1-yl |
| D-273. | 5-nitro-2,5-dihydro-imidazol-2-on-1-yl |
| D-274. | 4-methyl-2,5-dihydro-imidazol-2-on-1-yl |
| D-275. | 5-methyl-2,5-dihydro-imidazol-2-on-1-yl |
| D-276. | 4-trifluoromethyl-2,5-dihydro-imidazol-2-on-1-yl |
| D-277. | 5-trifluoromethyl-2,5-dihydro-imidazol-2-on-1-yl |
| D-278. | 4-methoxy-2,5-dihydro-imidazol-2-on-1-yl |
| D-279. | 5-methoxy-2,5-dihydro-imidazol-2-on-1-yl |
| D-280. | 4-trifluoromethoxy-2,5-dihydro-imidazol-2-on-1-yl |
| D-281. | 5-trifluoromethoxy-2,5-dihydro-imidazol-2-on-1-yl |
| D-282. | 4,5-dihydro-imidazol-1-yl |
| D-283. | 2-chloro-4,5-dihydro-imidazol-1-yl |
| D-284. | 4-chloro-4,5-dihydro-imidazol-1-yl |
| D-285. | 5-chloro-4,5-dihydro-imidazol-1-yl |
| D-286. | 2-bromo-4,5-dihydro-imidazol-1-yl |
| D-287. | 4-bromo-4,5-dihydro-imidazol-1-yl |
| D-288. | 5-bromo-4,5-dihydro-imidazol-1-yl |
| D-289. | 2-cyano-4,5-dihydro-imidazol-1-yl |
| D-290. | 4-cyano-4,5-dihydro-imidazol-1-yl |
| D-291. | 5-cyano-4,5-dihydro-imidazol-1-yl |
| D-292. | 2-nitro-4,5-dihydro-imidazol-1-yl |
| D-293. | 4-nitro-4,5-dihydro-imidazol-1-yl |
| D-294. | 5-nitro-4,5-dihydro-imidazol-1-yl |
| D-295. | 2-methyl-4,5-dihydro-imidazol-1-yl |
| D-296. | 4-methyl-4,5-dihydro-imidazol-1-yl |
| D-297. | 5-methyl-4,5-dihydro-imidazol-1-yl |
| D-298. | 2-trifluoromethyl-4,5-dihydro-imidazol-1-yl |
| D-299. | 4-trifluoromethyl-4,5-dihydro-imidazol-1-yl |
| D-300. | 5-trifluoromethyl-4,5-dihydro-imidazol-1-yl |
| D-301. | 2-methoxy-4,5-dihydro-imidazol-1-yl |
| D-302. | 4-methoxy-4,5-dihydro-imidazol-1-yl |
| D-303. | 5-methoxy-4,5-dihydro-imidazol-1-yl |
| D-304. | 2-trifluoromethoxy-4,5-dihydro-imidazol-1-yl |
| D-305. | 4-trifluoromethoxy-4,5-dihydro-imidazol-1-yl |
| D-306. | 5-trifluoromethoxy-4,5-dihydro-imidazol-1-yl |
| D-307. | 2,3-dihydro-oxazol-3-yl |
| D-308. | 2-chloro-2,3-dihydro-oxazol-3-yl |
| D-309. | 4-chloro-2,3-dihydro-oxazol-3-yl |
| D-310. | 5-chloro-2,3-dihydro-oxazol-3-yl |
| D-311. | 2-bromo-2,3-dihydro-oxazol-3-yl |
| D-312. | 4-bromo-2,3-dihydro-oxazol-3-yl |
| D-313. | 5-bromo-2,3-dihydro-oxazol-3-yl |
| D-314. | 2-cyano-2,3-dihydro-oxazol-3-yl |
| D-315. | 4-cyano-2,3-dihydro-oxazol-3-yl |
| D-316. | 5-cyano-2,3-dihydro-oxazol-3-yl |
| D-317. | 2-nitro-2,3-dihydro-oxazol-3-yl |
| D-318. | 4-nitro-2,3-dihydro-oxazol-3-yl |
| D-319. | 5-nitro-2,3-dihydro-oxazol-3-yl |
| D-320. | 2-methyl-2,3-dihydro-oxazol-3-yl |
| D-321. | 4-methyl-2,3-dihydro-oxazol-3-yl |
| D-322. | 5-methyl-2,3-dihydro-oxazol-3-yl |
| D-323. | 2-trifluoromethyl-2,3-dihydro-oxazol-3-yl |
| D-324. | 4-trifluoromethyl-2,3-dihydro-oxazol-3-yl |
| D-325. | 5-trifluoromethyl-2,3-dihydro-oxazol-3-yl |
| D-326. | 2-methoxy-2,3-dihydro-oxazol-3-yl |
| D-327. | 4-methoxy-2,3-dihydro-oxazol-3-yl |
| D-328. | 5-methoxy-2,3-dihydro-oxazol-3-yl |
| D-329. | 2-trifluoromethoxy-2,3-dihydro-oxazol-3-yl |
| D-330. | 4-trifluoromethoxy-2,3-dihydro-oxazol-3-yl |
| D-331. | 5-trifluoromethoxy-2,3-dihydro-oxazol-3-yl |
| D-332. | 2,3-dihydro-thiazol-3-yl |
| D-333. | 2-chloro-2,3-dihydro-thiazol-3-yl |
| D-334. | 4-chloro-2,3-dihydro-thiazol-3-yl |
| D-335. | 5-chloro-2,3-dihydro-thiazol-3-yl |
| D-336. | 2-bromo-2,3-dihydro-thiazol-3-yl |
| D-337. | 4-bromo-2,3-dihydro-thiazol-3-yl |
| D-338. | 5-bromo-2,3-dihydro-thiazol-3-yl |
| D-339. | 2-cyano-2,3-dihydro-thiazol-3-yl |
| D-340. | 4-cyano-2,3-dihydro-thiazol-3-yl |
| D-341. | 5-cyano-2,3-dihydro-thiazol-3-yl |
| D-342. | 2-nitro-2,3-dihydro-thiazol-3-yl |
| D-343. | 4-nitro-2,3-dihydro-thiazol-3-yl |
| D-344. | 5-nitro-2,3-dihydro-thiazol-3-yl |
| D-345. | 2-methyl-2,3-dihydro-thiazol-3-yl |
| D-346. | 4-methyl-2,3-dihydro-thiazol-3-yl |
| D-347. | 5-methyl-2,3-dihydro-thiazol-3-yl |
| D-348. | 2-trifluoromethyl-2,3-dihydro-thiazol-3-yl |
| D-349. | 4-trifluoromethyl-2,3-dihydro-thiazol-3-yl |
| D-350. | 5-trifluoromethyl-2,3-dihydro-thiazol-3-yl |
| D-351. | 2-methoxy-2,3-dihydro-thiazol-3-yl |
| D-352. | 4-methoxy-2,3-dihydro-thiazol-3-yl |
| D-353. | 5-methoxy-2,3-dihydro-thiazol-3-yl |
| D-354. | 2-trifluoromethoxy-2,3-dihydro-thiazol-3-yl |
| D-355. | 4-trifluoromethoxy-2,3-dihydro-thiazol-3-yl |
| D-356. | 5-trifluoromethoxy-2,3-dihydro-thiazol-3-yl |
| D-357. | 2,3-dihydro-isoxazol-2-yl |
| D-358. | 3-chloro-2,3-dihydro-isoxazol-2-yl |
| D-359. | 4-chloro-2,3-dihydro-isoxazol-2-yl |
| D-360. | 5-chloro-2,3-dihydro-isoxazol-2-yl |
| D-361. | 3-bromo-2,3-dihydro-isoxazol-2-yl |
| D-362. | 4-bromo-2,3-dihydro-isoxazol-2-yl |
| D-363. | 5-bromo-2,3-dihydro-isoxazol-2-yl |
| D-364. | 3-cyano-2,3-dihydro-isoxazol-2-yl |
| D-365. | 4-cyano-2,3-dihydro-isoxazol-2-yl |
| D-366. | 5-cyano-2,3-dihydro-isoxazol-2-yl |
| D-367. | 3-nitro-2,3-dihydro-isoxazol-2-yl |
| D-368. | 4-nitro-2,3-dihydro-isoxazol-2-yl |
| D-369. | 5-nitro-2,3-dihydro-isoxazol-2-yl |
| D-370. | 3-methyl-2,3-dihydro-isoxazol-2-yl |
| D-371. | 4-methyl-2,3-dihydro-isoxazol-2-yl |
| D-372. | 5-methyl-2,3-dihydro-isoxazol-2-yl |
| D-373. | 3-trifluoromethyl-2,3-dihydro-isoxazol-2-yl |
| D-374. | 4-trifluoromethyl-2,3-dihydro-isoxazol-2-yl |
| D-375. | 5-trifluoromethyl-2,3-dihydro-isoxazol-2-yl |
| D-376. | 3-trifluoromethyl-2,3-dihydro-isoxazol-2-yl |
| D-377. | 4-trifluoromethyl-2,3-dihydro-isoxazol-2-yl |
| D-378. | 5-methoxy-2,3-dihydro-isoxazol-2-yl |
| D-379. | 3-trifluoromethoxy-2,3-dihydro-isoxazol-2-yl |
| D-380. | 4-trifluoromethoxy-2,3-dihydro-isoxazol-2-yl |
| D-381. | 5-trifluoromethoxy-2,3-dihydro-isoxazol-2-yl |
| D-382. | 2,5-dihydro-isoxazol-2-yl |
| D-383. | 3-chloro-2,5-dihydro-isoxazol-2-yl |
| D-384. | 4-chloro-2,5-dihydro-isoxazol-2-yl |
| D-385. | 5-chloro-2,5-dihydro-isoxazol-2-yl |
| D-386. | 3-bromo-2,5-dihydro-isoxazol-2-yl |
| D-387. | 4-bromo-2,5-dihydro-isoxazol-2-yl |
| D-388. | 5-bromo-2,5-dihydro-isoxazol-2-yl |
| D-389. | 3-cyano-2,5-dihydro-isoxazol-2-yl |
| D-390. | 4-cyano-2,5-dihydro-isoxazol-2-yl |
| D-391. | 5-cyano-2,5-dihydro-isoxazol-2-yl |
| D-392. | 3-nitro-2,5-dihydro-isoxazol-2-yl |
| D-393. | 4-nitro-2,5-dihydro-isoxazol-2-yl |
| D-394. | 5-nitro-2,5-dihydro-isoxazol-2-yl |
| D-395. | 3-methyl-2,5-dihydro-isoxazol-2-yl |
| D-396. | 4-methyl-2,5-dihydro-isoxazol-2-yl |
| D-397. | 5-methyl-2,5-dihydro-isoxazol-2-yl |
| D-398. | 3-trifluoromethyl-2,5-dihydro-isoxazol-2-yl |
| D-399. | 4-trifluoromethyl-2,5-dihydro-isoxazol-2-yl |
| D-400. | 5-trifluoromethyl-2,5-dihydro-isoxazol-2-yl |
| D-401. | 4-methoxy-2,5-dihydro-isoxazol-2-yl |
| D-402. | 4-methoxy-2,5-dihydro-isoxazol-2-yl |
| D-403. | 5-methoxy-2,5-dihydro-isoxazol-2-yl |
| D-404. | 3-trifluoromethoxy-2,5-dihydro-isoxazol-2-yl |
| D-405. | 4-trifluoromethoxy-2,5-dihydro-isoxazol-2-yl |
| D-406. | 5-trifluoromethoxy-2,5-dihydro-isoxazol-2-yl |
| D-407. | 2,3-dihydro-isothiazol-2-yl |
| D-408. | 3-chloro-2,3-dihydro-isothiazol-2-yl |
| D-409. | 4-chloro-2,3-dihydro-isothiazol-2-yl |
| D-410. | 5-chloro-2,3-dihydro-isothiazol-2-yl |
| D-411. | 3-bromo-2,3-dihydro-isothiazol-2-yl |
| D-412. | 4-bromo-2,3-dihydro-isothiazol-2-yl |
| D-413. | 5-bromo-2,3-dihydro-isothiazol-2-yl |
| D-414. | 3-cyano-2,3-dihydro-isothiazol-2-yl |
| D-415. | 4-cyano-2,3-dihydro-isothiazol-2-yl |
| D-416. | 5-cyano-2,3-dihydro-isothiazol-2-yl |
| D-417. | 3-nitro-2,3-dihydro-isothiazol-2-yl |
| D-418. | 4-nitro-2,3-dihydro-isothiazol-2-yl |
| D-419. | 5-nitro-2,3-dihydro-isothiazol-2-yl |
| D-420. | 3-methyl-2,3-dihydro-isothiazol-2-yl |
| D-421. | 4-methyl-2,3-dihydro-isothiazol-2-yl |
| D-422. | 5-methyl-2,3-dihydro-isothiazol-2-yl |
| D-423. | 3-trifluoromethyl-2,3-dihydro-isothiazol-2-yl |
| D-424. | 4-trifluoromethyl-2,3-dihydro-isothiazol-2-yl |
| D-425. | 5-trifluoromethyl-2,3-dihydro-isothiazol-2-yl |
| D-426. | 3-trifluoromethyl-2,3-dihydro-isothiazol-2-yl |
| D-427. | 4-trifluoromethyl-2,3-dihydro-isothiazol-2-yl |
| D-428. | 5-methoxy-2,3-dihydro-isothiazol-2-yl |
| D-429. | 3-trifluoromethoxy-2,3-dihydro-isothiazol-2-yl |
| D-430. | 4-trifluoromethoxy-2,3-dihydro-isothiazol-2-yl |
| D-431. | 5-trifluoromethoxy-2,3-dihydro-isothiazol-2-yl |
| D-432. | 2,5-dihydro-isothiazol-2-yl |
| D-433. | 3-chloro-2,5-dihydro-isothiazol-2-yl |
| D-434. | 4-chloro-2,5-dihydro-isothiazol-2-yl |
| D-435. | 5-chloro-2,5-dihydro-isothiazol-2-yl |
| D-436. | 3-bromo-2,5-dihydro-isothiazol-2-yl |
| D-437. | 4-bromo-2,5-dihydro-isothiazol-2-yl |
| D-438. | 5-bromo-2,5-dihydro-isothiazol-2-yl |
| D-439. | 3-cyano-2,5-dihydro-isothiazol-2-yl |
| D-440. | 4-cyano-2,5-dihydro-isothiazol-2-yl |
| D-441. | 5-cyano-2,5-dihydro-isothiazol-2-yl |
| D-442. | 3-nitro-2,5-dihydro-isothiazol-2-yl |
| D-443. | 4-nitro-2,5-dihydro-isothiazol-2-yl |
| D-444. | 5-nitro-2,5-dihydro-isothiazol-2-yl |
| D-445. | 3-methyl-2,5-dihydro-isothiazol-2-yl |
| D-446. | 4-methyl-2,5-dihydro-isothiazol-2-yl |
| D-447. | 5-methyl-2,5-dihydro-isothiazol-2-yl |
| D-448. | 3-trifluoromethyl-2,5-dihydro-isothiazol-2-yl |
| D-449. | 4-trifluoromethyl-2,5-dihydro-isothiazol-2-yl |
| D-450. | 5-trifluoromethyl-2,5-dihydro-isothiazol-2-yl |
| D-451. | 4-methoxy-2,5-dihydro-isothiazol-2-yl |
| D-452. | 4-methoxy-2,5-dihydro-isothiazol-2-yl |
| D-453. | 5-methoxy-2,5-dihydro-isothiazol-2-yl |
| D-454. | 3-trifluoromethoxy-2,5-dihydro-isothiazol-2-yl |
| D-455. | 4-trifluoromethoxy-2,5-dihydro-isothiazol-2-yl |
| D-456. | 5-trifluoromethoxy-2,5-dihydro-isothiazol-2-yl |
| D-457. | pyrrol-1-yl |
| D-458. | 3-chloro-pyrrol-1-yl |
| D-459. | 2-bromo-pyrrol-1-yl |
| D-460. | 3-bromo-pyrrol-1-yl |
| D-461. | 2-cyano-pyrrol-1-yl |
| D-462. | 3-cyano-pyrrol-1-yl |
| D-463. | 2-nitro-pyrrol-1-yl |
| D-464. | 3-nitro-pyrrol-1-yl |
| D-465. | 2-methyl-pyrrol-1-yl |
| D-466. | 3-methyl-pyrrol-1-yl |
| D-467. | 2-trifluoromethyl-pyrrol-1-yl |
| D-468. | 3-trifluoromethyl-pyrrol-1-yl |
| D-469. | 2-methoxy-pyrrol-1-yl |
| D-470. | 3-methoxy-pyrrol-1-yl |
| D-471. | 2-trifluoromethoxy-pyrrol-1-yl |
| D-472. | 3-trifluoromethoxy-pyrrol-1-yl |
| D-473. | pyrazol-1-yl |
| D-474. | 3-chloro-pyrazol-1-yl |
| D-475. | 4-chloro-pyrazol-1-yl |
| D-476. | 5-chloro-pyrazol-1-yl |
| D-477. | 3-bromo-pyrazol-1-yl |
| D-478. | 4-bromo-pyrazol-1-yl |
| D-479. | 5-bromo-pyrazol-1-yl |
| D-480. | 3-cyano-pyrazol-1-yl |
| D-481. | 4-cyano-pyrazol-1-yl |
| D-482. | 5-cyano-pyrazol-1-yl |
| D-483. | 3-nitro-pyrazol-1-yl |
| D-484. | 4-nitro-pyrazol-1-yl |
| D-485. | 5-nitro-pyrazol-1-yl |
| D-486. | 3-methyl-pyrazol-1-yl |
| D-487. | 4-methyl-pyrazol-1-yl |
| D-488. | 5-methyl-pyrazol-1-yl |
| D-489. | 3-trifluoromethyl-pyrazol-1-yl |
| D-490. | 4-trifluoromethyl-pyrazol-1-yl |
| D-491. | 5-trifluoromethyl-pyrazol-1-yl |
| D-492. | 3-methoxy-pyrazol-1-yl |
| D-493. | 4-methoxy-pyrazol-1-yl |
| D-494. | 5-methoxy-pyrazol-1-yl |
| D-495. | 3-trifluoromethoxy-pyrazol-1-yl |
| D-496. | 4-trifluoromethoxy-pyrazol-1-yl |
| D-497. | 5-trifluoromethoxy-pyrazol-1-yl |
| D-498. | imidazol-1-yl |
| D-499. | 2-chloro-imidazol-1-yl |
| D-500. | 4-chloro-imidazol-1-yl |
| D-501. | 5-chloro-imidazol-1-yl |
| D-502. | 2-bromo-imidazol-1-yl |
| D-503. | 4-bromo-imidazol-1-yl |
| D-504. | 5-bromo-imidazol-1-yl |
| D-505. | 2-cyano-imidazol-1-yl |
| D-506. | 4-cyano-imidazol-1-yl |
| D-507. | 5-cyano-imidazol-1-yl |
| D-508. | 2-nitro-imidazol-1-yl |
| D-509. | 4-nitro-imidazol-1-yl |
| D-510. | 5-nitro-imidazol-1-yl |
| D-511. | 2-methyl-imidazol-1-yl |
| D-512. | 4-methyl-imidazol-1-yl |
| D-513. | 5-methyl-imidazol-1-yl |
| D-514. | 2-trifluoromethyl-imidazol-1-yl |
| D-515. | 4-trifluoromethyl-imidazol-1-yl |
| D-516. | 5-trifluoromethyl-imidazol-1-yl |
| D-517. | 2-methoxy-imidazol-1-yl |
| D-518. | 4-methoxy-imidazol-1-yl |
| D-519. | 5-methoxy-imidazol-1-yl |
| D-520. | 2-trifluoromethoxy-imidazol-1-yl |
| D-521. | 4-trifluoromethoxy-imidazol-1-yl |
| D-522. | 5-trifluoromethoxy-imidazol-1-yl |
| D-523. | [1,2,3]-triazol-1-yl |
| D-524. | 4-chloro-[1,2,3]-triazol-1-yl |
| D-525. | 5-chloro-[1,2,3]-triazol-1-yl |
| D-526. | 4-bromo-[1,2,3]-triazol-1-yl |
| D-527. | 5-bromo-[1,2,3]-triazol-1-yl |
| D-528. | 4-cyano-[1,2,3]-triazol-1-yl |
| D-529. | 5-cyano-[1,2,3]-triazol-1-yl |
| D-530. | 4-nitro-[1,2,3]-triazol-1-yl |
| D-531. | 5-nitro-[1,2,3]-triazol-1-yl |
| D-532. | 4-methyl-[1,2,3]-triazol-1-yl |
| D-533. | 5-methyl-[1,2,3]-triazol-1-yl |
| D-534. | 4-trifluoromethyl-[1,2,3]-triazol-1-yl |
| D-535. | 5-trifluoromethyl-[1,2,3]-triazol-1-yl |
| D-536. | 4-methoxy-[1,2,3]-triazol-1-yl |
| D-537. | 4-trifluoromethoxy-[1,2,3]-triazol-1-yl |
| D-538. | 5-trifluoromethoxy-[1,2,3]-triazol-1-yl |
| D-539. | [1,2,4]-triazol-1-yl |
| D-540. | 3-chloro-[1,2,4]-triazol-1-yl |
| D-541. | 5-chloro-[1,2,4]-triazol-1-yl |
| D-542. | 3-bromo-[1,2,4]-triazol-1-yl |
| D-543. | 5-bromo-[1,2,4]-triazol-1-yl |
| D-544. | 3-cyano-[1,2,4]-triazol-1-yl |
| D-545. | 5-cyano-[1,2,4]-triazol-1-yl |
| D-546. | 3-nitro-[1,2,4]-triazol-1-yl |
| D-547. | 5-nitro-[1,2,4]-triazol-1-yl |
| D-548. | 3-methyl-[1,2,4]-triazol-1-yl |
| D-549. | 5-methyl-[1,2,4]-triazol-1-yl |
| D-550. | 3-trifluoromethyl-[1,2,4]-triazol-1-yl |
| D-551. | 5-trifluoromethyl-[1,2,4]-triazol-1-yl |
| D-552. | 3-methoxy-[1,2,4]-triazol-1-yl |
| D-553. | 3-trifluoromethoxy-[1,2,4]-triazol-1-yl |
| D-554, | 5-trifluoromethoxy-[1,2,4]-triazol-1-yl |
| D-555. | [1,2,5]-triazol-1-yl |
| D-556. | 3-chloro-[1,2,5]-triazol-1-yl |
| D-557. | 4-chloro-[1,2,5]-triazol-1-yl |
| D-558. | 3-bromo-[1,2,5]-triazol-1-yl |
| D-559. | 4-bromo-[1,2,5]-triazol-1-yl |
| D-560. | 3-cyano-[1,2,5]-triazol-1-yl |
| D-561. | 4-cyano-[1,2,5]-triazol-1-yl |
| D-562. | 3-nitro-[1,2,5]-triazol-1-yl |
| D-563. | 4-nitro-[1,2,5]-triazol-1-yl |
| D-564. | 3-methyl-[1,2,5]-triazol-1-yl |
| D-565. | 4-methyl-[1,2,5]-triazol-1-yl |
| D-566. | 3-trifluoromethyl-[1,2,5]-triazol-1-yl |
| D-567. | 4-trifluoromethyl-[1,2,5]-triazol-1-yl |
| D-568. | 3-methoxy-[1,2,5]-triazol-1-yl |
| D-569. | 3-trifluoromethoxy-[1,2,5]-triazol-1-yl |
| D-570. | 4-trifluoromethoxy-[1,2,5]-triazol-1-yl |
| D-571. | piperidin-1-yl |
| D-572. | 2-chloro-piperidin-1-yl |
| D-573. | 3-chloro-piperidin-1-yl |
| D-574. | 4-chloro-piperidin-1-yl |
| D-575. | 2-bromo-piperidin-1-yl |
| D-576. | 3-bromo-piperidin-1-yl |
| D-577. | 4-bromo-piperidin-1-yl |
| D-578. | 2-cyano-piperidin-1-yl |
| D-579. | 3-cyano-piperidin-1-yl |
| D-580. | 4-cyano-piperidin-1-yl |
| D-581. | 2-nitro-piperidin-1-yl |
| D-582. | 3-nitro-piperidin-1-yl |
| D-583. | 4-nitro-piperidin-1-yl |
| D-584. | 2-methyl-piperidin-1-yl |
| D-585. | 3-methyl-piperidin-1-yl |
| D-586. | 4-methyl-piperidin-1-yl |
| D-587. | 2-trifluoromethyl-piperidin-1-yl |
| D-588. | 3-trifluoromethyl-piperidin-1-yl |
| D-589. | 4-trifluoroniethyl-piperidin-1-yl |
| D-590. | 2-methoxy-piperidin-1-yl |
| D-591. | 3-methoxy-piperidin-1-yl |
| D-592. | 4-methoxy-piperidin-1-yl |
| D-593. | 2-trifluoromethoxy-piperidin-1-yl |
| D-594. | 3-trifluoromethoxy-piperidin-1-yl |
| D-595. | 4-trifluoromethoxy-piperidin-1-yl |
| D-596. | morpholin-4-yl |
| D-597. | 2-chloro-morpholin-4-yl |
| D-598. | 3-chloro-morpholin-4-yl |
| D-599. | 2-bromo-morpholin-4-yl |
| D-600. | 3-bromo-morpholin-4-yl |
| D-601. | 2-cyano-morpholin-4-yl |
| D-602. | 3-cyano-morpholin-4-yl |
| D-603. | 2-nitro-morpholin-4-yl |
| D-604. | 3-nitro-morpholin-4-yl |
| D-605. | 2-methyl-morpholin-4-yl |
| D-606. | 3-methyl-morpholin-4-yl |
| D-607. | 2-trifluoromethyl-morpholin-4-yl |
| D-608. | 3-trifluoromethyl-morpholin-4-yl |
| D-609. | 2-methoxy-morpholin-4-yl |
| D-610. | 3-methoxy-morpholin-4-yl |
| D-611. | 2-trifluoromethoxy-morpholin-4-yl |
| D-612. | 3-trifluoromethoxy-morpholin-4-yl |
| D-613. | piperazin-1-yl |
| D-614. | 4-methyl-piperazin-1-yl |
| D-615. | 2-chloro-piperazin-1-yl |
| D-616. | 3-chloro-piperazin-1-yl |
| D-617. | 2-bromo-piperazin-1-yl |
| D-618. | 3-bromo-piperazin-1-yl |
| D-619. | 2-cyano-piperazin-1-yl |
| D-620. | 3-cyano-piperazin-1-yl |
| D-621. | 2-nitro-piperazin-1-yl |
| D-622. | 3-nitro-piperazin-1-yl |
| D-623. | 2-methyl-piperazin-1-yl |
| D-624. | 3-methyl-piperazin-1-yl |
| D-625. | 2-trifluoromethyl-piperazin-1-yl |
| D-626. | 3-trifluoromethyl-piperazin-1-yl |
| D-627. | 2-methoxy-piperazin-1-yl |
| D-628. | 3-methoxy-piperazin-1-yl |
| D-629. | 2-trifluoromethoxy-piperazin-1-yl |
| D-630. | 3-trifluoromethoxy-piperazin-1-yl |
| D-631. | C(═NOH)NH₂ |
| D-632. | C(═NOCH₃)NH₂ |
| D-633. | C(═NOH)NHCH₃ |
| D-634. | C(═NOCH₃)NHCH₃ |
| D-635. | C(═NOH)N(CH₃)₂ |
| D-636. | C(═NOCH₃)N(CH₃)₂ |
| D-637. | C(═NOH)NHCH₂CH₃ |
| D-638. | C(═NOCH₃)NHCH₂CH₃ |
| D-639. | C(═NOH)N(CH₂CH₃)₂ |
| D-640. | C(═NOCH₃)N(CH₂CH₃)₂ |
| D-641. | C(═NOH)NHCH₂CH₂CH₃ |
| D-642. | C(═NOCH₃)NHCH₂CH₂CH₃ |
| D-643. | C(═NOH)N(CH₂CH₂CH₃)₂ |
| D-644. | C(═NOCH₃)N(CH₂CH₂CH₃)₂ |
| D-645. | C(═NOH)NHCH(CH₃)₂ |
| D-646. | C(═NOCH₃)NHCH(CH₃)₂ |
| D-647. | C(═NOH)NHCH₂CH₂CH₂CH₃ |
| D-648. | C(═NOCH₃)NHCH₂CH₂CH₂CH₃ |
| D-649. | C(═NOH)N(CH₂CH₂CH₂CH₃)₂ |
| D-650. | C(═NOCH₃)N(CH₂CH₂CH₂CH₃)₂ |
| D-651. | isoxazol-3-yl |
| D-652. | 4-chloro-isoxazol-3-yl |
| D-653. | 5-chloro-isoxazol-3-yl |
| D-654. | 4-bromo-isoxazol-3-yl |
| D-655. | 5-bromo-isoxazol-3-yl |
| D-656. | 4-cyano-isoxazol-3-yl |
| D-657. | 5-cyano-isoxazol-3-yl |
| D-658. | 4-nitro-isoxazol-3-yl |
| D-659. | 5-nitro-isoxazol-3-yl |
| D-660. | 4-methyl-isoxazol-3-yl |
| D-661. | 5-methyl-isoxazol-3-yl |
| D-662. | 4-trifluoromethyl-isoxazol-3-yl |
| D-663. | 5-trifluoromethyl-isoxazol-3-yl |
| D-664. | 4-methoxy-isoxazol-3-yl |
| D-665. | 5-methoxy-isoxazol-3-yl |
| D-666. | 4-trifluoromethoxy-isoxazol-3-yl |
| D-667. | 5-trifluoromethoxy-isoxazol-3-yl |
| D-668. | 4,5-dihydroisoxazol-3-yl |
| D-669. | 4-chloro-4,5-dihydroisoxazol-3-yl |
| D-670. | 5-chloro-4,5-dihydroisoxazol-3-yl |
| D-671. | 4-bromo-4,5-dihydroisoxazol-3-yl |
| D-672. | 5-bromo-4,5-dihydroisoxazol-3-yl |
| D-673. | 4-cyano-4,5-dihydroisoxazol-3-yl |
| D-674. | 5-cyano-4,5-dihydroisoxazol-3-yl |
| D-675. | 4-nitro-4,5-dihydroisoxazol-3-yl |
| D-676. | 5-nitro-4,5-dihydroisoxazol-3-yl |
| D-677. | 4-methyl-4,5-dihydroisoxazol-3-yl |
| D-678. | 5-methyl-4,5-dihydroisoxazol-3-yl |
| D-679. | 4-trifluoromethyl-4,5-dihydroisoxazol-3-yl |
| D-680. | 5-trifluoromethyl-4,5-dihydroisoxazol-3-yl |
| D-681. | 4-methoxy-4,5-dihydroisoxazol-3-yl |
| D-682. | 5-methoxy-4,5-dihydroisoxazol-3-yl |
| D-683. | 4-trifluoromethoxy-4,5-dihydroisoxazol-3-yl |
| D-684. | 5-trifluoromethoxy-4,5-dihydroisoxazol-3-yl |
| D-685. | [1,2,4]-oxadiazol-3-yl* |
| D-686. | 5-chloro-[1,2,4]-oxadiazol-3-yl |
| D-687. | 5-bromo-[1,2,4]-oxadiazol-3-yl |
| D-688. | 5-cyano-[1,2,4]-oxadiazol-3-yl |
| D-689. | 5-nitro-[1,2,4]-oxadiazol-3-yl |
| D-690. | 5-methyl-[1,2,4]-oxadiazol-3-yl |
| D-691. | 5-trifluoromethyl-[1,2,4]-oxadiazol-3-yl |
| D-692. | 5-methoxy-[1,2,4]-oxadiazol-3-yl |
| D-693. | 5-trifluoromethoxy-[1,2,4]-oxadiazol-3-yl |
| D-694. | [1,2,5]-oxadiazol-3-yl* |
| D-695. | 4-chloro-[1,2,5]-oxadiazol-3-yl |
| D-696. | 4-bromo-[1,2,5]-oxadiazol-3-yl |
| D-697. | 4-cyano-[1,2,5]-oxadiazol-3-yl |
| D-698. | 4-nitro-[1,2,5]-oxadiazol-3-yl |
| D-699. | 4-methyl-[1,2,5]-oxadiazol-3-yl |
| D-700. | 4-trifluoromethyl-[1,2,5]-oxadiazol-3-yl |
| D-701. | 4-methoxy-[1,2,5]-oxadiazol-3-yl |
| D-702. | 4-trifluoromethoxy-[1,2,5]-oxadiazol-3-yl |

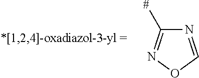

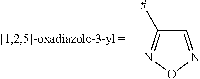

Compounds of formula I can be prepared by one or more of the following methods and variations as described in schemes 1 to 3. The variables are as defined above formula I.

Compounds of formula (I), wherein X is O, can be prepared by cycloaddition of styrene compounds of formula (II) with nitrile oxides derived from oximes of formula (III), wherein Y is OH and Z is H, as outlined in scheme 1. The reaction typically proceeds through the intermediacy of an in situ generated hydroxamic acid halogenide, normally chloride, by reaction with chlorine, hypochloride, N-succinimide, or chloramine-T. The halogenating agent is combined with the oxime before addition, or in the presence of the styrene II. Depending on the conditions, amine bases such as pyridine or triethylamine may be necessary. The reaction can be run in a wide variety of solvents including DMF, toluene, dichloromethane, chlorobenzene, acetonitrile, tetrahydrofuran, diehylether, ethylacetate or the like. Compounds of formula (II) can be prepared as described, for example, in WO 2005/085216, or in WO 2007/094313.

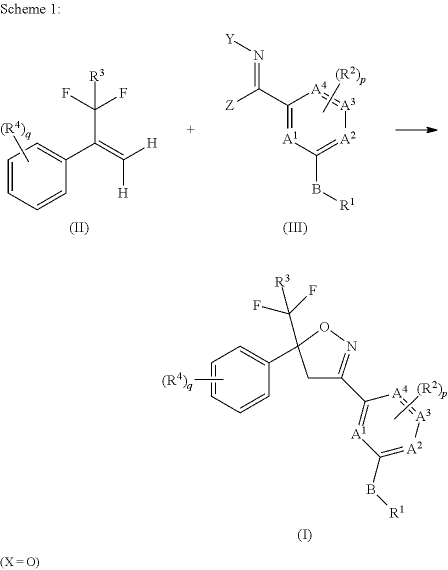

Compounds of formula I, wherein X is CH₂ (Y═CH₂SiR′₃ with R′ representing optionally substituted alkyl, Z═SH) can also be prepared as described e.g. in WO 2009/072621.

Compounds of formula I-a, can also be prepared as outlined in scheme 2 by reaction of a pyridine N-oxide of formula V with a nucleophile in the presence of an alkylation or acylation agent, as for example described by Huo et al, Tetrahedron Letters (2008), 49(28), 4369-4371. Alternatively, Compounds of formula I-a can also be prepared via halogen exchange reaction of compounds of formula VI (J may be a halogen as for example Cl, F, Br or I), as for example described by Swahn et al, Bioorganic & Medicinal Chemistry Letters (2006), 16(5), 1397-1401. Compounds of formula VI can be prepared from pyridine N-oxides of formula V, as for example described by Carabateas et al, Journal of Heterocyclic Chemistry (1984), 21(6), 1857-1863. The corresponding pyridine oxides of formula V can be prepared by oxidation of pyridines of formula IV, as for example described by Komoriya et al, Bioorganic & Medicinal Chemistry (2005), 13(12), 3927-3954 or by Wolfle et al, Journal of the American Chemical Society (1992), 114(24), 9304-9309. Compounds of formula IV have been for example described in WO 200/4018410.

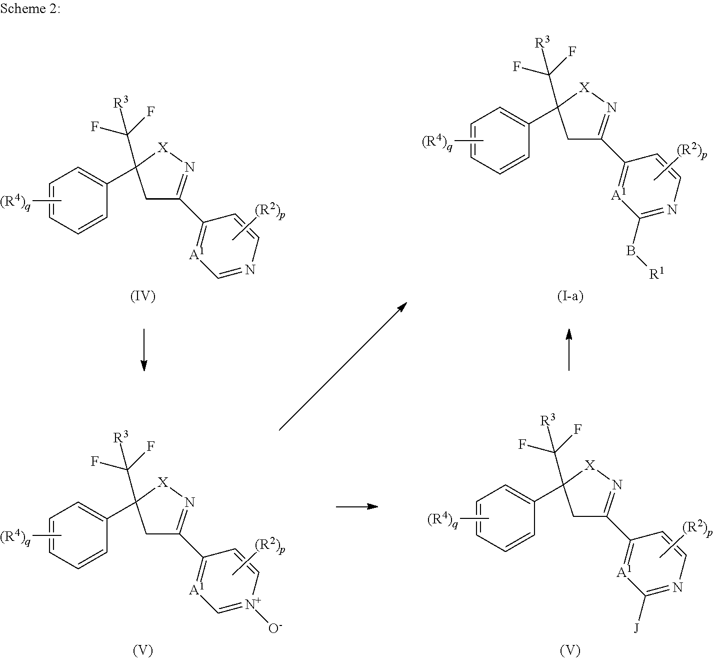

Compounds of formula I-b can be prepared as outlined in scheme 3 by reaction of pyridine oxides of formula VIII with a nucleophile in the presence of an alkylation or acylation agent, as for example described in WO 2009/037277. Alternatively, compounds of formula I-b can also be prepared via halogen exchange reaction of compounds of formula IX (J may be a halogen as for example Cl, F, Br or I), as for example described by Yang et al, Tetrahedron Letters (2009), 50(25), 3081-3083 or by Zhang et al, Journal of Medicinal Chemistry (2008), 51(22), 7099-7110. Compounds of formula X can be prepared from compounds of formula VIII, as for example described by Moran et al, Journal of Heterocyclic Chemistry (1986), 23(4), 1071-1077. Compounds of formula VIII can be prepared from compounds of formula VII, as for example described by Hrdina et al, Advanced Synthesis & Catalysis (2008), 350(10), 1449-1456. The corresponding compounds of formula VII have been described for example in WO 2004/018410.

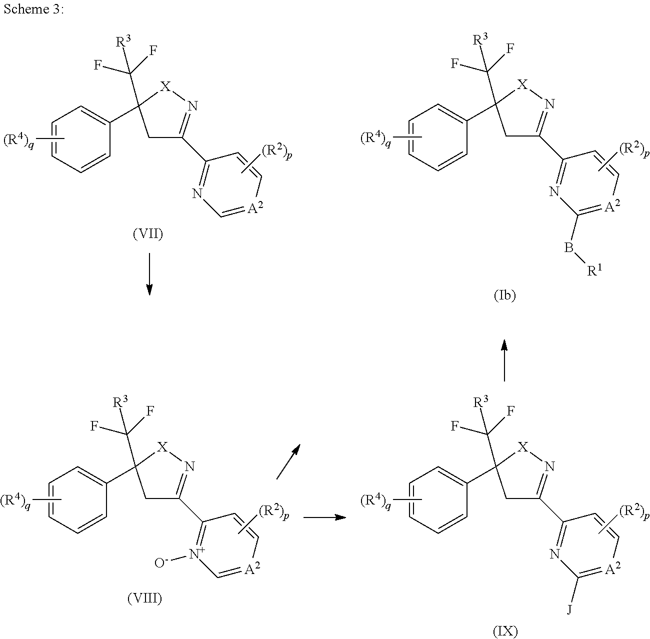

Compounds of formula III-a can also be prepared as outlined in scheme 4 by deprotonation of compounds of formula X followed by reaction with an alkyl nitrite, as for example described in WO 2007/133622 (Z may be hydrogen).

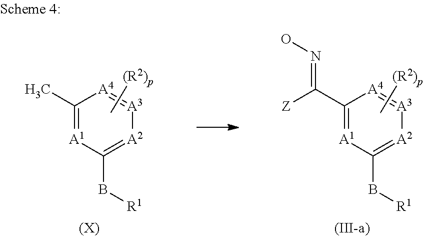

Compounds of formula I-c can also be prepared as outlined in scheme 5 by nucleophilic substitution of compounds of formula XI (J may be a halogen, as for example F, Cl, Br or I) with thiolates, as for example described by Sun et al, Tetrahedron (2006), 62, 8702-8706. The corresponding compounds of formula XI can be prepared as for example described in WO 2009/077197.

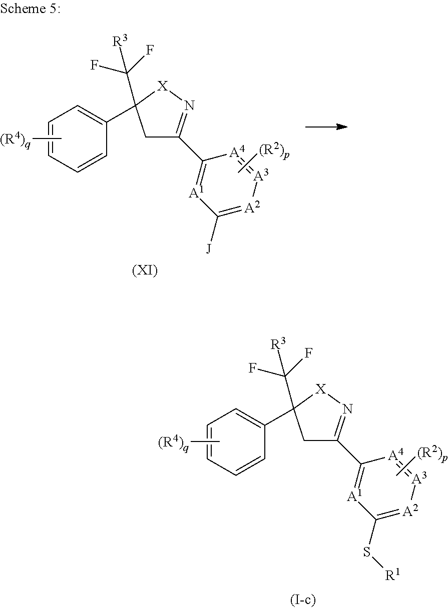

Compounds of formula I-d can also be prepared as outlined in scheme 6 by nucleophilic substitution of compounds of formula XI (J may be a halogen, as for example F, Cl, Br or I) with alcoholates, as for example described in WO 2009/010801.

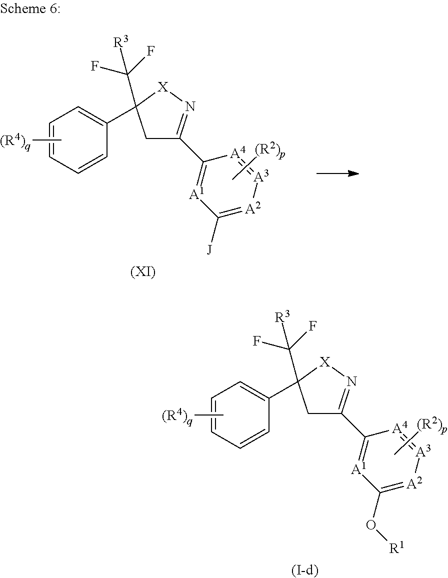

Compounds of formula I-e can also be prepared as outlined in scheme 7 by carbonylation reaction of compounds of formula XI (J may be a halogen, as for example F, Cl, Br or I) in the presence of an appropriate nucleophile, as for example described by Stefko et al, Tetrahedron (2009), 65(23), 4471-4483.

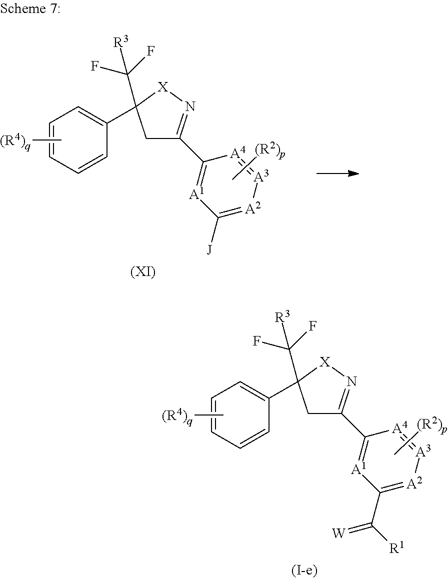

Compounds of formula I-f can also be prepared as outlined in scheme 8 by transition metal mediated cross coupling reaction of compounds of formula XI, as for example described by Hartwig et al, Journal of the American Chemical Society (2001), 123(19), 4641-4642. Compounds of formula I-f can also be prepared from compounds of formula I-e by the addition of organometallic reagents as for example described in WO 2008/020302.

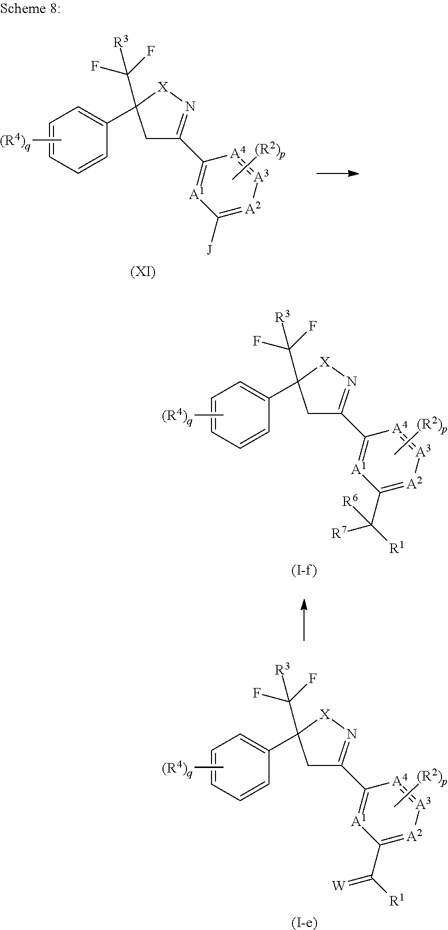

If individual compounds cannot be prepared via the above-described routes, they can be prepared by derivatization of other compounds I or by customary modifications of the synthesis routes described.

The reaction mixtures are worked up in the customary manner, for example by mixing with water, separating the phases, and, if appropriate, purifying the crude products by chromatography, for example on alumina or silica gel. Some of the intermediates and end products may be obtained in the form of colorless or pale brown viscous oils, which are freed or purified from volatile components under reduced pressure and at moderately elevated temperature. If the intermediates and end products are obtained as solids, they may be purified by recrystallization or digestion.

Due to their excellent activity, the compounds of formula I may be used for controlling invertebrate pests.

Accordingly, the present invention also provides an agricultural composition comprising at least one compound of the formula I, as defined above, a stereoisomer thereof and/or at least one agriculturally acceptable salt thereof, and at least one inert liquid and/or solid agriculturally acceptable carrier.

The present invention also provides a veterinary composition comprising at least one compound of the formula I, as defined above, a stereoisomer thereof and/or at least one veterinarily acceptable salt thereof, and at least one inert liquid and/or solid veterinarily acceptable carrier.

Such compositions may contain a single active compound of formula I or a salt thereof or a mixture of several active compounds of formula I or their salts according to the present invention. The composition according to the present invention may comprise an individual isomer or mixtures of isomers as well as individual tautomers or mixtures of tautomers.

The present invention further relates to the use of a compound as defined above, of a stereoisomer and/or of an agriculturally or veterinarily acceptable salt thereof for combating invertebrate pests.

The present invention further relates to the use of a compound as defined above, of a stereoisomer and/or of a veterinarily acceptable salt thereof, for treating or protecting an animal from infestation or infection by invertebrate pests.

Moreover the present invention also provides a method for controlling invertebrate pests which method comprises treating the pests, their food supply, their habitat or their breeding ground or a plant, plant propagation material, soil, area, material or environment in which the pests are growing or may grow, or the materials, plants, plant propagation material, soils, surfaces or spaces to be protected from invertebrate pest attack or infestation with a pesticidally effective amount of at least one compound of the formula I as defined above, a stereoisomer thereof and/or at least one agriculturally acceptable salt thereof.

Preferably, the method of the invention serves for protecting plants or plant propagation material (such as seed) and the plant which grows therefrom from animal pest attack or infestation and comprises treating the plants or the plant propagation material (such as seed) with a pesticidally effective amount of a compound of the formula I or an agriculturally acceptable salt thereof as defined above or with a pesticidally effective amount of an agricultural composition as defined above and below. The method of the invention is not limited to the protection of the “substrate” (plant, plant propagation materials, soil material etc.) which has been treated according to the invention, but also has a preventive effect, thus, for example, according protection to a plant which grows from a treated plant propagation materials (such as seed), the plant itself not having been treated.

The invention furthermore relates to plant propagation material (such as seeds), comprising at least one compound of the formula I as defined above, a stereoisomer thereof and/or at least one agriculturally acceptable salt thereof.

The invention also provides a method for treating or protecting an animal from infestation or infection by invertebrate pests which comprises bringing the animal in contact with a pesticidally effective amount of at least one compound of the formula I as defined above, a stereoisomer thereof and/or at least one veterinarily acceptable salt thereof.

The compounds of the formula I and the pestidicidal compositions comprising them are effective agents for controlling arthropod pests and nematodes. Invertebrate pests controlled by the compounds of formula I include for example:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lamb-dina fiscellaria, Laphygma exigua, Leucoptera coffeella, Leucoptera scitella, Lithocol-letis blancardella, Lobesia botrana, Loxostege sticticalis, Lymantria dispar, Lymantria monacha, Lyonetia clerkella, Malacosoma neustria, Mamestra brassicae, Orgyia pseu-dotsugata, Ostrinia nubilalis, Panolis flammea, Pectinophora gossypiella, Peridroma saucia, Phalera bucephala, Phthorimaea operculella, Phyllocnistis citrella, Pieris bras-sicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;
beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12 punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria;
dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa;
thrips (Thysanoptera), e.g. Dichromothrips corbetti, Frankliniella fusca, Frankliniella occidentalis, Frankliniella tritici, Scirtothrips citri, Thrips oryzae, Thrips palmi and Thrips tabaci;
hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta;
heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor;
homopterans (Homoptera), e.g. Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus persicae, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Sogatella furcifera Trialeurodes vaporariorum, Toxoptera aurantiiand, and Viteus vitifolii;
termites (Isoptera), e.g. Calotermes flavicollis, Leucotermes flavipes, Reticulitermes flavipes, Reticulitermes lucifugus und Termes natalensis;
orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;
Arachnoidea, such as arachnids (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis;
Siphonatera, e.g. Xenopsylla cheopsis, Ceratophyllus spp;

The compositions and compounds of formula I are useful for the control of nematodes, especially plant parasitic nematodes such as root knot nematodes, Meloidogyne hapla, Meloidogyne incognita, Meloidogyne javanica, and other Meloidogyne species;

cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Paratylen-chus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

In a preferred embodiment of the invention the compounds of formula I are used for controlling insects or arachnids, in particular insects of the orders Lepidoptera, Coleoptera, Thysanoptera and Homoptera and arachnids of the order Acarina. The compounds of the formula I according to the present invention are particularly useful for controlling insects of the order Thysanoptera and Homoptera.

The compounds of formula I or the pesticidal compositions comprising them may be used to protect growing plants and crops from attack or infestation by invertebrate pests, especially insects, acaridae or arachnids by contacting the plant/crop with a pesticidally effective amount of compounds of formula I. The term “crop” refers both to growing and harvested crops.

The compounds of formula I can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, anti-foaming agents, anti-freezing agents, for seed treatment formulation also optionally colorants and/or binders and/or gelling agents.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (N-methylpyrrolidone [NMP], N-octylpyrrolidone [NOP]), acetates (glycol diacetate), glycols, fatty acid dimethylamides, fatty acids and fatty acid esters. In principle, solvent mixtures may also be used.

Suitable emulsifiers are non-ionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

A suitable preservative is e.g. dichlorophen.

Seed treatment formulations may additionally comprise binders and optionally colorants.

Binders can be added to improve the adhesion of the active materials on the seeds after treatment. Suitable binders are block copolymers EO/PO surfactants but also polyvinylalcohols, polyvinylpyrrolidones, polyacrylates, polymethacrylates, polybute-nes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®).

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers.

Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compounds of formula I can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound(s) may also be used successfully in the ultra-low-volume process (ULV), it being possible to apply formulations comprising over 95% by weight of active compound, or even to apply the active compound without additives.

The following are examples of formulations:

1. Products for dilution with water for foliar applications. For seed treatment purposes, such products may be applied to the seed diluted or undiluted.

A) Water-Soluble Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formula-tion with 10% (w/w) of active compound(s) is obtained.

B) Dispersible Concentrates (DC)

20 parts by weight of the active compound(s) are dissolved in 70 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

C) Emulsifiable Concentrates (EC)

15 parts by weight of the active compound(s) are dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

D) Emulsions (EW, EO, ES)

25 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

F) Water-Dispersible Granules and Water-Soluble Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

H) Gel-Formulation (GF)

In an agitated ball mill, 20 parts by weight of the active compound(s) are comminuted with addition of 10 parts by weight of dispersants, 1 part by weight of a gelling agent wetters and 70 parts by weight of water or of an organic solvent to give a fine active compound(s) suspension. Dilution with water gives a stable suspension of the active compound(s), whereby a formulation with 20% (w/w) of active compound(s) is obtained.

2. Products to be applied undiluted for foliar applications. For seed treatment purposes, such products may be applied to the seed diluted or undiluted.

I) Dustable Powders (DP, DS)

5 parts by weight of the active compound(s) are ground finely and mixed intimately with 95 parts by weight of finely divided kaolin. This gives a dustable product having 5% (w/w) of active compound(s)

J) Granules (GR, FG, GG, MG)

0.5 parts by weight of the active compound(s) is ground finely and associated with 95.5 parts by weight of carriers, whereby a formulation with 0.5% (w/w) of active compound(s) is obtained. Current methods are extrusion, spray-drying or the fluidized bed. This gives granules to be applied undiluted for foliar use.

K) ULV Solutions (UL)

10 parts by weight of the active compound(s) are dissolved in 90 parts by weight of an organic solvent, for example xylene. This gives a product having 10% (w/w) of active compound(s), which is applied undiluted for foliar use.

The compounds of formula I are also suitable for the treatment of plant propagation materials (such as seed). Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Other preferred FS formulations of compounds of formula I for seed treatment comprise from 0.5 to 80 wt % of the active ingredient, from 0.05 to 5 wt % of a wetter, from 0.5 to 15 wt % of a dispersing agent, from 0.1 to 5 wt % of a thickener, from 5 to 20 wt % of an anti-freeze agent, from 0.1 to 2 wt % of an anti-foam agent, from 1 to 20 wt % of a pigment and/or a dye, from 0 to 15 wt % of a sticker/adhesion agent, from 0 to 75 wt % of a filler/vehicle, and from 0.01 to 1 wt % of a preservative.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds of formula I are effective through both contact (via soil, glass, wall, bed net, carpet, plant parts or animal parts), and ingestion (bait, or plant part).

For use against ants, termites, wasps, flies, mosquitoes, crickets, or cockroaches, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product which is sufficiently attractive to incite insects such as ants, termites, wasps, flies, mosquitoes, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g in spray cans), oil sprays or pump sprays are highly suitable for the non-professional user for controlling pests such as flies, fleas, ticks, mosquitoes or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N methylpyrrolidone, dimethyl sulphoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and their respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and their respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for exam-pie are N,N-diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and diethylenically unsaturated hydrocarbons, such as styrene, and aliphatic diens, such as butadiene.

The impregnation of curtains and bednets is done in general by dipping the textile material into emulsions or dispersions of the active compounds of formula I or spraying them onto the nets.

Methods which can be employed for treating the seed are, in principle, all suitable seed treatment and especially seed dressing techniques known in the art, such as seed coating (e.g. seed pelleting), seed dusting and seed imbibition (e.g. seed soaking). Here, “seed treatment” refers to all methods that bring seeds and the compounds of formula I into contact with each other, and “seed dressing” to methods of seed treatment which provide the seeds with an amount of the compounds of formula I, i.e. which generate a seed comprising the compound of formula I. In principle, the treatment can be applied to the seed at any time from the harvest of the seed to the sowing of the seed. The seed can be treated immediately before, or during, the planting of the seed, for example using the “planter's box” method. However, the treatment may also be carried out several weeks or months, for example up to 12 months, before planting the seed, for example in the form of a seed dressing treatment, without a substantially reduced efficacy being observed.

Expediently, the treatment is applied to unsown seed. As used herein, the term “unsown seed” is meant to include seed at any period from the harvest of the seed to the sowing of the seed in the ground for the purpose of germination and growth of the plant.

Specifically, a procedure is followed in the treatment in which the seed is mixed, in a suitable device, for example a mixing device for solid or solid/liquid mixing partners, with the desired amount of seed treatment formulations, either as such or after previous dilution with water, until the composition is distributed uniformly on the seed. If appropriate, this is followed by a drying step.

The compounds of formula I or veterinarily acceptable salts thereof are in particular also suitable for being used for combating parasites in and on animals.

A further object of the present invention is therefore to provide new methods for controlling parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula I or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula I or veterinarily acceptable salts thereof or a composition comprising it.

The present invention also provides a non-therapeutic method for treating, controlling, preventing and protecting animals against infestation and infection by parasites, which comprises applying to a locus a parasiticidally effective amount of a compound of formula I or the enantiomers or veterinarily acceptable salts thereof or a composition comprising it.

The invention also provides a process for the preparation of a composition for treating, controlling, preventing or protecting animals against infestation or infection by parasites which comprises including a parasiticidally effective amount of a compound of formula I or the veterinarily acceptable salts thereof or a composition comprising it.

The invention relates further to the use of compounds of formula I for treating, controlling, preventing or protecting animals against infestation or infection by parasites. The invention relates also to the use of a compound of formula I, or a composition comprising it, for the manufacture of a medicament for the therapeutic treatment of animals against infections or infestions by parasites.

Activity of compounds against agricultural pests does not suggest their suitability for control of endo- and ectoparasites in and on animals which requires, for example, low, nonemetic dosages in the case of oral application, metabolic compatibility with the animal, low toxicity, and a safe handling.

Surprisingly, it has been found that compounds of formula I are suitable for combating endo- and ectoparasites in and on animals.

Compounds of formula I or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula I or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I or veterinarily acceptable salts thereof and compositions comprising them are suitable for systemic and/or non-systemic control of ecto- and/or endoparasites. They are active against all or some stages of development.

The compounds of formula I are especially useful for combating ectoparasites.

The compounds of formula I are especially useful for combating endoparasites.

The compounds of formula I are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,
cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,
actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,
bugs (Heteropterida): Cimex lectularius, Cimex hemipterus, Reduvius senilis, Triatoma spp., Rhodnius ssp., Panstrongylus ssp. and Arilus critatus,
Anoplurida, e.g. Haematopinus spp., Linognathus spp., Pediculus spp., Phtirus spp., and Solenopotes spp,
Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,
Rhabditida, e.g. Rhabditis spp, Strongyloides spp., Helicephalobus spp,
Strongylida, e.g. Strongylus spp., Ancylostoma spp., Necator americanus, Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stepha-nurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,
Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,
Camallanida, e.g. Dracunculus medinensis (guinea worm)
Spirurida, e.g. Thelazia spp. Wuchereria spp., Brugia spp., Onchocerca spp., Dirofilari spp. a, Dipetalonema spp., Setaria spp., Elaeophora spp., Spirocerca lupi, and Habronema spp.,
Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,
Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula I and compositions containing them are particularly useful for the control of pests from the orders Diptera, Siphonaptera and Ixodida.

Moreover, the use of compounds of formula I and compositions containing them for combating mosquitoes is especially preferred.

The use of the compounds of formula I and compositions containing them for combating flies is a further preferred embodiment of the present invention.

Furthermore, the use of the compounds of formula I and compositions containing them for combating fleas is especially preferred.

The use of the compounds of formula I and of the compositions containing them for combating ticks is a further preferred embodiment of the present invention.

The compounds of formula I also are especially useful for combating endoparasites (roundworms nematoda, thorny headed worms and planarians).

The compounds of formula I can be effective through both contact (via soil, glass, wall, bed net, carpet, blankets or animal parts) and ingestion (e.g. baits).

The present invention relates to the therapeutic and the non-therapeutic use of compounds of formula I for controlling and/or combating parasites in and/or on animals.

The compounds of formula I may be used to protect the animals from attack or infestation by parasites by contacting them with a parasitically effective amount of compounds of formula I. As such, “contacting” includes both direct contact (applying the compounds/compositions directly on the parasite, e.g. also at it's locus, and optionally also administrating the compounds/composition directly on the animal) and indirect contact (applying the compounds/compositions to the locus of the parasite). The contact of the parasite through application to its locus is an example of a non-therapeutic use of compounds of formula I.

“Locus” as defined above means the habitat, food supply, breeding ground, area, material or environment in which a parasite is growing or may grow outside of the animal. The compounds of the invention can also be applied preventively to places at which occurrence of the pests or parasites is expected.

Administration to the animal can be carried out both prophylactically and therapeutically.

Administration of the active compounds is carried out directly or in the form of suitable preparations, orally, topically/dermally or parenterally.

For oral administration to warm-blooded animals, the compounds of formula I may be formulated as animal feeds, animal feed premixes, animal feed concentrates, pills, solutions, pastes, suspensions, drenches, gels, tablets, boluses and capsules. In addition, the compounds of formulae I may be administered to the animals in their drinking water. For oral administration, the dosage form chosen should provide the animal with 0.01 mg/kg to 100 mg/kg of animal body weight per day of the compounds of formula I, preferably with 0.5 mg/kg to 100 mg/kg of animal body weight per day.

Alternatively, the compounds of formula I may be administered to animals parenterally, for example, by intraruminal, intramuscular, intravenous or subcutaneous injection. The compounds of formula I may be dispersed or dissolved in a physiologically acceptable carrier for subcutaneous injection. Alternatively, the compounds of formula I may be formulated into an implant for subcutaneous administration. In addition the compounds of formula I may be transdermally administered to animals. For parenteral administration, the dosage form chosen should provide the animal with 0.01 mg/kg to 100 mg/kg of animal body weight per day of the compounds of formula I.

The compounds of formula I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds of formula I. In addition, the compounds of formula I may be formulated as ear tags for animals, particularly quadrupeds such as cattle and sheep.

Suitable preparations are:

Solutions such as oral solutions, concentrates for oral administration after dilution, solutions for use on the skin or in body cavities, pouring-on formulations, gels;

Emulsions and suspensions for oral or dermal administration; semi-solid preparations;

Formulations in which the active compound is processed in an ointment base or in an oil-in-water or water-in-oil emulsion base;

Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methylpyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents are water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N methylpyrrolidone, 2-pyrrolidone, 2,2-dimethyl-4-oxy-methylene-1,3-diox-olane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are:

liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyldodecanol, cetylstearyl alcohol, oleyl alcohol, and fatty acids such as oleic acid and mixtures thereof.

Suitable hydrophilic phases are: water, alcohols such as propylene glycol, glycerol, sorbitol and mixtures thereof.

Suitable emulsifiers are:

non-ionic surfactants, e.g. polyethoxylated castor oil, polyethoxylated sorbitan monooleate, sorbitan monostearate, glycerol monostearate, polyoxyethyl stearate, alkylphenol polyglycol ether;
ampholytic surfactants such as di-sodium N-lauryl-p-iminodipropionate or lecithin; anionic surfactants, such as sodium lauryl sulfate, fatty alcohol ether sulfates, mono/dialkyl polyglycol ether orthophosphoric acid ester monoethanolamine salt; cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compounds of formula I.

Generally, it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula I are applied dermally/topically.

In a further preferred embodiment, the topical application is conducted in the form of compound-containing shaped articles such as collars, medallions, ear tags, bands for fixing at body parts, and adhesive strips and foils.

Generally, it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

Compositions to be used according to this invention may also contain other active ingredients, for example other pesticides, insecticides, herbicides, fungicides, other pesticides, or bactericides, fertilizers such as ammonium nitrate, urea, potash, and super-phosphate, phytotoxicants and plant growth regulators, safeners and nematicides.

These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents can be admixed with the agents used according to the invention in a weight ratio of 1:10 to 10:1. Mixing the compounds of formula I or the compositions comprising them in the use form as pesticides with other pesticides frequently results in a broader pesticidal spectrum of action.

The following list M of pesticides together with which the compounds according to the invention can be used and with which potential synergistic effects might be produced, is intended to illustrate the possible combinations, but not to impose any limitation:

M.1. Organo(thio)phosphate compounds: acephate, azamethiphos, azinphos-ethyl, azinphos-methyl, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;
M.2. Carbamate compounds: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate;
M.3. Pyrethroid compounds: acrinathrin, allethrin, d-cis-trans allethrin, d-trans allethrin, bifenthrin, bioallethrin, bioallethrin S-cyclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, metofluthrin, permethrin, phenothrin, prallethrin, profluthrin, pyrethrin (pyrethrum), resmethrin, silafluofen, tefluthrin, tetramethrin, tralomethrin, transfluthrin;
M.4. Juvenile hormone mimics: hydroprene, kinoprene, methoprene, fenoxycarb, pyriproxyfen;
M.5. Nicotinic receptor agonists/antagonists compounds: acetamiprid, bensultap, cartap hydrochloride, clothianidin, dinotefuran, imidacloprid, thiamethoxam, nitenpyram, nicotine, spinosad (allosteric agonist), spinetoram (allosteric agonist), thiacloprid, thiocyclam, thiosultap-sodium and AKD1022.
M.6. GABA gated chloride channel antagonist compounds: chlordane, endosulfan, gamma-HCH (lindane); ethiprole, fipronil, pyrafluprole, pyriprole
M.7. Chloride channel activators: abamectin, emamectin benzoate, milbemectin, lepimectin;
M.8. METI I compounds: fenazaquin, fenpyroximate, pyrimidifen, pyridaben, tebufenpyrad, tolfenpyrad, flufenerim, rotenone;
M.9. METI II and III compounds: acequinocyl, fluacyprim, hydramethylnon;
M.10. Uncouplers of oxidative phosphorylation: chlorfenapyr, DNOC;
M.11. Inhibitors of oxidative phosphorylation: azocyclotin, cyhexatin, diafenthiuron, fenbutatin oxide, propargite, tetradifon;
M.12. Moulting disruptors: cyromazine, chromafenozide, halofenozide, methoxyfenozide, tebufenozide;
M.13. Synergists: piperonyl butoxide, tribufos;
M.14. Sodium channel blocker compounds: indoxacarb, metaflumizone;
M.15. Fumigants: methyl bromide, chloropicrin sulfuryl fluoride;
M.16. Selective feeding blockers: crylotie, pymetrozine, flonicamid;
M.17. Mite growth inhibitors: clofentezine, hexythiazox, etoxazole;
M.18. Chitin synthesis inhibitors: buprofezin, bistrifluoron, chlorfluazuron, diflubenzuron, flucycloxuron, flufenoxuron, hexaflumuron, lufenuron, novaluron, noviflumuron, teflubenzuron, triflumuron;
M.19. Lipid biosynthesis inhibitors: spirodiclofen, spiromesifen, spirotetramat;
M.20. Octapaminergic agonsits: amitraz;
M.21. Ryanodine receptor modulators: flubendiamide and the phtalamid compound (R)-, (S)-3-Chlor-N-1-{2-methyl-4-[1,2,2,2-tetrafluor-1-(trifluormethyl)ethyl]phenyl}-N2-(1-methyl-2-methylsulfonylethyl)phthalamid (M21.1)
M.22. Isoxazoline compounds: 4-[5-(3,5-Dichloro-phenyl)-5-trifluoromethyl-4,5-dihydro-isoxazol-3-yl]-2-methyl-N-pyridin-2-ylmethyl-benzamide (M22.1), 4-[5-(3,5-Dichloro-phenyl)-5-trifluoromethyl-4,5-dihydro-isoxazol-3-yl]-2-methyl-N-(2,2,2-trifluoro-ethyl)-benzamide (M22.2), 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide (M22.3), 4-[5-(3,5-Dichloro-phenyl)-5-trifluoromethyl-4,5-dihydro-isoxazol-3-yl]-naphthalene-1-carboxylic acid [(2,2,2-trifluoro-ethylcarbamoyl)-methyl]-amide (M22.4) 4-[5-(3,5-Dichlorophenyl)-5-trifluoromethyl-4,5-dihydro-isoxazol-3-yl]-N-[(methoxyimino)methyl]-2-methylbenzamide (M22.5), 4-[5-(3-Chloro-5-trifluoromethylphenyl)-5-trifluoromethyl-4,5-dihydro-isoxazol-3-yl]-2-methyl-N-[(2,2,2-trifluoro-ethylcarbamoyl)-methyl]-benzamide (M22.6), 4-[5-(3-Chloro-5-trifluoromethyl-phenyl)-5-trifluoromethyl-4,5-dihydro-isoxazol-3-yl]-naphthalene-1-carboxylic acid [(2,2,2-trifluoro-ethylcarbamoyl)-methyl]-amide (M22.7) and 5-[5-(3,5-Dichloro-4-fluorophenyl)-5-trifluoromethyl-4,5-dihydro-isoxazol-3-yl]-2-[1,2,4]triazol-1-yl-benzonitrile (M22.8);
M.23. Anthranilamide compounds: chloranthraniliprole, cyantraniliprole, 5-Bromo-2-(3-chloro-pyridin-2-yl)-2H-pyrazole-3-carboxylic acid [4-cyano-2-(1-cyclopropyl-ethylcarbamoyl)-6-methyl-phenyl]-amide (M23.1), 5-Bromo-2-(3-chloro-pyridin-2-yl)-2H-pyrazole-3-carboxylic acid [2-chloro-4-cyano-6-(1-cyclopropyl-ethylcarbamoyl)-phenyl]-amide (M23.2), 5-Bromo-2-(3-chloro-pyridin-2-yl)-2H-pyrazole-3-carboxylic acid [2-bromo-4-cyano-6-(1-cyclopropyl-ethylcarbamoyl)-phenyl]-amide (M23.3), 5-Bromo-2-(3-chloro-pyridin-2-yl)-2H-pyrazole-3-carboxylic acid [2-bromo-4-chloro-6-(1-cyclopropyl-ethylcarbamoyl)-phenyl]-amide (M23.4), 5-Bromo-2-(3-chloro-pyridin-2-yl)-2H-pyrazole-3-carboxylic acid [2,4-dichloro-6-(1-cyclopropyl-ethylcarbamoyl)-phenyl]-amide (M23.5), 5-Bromo-2-(3-chloro-pyridin-2-yl)-2H-pyrazole-3-carboxylic acid [4-chloro-2-(1-cyclopropyl-ethylcarbamoyl)-6-methyl-phenyl]-amide (M23.6), N′-(2-{[5-Bromo-2-(3-chloro-pyridin-2-yl)-2H-pyrazole-3-carbonyl]-amino}-5-chloro-3-methyl-benzoyl)-hydrazinecarboxylic acid methyl ester (M23.7), N′-(2-{[5-Bromo-2-(3-chloro-pyridin-2-yl)-2H-pyrazole-3-carbonyl]-amino}-5-chloro-3-methyl-benzoyl)-N′-methyl-hydrazinecarboxylic acid methyl ester (M23.8), N′-(2-{[5-Bromo-2-(3-chloro-pyridin-2-yl)-2H-pyrazole-3-carbonyl]-amino}-5-chloro-3-methyl-benzoyl)-N,N′-dimethyl-hydrazinecarboxylic acid methyl ester (M23.9), N′-(3,5-Dibromo-2-{[5-bromo-2-(3-chloro-pyridin-2-yl)-2H-pyrazole-3-carbonyl]-amino}-benzoyl)-hydrazinecarboxylic acid methyl ester (M23.10), N′-(3,5-Dibromo-2-{[5-bromo-2-(3-chloro-pyridin-2-yl)-2H-pyrazole-3-carbonyl]-amino}-benzoyl)-N′-methyl-hydrazinecarboxylic acid methyl ester (M23.11) and N′-(3,5-Dibromo-2-{[5-bromo-2-(3-chloro-pyridin-2-yl)-2H-pyrazole-3-carbonyl]-amino}-benzoyl)-N,N′-dimethyl-hydrazinecarboxylic acid methyl ester (M23.12);
M.24. Malononitrile compounds: 2-(2,2,3,3,4,4,5,5-octafluoropentyl)-2-(3,3,3-trifluoropropyl)malononitrile (CF₂H—CF₂—CF₂—CF₂—CH₂—C(CN)₂—CH₂—CH₂—CF₃) (M24.1) and 2-(2,2,3,3,4,4,5,5-octafluoropentyl)-2-(3,3,4,4,4-pentafluorobutyl)-malonodinitrile (CF₂H—CF₂—CF₂—CF₂—CH₂—C(CN)₂—CH₂—CH₂—CF₂—CF₃) (M24.2);
M.25. Microbial disruptors: Bacillus thuringiensis subsp. Israelensi, Bacillus sphaericus, Bacillus thuringiensis subsp. Aizawai, Bacillus thuringiensis subsp. Kurstaki, Bacillus thuringiensis subsp. Tenebrionis;
M.26. Aminofuranone compounds:
4-{[(6-Bromopyrid-3-yl)methyl](2-fluoroethyl)amino}furan-2(5H)-on (M26.1),
4-{[(6-Fluoropyrid-3-yl)methyl](2,2-difluoroethyl)amino}furan-2(5H)-on (M26.2),
4-{[(2-Chloro-1,3-thiazolo-5-yl)methyl](2-fluoroethyl)amino}furan-2(5H)-on (M26.3),
4-{[(6-Chloropyrid-3-yl)methyl](2-fluoroethyl)amino}furan-2(5H)-on (M26.4),
4-{[(6-Chloropyrid-3-yl)methyl](2,2-difluoroethyl)amino}furan-2(5H)-on (M26.5),
4-{[(6-Chloro-5-fluoropyrid-3-yl)methyl](methyl)amino}furan-2(5H)-on (M26.6),
4-{[(5,6-Dichloropyrid-3-yl)methyl](2-fluoroethyl)amino}furan-2(5H)-on (M26.7),
4-{[(6-Chloro-5-fluoropyrid-3-yl)methyl](cyclopropyl)amino}furan-2(5H)-on (M26.8),
4-{[(6-Chloropyrid-3-yl)methyl](cyclopropyl)amino}furan-2(5H)-on (M26.9) and
4-{[(6-Chloropyrid-3-yl)methyl](methyl)amino}furan-2(5H)-on (M26.10);
   M.27. Various compounds: aluminium phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, organic sulfur compounds, tartar emetic, sulfoxaflor, N—R′-2,2-dihalo-1-R″cyclopropanecarboxamide-2-(2,6-dichloro-α,α,α-trifluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, 4-But-2-ynyloxy-6-(3,5-dimethyl-piperidin-1-yl)-2-fluoro-pyrimidine (M27.1), Cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester (M27.2) and 8-(2-Cyclopropylmethoxy-4-trifluoromethyl-phenoxy)-3-(6-trifluoromethyl-pyridazin-3-yl)-3-aza-bicyclo[3.2.1]octane (M27.3).

The commercially available compounds of the group M may be found in The Pesticide Manual, 13th Edition, British Crop Protection Council (2003) among other publications.

Paraoxon and their preparation have been described in Farm Chemicals Handbook, Volume 88, Meister Publishing Company, 2001. Flupyrazofos has been described in Pesticide Science 54, 1988, p. 237-243 and in U.S. Pat. No. 4,822,779. AKD 1022 and its preparation have been described in U.S. Pat. No. 6,300,348. The anthranilamides M23.1 to M23.6 have been described in WO 2008/72743 and WO 200872783, those M23.7 to M23.12 in WO2007/043677. The phthalamide M 21.1 is known from WO 2007/101540. The alkynylether compound M27.1 is described e.g. in JP 2006131529. Organic sulfur compounds have been described in WO 2007060839. The isoxazoline compounds M 22.1 to M 22.8 have been described in e.g. WO2005/085216, WO 2007/079162, WO 2007/026965, WO 2009/126668 and WO2009/051956. The aminofuranone compounds M 26.1 to M 26.10 have been described eg. in WO 2007/115644. The pyripyropene derivative M 27.2 has been described in WO 2008/66153 and WO 2008/108491. The pyridazin compound M 27.3 has been described in JP 2008/115155.

Malononitrile compounds as those (M24.1) and (M24.2) have been described in WO 02/089579, WO 02/090320, WO 02/090321, WO 04/006677, WO 05/068423, WO 05/068432 and WO 05/063694.

Fungicidal mixing partners are those selected from the group consisting of acylalanines such as benalaxyl, metalaxyl, ofurace, oxadixyl,

amine derivatives such as aldimorph, dodine, dodemorph, fenpropimorph, fenpropidin, guazatine, iminoctadine, spiroxamin, tridemorph,
anilinopyrimidines such as pyrimethanil, mepanipyrim or cyrodinyl,
antibiotics such as cycloheximid, griseofulvin, kasugamycin, natamycin, polyoxin or streptomycin,
azoles such as bitertanol, bromoconazole, cyproconazole, difenoconazole, diniconazole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol,
dicarboximides such as iprodion, myclozolin, procymidon, vinclozolin,
dithiocarbamates such as ferbam, nabam, maneb, mancozeb, metam, metiram, propineb, polycarbamate, thiram, ziram, zineb,
heterocyclic compounds such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine,
copper fungicides such as Bordeaux mixture, copper acetate, copper oxychloride, basic copper sulfate,
nitrophenyl derivatives such as binapacryl, dinocap, dinobuton, nitrophthalisopropyl, phenylpyrroles such as fenpiclonil or fludioxonil,
sulfur,
other fungicides such as acibenzolar-S-methyl, benthiavalicarb, carpropamid, chlorothalonil, cyflufenamid, cymoxanil, diclomezin, diclocymet, diethofencarb, edifen-phos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofos-methyl, quintozene, zoxamid,
strobilurins such as azoxystrobin, dimoxystrobin, fluoxastrobin, kresoxim-methyl, metominostrobin, orysastrobin, picoxystrobin or trifloxystrobin,
sulfenic acid derivatives such as captafol, captan, dichlofluanid, folpet, tolylfluanid, cinnemamides and analogs such as dimethomorph, flumetover or flumorph.

The invertebrate pest, i.e. arthropodes and nematodes, the plant, soil or water in which the plant is growing can be contacted with the present compound(s) of formula I or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

Moreover, invertebrate pests may be controlled by contacting the target pest, its food supply, habitat, breeding ground or its locus with a pesticidally effective amount of compounds of formula I. As such, the application may be carried out before or after the infection of the locus, growing crops, or harvested crops by the pest.

“Locus” means a habitat, breeding ground, cultivated plants, plant propagation material (such as seed), soil, area, material or environ-ment in which a pest or parasite is growing or may grow.

In general “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of formula I and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of are applied not only to the surrounding soil surface or into the under-floor soil in order to protect wooden materials but it can also be applied to lumbered articles such as surfaces of the under-floor concrete, alcove posts, beams, plywood, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of formula I can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95% by weight, preferably from 0.1 to 45% by weight, and more preferably from 1 to 25% by weight of at least one repellent and/or insecticide.

For use in bait compositions, the typical content of active ingredient is from 0.001% by weight to 15% by weight, desirably from 0.001% by weight to 5% by weight of active compound.

For use in spray compositions, the content of active ingredient is from 0.001 to 80% by weight, preferably from 0.01 to 50% by weight and most preferably from 0.01 to 15% by weight.

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the active ingredients are generally from 0.1 g to 10 kg per 100 kg of seed, preferably from 1 g to 5 kg per 100 kg of seed, in particular from 1 g to 200 g per 100 kg of seed.

The present invention is now illustrated in further detail by the following examples.

### A. PREPARATION EXAMPLES

### S.1 Synthesis of 3-[5-(3,5-dichloro-phenyl)-5-trifluoromethyl-4,5-dihydro-isoxazol-3-yl]-benzylamine hydrochloride (compound 3-1 of table C.3)

### Step 1: Preparation of 3-(hydroxyimino-methyl)-benzonitrile

A solution of 3-formyl-benzonitrile (14 g, 106.8 mmol), TEA (21 g, 213.7 mmol) and hydroxylamine hydrochloride (7 g, 106.8 mmol) in CH₂Cl₂ was stirred at room temperature over night. The resulting solution was washed with water. The aqueous phase was extracted twice with CH₂Cl₂. The organic phase was dried over Na₂SO₄ and concentrated to give the title compound (13.7 g, 80%).

¹H NMR (400 MHz, CDCl₃): δ [delta]=8.03-8.05 (m, 2H), 7.54-7.56 (m, 2H), 6.84 (s, 1H)

### Step 2: Preparation of 3-cyano benzohydroxamic acid chloride

To a solution of 3-(hydroxyimino-methyl)-benzonitrile (i.e. the product of Step 1, 13.7 g, 94 mmol) in THF (200 mL) was added dropwise concentrated hydrochloric acid (30 g) at 0° C. Then 66 mL 8% NaOCl aqueous solution was added dropwise under ice cooling. After the completion of the addition, it was stirred 90 min at 0° C. The resulting solution was washed with water. The aqueous phase was extracted twice with EtOAc. The organic phase was dried over Na₂SO₄ and concentrated to give the title compound (15.6 g, 92%).

¹H NMR (400 MHz, CDCl₃): δ [delta]=8.08-8.12 (m, 2H), 7.72-7.74 (m, 1H), 7.52-7.57 (m, 1H).

### Step 3: Preparation of 3-[5-(3,5-dichloro-phenyl)-5-trifluoromethyl-4,5-dihydro-isoxazol-3-yl]-benzonitrile

A solution of 3-cyano benzohydroxamic acid chloride (i.e. the product of Step 2, 3.5 g, 19.1 mmol), NaHCO₃ (2.6 g, 30.6 mmol) and 1,3-dichloro-5-(1-trifluoromethyl-vinyl)benzene (5.0 g, 21 mmol) in THF (50 mL) was refluxed over night. The resulting solution was washed with water. The aqueous phase was extracted twice with ethyl acetate. The organic phase was dried over Na₂SO₄ and concentrated to give the title compound (7.1 g, 97%).

¹H NMR (400 MHz, CDCl₃): δ [delta]=7.82-7.88 (m, 2H), 7.66-7.69 (m, 1H,), 7.50-7.52 (m, 1H), 7.43-7.45 (m, 2H), 7.36-7.37 (m, 1H), 4.02 (d, 1H), 3.60 (d, 1H).

### Step 4: Preparation of 3-[5-(3,5-dichloro-phenyl)-5-trifluoromethyl-4,5-dihydro-isoxazol-3-yl]-benzylamine hydrochloride (compound 3-1 of Table C-3)

To a stirring suspension of 3-[5-(3,5-dichloro-phenyl)-5-trifluoromethyl-4,5-dihydro-isoxazol-3-yl]-benzonitrile (i.e. the product of Step 3, 1.0 g, 2.6 mmol) in THF (50 mL) was added LiAlH₄ (200 mg, 5.2 mmol) in portions at −10° C. under N₂ protection. The solution was stirred at 0° C. for 1 hour, before it was quenched with water. The mixture was filtered, and the filtration was concentrated and purified by preparative-HPLC to afford the title compound (310 mg, 30%).

¹H NMR (400 MHz, DMSO-d₆): δ [delta]=8.39 (s, 2H), 7.93 (s, 1H), 7.80 (m, 1H), 7.66-7.68 (m, 1H), 7.61-7.63 (m, 3H), 7.54-7.56 (m, 1H), 4.29-4.33 (m, 2H), 4.05-4.06 (m, 2H).

### S.2 Preparation of N-{3-[5-(3,5-dichloro-phenyl)-5-trifluoromethyl-4,5-dihydro-isoxazol-3-yl]-benzyl}-propionamide (compound 3-2 of table C.3)

To a solution of 3-[5-(3,5-dichloro-phenyl)-5-trifluoromethyl-4,5-dihydro-isoxazol-3-yl]-benzylamine hydrochloride (i.e. the product of Example S.1, Step 4, 2 g crude) and triethyl amine (1.0 g, 10.4 mmol) in CH₂Cl₂ (100 mL) was added propionic acid chloride (0.5 g, 5.7 mmol) dropwise with stirring at room temperature. The resulting solution was washed with NaHCO₃. The organic phase was dried over Na₂SO₄ and concentrated and purified by preparative-HPLC to afford the title compound (260 mg, 11%)

¹H NMR (400 MHz, CDCl₃): δ [delta]=7.52 (s, 1H), 7.48-7.49 (m, 1H), 7.44-7.47 (m, 2H), 7.30-7.35 (m, 3H), 5.74 (br. s, 1H), 4.39-4.40 (m, 2H), 4.01 (d, 1H), 3.62 (d, 1H), 2.20 (q, 2H), 1.11 (t, 3H).

### S.3 Preparation of N-{3-[5-(3,5-dichloro-phenyl)-5-trifluoromethyl-4,5-dihydro-isoxazol-3-yl]-benzyl}-acetamide (compound 3-3 of Table C.3)

To a solution of 3-[5-(3,5-dichloro-phenyl)-5-trifluoromethyl-4,5-dihydro-isoxazol-3-yl]-benzylamine hydrochloride (i.e. the product of Example S.1, Step 4, 2 g crude) and triethyl amine (1.0 g, 10.4 mmol) in CH₂Cl₂ (100 mL) was added acetyl chloride (0.5 g, 6 mmol) drop-wise with stirring at room temperature. The resulting solution was washed with NaHCO₃. The organic phase was dried over Na₂SO₄ and concentrated and purified by preparative-HPLC to afford the title compound (300 mg, 14%)

¹H NMR (400 MHz, CDCl₃): δ [delta]=7.53 (s, 1H), 7.49-7.50 (m, 1H), 7.44 (s, 2H), 7.35 (m, 1H), 7.32 (m, 2H), 5.73 (br. s, 1H), 4.38-4.40 (m, 2H), 4.01 (d, 1H), 3.62 (d, 1H), 1.98 (s, 3H).

### S.4 Preparation of N-{3-[5-(3,5-dichloro-phenyl)-5-trifluoromethyl-4,5-dihydro-isoxazol-3-yl]-benzyl}-2-methylsulfanyl-acetamide (compound 3-4 of Table C.3)

A solution of 3-[5-(3,5-dichloro-phenyl)-5-trifluoromethyl-4,5-dihydro-isoxazol-3-yl]-benzylamine hydrochloride (i.e. the product of Example S.1, Step 4, 500 mg, 1.3 mmol), methylsulfanyl acetic acid (140 mg, 1.3 mmol), diisopropyl carbodiimide (250 mg, 2.0 mmol) and 4-N,N-dimethylamino pyridine (80 mg, 0.7 mmol) in THF (50 mL) was stirred over night at room temperature. The resulting solution was washed with water. The organic phase was dried over Na₂SO₄ and concentrated and purified by preparative-HPLC to afford the title compound (350 mg, 65%).

¹H NMR (400 MHz, CDCl₃): δ [delta]=7.64 (s, 1H), 7.57-7.59 (m, 1H), 7.53 (s, 2H), 7.42-7.45 (m, 3H), 4.54 (d, 2H), 4.10 (d, 1H), 3.71 (d, 1H), 3.30 (s, 2H), 2.14 (s, 3H).

### S.5 Preparation of N-{3-[5-(3,5-dichloro-phenyl)-5-trifluoromethyl-4,5-dihydro-isoxazol-3-yl]-benzyl}-butyramide (compound 3-5 of table C.3)

To a solution of 3-[5-(3,5-dichloro-phenyl)-5-trifluoromethyl-4,5-dihydro-isoxazol-3-yl]-benzylamine hydrochloride (i.e. the product of Example S.1, Step 4, 2 g crude) and triethyl amine (1.0 g, 10.4 mmol) in CH₂Cl₂ (100 mL) was added butanoic acid chloride (0.6 g, 5.7 mmol) drop-wise with stirring at room temperature. The resulting solution was washed with NaHCO₃. The organic phase was dried over Na₂SO₄ and concentrated and purified by preparative-HPLC to afford the title compound (350 mg, 15%).

¹H NMR (400 MHz, CDCl₃): δ [delta]=7.52 (s, 1H), 7.51 (s, 1H), 7.44-7.49 (m, 2H), 7.32-7.36 (m, 1H), 7.18-7.19 (m, 2H), 4.41 (d, 2H), 4.01 (d, 1H), 3.62 (d, 1H), 2.16 (m, 2H), 1.60-1.66 (m, 2H), 0.94 (t, 3H).

### S.6 Preparation of 3-[5-(3,5-dichloro-phenyl)-5-trifluoromethyl-4,5-dihydro-isoxazol-3-yl]-N-hydroxy-benzamidine (compound 4-1 of Table C.4)

To a solution of 3-[5-(3,5-dichloro-phenyl)-5-trifluoromethyl-4,5-dihydro-isoxazol-3-yl]-benzonitrile (i.e. the product of Example S.1, Step 3, 0.8 g, 2.08 mmol) in ethanol (20 mL) was added hydroxylamine hydrochloride (0.16 g, 2.40 mmol) and triethyl amine (0.32 g, 3.17 mmol). The solution was stirred at 80° C. under N₂ overnight. The mixture was partitioned by EtOAc and water and extracted with EtOAc. The organic layer was dried and concentrated to give the crude product, which was purified by preparative-HPLC to afford the title compound (0.3 g, 35%).

¹H NMR: (400 MHz, CDCl₃): δ [delta]=7.64-7.83 (m, 3H), 7.47-7.35 (m, 4H), 4.96-4.95 (d, 1H), 4.09-4.03 (m, 1H), 3.71-3.65 (m, 1H).

### S.7 Preparation of 3-{3-[5-(3,5-dichloro-phenyl)-5-trifluoromethyl-4,5-dihydro-isoxazol-3-yl]-phenyl}-5-methyl-[1,2,4]oxadiazole (compound 4-2 of Table C.4)

To a solution of 3-[5-(3,5-dichloro-phenyl)-5-trifluoromethyl-4,5-dihydro-isoxazol-3-yl]-N-hydroxy-benzamidine (i.e. the product of Example S.6, 1.22 g, 2.93 mmol) in dioxane (20 mL) was added acetic anhydride (0.71 g, 6.73 mmol). The solution was stirred at 80° C. under N₂ overnight. The mixture was partitioned by EtOAc and water and extracted with EtOAc. The organic layer was dried and concentrated to give the crude product, which was purified by preparative-HPLC to give the title compound (264 mg, 20%).

¹H NMR: (400 MHz, CDCl₃): δ [delta]=8.22-8.19 (m, 1H), 8.18-8.16 (m, 1H), 7.97-7.94 (m, 1H), 7.59-7.43 (m, 3H), 7.43-7.44 (d, 1H), 4.18 (d, 1H), 3.78 (d, 1H), 2.69 (s, 3H).

### S.8 Preparation of 1-{3-[5-(3,5-dichloro-phenyl)-5-trifluoromethyl-4,5-dihydro-isoxazol-3-yl]-phenyl}-1H-[1,2,4]triazole (compound 1-2 of Table C.1)

### Step 1: Preparation of 3-[1,2,4]triazol-1-yl-benzaldehyde

A solution of 3-fluoro-benzaldehyde (20.0 g, 160 mmol), the 1,2,4-triazole (33.0 g, 480 mmol) and K₂CO₃ (44.0 g, 320 mmol) in DMSO was stirred over night at 150° C. The resulting solution was washed with water. The aqueous phase was extracted twice with EtOAc. The organic phase was dried over Na₂SO₄ and concentrated to give the title compound (3 g, 5.8%).

### Step 2: Preparation of 3-[1,2,4]triazol-1-yl-benzaldehyde oxime

A solution of 3-[1,2,4]triazol-1-yl-benzaldehyde (i.e. the product of Step 1, 3.0 g, 17.2 mmol), triethyl amine (3.5 g, 34.4 mmol) and hydroxylamine hydrochloride (1.2 g, 17.2 mmol) in CH₂Cl₂ was stirred at room temperature over night. The resulting solution was washed with water. The aqueous phase was extracted twice with CH₂Cl₂. The organic phase was dried over Na₂SO₄ and concentrated to give the title compound (2 g, 75%).

### Step 3: Preparation of N-hydroxy-3-[1,2,4]triazol-1-yl-benzimidic chloride

To a solution of 3-[1,2,4]triazol-1-yl-benzaldehyde oxime (i.e. the product of Step 2, 2.0 g, 10 mmol) in THF (50 mL) was added dropwise concentrated hydrochloric acid (3.4 g) at 000. Then 7.5 mL of 8% NaOCl aqueous solution was added dropwise under ice cooling. After the completion of the addition, it was stirred at 00° C. for 90 min. The resulting solution was washed with water. The aqueous phase was extracted twice with ethyl acetate. The organic phase was dried over Na₂SO₄ and concentrated to give the title compound (1.2 g, 55%).

### Step 4: Preparation of 1-{3-[5-(3,5-dichloro-phenyl)-5-trifluoromethyl-4,5-dihydro-isoxazol-3-yl]-phenyl}-1H-[1,2,4]triazole (compound 1-2 of Table C.1)

The solution of N-hydroxy-3-[1,2,4]triazol-1-yl-benzimidic chloride (i.e. the product of Step 3, 1.2 g, 5.4 mmol), NaHCO₃ (0.8 g, 8.6 mmol) and 1,3-dichloro-5-(1-trifluoromethyl-vinyl)-benzene (1.4 g, 6.0 mmol) in THF (50 mL) was refluxed over night. The resulting solution was washed with water. The aqueous phase was extracted twice with ethyl acetate. The organic phase was dried over Na₂SO₄ and purified by preparative-HPLC to afford the title compound (350 mg, 15%).

¹H NMR (400 MHz, CDCl₃): δ [delta]=8.70 (s, 1H), 8.16 (s, 1H), 8.05 (s, 1H,), 7.81-7.83 (m, 1H), 7.72-7.74 (m, 1H), 7.60-7.64 (m, 1H), 7.54 (m, 2H), 7.46 (m, 1H), 4.16 (d, 1H), 3.78 (d, 1H).

### S.9 Preparation of {3-[5-(3,5-dichloro-phenyl)-5-trifluoromethyl-4,5-dihydro-isoxazol-3-yl]-phenyl}-pyrimidin-2-yl-amine (compound 1-1 of Table C.1)

### Step 1: Preparation of 3-nitro benzaldehyde oxime

A solution of 3-nitro benzaldehyde (10.0 g, 65.8 mmol), triethylamine (13.3 g, 131 mmol) and hydroxylamine hydrochloride (6.4 g, 92.1 mmol) in CH₂Cl₂ was stirred at room temperature over night. The resulting solution was washed with water. The aqueous phase was extracted twice with CH₂Cl₂. The organic phase was dried over Na₂SO₄ and concentrated to give the title compound (9.8 g, 89%).

¹H NMR (400 MHz, CDCl₃): δ [delta]=8.42-8.43 (m, 1H), 8.22-8.27 (m, 1H), 8.19-8.20 (m, 1H), 7.88-7.91 (m, 1H), 7.54-7.58 (m, 1H).

### Step 2: Preparation of N-hydroxy-3-nitro-benzimidic acid chloride

To a solution of 3-nitro benzaldehyde oxime (i.e. the product of Step 1, 10 g, 63 mmol) in THF (250 mL) was added drop-wise concentrated hydrochloric acid (25 g) at 0° C. Then 60 mL of 8% NaOCl aqueous solution was added drop-wise under ice cooling. After the completion of the addition, it was stirred at 0° C. for 90 min. The resulting solution was washed with water. The aqueous phase was extracted twice with ethyl acetate. The organic phase was dried over Na₂SO₄ and concentrated to give the title compound (11.2 g, 88%).

¹H NMR (400 MHz, CDCl₃): δ [delta]=8.72-8.74 (m, 1H), 8.61 (m, 1H), 8.30 (s, 1H), 8.21-8.24 (m, 1H), 7.60-7.64 (m, 1H).

### Step 3: Preparation of 5-(3,5-dichloro-phenyl)-3-(3-nitro-phenyl)-5-trifluoromethyl-4,5-dihydro-isoxazole

A solution of N-hydroxy-3-nitro-benzimidic acid chloride (i.e. the product of Step 2, 4.0 g, 20 mmol), NaHCO₃ (2.7 g, 32 mmol) and 1,3-dichloro-5-(1-trifluoromethyl-vinyl)benzene (5.2 g, 220 mmol) in THF (125 mL) was refluxed over night. The resulting solution was washed with water. The aqueous phase was extracted twice with ethyl acetate. The organic phase was dried over Na₂SO₄ to afford the title compound (4.0 g, 50%).

### Step 4: Preparation of 3-[5-(3,5-dichloro-phenyl)-5-trifluoromethyl-4,5-dihydro-isoxazol-3-yl]-phenylamine

A solution of 5-(3,5-dichloro-phenyl)-3-(3-nitro-phenyl)-5-trifluoromethyl-4,5-dihydro-isoxazole (i.e. the product of Step 3, 2.0 g, 5 mmol), Fe powder (1.2 g, 20 mmol) and acetic acid (1 g, 15 mmol) in EtOH (50 mL) was refluxed over night. The resulting mixture was filtered. The filtrate was washed with aqueous NaHCO₃ solution. The organic phase was dried and concentrated to afford the title compound (1.2 g, 63%).

### Step 5 Preparation of {3-[5-(3,5-dichloro-phenyl)-5-trifluoromethyl-4,5-dihydro-isoxazol-3-yl]-phenyl}-pyrimidin-2-yl-amine (compound 1-1 of Table C.1)

A solution of 3-[5-(3,5-dichloro-phenyl)-5-trifluoromethyl-4,5-dihydro-isoxazol-3-yl]-phenylamine (i.e. the product of Step 4, 0.7 g, 1.87 mmol), 2-chloropyrimidine (0.61 g, 5.6 mmol) and p-TsOH (0.36 g, 2.8 mmol) in dioxane (50 mL) was stirred at room temperature over night. The resulting solution was washed with water and brine. The aqueous phase was extracted twice with ethyl acetate. The organic phase was dried over Na₂SO₄ and purified by preparative-HPLC to give the title compound (0.3 g, 36%).

¹H NMR (400 MHz, CDCl₃): δ [delta]=8.53 (s, 2H), 8.10-8.11 (m, 1H), 7.69-7.72 (m, 1H), 7.54 (s, 2H), 7.39-7.46 (m, 3H), 6.89-6.91 (m, 1H), 4.13 (d, 1H), 3.73 (d, 1H).

Following compounds were synthesized:

Compound examples 1-1 to 1-6 correspond to compound formula C.1:

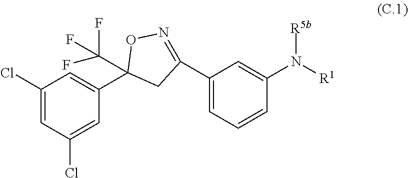

wherein R¹ and R^{5b} of each compound example is defined table C.1 below.

TABLE C.1

| Compound | | | HPLC-MS: Rₜ (min); [M + H]; |
|---|---|---|---|
| Ex. | R¹ | R^{5b} | ¹H NMR (400 MHz, CDCl₃) |
| 1-1 | H | 2-pyrimidin-2-yl | see Example S.9 |
| 1-2 | —N═CH—N═CH— | | see Example S.8 |
| 1-3 | —C(═O)—NH—CH₂—CH₂— | | δ = 7.51 (s, 2H), 7.42-7.14 (m, 5H), |
| | | | 4.91 (s, 1H), 4.09 (d, 1H), 3.69 (d, |
| | | | 1H), 3.51-3.40 (m, 2H), 2.94 (s, 3H), |
| | | | 2.60-2.58 (m, 1H), 2.01 (s, 1H). |
| 1-4 | —CH═C(CN)—CH═N— | | δ = 8.53 (s, 2H), 8.10-8.11 (m, 1H), |
| | | | 7.69-7.72 (m, 1H), 7.54 (s, 2H), 7.39- |
| | | | 7.46 (m, 3H), 4.10-4.15 (m, 1H), 3.71- |
| | | | 3.75 (m, 1H) |
| 1-5 | H | 2-oxazolidinyl | δ = 7.91 (s, 1H), 7.67-7.37 (m, 5H), |
| | | | 4.15-4.10 (m, 1H), 4.02-3.98 (m, 2H), |
| | | | 3.76-3.71 (m, 1H), 3.68-3.62 (m, 2H) |
| 1-6 | H | 2-thiazolidinyl | δ = 7.57-7.51 (m, 2H), 7.43-33 (m, |
| | | | 5H), 4.05-3.99 (m, 2H), 3.65-3.60 |
| | | | (m, 1H), 3.49-3.46 (m, 2H), 2.91- |
| | | | 2.82 (m, 1H) |

### C.2 Compound Examples 2

Compound examples 2-1 to 2-3 correspond to compound formula C.2:

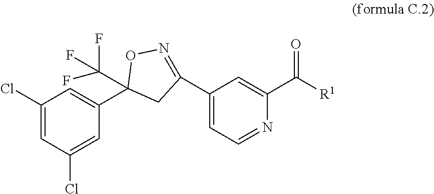

wherein R¹ of each compound example is defined table C.2 below.

TABLE C.2

| | | |
|---|---|---|
| Compound | | HPLC-MS: |
| Ex. | R¹ | Rₜ (min) and [M + H] |

| | | | |
|---|---|---|---|
| 2-1 | OH | 3.630 | 404.60 |
| 2-2 | NH—CH₂CH₂CH₃ | 4.345 | 445.60 |
| 2-3 | NH—CH₂CF₃ | 4.268 | 485.7 |

### C.3 Compound Examples 3

Compound examples 3-1 to 3-17 correspond to compound formula C.3:

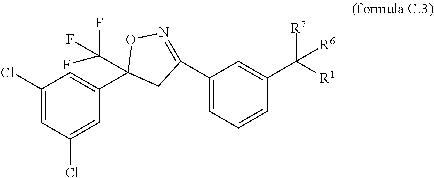

wherein R¹, R⁶ and R⁷ of each compound example is defined table C.3 below.

TABLE C.3

| Compound | | | | HPLC-MS: Rₜ (min) and [M + H] or | |
|---|---|---|---|---|---|
| Ex. | R⁶ | R⁷ | R¹ | ¹H NMR (400 MHz, CDCl₃) | |
| 3-1 | H | H | NH₂•HCl | see Example S.1 | |
| 3-2 | H | H | NH—C(═O)CH₂CH₃ | see Example S.2 | |
| 3-3 | H | H | NH—C(═O)CH₃ | see Example S.3 | |
| 3-4 | H | H | NH—C(═O)CH₂—S—CH₃ | see Example S.4 | |
| 3-5 | H | H | NH—C(═O)CH₂CH₂CH₃ | see Example S.5 | |
| 3-6 | H | H | H | 4.554 | 373.95 |
| 3-7 | H | H | O—C(═O)—CH₃ | 3.922 | 432.00 |
| 3-8 | H | H | O—C(═O)—CH₂CH₃ | 4.219 | 446.00 |
| 3-9 | H | H | O—C(═O)—CH₂CH₂CH₃ | 4.310 | 460.00 |
| 3-10 | H | H | O—CH₂—C(═O)—N(CH₃)₂ | 4.081 | 475.05 |

| | | | | |
|---|---|---|---|---|
| 3-11 | H | H | —C(═O)—NH—CH₂CH₃ | δ = 7.53-7.51 (m, 2H), 7.44 (m, |
| | | | | 2H), 7.36-7.30 (m, 3H), 5.28 (br. |
| | | | | s, 1H), 4.03 (d, 1H), 3.63 (d, |
| | | | | 1H), 3.49 (s, 2H), 3.24-3.17 (m, |
| | | | | 2H), 1.04-1.01 (m, 3H). |
| 3-12 | H | H | O—C(═O)—N(CH₂CH₃)— | δ = 8.26 (m, 1H), 7.51-7.48 (m, |
| | | | C(═O)—NH—CH₂CH₃ | 1H), 7.44-7.40 (m, 4H), 7.36- |
| | | | | 7.35 (m, 1H), 7.10-7.16 (m, 1H), |
| | | | | 4.01 (d, 1H), 4.97-4.92 (m, 2H), |
| | | | | 3.62 (d, 1H), 3.30-3.23 (m, 2H), |
| | | | | 1.27-1.23 (m, 3H), 1.19-1.17 |
| | | | | (m, 3H). |
| 3-13 | H | H | —C(═O)—NH—CH₂CH₂CH₃ | δ = 7.59-7.58 (m, 2H), 7.51 (m, |
| | | | | 2H), 7.44-7.37 (m, 3H), 5.41 (br. |
| | | | | s, 1H), 4.09 (d, 1H), 3.70 (d, 1H), |
| | | | | 3.57 (s, 2H), 3.22-3.17 (m, 2H), |
| | | | | 1.59-1.43 (m, 2H), 0.88-0.81 |
| | | | | (m, 3H). |
| 3-14 | H | H | —C(═O)—NH—CH₂-2- | δ = 8.40 (m, 1H), 7.59-7.54 (m, |
| | | | pyridyl | 3H), 7.44 (m, 2H), 7.37-7.32 (m, |
| | | | | 3H), 7.19-7.10 (m, 2H), 6.78 (br. |
| | | | | s, 1H), 4.47 (d, 2H), 4.01 (d, 1H), |
| | | | | 3.62 (d, 1H), 3.59 (s, 2H). |
| 3-15 | H | H | —C(═O)—NH—CH₃ | δ = 7.52 (m, 2H), 7.44 (m, 2H), |
| | | | | 7.44-7.19 (m, 3H), 5.40 (br. s, |
| | | | | 1H), 4.02 (d, 1H), 3.63 (d, 1H), |
| | | | | 3.50 (s, 2H), 2.71 (d, 3H). |
| 3-16 | H | H | —C(═O)—NH—CH₂CF₃ | δ = 7.55-7.54 (m, 2H), 7.52 (s, |
| | | | | 2H), 7.44-7.30 (m, 3H), 5.23 (br. |
| | | | | s, 1H), 4.04 (d, 1H), 3.84 (m, |
| | | | | 2H), 3.63 (d, 1H), 3.59 (s, 2H). |
| 3-17 | H | H | —S(═O)₂—CH₃ | δ = 7.71 (m, 2H), 7.52-7.49 (m, |
| | | | | 4H), 7.43 (s, 1H), 4.28 (s, 2H), |
| | | | | 4.10 (d, 1H), 3.71 (d, 1H), 2.82 |
| | | | | (s, 3H). |

### C.4 Compound Examples 4

Compound examples 4-1 to 4-5 correspond to compound formula C.4:

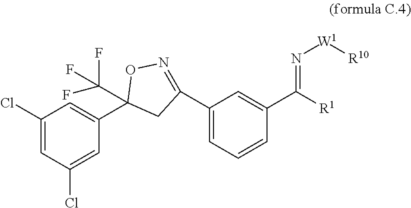

wherein R¹ and R¹⁰ of each compound example is defined table C.4 below.

TABLE C.4

| Compound | | | | HPLC-MS: Rₜ (min) and [M + H] or |
|---|---|---|---|---|
| Ex. | R¹ | R¹⁰ | W¹ | ¹H NMR (400 MHz, CDCl₃) |
| 4-1 | NH₂ | H | O | see Example S.6 |
| 4-2 | —N═C(CH₃)— | | O | see Example S.7 |
| 4-3 | H | CH₃ | O | δ = 8.06 (s, 1H), 7.82 (m, 1H), 7.74 (m, 1H), |
| | | | | 7.65 (m, 1H), 7.53-7.52 (m, 2H), 7.48-7.43 |
| | | | | (m, 2H), 4.12 (d, 1H), 4.01 (s, 3H), 3.71 (d, |
| | | | | 1H). |
| 4-4 | H | —C(═O)—CH₃ | NH | δ = 9.48 (s, 1H), 7.94 (s, 1H), 7.80 (s, 1H), |
| | | | | 7.76 (d, 1H), 7.67 (d, 1H), 7.53-7.43 (m, |
| | | | | 4H), 4.11 (d, 1H), 3.72 (d, 1H), 2.41 (s, 3H). |
| 4-5 | H | —C(═O)—NH—CH₃ | NH | δ = 8.74 (s, 1H), 7.93 (s, 1H), 7.70 (s, 1H), |
| | | | | 7.66 (d, 1H), 7.59 (d, 1H), 7.50 (s, 2H), |
| | | | | 7.47-7.40 (m, 2H), 6.08 (m, 1H), 4.10 (d, |
| | | | | 1H), 3.70 (d, 1H), 2.96 (d, 3H). |

### C.5 Compound Examples 5

Compound examples 5-1 to 5-4 correspond to compound formula C.5:

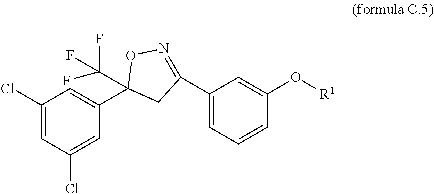

wherein R¹ and R¹⁰ of each compound example is defined table C.5 below.

TABLE C.5

| Compound | | |
|---|---|---|
| Ex. | R¹ | ¹H-NMR (400 MHz, CDCl₃) |
| 5-1 | 3-(1-methyl-pyrrolidin-2-one) | δ = 7.51 (s, 2H), 7.42-7.14 (m, 5H), 4.91 |
| | | (s, 1H), 4.11-4.06 (m, 1H), 3.72-3.66 (m, |
| | | 1H), 3.51-3.40 (m, 2H), 2.94 (s, 3H), |
| | | 2.60-2.58 (m, 1H), 2.01 (s, 1H). |
| 5-2 | C(═O)—CH₃ | δ = 7.53-7.49 (m, 3H), 7.45-7.40 (m, 3H), |
| | | 7.20-7.18 (m, 1H), 4.10-4.15 (m, 1H), |
| | | 3.71-3.75 (m, 1H), 2.31 (s, 3H). |
| 5-3 | C(═O)—NH—CH(CH₃)₂ | Spectrum measured in MeOD, 400 MHz; |
| | | δ = 7.61-7.56 (m, 4H), 7.49-7.40 (m, 2H), |
| | | 7.23-7.20 (m, 1H), 4.23 (d, 1H), 3.97 (d, |
| | | 1H), 3.75 (m, 1H), 1.21 (d, 6H). |
| 5-4 | C(═O)—NH—CH₂CH₃ | Spectrum measured in MeOD, 400 MHz; |
| | | δ = 7.60-7.55 (m, 4H), 7.49-7.43 (m, 2H), |
| | | 7.23-7.21 (m, 1H), 4.24 (d, 1H), 3.97 (d, |
| | | 1H), 3.24-3.19 (m, 2H), 1.20-1.16 (m, |
| | | 3H). |

### C.6 Compound Examples 6

Compound examples 6-1 to 6-2 correspond to compound formula C.6:

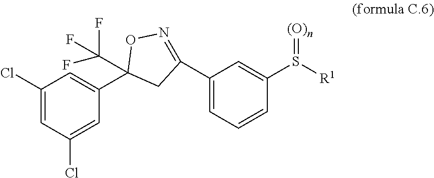

wherein R¹ and R¹⁰ of each compound example is defined table C.6 below.

TABLE C.6

| Compound | | | |
|---|---|---|---|
| Ex. | n | R¹ | ¹H-NMR (400 MHz, CDCl₃) |
| 6-1 | 0 | C(═O)—CH₃ | δ = 7.75-7.73 (m, 1H), 7.65 (s, 1H), 7.50-7.25 (m, |
| | | | 5H), 4.13 (d, 1H), 3.73 (d, 1H), 2.44 (s, 3H). |
| 6-2 | 1 | CF₃ | δ = 7.96 (m, 2H), 7.79 (m, 1H), 7.64 (m, 1H), 7.47 |
| | | | (m, 2H), 7.38 (m, 1H), 4.10 (d, 1H), 3.68 (d, 1H). |

### B. BIOLOGICAL EXAMPLES

The activity of the compounds of formula I of the present invention can be demonstrated and evaluated by the following biological test.

If not otherwise specified the test solutions were prepared as follow:

The active compound was dissolved at the desired concentration in a mixture of 1:1 (vol:vol) distilled water:acteon. The test solution was prepared the day it was used and in general at concentrations of ppm (wt/vol).

B.1 Cotton Aphid (Aphis gossypi)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Cotton plants at the cotyledon stage were infested with aphids prior to treatment by placing a heavily infested leaf from the main aphid colony on top of each cotyledon. Aphids were allowed to transfer overnight to accomplish an infestation of 80-100 aphids per plant and the host leaf was removed. The infested plants were then sprayed by an automated electrostatic plant sprayer equipped with an atomizing spray nozzle. The plants were dried in the sprayer fume hood, removed from the sprayer, and then maintained in a growth room under fluorescent lighting in a 24-hr photoperiod at 25° C. and 20-40% relative humidity. Aphid mortality on the treated plants, relative to mortality on untreated control plants, was determined after 5 days.

B.2 Cowpea Aphid (Aphis craccivora)

Potted cowpea plants colonized with approximately 100-150 aphids of various stages were sprayed after the pest population has been recorded. Population reduction was assessed after 24, 72, and 120 hours.

In this test, the compounds 3-13, 3-14, 4-4 and 6-2, respectively, at 500 ppm showed a mortality of at least 75% in comparison with untreated controls.

In this test, the compound 5-1 at 100 ppm showed a mortality of at least 75% in comparison with untreated controls.

B.3 Diamond Back Moth (Plutella xylostella)

Leaves of Chinese cabbage were dipped in test solution and air-dried. Treated leaves were placed in petri dished lined with moist filter paper. Mortality was recorded 24, 72, and 120 hours after treatment.

In this test, the compounds 1-3, 2-3, 5-4 and 6-2, respectively, at 500 ppm showed a mortality of at least 75% in comparison with untreated controls.

B.4 Mediterranean Fruitfly (Ceratitis capitata)

For evaluating control of Mediterranean fruitfly (Ceratitis capitata) the test unit consisted of microtiter plates containing an insect diet and 50-80 C. capitata eggs. The compounds were formulated using a solution containing 75% v/v water and 25% v/v DMSO. Different concentrations of formulated compounds were sprayed onto the insect diet at 5 μl, using a custom built micro atomizer, at two replications. After application, microtiter plates were incubated at about 28±1° C. and about 80±5% relative humidity for 5 days. Egg and larval mortality was then visually assessed.

In this test, the compounds 3-1, 3-2, 3-3, 3-4, 3-5, 3-13, 3-16, 3-17 and 6-2, respectively, at 2500 ppm showed a mortality of at least 75% in comparison with untreated controls.

B.5 Orchid Thrips (Dichromothrips corbetti)

Dichromothrips corbetti adults used for bioassay are obtained from a colony maintained continuously under laboratory conditions. For testing purposes, the test compound is diluted to a concentration of 300 ppm (wt compound: vol diluent) in a 1:1 mixture of acetone:water (vol:vol), plus 0.01% vol/vol Kinetic® surfactant.

Thrips potency of each compound is evaluated by using a floral-immersion technique. Plastic petri dishes are used as test arenas. All petals of individual, intact orchid flowers are dipped into treatment solution and allowed to dry. Treated flowers are placed into individual petri dishes along with 10-15 adult thrips. The petri dishes are then covered with lids. All test arenas are held under continuous light and a temperature of about 28° C. for duration of the assay. After 4 days, the numbers of live thrips are counted on each flower, and along inner walls of each petri dish. The level of thrips mortality is extrapolated from pre-treatment thrips numbers.

In this test, the compound 2-3, at 500 ppm showed a mortality of at least 75% in comparison with untreated controls.

B.6 Silverleaf Whitefly (Bemisia argentifoli)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, was then gently inserted into the soil containing the treated plant, allowing insects to crawl out of the tip to reach the foliage for feeding. Cups were covered with a reusable screened lid. Test plants were maintained in a growth room at about 25° C. and about 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, the compound 3-9, at 300 ppm showed a mortality of at least 75% in comparison with untreated controls.

B.7 Southern Armyworm (Spodoptera eridania)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Lima bean plants (variety Sieva) were grown 2 plants to a pot and selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, the compounds 3-7, 3-16, 5-3, 5-4 and 6-2, respectively, at 500 ppm showed a mortality of at least 75% in comparison with untreated controls.

B.8 Red Spider Mite (Tetranychus kanzawai)

The active compound was dissolved at the desired concentration in a mixture of 1:1 (v/v) distilled water:acetone. A surfactant (Alkamuls® EL 620) was added at the rate of 0.1% (v/v).

Potted cowpea beans of 7-10 days of age were cleaned with tap water and sprayed with 5 ml of the test solution using air driven hand atomizer. The treated plants were allowed to air dry and afterwards incubated with 20 or more mites by clipping a cassava leaf section with known mite population. Treated plants were placed inside a holding room at about 25-27° C. and about 50-60% relative humidity. ortality by counting the live mites 72 HAT. Percent mortality was assessed after 72 h.

B.9 Vetch Aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications.

After application, the leaf disks were air-dried and 5-8 adult aphids placed on the leaf disks inside the microtiter plate wells. The aphids were then allowed to suck on the treated leaf disks and incubated at about 23±1° C. and about 50±5% relative humidity for 5 days. Aphid mortality and fecundity was then visually assessed.

In this test, the compounds 3-1, 3-2, 3-3 and 3-5, respectively, at 2500 ppm showed a mortality of at least 75% in comparison with untreated controls.

B.10 Tobacco Budworm (Heliothis virescens) I

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Cotton plants were grown 2 plants to a pot and selected for treatment at the cotyledon stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 budworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, the compound 6-2 at 2500 ppm showed a mortality of at least 75% in comparison with untreated controls.

B.11 Boll Weevil (Anthonomus grandis)

For evaluating control of boll weevil (Anthonomus grandis) the test unit consisted of 24-well-microtiter plates containing an insect diet and 20-30 A. grandis eggs.

The compounds were formulated using a solution containing 75% v/v water and 25% v/v DMSO. Different concentrations of formulated compounds were sprayed onto the insect diet at 20 μl, using a custom built micro atomizer, at two replications.

After application, microtiter plates were incubated at about 23±1° C. and about 50±5% relative humidity for 5 days. Egg and larval mortality was then visually assessed.

In this test, the compounds 2-3, 3-1, 3-2, 3-3, 3-5, and 3-6 and 6-2, respectively at 2500 ppm showed a mortality of at least 75% in comparison with untreated controls. B.12 Western Flower Thrips (Frankliniella occidentalis)

B.12 Western Flower Thrips (Frankliniella occidentalis)

Serial dilutions of each technical grade Al were made in pure acetone. 0.5 ml of the treatment solution was deposited into the bottom of a glass vial (scintillation vial). The cap was screwed back onto the vial and inverted for about five seconds. The cap was subsequently removed and the vial laid on its side and rolled constantly, on a hot dog roller, until all the acetone had flashed off and the inner surface of the vial was dry. Cotton leave discs were also dipped simultaneously into the treatment solutions and allowed to dry. After the vials were dried, the leave discs are placed into the vials to serve as a food/water source for the thrips. Each treatment was replicated 5-fold. Western flower thrips were aspirated into the vials, approximately 5 larvae or adults/vial. Following treatment application the vials were held in a holding room under fluorescent light and constant 26° C. Thrips mortality was assessed at 2 DAT (days after treatment), counting all thrips both dead and alive.

In this test, the compounds 2-3 and 6-2, respectively at 2500 ppm showed a mortality of at least 75% in comparison with untreated controls.

1-35. (canceled)

36. A compound of formula (I): 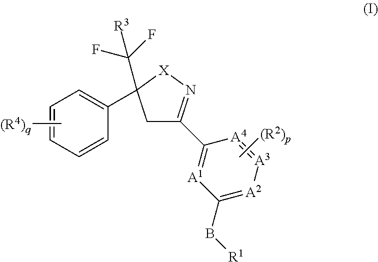
wherein
A¹, A², A³ and A⁴ are independently of each other N or CH;
X is O, S, NR^{5a} or CH₂;
B is O, S, SO, SO₂, NR^{5b}, CR⁶R⁷, C(═W) or NR^{5c}—C(═W);
with the proviso that B is not NR^{5c}—C(═W) if X is O and simultaneously A¹, A², A³ and A⁴ are CH;
W is O or S;
R¹ is selected from the group consisting of hydrogen, nitro, cyano, C₁-C₆-alkyl which may be partially or fully halogenated or may be substituted by one or more radicals R⁸, C₃-C₈-cycloalkyl which may be partially or fully halogenated or may be substituted by one or more radicals R⁹, C₂-C₆-alkenyl which may be partially or fully halogenated or may be substituted by one or more radicals R⁸, C₂-C₆-alkynyl which may be partially or fully halogenated or may be substituted by one or more radicals R⁸,
—NR^{11a}R^{11b}, —OR¹⁰, —S(O)ₙR¹⁰, —S(O)ₙNR^{11a}R^{11b}, —C(═O)R¹², —C(═O)OR¹³, —C(═O)NR^{11a}R^{11b}, —C(═S)R¹², —C(═S)OR¹³, —C(═S)SR¹³, —C(═S)NR^{11a}R^{11b}, —C(═NR^{11a})R¹², —Si(R¹⁴)₂R¹⁵, phenyl which may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶;
with the proviso that R¹ is not —OR¹⁰ if B is O and that R¹ is not halogen if B is O, S or NR^{5a};
with the proviso that R¹ is selected from hydrogen, nitro, cyano, C₁-C₆-alkylthio, C₁-C₆-haloalkylthio, C₃-C₈-cycloalkyl which may be partially or fully halogenated or may be substituted by one or more radicals R⁹, C₂-C₆-alkenyl which may be partially or fully halogenated or may be substituted by one or more radicals R⁸, C₂-C₆-alkynyl which may be partially or fully halogenated or may be substituted by one or more radicals R⁸, —NR^{11a}R^{11b}, —S(O)ₙR¹⁰, —S(O)ₙNR^{11a}R^{11b}, —C(═O)R¹², —C(═O)NR^{11a}R^{11b}, —C(═S)R¹², —C(═S)OR¹³, —C(═S)SR¹³, —C(═S)NR^{11a}R^{11b}, —C(═NR^{11a})R¹², and —Si(R¹⁴)₂R¹⁵ if B is NR^{5c}—C(═W), X is O and simultaneously one or two of A¹, A², A³ and A⁴ are N;
with the proviso that B—R¹ is not CH₂OH, CH₂C(═O)CH₃, COOCH₃, CONH₂ or CONHCH₃ if p is 1, the radical R² is bound to A² and is selected from the group consisting of CH₂Br, CH₂OH, CH₂C(═O)CH₃, COOCH₃, CONH₂ and CONHCH₃, A¹, A², A³ and A⁴ are CH, X is O or NR^{5a}, R³ is F, q is 2 and R⁴ is Cl; and
with the proviso that B—R¹ is not OH, OCH₃, OCH₂CH₃, OCH₂CH₂CH₃, OCH(CH₃)₂, NH₂, NHCH₃, N(CH₃)₂, NHCH₂CH₃, N(CH₂CH₃)₂, NHCH₂CH₂CH₃, N(CH₂CH₂CH₃)₂ or C₁-C₄-alkyl;
each R² is independently selected from the group consisting of halogen, cyano, azido, nitro, —SCN, —SF₅, C₁-C₆-alkyl which may be partially or fully halogenated or may be substituted by one or more radicals R⁸, C₃-C₈-cycloalkyl which may be partially or fully halogenated or may be substituted by one or more radicals R⁹, C₂-C₆-alkenyl which may be partially or fully halogenated or may be substituted by one or more radicals R⁸, C₂-C₆-alkynyl which may be partially or fully halogenated or may be substituted by one or more radicals R⁸, —Si(R¹⁴)₂R¹⁵, —OR¹⁰, —OS(O)ₙR¹⁰, —S(O)ₙR¹⁰, NR^{11a}R^{11b}, —N(R^{11a})C(═O)R¹², —C(═O)R¹², —C(═O)OR¹³, —C(═NR^{11a})R¹², —C(═S)R¹², phenyl which may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶,
with the proviso that R² is not —OR¹⁰, —OS(O)ₙR¹⁰, —S(O)ₙR¹⁰, —NR^{11a}R^{11b}, N(R^{11a})C(═O)R¹² or a 5 or 6-membered aromatic heterocyclic ring comprising 1, 2 or 3 heteroatoms selected from the group consisting of N, O and S as ring members, if R² is bound to A²;
R³ is selected from the group consisting of hydrogen, halogen, C₁-C₄-alkyl and C₁-C₄-haloalkyl,
each R⁴ is independently selected from the group consisting of halogen, cyano, azido, nitro, —SCN, —SF₅, C₁-C₆-alkyl which may be partially or fully halogenated or may be substituted by one or more radicals R⁸, C₃-C₈-cycloalkyl which may be partially or fully halogenated or may be substituted by one or more radicals R⁹, C₂-C₆-alkenyl which may be partially or fully halogenated or may be substituted by one or more radicals R⁸, C₂-C₆-alkynyl which may be partially or fully halogenated or may be substituted by one or more radicals R⁸, —Si(R¹⁴)₂R⁵, —OR¹⁰, —OS(O)ₙR¹⁰, —S(O)ₙR¹⁰, —NR^{11a}R^{11b}, —N(R^{11a})C(═O)R¹², —C(═O)R¹², —C(═O)OR¹³, —C(═NR^{11a})R¹², C(═S)R¹², phenyl which may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶;
R^{5a} is selected from the group consisting of hydrogen, C₁-C₄-alkyl, C₁-C₄-haloalkyl, C₁-C₄-alkoxy, C₁-C₄-haloalkoxy, C₁-C₄-alkylcarbonyl, C₁-C₄-haloalkylcarbonyl, C₁-C₄-alkoxycarbonyl, C₁-C₄-haloalkoxycarbonyl and benzyl;
R^{5b} and R^{5c}, independently of each other, are selected from the group consisting of hydrogen, cyano, nitro, nitroso, C₁-C₁₀-alkyl which may be partially or fully halogenated or may be substituted by one or more radicals R⁸, C₃-C₈-cycloalkyl which may be partially or fully halogenated or may be substituted by one or more radicals R⁹, C₂-C₁₀-alkenyl which may be partially or fully halogenated or may be substituted by one or more radicals R⁸, C₂-C₁₀-alkynyl which may be partially or fully halogenated or may be substituted by one or more radicals R⁸, —S(O)ₙR¹⁰, —S(O)ₙNR^{11a}R^{11b}, —C(═O)R¹², —C(═O)OR¹³, —C(═O)NR^{11a}R^{11b}, —C(═S)R¹², —C(═S)SR¹³, —C(═S)NR^{11a}R^{11b}, —C(═NR^{11a})R¹²; phenyl which may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶;
or
R^{5b} and R¹, together with the nitrogen atom to which they are bound, may form a 3-, 4-, 5-, 6-, 7- or 8-membered saturated, partially unsaturated or aromatic heterocyclic ring which may comprise 1 or 2 further heteroatoms or heteroatom groups selected from the group consisting of N, O, S, NO, SO and SO₂, as ring members, where the heterocyclic ring may be substituted by one or more radicals R¹⁶;
or
R^{5c} and R¹, together with the atoms to which they are bound, may form a 3-, 4-, 5-, 6-, 7- or 8-membered saturated, partially unsaturated or aromatic heterocyclic ring which may comprise 1 or 2 further heteroatoms or heteroatom groups selected from the group consisting of N, O, S, NO, SO and SO₂, as ring members, where the heterocyclic ring may be substituted by one or more radicals R¹⁶;
R⁶ and R⁷, independently of each other, are selected from the group consisting of hydrogen, halogen, cyano, azido, nitro, —SCN, —SF₅, C₁-C₆-alkyl which may be partially or fully halogenated or may be substituted by one or more radicals R⁸, C₃-C₈-cycloalkyl which may be partially or fully halogenated or may be substituted by one or more radicals R⁹, C₂-C₆-alkenyl which may be partially or fully halogenated or may be substituted by one or more radicals R⁸, C₂-C₆-alkynyl which may be partially or fully halogenated or may be substituted by one or more radicals R⁸, —Si(R¹⁴)₂R⁵, —OR¹⁰, OSO₂R¹⁰, —S(O)ₙR¹⁰, —S(O)ₙNR^{11a}R^{11b}, —NR^{11a}R^{11b}, —C(═O)NR^{11a}R^{11b}, —C(═S)NR^{11a}R^{11b}, —C(═O)OR¹³, phenyl which may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶,
or
R⁶ and R⁷ together form a group selected from the group consisting of ═CR¹⁷R¹⁸; ═S(O)ₙR¹⁰; ═S(O)ₙNR^{11a}R^{11b}, ═NR^{11a}, ═NOR¹⁰ and ═NNR^{11a}R^{11b}, where R¹⁰, R^{11a}, R^{11b}, R¹⁷, R¹⁸ and n are as defined below; or R¹⁰ and R¹ or R^{11a} and R¹ or R¹⁷ and R¹, together with the atoms to which they are bound, form a 3-, 4-, 5-, 6-, 7- or 8-membered saturated, partially unsaturated or aromatic carbocyclic or heterocyclic ring containing 1, 2 or 3 heteroatoms or heteroatom groups selected from the group consisting of N, O, S, NO, SO and SO₂, as ring members, where the carbocyclic or heterocyclic ring may be substituted by one or more radicals R;
or
R⁶ and R⁷, together with the carbon atom to which they are bound, form a 3-, 4-, 5-, 6-, 7- or 8-membered saturated or partly unsaturated carbocyclic or heterocyclic ring comprising 1, 2 or 3 heteroatoms or heteroatom groups selected from the group consisting of N, O, S, NO, SO and SO₂, as ring members, where the carbocyclic or heterocyclic ring may be substituted by one or more radicals R¹⁶;
each R⁸ is independently selected from the group consisting of cyano, azido, nitro, —SCN, —SF₅, C₃-C₈-cycloalkyl, C₃-C₈-halocycloalkyl, C₃-C₈-cycloalkyl substituted by a phenyl group or substituted by a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the phenyl group or the heterocyclic ring may carry one or more radicals R¹⁶; —Si(R¹⁴)₂R¹⁵, —OR¹⁰, —OSO₂R¹⁰, —S(O)ₙR¹⁰, —S(O)ₙN(R^{11a})R^{11b}, —N(R^{11a})R^{11b}, —C(═O)N(R^{11a})R^{11b}, —C(═S)N(R^{11a})R^{11b}, —C(═O)R¹², —C(═O)OR¹³, phenyl which may be substituted by 1, 2, 3, 4 or 5 radicals R¹⁶, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁶;
or two geminally bound radicals R⁸ together form a group selected from the group consisting of ═O, ═CR¹⁷R¹⁸, ═S(O)ₙR¹⁰, ═S(O)ₙN(R^{11a})R^{11b}, ═NR^{11a}═NOR¹⁰ and ═NNR^{11a}R^{11b};
or two radicals R⁸, together with the carbon atoms to which they are bound, form a 3-, 4-, 5-, 6-, 7- or 8-membered saturated, partially unsaturated or aromatic carbocyclic or heterocyclic ring containing 1, 2 or 3 heteroatoms or heteroatom groups selected from the group consisting of N, O, S, NO, SO and SO₂, as ring members, where the carbocyclic or heterocyclic ring may be substituted by one or more radicals R¹⁶;
each R⁹ is independently selected from the group consisting of cyano, azido, nitro, —SCN, —SF₅, C₁-C₆-alkyl, C₁-C₆-haloalkyl, C₁-C₆-alkoxy-C₁-C₆-alkyl, C₁-C₆-haloalkoxy-C₁-C₆-alkyl, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, C₂-C₆-alkenyl, C₂-C₆-haloalkenyl, C₂-C₆-alkynyl, C₂-C₆-haloalkynyl, —Si(R¹⁴)₂R¹⁵, —OR¹⁰, —OSO₂R¹⁰, —S(O)ₙR¹⁰, —S(O)ₙN(R^{11a})R^{11b}, —N(R^{11a})R^{11b}, —C(═O)N(R^{11a})R^{11b}, —C(═S)N(R^{11a})R^{11b}, —C(═O)R¹², —C(═O)OR¹³, phenyl, benzyl, where the phenyl moieties in the two last-mentioned radicals may be substituted by 1, 2, 3, 4 or 5 radicals R¹⁶, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁶;
or two geminally bound radicals R⁹ together form a group selected from the group consisting of ═O, ═CR¹⁷R¹⁸, ═S(O)ₙR¹⁰, ═S(O)ₙN(R^{11a})R^{11b}, ═NR^{11a}, ═NOR¹⁰ and ═NNR^{11a}R^{11b};
or two radicals R⁹, together with the carbon atoms to which they are bound, form a 3-, 4-, 5-, 6-, 7- or 8-membered saturated, partially unsaturated or aromatic carbocyclic or heterocyclic ring containing 1, 2 or 3 heteroatoms or heteroatom groups selected from the group consisting of N, O, S, NO, SO and SO₂, as ring members, where the carbocyclic or heterocyclic ring may be substituted by one or more radicals R¹⁶;
each R¹⁰ is independently selected from the group consisting of hydrogen, cyano, C₁-C₆-alkyl which may be partially or fully halogenated or may carry one or more radicals R²², C₃-C₈-cycloalkyl which may be partially or fully halogenated or may carry one or more radicals R²³, C₂-C₆-alkenyl which may be partially or fully halogenated or may carry one or more radicals R²², C₂-C₆-alkynyl which may be partially or fully halogenated or may carry one or more radicals R²², —Si(R¹⁴)₂R¹⁵, —OR¹⁹, —S(O)ₙR¹⁹, —S(O)ₙNR^{11a}R^{11b}, NR^{11a}R^{11b}, —N═CR¹⁷R¹⁸, —C(═O)R¹², —C(═O)NR^{11a}R^{11b}, —C(═S)NR^{11a}R^{11b}, —C(═O)OR¹³, phenyl, benzyl, where the phenyl moieties in the two last-mentioned radicals may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶;
with the proviso that in OR¹⁰, R¹⁰ is not —OR¹⁹;
R^{11a} and R^{11b}, independently of each occurrence and independently of each other, are selected from the group consisting of hydrogen, C₁-C₆-alkyl which may be partially or fully halogenated or may carry one or more radicals R²², C₃-C₈-cycloalkyl which may be partially or fully halogenated or may carry one or more radicals R²³, C₃-C₈-cycloalkyl-C₁-C₄-alkyl, C₃-C₈-halocycloalkyl-C₁-C₄-alkyl, where the cycloalkyl moieties in the two last-mentioned radicals may be partially or fully halogenated or may carry one or more radicals R²³, C₂-C₆-alkenyl which may be partially or fully halogenated or may carry one or more radicals R²², C₂-C₆-alkynyl which may be partially or fully halogenated or may carry one or more radicals R²², OR¹⁹, —S(O)ₙR¹⁹, —S(O)ₙNR^{20a}R^{20b}, —C(═O)R¹², —C(═O)OR¹³, —C(═O)NR^{20a}R^{20b}, —C(═S)R¹², —C(═S)SR¹³, —C(═S)NR^{20a}R^{20b}, —C(═NR^{20a})R¹²; phenyl, benzyl, where the phenyl moieties in the two last-mentioned radicals may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶;
or,
R^{11a} and R^{11b}, together with the nitrogen atom to which they are bound, form a 3-, 4-, 5-, 6-, 7- or 8-membered saturated, partially unsaturated or aromatic ring,
wherein the ring may contain one or two further heteroatoms or heteroatom groups selected from the group consisting of N, O, S, NO, SO and SO₂, as ring members, or wherein the ring may be substituted with one or more substituents selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, phenyl which may be substituted by 1, 2, 3, 4 or 5 radicals R¹⁶, and a 3-, 4-, 5-, 6,- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R⁶;
each R¹² is independently selected from the group consisting of hydrogen, halogen, cyano, azido, nitro, —SCN, SF₅, C₁-C₆-alkyl which may be partially or fully halogenated or may be substituted by one or more radicals R²², C₃-C₈-cycloalkyl which may be partially or fully halogenated or may be substituted by one or more radicals R²³, C₂-C₆-alkenyl which may be partially or fully halogenated or may be substituted by one or more radicals R²², C₂-C₆-alkynyl which may be partially or fully halogenated or may be substituted by one or more radicals R²², —Si(R¹⁴)₂R⁵—OR¹⁹, —OSO₂R¹⁹, —S(O)ₙR¹⁹, —S(O)ₙNR^{20a}R^{20b}, —NR^{20a}R^{20b}, —C(═O)NR^{20a}R^{20b}, —C(═S)NR^{20a}R^{20b}, —C(═O)OR¹³, phenyl which may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶,
each R¹³ is independently selected from the group consisting of hydrogen, cyano, C₁-C₆-alkyl which may be partially or fully halogenated or may be substituted by one or more radicals R²², C₃-C₈-cycloalkyl which may be partially or fully halogenated or may be substituted by one or more radicals R²³, C₂-C₆-alkeny which may be partially or fully halogenated or may be substituted by one or more radicals R²², C₂-C₆-alkynyl which may be partially or fully halogenated or may be substituted by one or more radicals R²², —Si(R¹⁴)₂R¹⁵, —S(O)ₙR¹⁹, —S(O)ₙNR^{20a}R^{20b}, —NR^{20a}R^{20b}, —N═CR¹⁷R¹⁸, —C(═O)R¹⁹, —C(═O)NR^{20a}R^{20b}, —C(═S)NR^{20a}R^{20b}, —C(═O)OR²¹, phenyl which may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶;
R¹⁴ and R¹⁵, independently of each other and independently of each occurrence, are selected from the group consisting of halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₄-alkyl, C₁-C₆-haloalkoxy-C₁-C₆-alkyl, phenyl, benzyl, where the phenyl moiety in the two last-mentioned radicals may be substituted by 1, 2, 3, 4 or 5 radicals selected from the group consisting of halogen, cyano, nitro, C₁-C₆-alkyl, C₁-C₆-haloalkyl, C₁-C₆-alkoxy and C₁-C₆-haloalkoxy; and a 3-, 4-, 5-, 6- to 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals selected from the group consisting of halogen, cyano, nitro, C₁-C₆-alkyl, C₁-C₆-haloalkyl, C₁-C₆-alkoxy and C₁-C₆-haloalkoxy;
each R¹⁶ is independently selected from the group consisting of halogen, cyano, azido, nitro, SCN, SF₅, C₁-C₁₀-alkyl which may be partially or fully halogenated or may be substituted by one or more radicals R²², C₃-C₈-cycloalkyl which may be partially or fully halogenated or may be substituted by one or more radicals R²³C₂-C₁₀-alkenyl which may be partially or fully halogenated or may be substituted by one or more radicals R²², C₂-C₁₀-alkynyl which may be partially or fully halogenated or may be substituted by one or more radicals R²², Si(R¹⁴)₂R⁵, —OR¹⁹, —OS(O)ₙR¹⁹, —S(O)ₙR¹⁹, —S(O)ₙNR^{20a}R^{20b}, NR^{20a}R^{20b}, —C(═O)R¹⁹ —C(═O)OR²¹, —C(═NR^{20a})R¹⁹, —C(═O)NR^{20a}R^{20b}, —C(═S)NR^{20a}R^{20b}, phenyl which may be substituted by 1, 2, 3, 4 or 5 radicals selected from the group consisting of halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals selected from the group consisting of halogen, cyano, nitro, C₁-C₆-alkyl, C₁-C₆-haloalkyl, C₁-C₆-alkoxy and C₁-C₆-haloalkoxy;
or
two radicals R¹⁶ bound geminally on one ring atom of a saturated or partially unsaturated ring may form together a group the group consisting of ═O, ═CR¹⁷R¹⁸; ═S(O)ₙR¹⁹; ═S(O)ₙNR^{20a}R^{20b}, ═NR^{20a}, ═NOR¹⁹ or ═NNR^{20a},
or
two radicals R¹⁶ bound on adjacent atoms form a bridge group selected from the group consisting of —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR^{20a}—, —CH₂CH═N—, —CH═CH—NR^{20a}—, —OCH═N—, —SCH═N— and thus form together with the atoms to which they are bound to a 5-membered or 6-membered saturated, partially unsaturated or aromatic carbocyclic or heterocyclic ring, wherein the ring may optionally be substituted with one or two substituents selected from the group consisting of ═O, OH, CH₃, OCH₃, halogen, halomethyl and halomethoxy;
R¹⁷ and R¹⁸, independently of each other and independently of each occurrence, are selected from the group consisting of hydrogen, C₁-C₄-alkyl, C₁-C₆-cycloalkyl, C₁-C₄-alkoxy-C₁-C₄-alkyl, phenyl and benzyl;
R¹⁹ is selected from the group consisting of hydrogen, halogen, cyano, azido, nitro, OH, SH, SCN, SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl, C₁-C₆-alkyl, C₂-C₆-alkenyl, C₂-C₆-alkynyl, C₃-C₈-cycloalkyl, wherein the four last-mentioned aliphatic and cycloaliphatic radicals may be partially or fully halogenated or wherein in the four last-mentioned aliphatic and cycloaliphatic radicals one or two CH₂ groups may be replaced by a CO group or wherein the four last-mentioned aliphatic and cycloaliphatic radicals may carry 1 or 2 radicals selected from the group consisting of C₁-C₄-alkoxy, phenyl, benzyl, pyridyl and phenoxy, wherein the four last-mentioned radicals may in turn be partially or fully halogenated or carry 1, 2 or 3 substituents selected from the group consisting of C₁-C₆-alkyl, C₁-C₆-haloalkyl, C₁-C₆-alkoxy, C₁-C₆-haloalkoxy, C₁-C₆-alkoxycarbonyl, C₁-C₆-alkylamino and di-(C₁-C₆-alkyl)-amino;
with the proviso that R¹⁹ in OR¹⁹ is not halogen, OH, C₁-C₆-alkoxy or C₁-C₆-haloalkoxy;
R^{20a} and R^{20b}, independently of each other and independently of each occurrence, are selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl, C₁-C₆-alkyl, C₂-C₆-alkenyl, C₂-C₆-alkynyl, C₃-C₈-cycloalkyl, wherein the four last-mentioned aliphatic and cycloaliphatic radicals may be partially or fully halogenated or wherein in the four last-mentioned aliphatic and cycloaliphatic radicals one or two CH₂ groups may be replaced by a CO group or wherein the four last-mentioned aliphatic and cycloaliphatic radicals may carry 1 or 2 radicals selected from the group consisting of C₁-C₄-alkoxy, phenyl, benzyl, pyridyl, and phenoxy, wherein the four last mentioned radicals may in turn be partially or fully halogenated or carry 1, 2 or 3 substituents selected from the group consisting of C₁-C₆-alkyl, C₁-C₆-haloalkyl, C₁-C₆-alkoxy, C₁-C₆-haloalkoxy and C₁-C₆-alkoxycarbonyl,
or
R^{20a} and R^{20b}, together with the nitrogen atom to which they are bound, form a 3-, 4-, 5-, 5-, 6- or 7-membered saturated, partially unsaturated or completely unsaturated heterocyclic ring, wherein the ring may contain 1 or 2 further heteroatoms or heteroatom groups selected from the group consisting of N, O, S, NO, SO and SO₂, as ring members, or wherein the ring may be substituted with one or more substituents selected from the group consisting of halogen, C₁-C₄-haloalkyl, C₁-C₄-alkoxy and C₁-C₄-haloalkoxy;
each R²¹ is independently selected from the group consisting of hydrogen, cyano, C₁-C₆-alkyl, C₂-C₆-alkenyl, C₂-C₆-alkynyl, C₃-C₈-cycloalkyl, wherein the four last-mentioned aliphatic and cycloaliphatic radicals may be partially or fully halogenated or wherein in the four last-mentioned aliphatic and cycloaliphatic radicals one or two CH₂ groups may be replaced by a CO group or wherein the four last-mentioned aliphatic and cycloaliphatic radicals may carry 1 or 2 radicals selected from the group consisting of C₁-C₄-alkoxy, phenyl, benzyl, pyridyl, and phenoxy, wherein the four last-mentioned radicals may in turn be partially or fully halogenated or carry 1, 2 or 3 substituents selected from the group consisting of C₁-C₆-alkyl, C₁-C₆-haloalkyl, C₁-C₆-alkoxy, C₁-C₆-haloalkoxy and C₁-C₆-alkoxycarbonyl;
each R²² is independently is selected from the group consisting of cyano; azido, nitro, —SCN, SF₅, C₃-C₈-cycloalkyl, C₃-C₈-halocycloalkyl, —Si(R¹⁴)₂R¹⁵, —OR¹⁹, —OSO₂R¹⁹, —S(O)ₙR¹⁹, —S(O)ₙN(R^{20a})R^{20b}, —N(R^{20a})R^{20b}, —C(═O)N(R^{20a})R^{20b}, —C(═S)N(R^{20a})R^{20b}, —C(═O)OR²¹, phenyl which may be substituted by 1, 2, 3, 4 or radicals independently selected from the group consisting of halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
each R²³ is independently is selected from the group consisting of cyano; azido, nitro, —SCN, SF₅, C₁-C₆-alkyl, C₁-C₆-haloalkyl, C₁-C₆-alkoxy-C₁-C₆-alkyl, C₁-C₆-haloalkoxy-C₁-C₆-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, —Si(R¹⁴)₂R¹, —OR¹⁹, —OSO₂R¹⁹, —S(O)ₙR¹⁹, —S(O)ₙN(R^{20a})R^{20b}, —N(R^{20a})R^{20b}, —C(═O)N(R^{20a})R^{20b}, —C(═S)N(R^{20a})R^{20b}, —C(═O)OR²¹, phenyl, benzyl, where the phenyl moieties in the two last-mentioned radicals may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
n is 0, 1 or 2;
p is 0, 1, 2, 3 or 4; and
q is 0, 1, 2, 3, 4 or 5;
or the stereoisomers or agriculturally or veterinarily acceptable salts thereof.

37. The compound according to claim 36, wherein A¹ and A² are N or CH and A³ and A⁴ are CH.

38. The compound according to claim 37, wherein A² is N or CH and A¹, A³ and A⁴ are CH.

39. The compound according to claim 36, wherein B is selected from the group consisting of O, S, NR^{5b}, CR⁶R⁷ and C(═W).

40. The compounds according to claim 36, wherein B is SO.

41. The compound according to claim 36, wherein R¹ is selected from the group consisting of —NR^{11a}R^{11b}, —OR¹⁰, —S(O)ₙR¹⁰, —S(O)ₙNR^{11a}R^{11b}, —C(═O)R¹², —C(═O)OR¹³, —C(═O)NR^{11a}R^{11b}, —C(═S)R¹², —C(═S)OR¹³, —C(═S)SR¹³, —C(═S)NR^{11a}R^{11b}, —C(═NR^{11a})R¹², —Si(R¹⁴)₂R¹⁵, phenyl which may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶;
or
R¹ forms together with R^{5b} and the nitrogen atom to which they are bound a 5- or 6-membered saturated, partially unsaturated or aromatic heterocyclic ring which may comprise 1 or 2 further heteroatoms or heteroatom groups selected from the group consisting of N, O, S, NO, SO and SO₂, as ring members, where the heterocyclic ring may be substituted by one or more radicals R¹⁶;
or
in case B is CR⁶R⁷ and R⁶ and R⁷ form together a group ═NR^{11a}, ═NOR¹⁰ or ═NNR^{11a}R^{11b}, R¹ forms together with R¹⁰ to or R^{11a} and the atoms to which they are bound a 5- or 6-membered saturated, partially unsaturated or aromatic heterocyclic ring which may comprise 1 or 2 further heteroatoms or heteroatom groups selected from the group consisting of N, O, S, NO, SO and SO₂, as ring members, where the heterocyclic ring may be substituted by one or more radicals R¹⁶.

42. The compound according to claim 41, wherein R¹ is selected from the group consisting of —NR^{11a}R^{11b}, —OR¹⁰ and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶;
or
R¹ forms together with R^{5b} and the nitrogen atom to which they are bound a 5- or 6-membered partially unsaturated or aromatic heterocyclic ring which may comprise 1 or 2 further heteroatoms or heteroatom groups selected from the group consisting of N, O, S, NO, SO and SO₂, as ring members, where the heterocyclic ring may be substituted by one or more radicals R¹⁶;
or
in case B is CR⁶R⁷ and R⁶ and R⁷ form together a group ═NR^{11a}, ═NOR¹⁰ or ═NNR^{11a}R^{11b}, R¹ forms together with R¹⁰ or R^{11a} and the atoms to which they are bound a 5- or 6-membered partially unsaturated or aromatic heterocyclic ring which may comprise 1 further heteroatom or heteroatom group selected from the group consisting of N, O, S, NO, SO and SO₂, as ring member, where the heterocyclic ring may be substituted by one or more radicals R⁶.

43. The compound according to claim 42, wherein in case B is O, S or NR^{5b}, R¹ is a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶; or in case B is NR^{5b}, R¹ forms together with R^{5b} and the nitrogen atom to which they are bound a 5- or 6-membered aromatic heterocyclic ring which may comprise 1 or 2 further heteroatoms selected from the group consisting of N, O and S as ring members, where the heterocyclic ring may be substituted by one or more radicals R¹⁶;
and in case B is CR⁶R⁷, C(═W) or NR^{5c}—C(═W), R¹ is selected from the group consisting of —NR^{11a}R^{11b} and —OR¹⁰; or, in case B is CR⁶R⁷ and R⁶ and R⁷ form together a group ═NR^{11a}, ═NOR¹⁰ or ═NNR^{11a}R^{11b}, R¹ forms together with R¹⁰ or R^{11a} and the atoms to which they are bound a 5- or 6-membered aromatic heterocyclic ring which may comprise 1 further heteroatom selected from the group consisting of N, O and S as ring member, where the heterocyclic ring may be substituted by one or more radicals R¹⁶.

44. The compound according to claim 42, wherein R^{11a} in the group —NR^{11a}R^{11b} is selected from the group consisting of hydrogen, C₁-C₁₀-alkyl which may be partially or fully halogenated or may carry one substituent R²²; and —C(═O)R¹², where R¹² is selected from the group consisting of hydrogen, C₁-C₆-alkyl, C₁-C₄-haloalkyl and C₁-C₄-alkyl which carries one substituent R²²; and R^{11b} is selected from the group consisting of hydrogen and C₁-C₄-alkyl.

45. The compound according to claim 44, wherein R²² is selected from the group consisting of cyano, C₃-C₈-cycloalkyl, C₃-C₈-halocycloalkyl, —OR¹⁹, —OSO₂R¹⁹, —S(O)ₙR¹⁹, —S(O)ₙN(R^{20a})R^{20b}, —N(R^{20a})R^{20b}, —C(═O)N(R^{20a})R^{20b}, —C(═S)N(R^{20a})R^{20b}, —C(═O)OR²¹, phenyl which may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy.

46. The compound according to claim 36, where R¹ is H or C₁-C₄-haloalkyl.

47. The compound according to claim 36, where R¹ is C(═O)R¹², C(═O)NR^{11a}R^{11b} or SO₂C₁-C₄-alkyl, where R¹² is C₁-C₄-alkyl, R^{11a} is H or R^{11b} is C₁-C₄-alkyl.

48. The compound according to claim 36, wherein each R² is independently selected from the group consisting of halogen, cyano, azido, nitro, —SCN, —SF₅, C₁-C₆-alkyl which may be partially or fully halogenated or may be substituted by one or more radicals R⁸, C₃-C₈-cycloalkyl which may be partially or fully halogenated or may be substituted by one or more radicals R⁹, C₂-C₆-alkenyl which may be partially or fully halogenated or may be substituted by one or more radicals R⁸, C₂-C₆-alkynyl which may be partially or fully halogenated or may be substituted by one or more radicals R⁸, —Si(R¹⁴)₂R⁵, —C(═O)R¹², —C(═O)OR¹³, —C(═NR^{11a})R¹², —C(═S)R¹², phenyl which may be substituted by 1, 2, 3, 4 or 5 radicals R¹⁶, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶.

49. The compound according to claim 48, wherein R² is selected from the group consisting of halogen, cyano, nitro, C₁-C₆-alkyl which may be partially or fully halogenated or may be substituted by one or more radicals R⁸, —C(═O)R¹², —C(═O)OR¹³, and a 3-, 4-, 5-, 6- or 7-membered saturated, partially unsaturated or aromatic heterocyclic ring comprising 1, 2 or 3 heteroatoms or heteroatom groups selected from the group consisting of N, O, S, NO, SO and SO₂, as ring members, where the heterocyclic ring may be substituted by one or more radicals R¹⁶.

50. The compound according to claim 36, wherein p is 0 or 1.

51. The compound according to claim 36, wherein R³ is hydrogen or halogen.

52. The compound according to claim 36, wherein each R⁴ is independently selected from the group consisting of halogen, cyano, nitro, —SCN, —SF₅, C₁-C₆-alkyl which may be partially or fully halogenated or may be substituted by one or more radicals R⁸, C₃-C₈-cycloalkyl which may be partially or fully halogenated or may be substituted by one or more radicals R⁹, C₂-C₆-alkenyl which may be partially or fully halogenated or may be substituted by one or more radicals R⁸, C₂-C₆-alkynyl which may be partially or fully halogenated or may be substituted by one or more radicals R⁸, —Si(R¹⁴)₂R¹⁵, —OR¹⁰, —OS(O)ₙR¹⁰, —S(O)ₙR¹⁰, —NR^{11a}R^{11b}, —N(R^{11a})C(═O)R¹², —C(═O)R¹², —C(═O)OR¹³, —C(═NR^{11a})R¹², —C(═S)R¹², phenyl which may be substituted by 1, 2, 3, 4 or 5 radicals R¹⁶; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁶.

53. The compound according to claim 52, wherein each R⁴ is independently selected from the group consisting of halogen, cyano, nitro, hydroxyl, C₁-C₄-alkyl, C₁-C₄-haloalkyl, C₁-C₄-alkoxy and C₁-C₄-haloalkoxy.

54. The compound according to claim 36, wherein q is 0, 1, 2 or 3.

55. The compound according to claim 36, wherein R^{5b} and R^{5c}, independently of each other, are selected from the group consisting of hydrogen, C₁-C₆-alkyl, C₁-C₆-haloalkyl, OH, C₁-C₄-alkoxy, C₁-C₄-haloalkoxy, C₁-C₆-alkylcarbonyl, C₁-C₆-haloalkylcarbonyl, C₁-C₄-alkoxycarbonyl, C₁-C₄-haloalkoxycarbonyl and benzyl.

56. The compound according to claim 36, wherein R⁶ and R⁷, independently of each other, are selected from the group consisting of hydrogen, C₁-C₄-alkyl, C₁-C₄-haloalkyl, OH, C₁-C₄-alkoxy and C₁-C₄-haloalkoxy.

57. The compound according to claim 36, of formula I-1 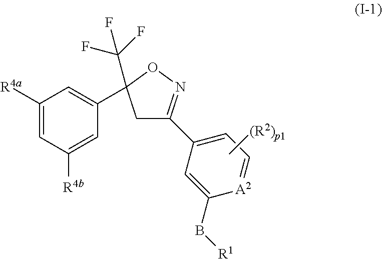
wherein
R^{4a} and R^{4b} are hydrogen or have the meanings given in claim 36 for R⁴, with the proviso that at most one of R^{4a} and R^{4b} is hydrogen; and
p1 is 0 or 1.

58. The compound according to claim 57, of formula I-1.1 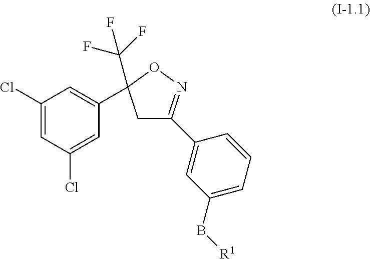
wherein
B is NH; and
R¹ is a 5- or 6-membered saturated, partially unsaturated or aromatic heterocyclic ring containing 1, 2 or 3 heteroatoms selected from the group consisting of N, O and S as ring members;
or
B is NR^{5b}; and
R¹ forms together with R^{5b} and the N-atom to which they are bound an N-bound 5- or 6-membered saturated, partially unsaturated or aromatic heterocyclic ring optionally containing 1 or 2 further heteroatoms selected from the group consisting of N, O and S as ring members, where the heterocyclic ring may carry 1 or 2 substituents R¹⁶; where
R¹⁶ is selected from the group consisting of halogen, CN, C₁-C₄-alkyl and oxo;
or
B is CO; and
R¹ is selected from the group consisting of OR¹⁰ and NR^{11a}R^{11b}, where
R¹⁰, R^{11a} and R^{11b}, independently of each other, are selected from the group consisting of hydrogen, C₁-C₄-alkyl and C₁-C₄-haloalkyl;
or
B is CH₂; and
R¹ is selected from the group consisting of NR^{11a}R^{11b}, OR^{10a}, C(═O)NR^{11a}R^{11b} and SO₂R^{10b}, where
R^{10a} is selected from the group consisting of C(═O)R¹², C(O)NR^{11a}R^{11b} and C₁-C₄-alkyl which carries 1 radical C(═O)NR^{20a}R^{20b}, where
R^{20a} and R^{20b} are independently selected from the group consisting of hydrogen and C₁-C₄-alkyl;
R^{10b} is selected from the group consisting of C₁-C₄-alkyl and C₁-C₄-haloalkyl;
R^{11a} and R^{11b} are independently selected from the group consisting of hydrogen, C₁-C₄-alkyl which may carry 1 substituent R²², where
R²² is a 5- or 6-membered saturated, partially unsaturated or aromatic heterocyclic ring containing 1, 2 or 3 heteroatoms selected from the group consisting of N, O and S as ring members; C₁-C₄-haloalkyl and C(═O)R¹²; and
R¹² is C₁-C₄-alkyl;
or
B is C═N—W¹—R¹⁰, where W¹ is O or NH;
R¹ is H or NH₂; and
R¹⁰ is H, C₁-C₄-alkyl, C(═O)R¹² or C(═O)NR^{11a}R^{11b}; where
R¹² is C₁-C₄-alkyl; and
R^{11a} and R^{11b} are independently H or C₁-C₄-alkyl; or
R¹ and R¹⁰, together with the atoms to which they are bound, form a 5- or 6-membered saturated, partially unsaturated or aromatic heterocyclic ring optionally containing 1 further heteroatom selected from the group consisting of N, O and S as ring member, where the heterocyclic ring may carry 1 or 2 substituents R¹⁶; where
R¹⁶ is selected from the group consisting of halogen, CN and C₁-C₄-alkyl;
or
B is O; and
R¹ is C(═O)R¹², C(═O)NR^{11a}R^{11b} or a 5- or 6-membered saturated, partially unsaturated or aromatic heterocyclic ring containing 1, 2 or 3 heteroatoms selected from the group consisting of N, O and S as ring members, where the heterocyclic ring may carry 1 or 2 substituents R¹⁶; where
R^{11a} and R^{11b} are independently H or C₁-C₄-alkyl;
R¹² is C₁-C₄-alkyl; and
R¹⁶ is selected from the group consisting of halogen, CN and C₁-C₄-alkyl;
or
B is S; and
R¹ is C(═O)R¹²; where
R¹² is C₁-C₄-alkyl or C₁-C₄-haloalkyl;
or
B is SO; and
R¹ is C₁-C₄-alkyl or C₁-C₄-haloalkyl.

59. The compound according to claim 58, where in case that B is CH₂, R¹ is selected from the group consisting of OR^{10a}, C(═O)NR^{11a}R^{11b} and SO₂R^{10b}.

60. The compounds according to claim 57, of formula I-1.1, wherein
B is NH; and
R¹ is 2-pyrimidinyl, 2-oxazolidinyl or 2-thiazolidinyl;
or
BR¹ is 1H-[1,2,4]-triazol-1-yl, 4-cyanopyrazol-1-yl or imidazolidin-2-on-1-yl;
or
B is CH₂; and
R¹ is H, NH₂, NH—CO—CH₃, NH—CO—CH₂CH₃, NH—CO—CH₂CH₂CH₃, NH—CO—CH₂—S—CH₃, O—CO—CH₃, O—CO—CH₂CH₃, O—CO—CH₂CH₂CH₃, O—CH₂—CO—N(CH₃)₂, O—CO—NHCH₃, O—CO—NHCH₂CH₃, O—CO—NHCH₃CH₂CH₃, O—CO—NHCH(CH₃)₂, CO—NHCH₃, CO—NHCH₂CH₃, CO—NHCH₃CH₂CH₃, CO—NHCH(CH₃)₂, CO—NHCH₂CF₃, CO—NHCH₂-2-pyridyl, O—CO—N(CH₂CH₃)—CO—NHCH₂CH₃ or SO₂CH₃;
or
B is C═N—OH; and
R¹ is NH₂;
or
B is C═N—OCH₃, C═N—NH—COCH₃ or C═N—NH—CONHCH₃; and
R¹ is H;
or
BR¹ is 5-methyl-[1,2,4]-oxadiazol-3-yl;
or
B is O and
R¹ is N-methylpyrrolidin-2-on-3-yl, COCH₃, CONH—CH₃, CONH—CH₂CH₃ or CONH—CH(CH₃)₂;
or
B is S; and
R¹ is COCH₃;
or
B is SO; and
R¹ is CF₃.

61. The compounds according to claim 57, of formula II-1.2 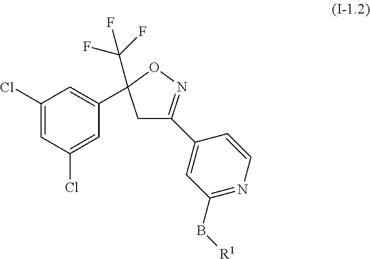
wherein B is CO and R¹ is OH, NH—CH₂CH₂CH₃ or NH—CH₂CF₃.

62. An agricultural composition comprising at least one compound of the formula I, as defined in any of claims 1 to 26, a stereoisomer thereof or at least one agriculturally acceptable salt thereof, and at least one inert liquid or solid agriculturally acceptable carrier.

63. A veterinary composition comprising a compound of claim 36, a stereoisomer thereof or at least one veterinarily acceptable salt thereof, and at least one inert liquid or solid veterinarily acceptable carrier.

64. A method for controlling invertebrate pests which method comprises treating the pests, their food supply, their habitat or their breeding ground or a plant, plant propagation material, soil, area, material or environment in which the pests are growing or may grow, or the materials, plants, plant propagation material, soils, surfaces or spaces to be protected from invertebrate pest attack or infestation with a pesticidally effective amount of a compound of claim 36.

65. A method for protecting plants from attack or infestation by invertebrate pests, which method comprises treating the plants with a pesticidally effective amount of the compound of claim 36.

66. A for protecting plant propagation material or the plants which grow therefrom from attack or infestation by invertebrate pests, which method comprises treating the plant propagation material with a pesticidally effective amount of a the compound of claim 36.

67. Plant propagation material treated with the compound of claim 36.

68. A method for treating, controlling, preventing or protecting animals against infestation or infection by parasites which comprises orally, topically or parenterally administering or applying to the animals a parasiticidally effective amount of the compound of claim 36.
